(19) United States
(12) Patent Application Publication
(10) Pub. No.: US 2005/0020505 A1
(43) Pub. Date: Jan. 27, 2005

(54) R-isomer of beta amino acid compounds as integrin receptor antagonists derivatives

(76) Inventors: Thomas Rogers, Ballwin, MO (US); Peter Ruminski, Ballwin, MO (US); Srinivasan Raj Nagarajan, Chesterfield, MO (US); Balekudru Devadas, Chesterfield, MO (US); James W. Malecha, San Diego, CA (US); Lawrence M. Miller, Stow, MA (US); Joseph G. Rico, O'Fallon, MO (US); Hwang-Fun Lu, Ballwin, MO (US); Laura D. Marrufo, Ellisville, MO (US); Joe T. Collins, Ballwin, MO (US)

Correspondence Address: Pharmacia Corporation;Global Patent Department, 5th Floor, 575 Maryville Centre Drive, St. Louis, MO, 63141, US

(21) Appl. No.: 10/741,768

(22) Filed: Dec. 19, 2003

Related U.S. Application Data
(60) Provisional application No. 60/435,006, filed on Dec. 20, 2002.

(51) Int. Cl. A61K 38/04; C07K 5/04; C07D 281/02; C07D 267/02

(52) U.S. Cl. 514/19; 540/544; 544/88

(57) ABSTRACT

The present invention relates to a class of compounds represented by the Formula I 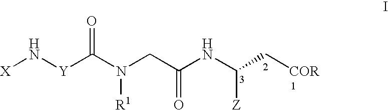
or a pharmaceutically acceptable salt thereof, pharmaceutical compositions comprising compounds of the Formula I, and methods of selectively inhibiting or antagonizing the αᵥβ₃ and/or the αᵥβ₅ integrin without significantly inhibiting the αᵥβ₆ integrin.

### FIELD OF THE INVENTION

The present invention relates to pharmaceutical compounds and processes of making compounds which are αᵥβ₃ and/or αᵥβ₅ integrin antagonists and as such are useful in pharmaceutical compositions and in methods for treating conditions mediated by αᵥβ₃ and/or αᵥβ₅ integrins.

### BACKGROUND OF THE INVENTION

Integrins are a group of cell surface glycoproteins which mediate cell adhesion and therefore are useful mediators of cell adhesion interactions which occur during various biological processes. The integrin identified as αᵥβ₃ (also known as the vitronectin receptor) is expressed in a number of cell types, including osteoclasts, platelets, megakaryocytes, proliferating endothelium, arterial smooth muscle, and some transformed tissue cells. A number of processes are mediated by activated αᵥβ₃ receptor, including the adhesion of osteoclasts to bone matrix, smooth muscle cell migration, and angiogenesis.

Antagonists of another integrin, αᵥβ₅, will also inhibit neovascularization, and will be useful for treating and preventing angiogenesis metastasis, tumor growth, macular degeneration and diabetic retionopathy.

Therefore, it is useful to antagonize both the αᵥβ₅ and the αᵥβ₃ receptor. Such “mixed αᵥβ₅/αᵥβ₃ antagonists” or “dual αᵥβ₃/αᵥβ₅ antagonists” are useful for treating or preventing angiogenesis, tumor metastasis, tumor growth, diabetic retinopathy, macular degeneration, atherosclerosis and osteoporosis.

Antagonists of αᵥβ₃ have been published in the literature. For instance, peptidyl as well as peptidomimetic antagonists containing the RGD sequence, have been described both in the scientific and patent literature. For example, reference is made to W. J. Hoekstra and B. L. Poulter, Curr. Med. Chem. 5: 195-204 (1998) and references cited therein review combinatorial organic syntheses of RGD compounds.

Such compounds that contain the RGD sequence mimic extracellular matrix ligands so as to bind to cell surface receptors. It is known that RGD peptides, in general, are non-selective for RGD dependent integrins. For example, most RGD peptides which bind to αᵥβ₃ also bind to αᵥβ₅, αᵥβ₁, αᵥβ₆ and α_{llb}β₃. Antagonism of platelet α_{llb}β₃ (also known as the fibrinogen receptor) is known to block platelet aggregation in humans, thereby causing a bleeding side effect

Small-molecule antagonists of αᵥβ₃ are also known. For example, U.S. Pat. No. 6,013,651 (incorporated by reference in its entirety) provides racemic meta-azacyclic amino benzoic acid compounds useful as αᵥβ₃ and/or αᵥβ₅ inhibitors.

WO 01/96334 (herein incorporated by reference) provides heteroarylalkanoic acid compounds useful as αᵥβ₃ and/or αᵥβ₅ inhibitors.

WO 97/08145 provides meta-gaunidine, urea, thiourea or azcyclic amino benzoic acid compounds and derivatives useful as αᵥβ₃ and/or αᵥβ₅ inhibitors.

WO 97/36859 provides para-substituted phenylene derivatives useful as αᵥβ₃ and/or αᵥβ₅ inhibitors.

WO 97/36861 provides meta-substituted sulphoamide phenylene derivatives useful as αᵥβ₃ and/or αᵥβ₅ inhibitors.

WO 97/36860 provides cinnamic acid derivatives useful as αᵥβ₃ and/or αᵥβ₅ inhibitors.

WO 97/36858 provides cyclopropyl alkanoic acid derivatives useful as αᵥβ₃ and/or αᵥβ₅ inhibitors.

WO 97/36862 provides meta-substituted phenylene derivatives useful as αᵥβ₃ and/or αᵥβ₅ inhibitors.

WO 99/52896 provides heterocyclic glycyl-beta alanine derivatives useful as αᵥβ₃ and/or αᵥβ₅ inhibitors.

WO 00/51968 provides meta-azacyclic amino benzoic acid compounds and derivatives useful as αᵥβ₃ and/or αᵥβ₅ inhibitors.

WO 01/96310 provides dihydrostilbene alkanoic acid derivatives useful as αᵥβ₃ and/or αᵥβ₅ inhibitors.

WO 02/18340 provides cycloalkyl compounds useful as αᵥβ₃ and/or αᵥβ₅ inhibitors.

WO 02/18377 provides bicyclic compounds useful as αᵥβ₃ and/or αᵥβ₅ inhibitors.

WO 02/026717 provides hydroxy acid compounds useful as αᵥβ₃ and/or αᵥβ₅ inhibitors.

WO 02/26227 provides lactone compounds useful as αᵥβ₃ and/or αᵥβ₅ inhibitors.

Methods of synthesis of substituted 3-guanidinobenzoic acid via unsubstitued thiourea are described in U.S. Pat. No. 6,100,423 and WO 99/44996.

### SUMMARY OF THE INVENTION

As evidenced by the continuing research in integrin antagonists and by the shortcomings of the compounds and methods of the art, there still remains a need for small-molecule, non-peptidic selective αᵥβ₃ and/or αᵥβ₅ antagonist that displays decreased side-effects, and improved potency, pharmacodynamic, and pharmacokinetic properties, such as oral bioavailability and duration of action, over already described compounds. Such compounds would prove to be useful for the treatment, prevention, or suppression of various pathologies enumerated above that are mediated by αᵥβ₃ and/or αᵥβ₅ receptor binding and cell adhesion and activation.

The compounds of the present invention further show greater selectivity for the αᵥβ₃ and/or αᵥβ₅ integrin than for the αᵥβ₆ integrin. It has been found that the selective antagonism of the αᵥβ₃ integrin is desirable in that the αᵥβ₆ integrin may play a role in normal physiological processes of tissue repair and cellular turnover that routinely occur in the skin and pulmonary tissue, and the inhibition of this function can be deleterious (Huang et al., Am J Respir Cell Mol Biol 1998, 19(4): 636-42). Therefore, compounds of the present invention which selectively inhibit the αᵥβ₃ integrin as opposed to the αᵥβ₆ integrin have reduced side effects associated with inhibition of the αᵥβ₆ integrin.

The compounds of the present invention comprise the R-isomers of the carbon of the beta amino acid. Other isomers may result from additional chiral centers, depending on the substitution of the parent structure.

The present invention relates to a class of compounds represented by the Formula I: 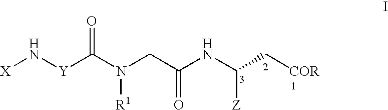
or a pharmaceutically acceptable salt or tautomer thereof;
wherein X has the structure of formula Ia: 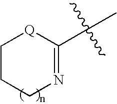
and wherein X is optionally substituted with one or more substituents independently selected from the group consisting of OH, alkyl, alkenyl, alkynyl, haloalkyl, alkylaryl, arylalkyl, alkoxy, dialkylamino, thioalkyl, cycloalkyl, CN, NO₂, and halogen;
or, in an alternative embodiment, X is a monocyclic heterocycle containing a N as shown, optionally substituted with one to ten, or alternatively 1-3, substituents independently selected from the group consisting of H, OH, alkyl, CN, NO₂, aminoalkyl, halogen, haloalkyl, and alkoxy;
Y is a six-membered aryl; or alternatively, a six-membered heterocycyl ring containing 1 to 2 heteroatoms, selected from the group consisting of O, N or S; wherein the six-membered ring is optionally substituted with one or more substitutents independently selected from the group consisting of OH, alkyl, alkoxy, NO₂, NH₂, CN, NHCOCF₃, COCF₃, haloalkyl, aryl, methylenedioxy, ethylenedioxy, heterocycyl, halogen, alkoxyalkyl, aminoalkyl, hydroxyalkyl, thioalkyl, alkylamino, arylamino, alkylsulfonamido, acyl, acylamino, alkylsulfone, sulfonamido, allyl, alkenyl, alkynyl, carboxamide, NHCOCF₃, and —(CH₂)ₘCOR²;
m is a number from 0 to 2;
R² is hydroxy, alkoxy, or amino;

Z is a 5 to 6-membered monocyclic, or a 9 to 12-membered bicyclic, aryl or heterocycyl ring; optionally containing 1 to 5 heteroatoms, selected from the group consisting of O, N or S; optionally saturated or unsaturated, optionally substituted with one or more substituents selected from the group consisting of alkyl, haloalkyl, aryl, heterocycyl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, NHCOCF₃, and —(CH₂)ₘCOR²; wherein the aryl and heterocycyl substituents are also optionally substituted with one or more substituents selected from the group consisting of alkyl, cycloalkyl, haloalkyl, halogen, alkoxyalkyl, aminoalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, and —(CH₂)ₘCOR²;
Q is NH or CH₂;
R is selected from the group consisting of OH, alkoxy, and NHR³;
R³ is H or an alkyl group;
R¹ is H, CN, NO₂, acyl, haloalkyl, alkenyl, alkynyl, or alkyl;
n is 0, 1, or 2,
and carbon atom 3 is in the (R) conformation.

It is another embodiment of the invention to provide pharmaceutical compositions comprising compounds of the Formula I. Such compounds and compositions are useful in selectively inhibiting or antagonizing the αᵥβ₃ and/or αᵥβ₅ integrins and therefore in another embodiment the present invention relates to a method of selectively inhibiting or antagonizing the αᵥβ₃ and/or αᵥβ₅ integrin. The invention further embodies treating or inhibiting pathological conditions associated therewith such as osteoporosis, humoral hypercalcemia of malignancy, Paget's disease, tumor metastasis, solid tumor growth (neoplasia), angiogenesis, including tumor angiogenesis, retinopathy including macular degeneration and diabetic retinopathy, arthritis, including rheumatoid arthritis and osteoarthritis, periodontal disease, psoriasis, smooth muscle cell migration and restenosis in a mammal in need of such treatment. Additionally, such pharmaceutical agents are useful as antiviral agents, and antimicrobials. The compounds of the present invention may be used alone or in combination with other pharmaceutical agents.

### DETAILED DESCRIPTION

The compounds of this invention include 1) αᵥβ₃ integrin antagonists; or 2) αᵥβ₅ integrin antagonists; or 3) mixed or dual αᵥβ₃/αᵥβ₅ antagonists. The present invention includes compounds which inhibit the respective integrins and also includes pharmaceutical compositions comprising such compounds.

In order to prevent bleeding side effects associated with the inhibition of α_{llb}β₃, it would bebeneficial to have a high selectivity ratio of αᵥβ₃ and αᵥβ₅ over α_{llb}β₃. The compounds of the present invention include selective antagonists of αᵥβ₃ over α_{llb}β₃. Further, compounds of the present invention selectively inhibit the αᵥβ₃ integrin as opposed to the αᵥβ₆ integrin.

The compounds of this invention include 1) αᵥβ₃ integrin antagonists; or 2) αᵥβ₅ integrin antagonists; or 3) mixed or dual αᵥβ₃/αᵥβ₅ antagonists. The present invention includes compounds which inhibit the respective integrins and also includes pharmaceutical compositions comprising such compounds. The present invention further provides for methods for treating or preventing conditions mediated by the αᵥβ₃ and/or αᵥβ₅ receptors in a mammal in need of such treatment comprising administering a therapeutically effective amount of the compounds of the present invention and pharmaceutical compositions of the present invention.

Compounds

The present invention comprises R-isomers of the carbon of the beta amino acid.

In one embodiment, the present invention relates to a class of compounds represented by the Formula I 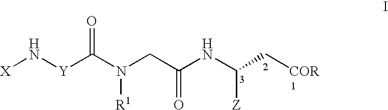
or a pharmaceutically acceptable salt or tautomer thereof;
wherein X has the structure of formula Ia: 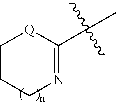
and wherein X is optionally substituted with one or more substituents independently selected from the group consisting of OH, alkyl, alkenyl, alkynyl, haloalkyl, alkylaryl, arylalkyl, alkoxy, dialkylamino, thioalkyl, cycloalkyl, CN, NO₂, and halogen;
or, in an alternative embodiment, X is a monocyclic heterocycle containing a N as shown, optionally substituted with one to ten, or alternatively 1-3, substituents independently selected from the group consisting of H, OH, alkyl, CN, NO₂, aminoalkyl, halogen, haloalkyl, and alkoxy;
Y is a six-membered aryl, or, alternatively, a six-membered heterocycyl ring containing 1 to 2 heteroatoms, selected from the group consisting of O, N or S; the ring optionally substituted with one or more substitutents independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, thioalkyl, alkylamino, methylenedioxy, ethylenedioxy, arylamino, alkylsulfonamido, acyl, acylamino, alkylsulfone, sulfonamido, alkenyl, alkynyl, carboxamide, NHCOCF₃, and —(CH₂)ₘCOR²;
m is a number from 0 to 2;
R² is hydroxy, alkoxy, or amino;

Z is a 5 to 6-membered monocyclic, or a 9 to 12-membered bicyclic, aryl or heterocycyl ring; optionally containing 1 to 5 heteroatoms, selected from the group consisting of O, N or S; optionally saturated or unsaturated, optionally substituted with one or more substituents selected from the group consisting of alkyl, haloalkyl, aryl, heterocycyl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, NHCOCF₃, and —(CH₂)ₘCOR²; wherein the aryl and heterocycyl substituents are also optionally substituted with one or more substituents selected from the group consisting of alkyl, cycloalkyl, haloalkyl, halogen, alkoxyalkyl, aminoalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)ₘCOR²;
Q is NH or CH₂;
R is selected from the group consisting of OH, alkoxy, and NHR³;
R³ is H or an alkyl group;
R¹ is H, CN, NO₂, acyl, haloalkyl, alkenyl, alkynyl, or alkyl;
n is a number from 0 to 2
and carbon atom 3 is in the (R) conformation.

In one embodiment, Z is a substituted phenyl ring.

In another embodiment, Y is a six-membered heterocycyl ring. In another embodiment, Y is substituted with at least one moiety selected from the group consisting of O, NH₂, NO₂, OH and CH₃. In another embodiment, the ring Y contains zero to two nitrogen atoms. In yet another embodiment, Y is selected from the group consisting of phenyl and pyridine, optionally substituted with O, NH₂, NO₂, OH or CH₃.

In one embodiment, n is one or two.

In another embodiment, X contains two nitrogen atoms. In another embodiment, X is azepine or diazepine. In yet another embodiment, X is pyrimidinyl or imidazolyl. In another embodiment, X is substituted with at least one moiety selected from the group consisting of H, OH, alkyl, CN, NO₂, aminoalkyl, halogen, haloalkyl, and alkoxy.

In another embodiment of the present invention, the compounds of the present invention having the structure of formula II 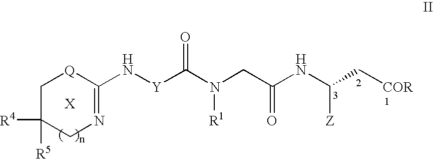
or a pharmaceutically acceptable salt, positional isomer, tautomer, or racemate thereof;
X is a 5 to 7-membered heterocyclic ring, wherein R⁴ and R⁵ are independently selected from the group consisting of H, OH, alkyl, CN, NO₂, aminoalkyl, halogen, haloalkyl, and alkoxy;
Y is a six-membered aryl ring; optionally substituted with one or more substitutents independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, thioalkyl, alkylamino, arylamino, methylenedioxy, ethylenedioxy, alkylsulfonamido, acyl, acylamino, alkylsulfone, sulfonamido, allyl, alkenyl, alkynyl, carboxamide, NHCOCF₃, and —(CH₂)ₘCOR²
m is a number from 0 to 2;
R² is hydroxy, alkoxy, or amino;
Z is a 5 to 6-membered monocyclic, or a 9 to 12-membered bicyclic, aryl ring; containing 1 to 5 heteroatoms, selected from the group consisting of O, N or S; optionally saturated or unsaturated, optionally substituted with one or more substituents selected from the group consisting of alkyl, haloalkyl, aryl, heteroaryl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, NHCOCF₃, and —(CH₂)ₘCOR²; wherein the aryl and heterocycyl substituents are optionally substituted with one or more substituents selected from the group consisting of alkyl, haloalkyl, halogen, alkoxyalkyl, aminoalkyl, cycloalakyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, and —(CH₂)ₘCOR²;
Q is NH or CH₂;
R is selected from the group consisting of OH, alkoxy, and NHR³;
R³ is H or an alkyl group;
R¹ is H, CN, NO₂, acyl, haloalkyl, alkenyl, alkynyl, or alkyl.
n is a number from 0 to 2
and carbon atom 3 is in the (R) conformation.

In another embodiment, R⁴ and R⁵ are independently selected from the group consisting of H, OH, F and CH₃.

In another embodiment of the present invention, the compounds of the present invention having the structure of formula III 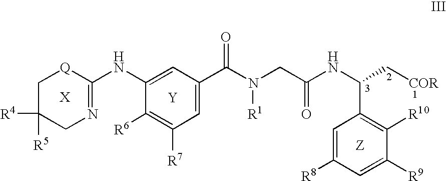
or a pharmaceutically acceptable salt, positional isomer, tautomer, or racemate thereof;
X is a 6-membered heterocyclic ring;
R⁴ and R⁵ are independently selected from the group consisting of H, OH, F, and CH₃;
Y is a 6-membered aryl ring;
R⁶ and R⁷ are independently selected from the group consisting of OH, CH₃, NO₂, NH₂, COOH, CONH₂, COCF₃, and NHCOCF₃; or R⁶ and R⁷ are linked together with a methylenedioxy and ethylenedioxy group to form a five- or six-membered ring, respectively;
Z is a 6-membered aryl ring;
R⁸, R⁹ and R¹⁰ are independently selected from the group consisting of H, OH, methyl, or halogen;
Q is NH or CH₂;
R is selected from the group consisting of OH, alkoxy, and NHR³;
R³ is H or an alkyl group;
R¹ is H or methyl
and carbon atom 3 is in the (R) conformation.

In another embodiment of the present invention, the compounds of the present invention having the structure of formula IV 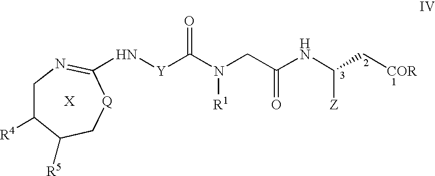
or a pharmaceutically acceptable salt, positional isomer or tautomer thereof;
X is a 7-membered heterocyclic ring;
R⁴ and R⁵ are independently selected from the group consisting of H, OH, alkyl, CN, NO₂, aminoalkyl, halogen, haloalkyl, and alkoxy;
Y is a 6-membered aryl or heterocycyl ring containing 1 to 2 heteroatoms, selected from the group consisting of O, N or S; the ring optionally substituted with one or more substitutents independently selected from the group consisting of OH, alkyl, alkoxy, NO₂, NH₂, CONH₂, NHCOCF₃, COCF₃, haloalkyl, aryl, heterocycyl, halogen, alkoxyalkyl, aminoalkyl, hydroxyalkyl, thioalkyl, methylenedioxy, ethylenedioxy, alkylamino, arylamino, alkylsulfonamido, acyl, acylamino, alkylsulfone, sulfonamido, allyl, alkenyl, alkynyl, carboxamide, NHCOCF₃, and —(CH₂)ₘCOR²
m is a number from 0 to 2;
R² is hydroxy, alkoxy, or amino;
Z is a 5 to 6-membered monocyclic, or a 9 to 12-membered bicyclic, aryl ring; containing 1 to 5 heteroatoms, selected from the group consisting of O, N or S; optionally saturated or unsaturated, optionally substituted with one or more substituents selected from the group consisting of alkyl, haloalkyl, aryl, heteroaryl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, NHCOCF₃, and —(CH₂)ₘCO R², wherein aryl and heterocycyl are optionally substituted with one or more substituents selected from the group consisting of alkyl, haloalkyl, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, and —(CH₂)ₘCOR²;
m is a number from 0 to 2;
R² is hydroxy, alkoxy, or amino;
Q is NH or CH₂;
R is selected from the group consisting of OH, alkoxy, and NHR³;
R³ is H or an alkyl group;
R¹ is H, CN, NO₂, acyl, haloalkyl, alkenyl, alkynyl, or alkyl;
and carbon atom 3 is in the (R) conformation.

In another embodiment, R⁴ and R⁵ are OH. In another embodiment, Y is a 6-membered aryl ring. In another embodiment, Z is a 6-membered aryl ring.

In another embodiment of the present invention, the compounds of the present invention having the structure of formula V 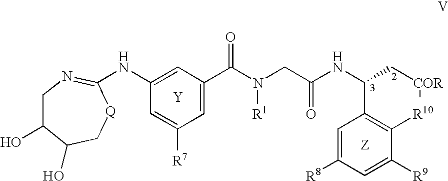
or a pharmaceutically acceptable salt, positional isomer, tautomer, or racemate thereof;
Y is a 6-membered aryl ring;
R⁷ is OH or CH₃;
Z is a 6-membered aryl ring;
R⁸ is H or OH;
R⁹, R¹⁰ are halogen;
Q is NH or CH₂;
R is selected from the group consisting of OH, alkoxy, and NHR³;
R³ is H or an alkyl group;
R¹ is H or methyl;
and carbon atom 3 is in the (R) conformation.

In another embodiment of the present invention, the compounds of the present invention having the structure of formula VI 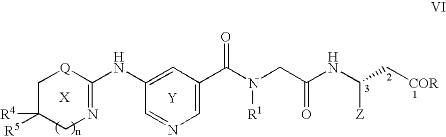
or a pharmaceutically acceptable salt, positional isomer, tautomer, or racemate thereof;
X is a monoheterocyclic ring;
R⁴ and R⁵ are independently selected from the group consisting of H, OH, alkyl, CN, NO₂, aminoalkyl, halogen, haloalkyl, and alkoxy;
Y is a pyridine; optionally substituted with one or more substitutents independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, thioalkyl, alkylamino, arylamino, alkylsulfonamido, acyl, acylamino, alkylsulfone, sulfonamido, allyl, alkenyl, alkynyl, carboxamide, NHCOCF₃, and —(CH₂)ₘCOR²;
m is a number from 0 to 2;
R² is hydroxy, alkoxy, or amino;
Z is a 5 to 6-membered monocyclic, or a 9 to 12-membered bicyclic, aryl ring, containing 1 to 5 heteroatoms, selected from the group consisting of O, N or S; optionally saturated or unsaturated, optionally substituted with one or more substituents selected from the group consisting of alkyl, haloalkyl, aryl, heteroaryl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, NHCOCF₃, and —(CH₂)ₘCOR²; wherein aryl and heterocycyl are optionally substituted with one or more substituents selected from the group consisting of alkyl, haloalkyl, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, and —(CH₂)ₘCOR²;
Q is NH or CH₂;
R is selected from the group consisting of OH, alkoxy, and NHR³;
R³ is H or an alkyl group;
R¹ is H, CN, NO₂, acyl, haloalkyl, alkenyl, alkynyl, or alkyl;
n is a number from 0 to 2;
and carbon atom 3 is in the (R) conformation.

In another embodiment of the present invention, the compounds of the present invention having the structure of formula VII 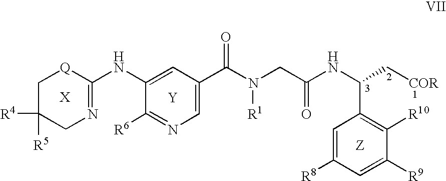
or a pharmaceutically acceptable salt, positional isomer, tautomer, or racemate thereof;
X is a 6-membered heterocyclic ring, wherein R⁴ and R⁵ are independently selected from the group consisting of H, OH, F, and CH₃;
Y is a pyridine;
R⁶ is H or OH;
Z is a 6-membered aryl ring;
R⁸, R⁹, and R¹⁰ are independently selected from the group consisting of H, OH, methyl, or halogen;
Q is NH or CH₂;
R is selected from the group consisting of OH, alkoxy, and NHR³;
R³ is H or an alkyl group;
R¹ is H or methyl;
and carbon atom 3 is in the (R) conformation.

In another embodiment of the present invention, the compounds of the present invention having the structure of formula VIII 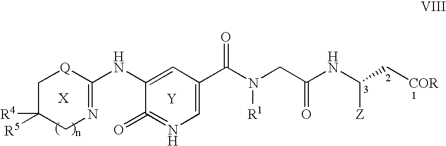
or a pharmaceutically acceptable salt, positional isomer, tautomer, or racemate thereof;
A is a monoheterocyclic ring, wherein R⁴ and R⁵ are independently selected from the group consisting of H, OH, alkyl, CN, NO₂, aminoalkyl, halogen, haloalkyl, and alkoxy;
Y is a pyridone; optionally substituted with one or more substitutents independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, thioalkyl, alkylamino, arylamino, alkylsulfonamido, acyl, acylamino, alkylsulfone, sulfonamido, allyl, alkenyl, alkynyl, carboxamide, NHCOCF₃, and —(CH₂)ₘCOR²;
wherein m is a number from 0 to 2;
R² is hydroxy, alkoxy, or amino;
Z is a 5 to 6-membered monocyclic, or a 9 to 12-membered bicyclic, aryl ring, containing 1 to 5 heteroatoms, selected from the group consisting of O, N or S; optionally saturated or unsaturated, optionally substituted with one or more substituents selected from the group consisting of alkyl, haloalkyl, aryl, heteroaryl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, NHCOCF₃, and —(CH₂)ₘCOR²; wherein aryl and heterocycyl are optionally substituted with one or more substituents selected from the group consisting of alkyl, haloalkyl, halogen, alkoxyalkyl, aminoalkyl, cycloalkyl, hydroxy, nitro, alkoxy, hydroxyalkyl, thioalkyl, amino, alkylamino, arylamino, alkylsulfonamido, acyl, acylamino, alkylsulfone, sulfonamido, allyl, alkenyl, methylenedioxy, ethylenedioxy, alkynyl, carboxamide, cyano, and —(CH₂)ₘCOR²;
Q is NH or CH₂;
R is selected from the group consisting of OH, alkoxy, and NHR³;
R³ is H or an alkyl group;
R¹ is H, CN, NO₂, acyl, haloalkyl, alkenyl, alkynyl, or alkyl.

n is a number from 0 to 2;
and carbon atom 3 is in the (R) conformation.

In another embodiment of the present invention, the compounds of the present invention having the structure of formula IX 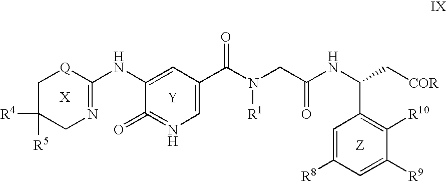
or a pharmaceutically acceptable salt, positional isomer, tautomer, or racemate thereof;
X is a 6-membered heterocyclic ring, wherein R⁴ and R⁵ are independently selected from the group consisting of H, OH, F, and CH₃;
Y is pyridone, optionally substituted with one or more substitutents independently selected from the group consisting of OH, alkyl, alkoxy, NO₂, NH₂, CN, NHCOCF₃, COCF₃, haloalkyl, aryl, heteroaryl, halogen, alkoxyalkyl, aminoalkyl, hydroxyalkyl, thioalkyl, alkyamino, arylamino, alkylsulfonamide, acyl, acylamino, alkylsulfone, sulfonamide, allyl, alkenyl, alkylnyl, carboxamide, NHCOCF₃, and —(CH₂)ₘCOR′;;
Z is a 6-membered aryl ring;
R⁸ is H or OH;
R⁹ and R¹⁰ are methyl or halogen;
Q is NH or CH₂;
R is selected from the group consisting of OH, alkoxy, and NHR³
R³ is H or an alkyl group;
R¹ is H or methyl;
and carbon atom 3 is in the (R) conformation.

The invention further relates to pharmaceutical compositions containing therapeutically effective amounts of the compounds of Formula I-IX. The compounds of Formula I can be represented by the follwing Formulas:

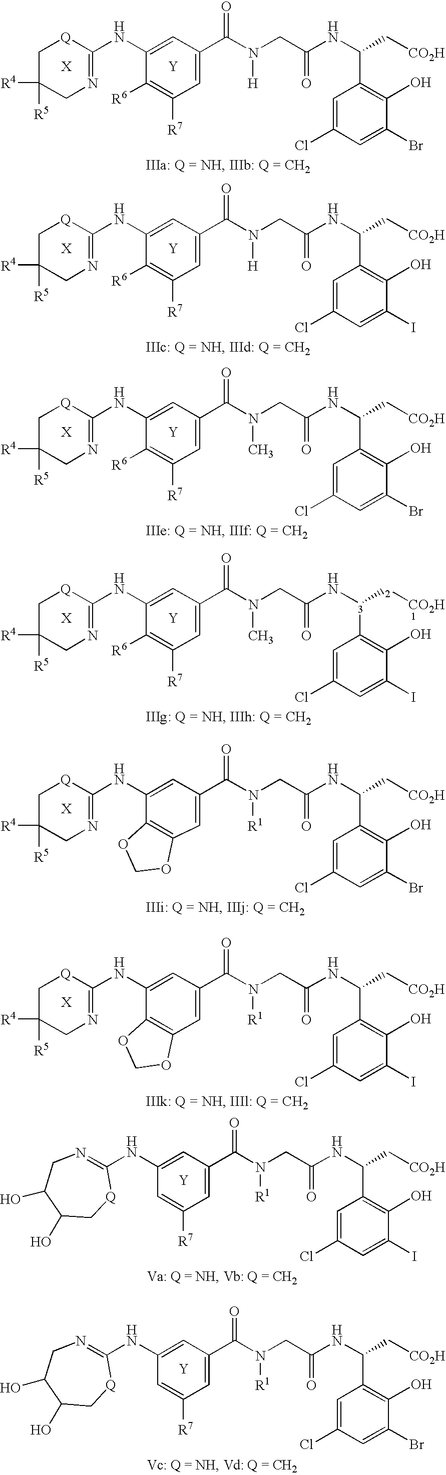

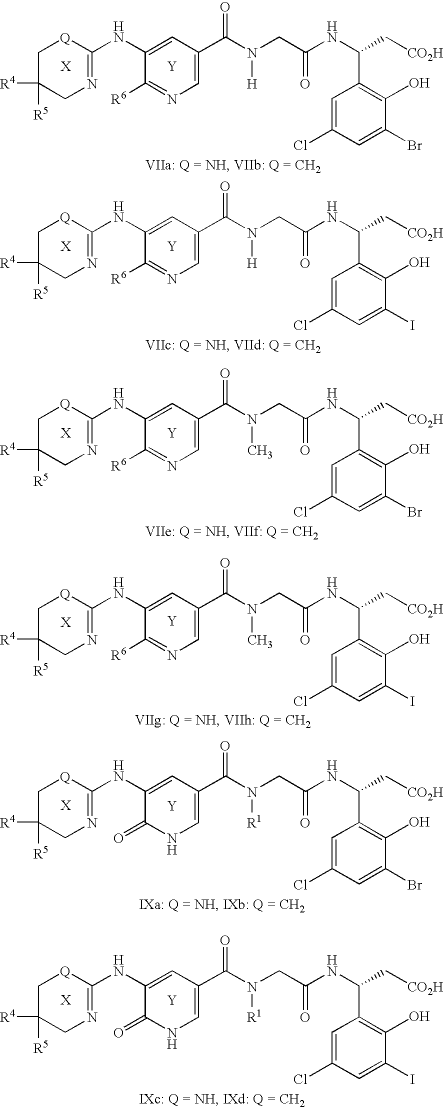

A family of specific compounds of particular interest within Formula I consists of compounds and pharmaceutically-acceptable salts thereof as shown in the following Tables.

TABLE I
General Formula IIIa-IIIh

| ID# | R⁴ | R⁵ | R⁶ | R⁷ |
|---|---|---|---|---|
| 1 | H | H | H | H |
| 2 | CH₃ | H | H | H |
| 3 | F | H | H | H |
| 4 | CH₃ | CH₃ | H | H |
| 5 | H | OH | H | H |
| 6 | F | OH | H | H |
| 7 | CH₃ | OH | H | H |
| 8 | F | F | H | H |
| 9 | H | H | CH₃ | H |
| 10 | CH₃ | H | CH₃ | H |
| 11 | F | H | CH₃ | H |
| 12 | CH₃ | CH₃ | CH₃ | H |
| 13 | H | OH | CH₃ | H |
| 14 | F | OH | CH₃ | H |
| 15 | CH₃ | OH | CH₃ | H |
| 16 | F | F | CH₃ | H |
| 17 | H | H | CF₃ | H |
| 18 | CH₃ | H | CF₃ | H |
| 19 | F | H | CF₃ | H |
| 20 | CH₃ | CH₃ | CF₃ | H |
| 21 | H | OH | CF₃ | H |
| 22 | F | OH | CF₃ | H |
| 23 | CH₃ | OH | CF₃ | H |
| 24 | F | F | CF₃ | H |
| 25 | H | H | OCH₃ | H |
| 26 | CH₃ | H | OCH₃ | H |
| 27 | F | H | OCH₃ | H |
| 28 | CH₃ | CH₃ | OCH₃ | H |
| 29 | H | OH | OCH₃ | H |
| 30 | F | OH | OCH₃ | H |
| 31 | CH₃ | OH | OCH₃ | H |
| 32 | F | F | OCH₃ | H |
| 33 | H | H | OH | H |
| 34 | CH₃ | H | OH | H |
| 35 | F | H | OH | H |
| 36 | CH₃ | CH₃ | OH | H |
| 37 | H | OH | OH | H |
| 38 | F | OH | OH | H |
| 39 | CH₃ | OH | OH | H |
| 40 | F | F | OH | H |
| 41 | H | H | CN | H |
| 42 | CH₃ | H | CN | H |
| 43 | F | H | CN | H |
| 44 | CH₃ | CH₃ | CN | H |
| 45 | H | OH | CN | H |
| 46 | F | OH | CN | H |
| 47 | CH₃ | OH | CN | H |
| 48 | F | F | CN | H |
| 49 | H | H | Cl | H |
| 50 | CH₃ | H | Cl | H |
| 51 | F | H | Cl | H |
| 52 | CH₃ | CH₃ | Cl | H |
| 53 | H | OH | Cl | H |
| 54 | F | OH | Cl | H |
| 55 | CH₃ | OH | Cl | H |
| 56 | F | F | Cl | H |
| 57 | H | H | H | OH |
| 58 | CH₃ | H | H | OH |
| 59 | F | H | H | OH |
| 60 | CH₃ | CH₃ | H | OH |
| 61 | H | OH | H | OH |
| 62 | F | OH | H | OH |
| 63 | CH₃ | OH | H | OH |
| 64 | F | F | H | OH |
| 65 | H | H | H | NH₂ |
| 66 | CH₃ | H | H | NH₂ |
| 67 | F | H | H | NH₂ |
| 68 | CH₃ | CH₃ | H | NH₂ |
| 69 | H | OH | H | NH₂ |
| 70 | F | OH | H | NH₂ |
| 71 | CH₃ | OH | H | NH₂ |
| 72 | F | F | H | NH₂ |
| 73 | H | H | H | NO₂ |
| 74 | CH₃ | H | H | NO₂ |
| 75 | F | H | H | NO₂ |
| 76 | CH₃ | CH₃ | H | NO₂ |
| 77 | H | OH | H | NO₂ |
| 78 | F | OH | H | NO₂ |
| 79 | CH₃ | OH | H | NO₂ |
| 80 | F | F | H | NO₂ |
| 81 | H | H | H | COOH |
| 82 | CH₃ | H | H | COOH |
| 83 | F | H | H | COOH |
| 84 | CH₃ | CH₃ | H | COOH |
| 85 | H | OH | H | COOH |
| 86 | F | OH | H | COOH |
| 87 | CH₃ | OH | H | COOH |
| 88 | F | F | H | COOH |
| 89 | H | H | H | NHCOR |
| 90 | CH₃ | H | H | NHCO |
| 91 | F | H | H | NHCOR |
| 92 | CH₃ | CH₃ | H | NHCOR |
| 93 | H | OH | H | NHCOR |
| 94 | F | OH | H | NHCOR |
| 95 | CH₃ | OH | H | NHCOR |
| 96 | F | F | H | NHCOR |
| 97 | H | H | H | CONH |
| 98 | CH₃ | H | H | CONH |
| 99 | F | H | H | CONH |
| 100 | CH₃ | CH₃ | H | CONH₂ |
| 101 | H | OH | H | CONH₂ |
| 102 | F | OH | H | CONH₂ |
| 103 | CH₃ | OH | H | CONH₂ |
| 104 | F | F | H | CONH₂ |
| 105 | H | H | H | CF₃ |
| 106 | CH₃ | H | H | CF₃ |
| 107 | F | H | H | CF₃ |
| 108 | CH₃ | CH₃ | H | CF₃ |
| 109 | H | OH | H | CF₃ |
| 110 | F | OH | H | CF₃ |
| 111 | CH₃ | OH | H | CF₃ |
| 112 | F | F | H | CF₃ |
| 113 | H | H | H | OCH₃ |
| 114 | CH₃ | H | H | OCH₃ |
| 115 | F | H | H | OCH₃ |
| 116 | CH₃ | CH₃ | H | OCH₃ |
| 117 | H | OH | H | OCH₃ |
| 118 | F | OH | H | OCH₃ |
| 119 | CH₃ | OH | H | OCH₃ |
| 120 | F | F | H | OCH₃ |
| 121 | H | H | H | Br |
| 122 | CH₃ | H | H | Br |
| 123 | F | H | H | Br |
| 124 | CH₃ | CH₃ | H | Br |
| 125 | H | OH | H | Br |
| 126 | F | OH | H | Br |
| 127 | CH₃ | OH | H | Br |
| 128 | F | F | H | Br |
| 129 | H | H | H | Cl |
| 130 | CH₃ | H | H | Cl |
| 131 | F | H | H | Cl |
| 132 | CH₃ | CH₃ | H | Cl |
| 133 | H | OH | H | Cl |
| 134 | F | OH | H | Cl |
| 135 | CH₃ | OH | H | Cl |
| 136 | F | F | H | Cl |
| 137 | H | H | H | I |
| 138 | CH₃ | H | H | I |
| 139 | F | H | H | I |
| 140 | CH₃ | CH₃ | H | I |
| 141 | H | OH | H | I |
| 142 | F | OH | H | I |
| 143 | CH₃ | OH | H | I |
| 144 | F | F | H | I |

TABLE II
General Formula IIIi-IIIl

| ID# | R¹ | R⁴ | R⁵ |
|---|---|---|---|
| 145 | H | H | H |
| 146 | H | CH₃ | H |
| 147 | H | F | H |
| 148 | H | H | OH |
| 149 | H | F | F |
| 150 | H | CH₃ | CH₃ |
| 151 | H | F | CH₃ |
| 152 | H | F | OH |
| 153 | CH₃ | H | H |
| 154 | CH₃ | CH₃ | H |
| 155 | CH₃ | F | H |
| 156 | CH₃ | H | OH |
| 157 | CH₃ | F | F |
| 158 | CH₃ | CH₃ | CH₃ |
| 159 | CH₃ | F | CH₃ |
| 160 | CH₃ | F | OH |

TABLE III
General Formula Va-Vd

| ID# | R¹ | R⁷ |
|---|---|---|
| 161 | H | H |
| 162 | H | OH |
| 163 | H | NH₂ |
| 164 | H | NO₂ |
| 165 | H | COOH |
| 166 | H | NHCOR |
| 167 | H | CONH₂ |
| 168 | H | CF₃ |
| 169 | H | Br |
| 170 | H | Cl |
| 171 | H | I |
| 172 | H | OCH₃ |
| 173 | CH₃ | H |
| 174 | CH₃ | OH |
| 175 | CH3 | NH₂ |
| 176 | CH₃ | NO₂ |
| 177 | CH₃ | COOH |
| 178 | CH₃ | NHCOR |
| 179 | CH₃ | CONH₂ |
| 180 | CH₃ | CF₃ |
| 181 | CH₃ | Br |
| 182 | CH₃ | Cl |
| 183 | CH₃ | I |
| 184 | CH₃ | OCH₃ |

TABLE IV
General Formula VIIa-VIIh

| ID# | R⁴ | R⁵ | R⁶ |
|---|---|---|---|
| 185 | H | H | H |
| 186 | CH₃ | H | H |
| 187 | F | H | H |
| 188 | CH₃ | CH₃ | H |
| 189 | H | OH | H |
| 190 | F | OH | H |
| 191 | CH₃ | OH | H |
| 192 | F | F | H |
| 193 | H | H | CH₃ |
| 194 | CH₃ | H | CH₃ |
| 195 | F | H | CH₃ |
| 196 | CH₃ | CH₃ | CH₃ |
| 197 | H | OH | CH₃ |
| 198 | F | OH | CH₃ |
| 199 | CH₃ | OH | CH₃ |
| 200 | F | F | CH₃ |
| 201 | H | H | CF₃ |
| 202 | CH₃ | H | CF₃ |
| 203 | F | H | CF₃ |
| 204 | CH₃ | CH₃ | CF₃ |
| 205 | H | OH | CF₃ |
| 206 | F | OH | CF₃ |
| 207 | CH₃ | OH | CF₃ |
| 208 | F | F | CF₃ |
| 209 | H | H | OCH₃ |
| 210 | CH₃ | H | OCH₃ |
| 211 | F | H | OCH₃ |
| 212 | CH₃ | CH₃ | OCH₃ |
| 213 | H | OH | OCH₃ |
| 214 | F | OH | OCH₃ |
| 215 | CH₃ | OH | OCH₃ |
| 216 | F | F | OCH₃ |
| 217 | H | H | OH |
| 218 | CH₃ | H | OH |
| 219 | F | H | OH |
| 220 | CH₃ | CH₃ | OH |
| 221 | H | OH | OH |
| 222 | F | OH | OH |
| 223 | CH₃ | OH | OH |
| 224 | F | F | OH |
| 225 | H | H | CN |
| 226 | CH₃ | H | CN |
| 227 | F | H | CN |
| 228 | CH₃ | CH₃ | CN |
| 229 | H | OH | CN |
| 230 | F | OH | CN |
| 231 | CH₃ | OH | CN |
| 232 | F | F | CN |
| 233 | H | H | Cl |
| 234 | CH₃ | H | Cl |
| 235 | F | H | Cl |
| 236 | CH₃ | CH₃ | Cl |
| 237 | H | OH | Cl |
| 238 | F | OH | Cl |
| 239 | CH₃ | OH | Cl |
| 240 | F | F | Cl |

TABLE V
General Formula IXa-IXd

| ID# | R¹ | R⁴ | R⁵ |
|---|---|---|---|
| 241 | H | H | H |
| 242 | H | CH₃ | H |
| 243 | H | F | H |
| 244 | H | H | OH |
| 245 | H | F | F |
| 246 | H | CH₃ | CH₃ |
| 247 | H | F | CH₃ |
| 248 | H | F | OH |
| 249 | CH₃ | H | H |
| 250 | CH₃ | CH₃ | H |
| 251 | CH₃ | F | H |
| 252 | CH₃ | H | OH |
| 253 | CH₃ | F | F |
| 254 | CH₃ | CH₃ | CH₃ |
| 255 | CH₃ | F | CH₃ |
| 256 | CH₃ | F | OH |

The compounds as shown above can exist in various isomeric forms, except as to the carbon of the beta amino acid. As used herein, the term “isomer” refers to all isomers except enantiomers. Tautomeric forms are also included as well as pharmaceutically acceptable salts of such isomers and tautomers.

In the structures and formulas herein, a bond drawn across a bond of a ring can be to any available atom on the ring.

The term “pharmaceutically acceptable salt” refers to a salt prepared by combining a compound of Formula I-IX with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid. Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. In a further embodiment, representative salts include the following: benzenesulfonate, hydrobromide and hydrochloride. The chloride salt is particularly preferred for medical purposes. Furthermore, where the compounds of the invention carry an acidic moiety, suitable pharmaceutically acceptable salts thereof may include alkali metal salts, e.g., sodium or potassium salts; alkaline earth metal salts, e.g., sodium, potassium, calcium or magnesium salts; and salts formed with suitable organic ligands, e.g., quaternary ammonium salts.

All of the pharmacologically acceptable salts may be prepared by conventional means. (See Berge et al., J Pharm. Sci., 1977, 66(1): 1-19 for additional examples of pharmaceutically acceptable salts, which is incorporated by reference herein in its entirety.)

The compounds of the present invention can have additional chiral centers and occur as diastereomeric mixtures, and as isomers as defined above. Also included within the scope of the invention are polymorphs, or hydrates or other modifiers of the compounds of invention.

The present invention includes within its scope prodrugs of the compounds of this invention. In general, such prodrugs will be functional derivatives of the compounds of this invention which are readily convertible in vivo into the required compound. For example, prodrugs of a carboxylic acid may include an ester, an amide, or an ortho-ester. Thus, in the methods of treatment of the present invention, the term “administering” shall encompass the treatment of the various conditions described with the compound specifically disclosed or with a compound which may not be specifically disclosed, but which converts to the compound of Formula I in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

Definitions

The following is a list of definitions of various terms used herein:

As used herein, the term “alkyl” refers to a straight chain or branched chain hydrocarbon radicals having from about 1 to about 10 carbon atoms, and alternatively, 1 to about 6 carbon atoms. Examples of such alkyl radicals are methyl, ethyl, n-propyl, isopropyl, n-butyl, isobutyl, sec-butyl, t-butyl, pentyl, neopentyl, hexyl, isohexyl, and the like.

As used herein the term “alkenyl” refers to unsaturated acyclic hydrocarbon radicals containing at least one double bond and 2 to about 6 carbon atoms, which carbon-carbon double bond may have either cis or trans geometry within the alkenyl moiety, relative to groups substituted on the double bond carbons. Examples of such groups are ethenyl, propenyl, butenyl, isobutenyl, pentenyl, hexenyl and the like.

As used herein, the term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

As used herein the term “alkynyl” refers to acyclic hydrocarbon radicals containing one or more triple bonds and 2 to about 6 carbon atoms. Examples of such groups are ethynyl, propynyl, butynyl, pentynyl, hexynyl and the like.

The term “cycloalkyl” as used herein means saturated or partially unsaturated cyclic carbon radicals containing 3 to about 8 carbon atoms and more preferably 4 to about 6 carbon atoms. Examples of such cycloalkyl radicals include cyclopropyl, cyclopropenyl, cyclobutyl, cyclopentyl, cyclohexyl, 2-cyclohexene-1-yl, and the like.

As used herein, the term “cyano” is represented by a radical of the formula

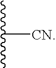

The terms “hydroxy” and “hydroxyl” as used herein are synonymous and are represented by a radical of the formula

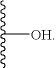

The term “alkylene” as used herein refers to divalent linear or branched saturated hydrocarbon radicals of 1 to about 6 carbon atoms.

The term “alkylaryl” refers to a radical of the formula 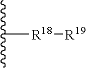
wherein R¹⁸ is alkyl as defined above and R¹⁹ is an alkylene as defined above. As used herein, alkylaryl includes both mono- and poly-alkyl aryl.

As used herein the term “alkoxy” refers to straight or branched chain oxy containing radicals of the formula —OR²⁰, wherein R²⁰ is an alkyl group as defined above. Examples of alkoxy groups encompassed include methoxy, ethoxy, n-propoxy, n-butoxy, isopropoxy, isobutoxy, sec-butoxy, t-butoxy and the like.

As used herein the terms “arylalkyl” refer to a radical of the formula 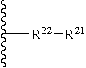
wherein R²¹ is aryl as defined above and R²² is an alkylene as defined above. Examples of aralkyl groups include benzyl, pyridylmethyl, naphthylpropyl, phenethyl and the like.

As used herein the term “nitro” is represented by a radical of the formula

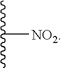

As used herein the term “halogen” refers to bromo, chloro, fluoro or iodo.

As used herein the term “haloalkyl” refers to alkyl groups as defined above substituted with one or more of the same or different halo groups at one or more carbon atom. Examples of haloalkyl groups include trifluoromethyl, dichloroethyl, fluoropropyl and the like.

As used herein the term “carboxyl” or “carboxy” refers to a radical of the formula —COOH.

As used herein the term “carboxyl ester” refers to a radical of the formula —COOR²³ wherein R²³ is selected from the group consisting of H, alkyl, aralkyl or aryl as defined above.

As used herein the term “amino” is represented by a radical of the formula —NH₂.

As used herein the term “alkylsulfonyl” or “alkylsulfone” refers to a radical of the formula 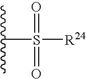
wherein R²⁴ is alkyl as defined above.

As used herein the term “alkylthio” refers to a radical of the formula —SR²⁴ wherein R²⁴ is alkyl as defined above.

As used herein the term “sulfonamide” or “sulfonamido” refers to a radical of the formula 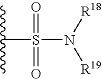
wherein R¹⁸ and R¹⁹ are alkyl as defined above.

As used herein the terms “monocyclic heterocycle” or “monocyclic heterocyclic” refer to a monocyclic ring containing from 4 to about 12 atoms, and more preferably from 5 to about 10 atoms, containing at least 1 carbon, and up to 11 additional members independently selected from the group consisting of carbon, oxygen, nitrogen and sulfur with the understanding that if two or more different heteroatoms are present at least one of the heteroatoms must be nitrogen. In a preferred embodiment, one to three members of the moncylic ring are independently selected from the group consisting of nitrogen, sulfur, and oxygen. Representative of such monocyclic heterocycles are pyridine, pyrimidine, imidazole, furan, pyridine, oxazole, pyran, triazole, thiophene, pyrazole, thiazole, thiadiazole, and the like.

As used herein the term “heterocyclic” or “heterocycle” means a saturated or unsaturated mono- or multi-ring carbocycle wherein one or more carbon atoms can be replaced by N, S, P, or O. This includes, for example, the following structures: 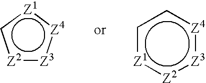
wherein Z¹, Z², Z³ or Z⁴ is C, S, P, O, or N, with the proviso that one of Z¹, Z², Z³ or Z⁴ is other than carbon, but is not O or S when attached to another Z atom by a double bond or when attached to another O or S atom. Furthermore, the optional substituents are understood to be attached to Z¹, Z², Z³ or Z⁴ only when each is C. “Heterocyclic” includes, furanyl, thienyl, pyrrolyl, 2-isopyrrolyl, 3-isopyrrolyl, pyrazolyl, 2-isoimidazolyl, 1,2,3-triazolyl, 1,2,4-triazolyl, 1,2-dithiolyl, 1,3-dithiolyl, 1,2,3-oxathiolyl, isoxazolyl, oxazolyl, thiazolyl, isothiazolyl, 1,2,3-oxadiazolyl, 1,2,4-oxadiazolyl, 1,2,5-oxadiazolyl, 1,3,4-oxadiazolyl, 1,2,3,4-oxatriazolyl, 1,2,3,5-oxatriazolyl, 1,2,3-dioxazolyl, 1,2,4-dioxazolyl, 1,3,2-dioxazolyl, 1,3,4-dioxazolyl, 1,2,5-oxathiazolyl, 1,3-oxathiolyl, 1,2-pyranyl, 1,4-pyranyl, 1,2-pyranonyl, 1,4-pyranonyl, 1,2-dioxinyl, 1,3-dioxinyl, pyridyl, pyridazyl, pyrimidyl, pyrazinyl, piperazyl, 1,3,5-triazinyl, 1,2,4-triazinyl, 1,2,3-triazinyl, 1,2,4-oxazinyl, 1,3,2-oxazinyl, 1,3,6-oxazinyl, 1,2,6-oxazinyl, 1,4-oxazinyl, o-isoxazinyl, p-isoxazinyl, 1,2,5-oxathiazinyl, 1,4-oxazinyl, o-isoxazinyl, p-isoxazinyl, 1,2,5-oxathianizyl, 1,2,6-oxathiainzyl, 1,4,2-oxadiainzyl, 1,3,5,2-oxadiainzyl, morpholino, azepinyl, oxepinyl, thiepinyl, 1,2,4-diazepinyl, benzofuranyl, isobenzofuranyl, benzothiofuranyl, isobenzothiofuranyl, indolyl, indoleninyl, 2-isobenzazolyl, 1,5-pyrindinyl, pyrano[3,4-b]pyrrolyl, isoindazolyl, indoxazinyl, benzoxazolyl, anthranilyl, 1,2-benzopyranyl, quinolyl, isoquinolyl, cinnolyl, quinazolyl, naphthyridyl, pyrido[3,4-b]pyridyl, pyrido[3,2-b]pyridyl, pyrido[4,3-b]pyridyl, 1,3,2-benzoxazyl, 1,4,2-benzoxazyl, 2,1,3-benzoxazyl, 3,1,4-benzoxazyl, 1,2-benzoisoxazyl, 1,4-benzoisoxazyl, carbazolyl, xanthenyl, acridinyl, purinyl, thiazolidyl, piperidyl, pyrrolidyl, 1,2-dihydroazinyl, 1,4-dihydroazinyl, 1,2,3,6-tetrahydro-1,3-diazinyl, perhydro-1,4-diazinyl, 1,2-thiayranyl, and 1,4-thiapyranyl.

As used herein the term “methylenedioxy” refers to the radical 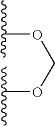
and the term “ethylenedioxy” refers to the radical

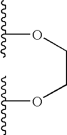

As used herein the term “bicycloalkyl” refers to a bicyclic hydrocarbon radical containing 6 to about 12 carbon atoms which is saturated or partially unsaturated.

As used herein the term “acyl” refers to a radical of the formula 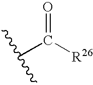
wherein R²⁶ is alkyl, alkenyl, alkynyl, aryl or aralkyl and optionally substituted thereon as defined above. Encompassed by such radical are the groups acetyl, benzoyl and the like.

As used herein the term “sulfonyl” refers to a radical of the formula 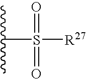
wherein R²⁷ is alkyl, aryl or aralkyl as defined above.

As used herein the term “haloalkylthio” refers to a radical of the formula —S—R²⁸ wherein R²⁸ is haloalkyl as defined above.

As used herein the term “aryloxy” refers to a radical of the formula 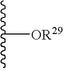
wherein R²⁹ is aryl as defined above.

As used herein the term “alkylamino” refers to a radical of the formula —NHR³² wherein R³² is alkyl as defined above.

As used herein the term “4-12 membered mono-nitrogen containing monocyclic or bicyclic ring” refers to a saturated or partially unsaturated monocyclic or bicyclic ring of 4-12 atoms and more preferably a ring of 4-9 atoms wherein one atom is nitrogen. Such rings may optionally contain additional heteroatoms selected from nitrogen, oxygen or sulfur. Included within this group are pyridine, pyrimidine, indole, morpholine, piperidine, piperazine, thiomorpholine, pyrrolidine, proline, azacycloheptene and the like.

As used herein the term “benzyl” refers to the radical

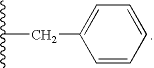

As used herein the term “phenethyl” refers to the radical

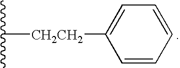

As used herein the term “arylsulfonyl” or “arylsulfone” refers to a radical of the formula 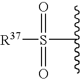
wherein R³⁷ is aryl as defined above.

As used herein the terms “alkylsulfoxide” or “arylsulfoxide” refer to radicals of the formula 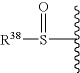
wherein R³⁸ is, respectively, alkyl or aryl as defined above.

As used herein the term “arylthio” refers to a radical of the formula 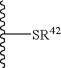
wherein R⁴² is aryl as defined above.

As used herein the term “monocyclic heterocycle thio” refers to a radical of the formula 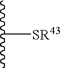
wherein R⁴³ is a monocyclic heterocycle radical as defined above.

As used herein the terms “monocyclic heterocycle sulfoxide” and “monocyclic heterocycle sulfone” refer, respectively, to radicals of the formula 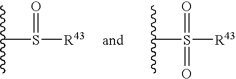
wherein R⁴³ is a monocyclic heterocycle radical as defined above.

As used herein the term “alkylcarbonyl” refers to a radical of the formula 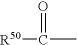
wherein R⁵⁰ is alkyl as defined above.

As used herein the term “arylcarbonyl” refers to a radical of the formula 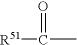
wherein R⁵¹ is aryl as defined above.

As used herein the term “alkoxycarbonyl” refers to a radical of the formula 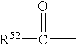
wherein R⁵² is alkoxy as defined above.

As used herein the term “aryloxycarbonyl” refers to a radical of the formula 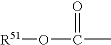
wherein R⁵¹ is aryl as defined above.

As used herein the term “haloalkylcarbonyl” refers to a radical of the formula 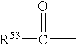
wherein R⁵³ is haloalkyl as defined above.

As used herein the term “haloalkoxycarbonyl” refers to a radical of the formula 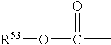
wherein R⁵³ is haloalkyl as defined above.

As used herein the term “alkylthiocarbonyl” refers to a radical of the formula 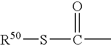
wherein R⁵⁰ is alkyl as defined above.

As used herein the term “arylthiocarbonyl” refers to a radical of the formula 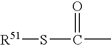
wherein R⁵¹ is aryl as defined above.

As used herein the term “acyloxymethoxycarbonyl” refers to a radical of the formula 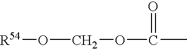
wherein R⁵⁴ is acyl as defined above.

As used herein the term “arylamino” refers to a radical of the formula R⁵¹—NH— wherein R⁵¹ is aryl as defined above.

As used herein the term “acyloxy” refers to a radical of the formula R⁵⁵—O— wherein R⁵⁵ is acyl as defined above.

As used herein the term “alkenylalkyl” refers to a radical of the formula R⁵⁰—R⁵⁷—wherein R⁵⁰ is an alkenyl as defined above and R⁵⁷ is alkylene as defined above.

As used herein the term “alkenylene” refers to a linear hydrocarbon radical of 1 to about 8 carbon atoms containing at least one double bond.

As used herein the term “alkoxyalkyl” refers to a radical of the formula R⁵⁶—R⁵⁷— wherein R⁵⁶ is alkoxy as defined above and R⁵⁷ is alkylene as defined above.

As used herein the term “alkynylalkyl” refers to a radical of the formula R⁵⁹—R⁶⁰— wherein R⁵⁹ is alkynyl as defined as above and R⁶⁰ is alkylene as defined as above.

As used herein the term “alkynylene” refers to divalent alkynyl radicals of 1 to about 6 carbon atoms.

As used herein the term “allyl” refers of a radical of the formula —CH₂CH═CH₂.

As used herein the term “aminoalkyl” refers to a radical of the formula H₂N—R⁶¹ wherein R⁶¹ is alkylene as defined above.

As used herein the term “benzoyl” refers to the aryl radical C₆H₅—CO—.

As used herein the term “carboxamide” or “carboxamido” refer to a radical of the formula —CO—NH₂.

As used herein the term “carboxyalkyl” refers to a radical HOOC—R⁶²— wherein R⁶² is alkylene as defined as above.

As used herein the term “carboxylic acid” refers to the radical —COOH.

As used herein the term “ether” refers to a radical of the formula R⁶³—O— wherein R⁶³ is selected from the group consisting of alkyl, aryl and heterocycyl.

As used herein the term “haloalkylsulfonyl” refers to a radical of the formula 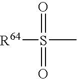
wherein the R⁶⁴ is haloalkyl as defined above.

As used herein the term “heteroaryl” refers to an aryl radical containing at least one heteroatom.

As used herein the term “hydroxyalkyl” refers to a radical of the formula HO—R⁶⁵ wherein R⁶⁵ is alkylene as defined above.

As used herein the term “keto” refers to a carbonyl group joined to 2 carbon atoms.

As used herein the term “lactone” refers to an anhydro cyclic ester produced by intramolecular condensation of a hydroxy acid with the elimination of water.

As used herein the term “olefin” refers to an unsaturated hydrocarbon radical of the type CₙH₂ₙ.

As used herein the term “R-isomer of beta amino acid” refers to the carbon of the beta-amino acid. Other additional chrial centers may exist depending on the substitutions in the parent structures. Thus, other isomers not including the R-isomer of the beta amino acid are contemplated by the present invention.

As used herein the term “sulfone” refers to a radical of the formula R⁶⁶—SO₂—.

As used herein the term “thioalkyl” refers to a radical of the formula R⁷⁷—S— wherein R⁷⁷ is alkyl as defined above.

As used herein the term “thioether” refers to a radical of the formula R⁷⁸—S— wherein R⁷⁸ is alkyl, aryl or heterocycyl.

As used herein the term “trifluoroalkyl” refers to an alkyl radical as defined above substituted with three halo radicals as defined above.

The term “composition” as used herein means a product which results from the mixing or combining of more than one element or ingredient.

The term “pharmaceutically acceptable carrier”, as used herein means a pharmaceutically acceptable material, composition or vehicle, such as a liquid or solid filler, diluent, excipient, solvent or encapsulating material, involved in carrying or transporting a chemical agent.

The term “selectivity ratio” shall mean the ratio of the inhibition of 50% of the maximum binding (IC₅₀ value) of αᵥβ₃ or αᵥβ₅ over the IC₅₀ value of αᵥβ₆. In one e

Abbreviations

The following is a list of abbreviations and the corresponding meanings as used interchangeably herein:
¹H-NMR=proton nuclear magnetic resonance
AcOH=acetic acid
BOC=tert-butoxycarbonyl
BuLi=butyl lithium
Cat.=catalytic amount
CDI=Carbonyldiimidazole
CH₂Cl₂=dichloromethane
CH₃CN=acetonitrile
CH₃I=iodomethane
CHN analysis=carbon/hydrogen/nitrogen elemental analysis
CHNCl analysis=carbon/hydrogen/nitrogen/chlorine elemental analysis
CHNS analysis=carbon/hydrogen/nitrogen/sulfur elemental analysis
DEAD=diethylazodicarboxylate
DIAD=diisopropylazodicarboxylate
DI water=deionized water
DMA=N,N-dimethylacetamide
DMAC=N,N-dimethylacetamide
DMF=N,N-dimethylformamide
EDC=1-(3-dimethylaminopropyl)-3-ethylcarbodiimide hydrochloride
Et=ethyl
Et₂O=diethyl ether
Et₃N=triethylamine
EtOAc=ethyl acetate
EtOH=ethanol
FAB MS=fast atom bombardment mass spectroscopy
g=gram(s)
HOBT=1-hydroxybenzotriazole hydrate
HPLC=high performance liquid chromatography
i-Pr=iso propyl
i-Prop=iso propyl
K₂CO₃=potassium carbonate
KMnO₄=potassium permanganate
KOH=potassium hydroxide
KSCN=potassium thiocyanate
L=Liter
LiOH=lithium hydroxide
Me=methyl
MeOH=methanol
mg=milligram
MgSO₄=magnesium sulfate
ml=milliliter
mL=milliliter
MS=mass spectroscopy
NaH—sodium hydride
NaHCO₃=sodium bicarbonate
NaOH=sodium hydroxide
NaOMe=sodium methoxide
NH₄⁺HCO₂⁻=ammonium formate
NMR=nuclear magnetic resonance
Pd=palladium
Pd/C=palladium on carbon
Ph=phenyl
Pt=platinum
Pt/C=platinum on carbon
RPHPLC=reverse phase high performance liquid chromatography
RT=room temperature
t-BOC=tert-butoxycarbonyl
TFA=trifluoroacetic acid
THF=tetrahydrofuran
TLC—thin layer chromatography
TMS=trimethylsilyl
Δ=heating the reaction mixture
   Indications

In one embodiment, compounds of the present invention are useful for treating an αᵥβ₃ integrin-mediated condition. The integrin identified as αᵥβ₃ (also known as the vitronectin receptor) has been identified as an integrin which plays a role in various conditions or disease states.

Antagonists of αᵥβ₃ have been shown to be potent inhibitors of osteoclastic activity both in vitro and in vivo. Antagonism of αᵥβ₃ leads to decreased bone resorption and therefore restores a normal balance of bone forming and resorbing activity. Thus it will be beneficial to provide antagonists of osteoclast αᵥβ₃ that are effective inhibitors of bone resorption and therefore are useful in the treatment or prevention of osteopenia or osteoporosis, or other bone disorders, such as Paget's disease or humoral hypercalcemia of malignancy.

The role of the αᵥβ₃ integrin in smooth muscle cell migration also makes it a therapeutic target for prevention or inhibition of neointimal hyperplasia which is a leading cause of restenosis after vascular procedures (Choi et al., J. Vasc. Surg. 1994, 19(1): 125-34).

The attachment of human periodontal ligament cells to anorganic bone matrix is mediated by interaction between a BSP-like molecule and integrin alpha(v)beta3 on the cell surface. Therefore, antagonists of αᵥβ₃ will also be useful in treating and preventing periodontal disease.

Many viruses contain a RGD domain in the penton base which promotes efficient infection of host cells via interaction with αᵥβ₃. Also, attachment of other pathogens (such as Candida albicans and Pneumocystis carinii) to cell surfaces is attenuated through antibodies to αᵥ. Thus, inhbition of of αᵥβ₃ will be useful for the treatment and prevention of viral and other infections.

The integrin αᵥβ₃ was identified as a marker of angiogenic blood vessels in chick and man and plays a critical role in angiogenesis or neovascularization. Antagonists of αᵥβ₃ inhibit this process by selectively promoting apoptosis of cells in neovasculature. The growth of new blood vessels, or angiogenesis, contributes to pathological conditions such as diabetic retinopathy, macular degeneration, rheumatoid arthritis, osteoarthritis, or tumor angiogenesis. Therefore, αᵥβ₃ antagonists will be useful therapeutic agents for treating such conditions associated with neovascularization.

Brooks et al. (Cell, 1994, 79: 1157-1164) have demonstrated that certain antagonists of αᵥβ₃ may provide a therapeutic approach for the treatment of neoplasia (inhibition of solid tumor growth) since systemic administration of αᵥβ₃ antagonists causes dramatic regression of various histologically distinct human tumors. Further, establishment of skeletal metastases in advanced breast cancer patients is thought to be mediated by the αᵥβ₃ integrin receptor.

The integrin αᵥβ₅ also plays a role in neovascularization. M. C. Friedlander, et al., Science, 270: 1500-1502 (1995) disclose that a monoclonal antibody for αᵥβ₅ inhibits VEFG-induced angiogenesis in the rabbit cornea and the chick chorioallantoic membrane model. Antagonists of the αᵥβ₅ integrin will inhibit neovascularization, and will be useful for treating and preventing angiogenesis metastasis, tumor growth, macular degeneration and diabetic retionopathy.

The invention also relates to a method of selectively inhibiting or antagonizing the αᵥβ₃ integrin and/or the αᵥβ₅ integrin and more specifically relates to a method of inhibiting bone resorption, periodontal disease, osteoporosis, humoral hypercalcemia of malignancy, Paget's disease, tumor metastasis, solid tumor growth (neoplasia), angiogenesis, including tumor angiogenesis, retinopathy including macular degeneration and diabetic retinopathy, arthritis, including rheumatoid arthritis, smooth muscle cell migration and restenosis by administering a therapeutically effective amount of a compound of the Formula I to achieve such inhibition together with a pharmaceutically acceptable carrier. More specifically it has been found that it is advantageous to administer compounds which are αᵥβ₃ integrin and/or αᵥβ₅ selective and that such selectivity is beneficial in reducing unwanted side-effects.

The compounds of the present invention can be used, alone or in combination with other therapeutic agents, in the treatment or modulation of various conditions or disease states including tumor metastasis, solid tumor growth (neoplasia), osteoporosis, Paget's disease, humoral hypercalcemia of malignancy, osteopenia, endometriosis, angiogenesis, including tumor angiogenesis, skeletal malignancy of breast cancer, retinopathy including macular degeneration, arthritis, including rheumatoid arthritis, periodontal disease, psoriasis and smooth muscle cell migration (e.g. restenosis and artherosclerosis), and microbial or viral diseases. Thus, in one embodiment, compounds of the present invention are beneficial for treating such conditions.

Methods of Treatment

In one embodiment, the present invention relates to a method of selectively inhibiting or antagonizing the αᵥβ₃ integrin and/or the αᵥβ₅ integrin and more specifically relates to a method of inhibiting an αᵥβ₃ integrin and/or an αᵥβ₅ integrin-mediated condition by administering a therapeutically effective amount of a compound of Formulas I-IXd to achieve such inhibition together with a pharmaceutically acceptable carrier.

In one embodiment, the present invention is directed towards of treating an αᵥβ₃ integrin-mediated condition. In another embodiment, the treatment is ameliorative treatment. In another embodiment, the treatment is palliative treatment. In yet another embodiment, the treatment is preventive treatment.

More specifically it has been found that it is advantageous to administer compounds which are αᵥβ₃ integrin and/or αᵥβ₅ selective and that such selectivity is beneficial in reducing unwanted side-effects. The selective antagonism of the αᵥβ₃ and/or αᵥβ₅ integrin over the αᵥβ₆ integrin is viewed as desirable in this class of compounds, as αᵥβ₆ may also play a role in normal physiological processes of tissue repair and cellular turnover that routinely occur in the skin and pulmonary tissues. In one embodiment, the selectivity ratio of the αᵥβ₃ and the αᵥβ₅ integrins over the αᵥβ₆ integrin is at least about 10 to at least about 1000. In another embodiment, the selectivity ratio is about 10 to about 100. In yet another embodiment, the selectivity ratio is at least about 5 to about 100. In a further embodiment, the selectivity ratio is at least about 1000.

For the selective inhibition or antagonism of αᵥβ₃ and/or αᵥβ₅ integrins, compounds of the present invention may be administered orally (such as by tablets, capsules [each of which includes sustained release or timed release formulations], pills powders, granules, elixirs, tinctures, suspensions, syrups and emulsions), parenterally, by inhalation spray, topically (e.g., ocular eyedrop), or transdermally (e.g., patch), all in unit dosage formulations containing conventional pharmaceutically acceptable carriers, adjuvants and vehicles. The term parenteral as used herein includes, for example, subcutaneous, intravenous (bolus or infusion), intramuscular, intrasternal, transmuscular infusion techniques or intraperitonally, all using forms well known to those of ordinary skill in the art.

Compounds of the present invention can also be administered via liposomes (e.g., unilamellar vesicles, large unilamellar vesicles, and multilamellar vesicles), and can be formed from a variety of phospholipids. Further, compounds of the present invention can be coupled to an antibody, such as a monoclonal antibody or fragment thereof, or to a soluble polymer for targeted drug delivery.

The compounds of the present invention are administered by any suitable route in the form of a pharmaceutical composition adapted to such a route, and in a dose effective for the treatment intended. Therapeutically effective doses of the compounds required to prevent or arrest the progress of or to treat the medical condition are readily ascertained by one of ordinary skill in the art using preclinical and clinical approaches familiar to the medicinal arts.

Accordingly, the present invention provides a method of treating conditions mediated by selectively inhibiting or antagonizing the αᵥβ₃ and/or αᵥβ₅ cell surface receptor which method comprises administering a therapeutically effective amount of a compound selected from the class of compounds depicted in the above formulas, wherein one or more compound is administered in association with one or more non-toxic, pharmaceutically acceptable carriers and/or diluents and/or adjuvants (collectively referred to herein as “carrier” materials) and if desired other active ingredients. More specifically, the present invention provides a method for selective antagonism of the αᵥβ₃ and/or αᵥβ₅ cell surface receptors over α_{llb}β₃ or αᵥβ₆ integrin receptors.

Based upon standard laboratory experimental techniques and procedures well known and appreciated by those skilled in the art, as well as comparisons with compounds of known usefulness, the compounds of Formulas I-IXd can be used in the treatment of patients suffering from the above pathological conditions. One skilled in the art will recognize that selection of the most appropriate compound of the invention is within the ability of one with ordinary skill in the art and will depend on a variety of factors including assessment of results obtained in standard assay and animal models.

Treatment of a patient afflicted with one of the pathological conditions comprises administering to such a patient an amount of compound of Formulas I-IX which is therapeutically effective in controlling the condition or in prolonging the survivability of the patient beyond that expected in the absence of such treatment. As used herein, the term “inhibition” of the condition refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent.

As stated previously, the compounds of the invention can be used in a variety of biological, prophylactic or therapeutic areas. It is contemplated that these compounds are useful in prevention or treatment of any disease state or condition wherein the αᵥβ₃ and/or αᵥβ₅ integrin plays a role.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions.

Oral delivery of an αᵥβ₃ and/or αᵥβ₅ inhibitor of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 1.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 200 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion.

Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regiment.

For administration to a mammal in need of such treatment, the compounds in a therapeutically effective amount are ordinarily combined with one or more adjuvants appropriate to the indicated route of administration. The compounds may be admixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulphuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The pharmaceutical compositions useful in the present invention may be subjected to conventional pharmaceutical operations such as sterilization and/or may contain conventional pharmaceutical adjuvants such as preservatives, stabilizers, wetting agents, emulsifiers, buffers, etc.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection. Such preparations can conveniently be prepared by admixing the compound with water and rendering the resulting solution sterile and isotonic with the blood. Injectable compositions according to the invention will generally contain from 0.1 to 5% w/w of a compound disclosed herein.

Pharmaceutical compositions suitable for topical application to the skin preferably take the form of an ointment, cream, lotion, paste, gel, spray, aerosol, or oil. Carriers which can be used include Vaseline, lanolin, polyethylene glycols, alcohols, and combinations of two or more thereof. The active compound is generally present at a concentration of from 0.1 to 15% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound of the present invention in an optionally buffered, aqueous solution, dissolved and/or dispersed in an adhesive, or dispersed in a polymer. A suitable concentration of the active compound is about 1% to 35%, preferably about 3% to 15%. As one particular possibility, the compound can be delivered from the patch by electrotransport or iontophoresis, for example, as described in Pharmaceutical Research, 3(6), 318 (1986).

In any case, the amount of active ingredient that can be combined with carrier materials to produce a single dosage form to be administered will vary depending upon the host treated and the particular mode of administration.

The solid dosage forms for oral administration including capsules, tablets, pills, powders, and granules noted above comprise one or more compounds of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

The term “therapeutically effective amount” shall mean that amount of drug or pharmaceutical agent that will elicit the biological or medical response of a tissue, system or animal that is being sought by a researcher or clinician.

Synthetic Methods

In another embodiment, the present invention provides a method of synthesizing substituted 3-guanidinoaryl and 3-guanidinoheteroaryl carboxylic acids useful for the preparation of, for example, compounds of the present invention. This synthetic scheme is described in Schemes AA and BB, and Examples AA-QQ. Surprisingly, the method of synthesis of the present invention provides shorter reaction times and higher yields than the previously described methods of synthesis of substituted 3-guanidinobenzoic acid via unsubstitued thiourea.

### EXAMPLES

13. The general synthetic sequences for preparing the compounds useful in the present invention are outlined in Schemes A-C, and, more specifically, in Schemes 1-8, and Examples 1-71. Both an explanation of, and the actual procedures for, the various embodiments of the present invention are described where appropriate. The following Schemes and Examples are intended to be illustrative of the present invention. Those with skill in the art will readily understand that known variations of the conditions and processes described in the Schemes and Examples can be used to synthesize the compounds of the present invention.

In one embodiment, the present invention contemplates a method for the preparation of a compound having the structure of Formula A: 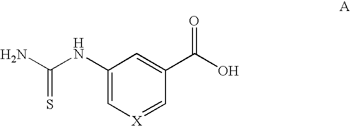
wherein x is CH, COH, or N;
wherein the method comprises contacting a compound having the structure of Formula B: 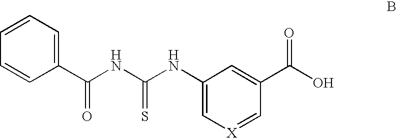
   with a base alkoxide and an alcoholic solvent, thereby producing the compound of Formula A. In another embodiment, the base alkoxide is selected from a sodium or potassium alkoxide. In yet another embodiment, the base alkoxide is NaOMe, NaOEt, KOMe, KOEt, or Kotert-Butyl. In one embodiment, the alcholic solvent is selected from the group consisting of butanol, propanol, MeOH, ethanol, and isopropyl alcohol.

In another embodiment, the present invention comprises a method for the preparation of a compound having the structure of Formula A: 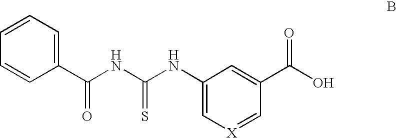
wherein x is CH, COH, or N;
wherein the method comprises contacting a compound having the structure of Formula C: 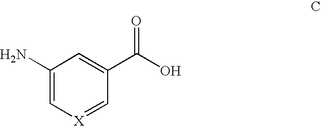
   with benzoylisothiocyanate and a solvent, thereby producing the compound of Formula B. In another embodiment, the solvent is selected from the group consisting of acetonitrile, tetrahydrofuran, dioxane, DMF, and DMSO.

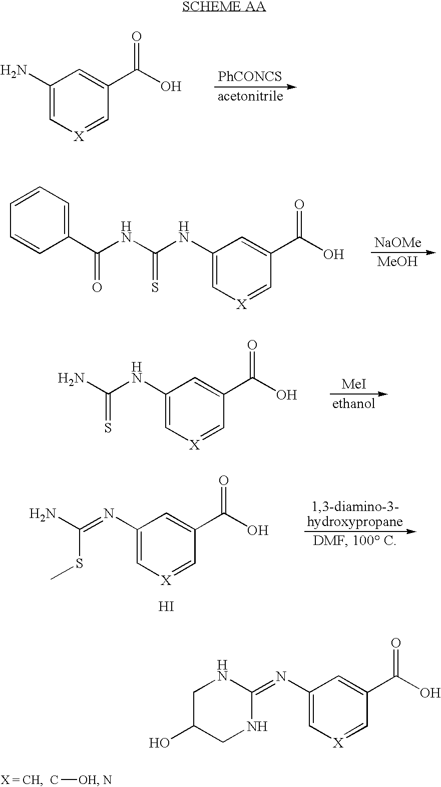

SCHEME 1 illustrates methodology useful for preparing various substituted tetrahydropyrimidinylaryl acid portion of the αᵥβ₃ integrin antagonists described herein which can be coupled to a gly-β-amino acid ester. Briefly, this entails the reaction of benzoylisothiocyanate with substituted aminoaryl acid to give the N-benzoylthiourea in quantitative yield. The N-benzoyl group can be removed by reaction with sodium methoxide to give the thiourea. The N-benzoyl group is removed as the volatile methyl benzoate. The thiourea can be isolated and treated with iodomethane or the crude reaction mixture (as shown in EXAMPLE D) can be converted to the isothiourea by reacting with iodomethane. The isothiourea is then treated with various diamino compounds to afford the desired substituted tetrahydropyrimidinylaminoaryl acids. The method can also be extended for the synthesis of tetrahydrodiazepines by reacting with substituted ω,ω′-diaminobutanes. This method has been found to be general in scope as shown in EXAMPLES A-I and SCHEMES 1-8.

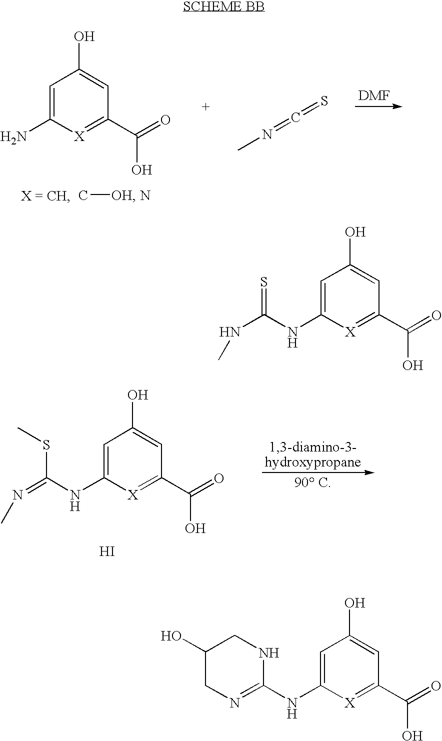

SCHEME 2 illustrates a modified methodology useful for preparing various substituted tetrahydropyrimidinylaryl acid portion of the αᵥβ₃ integrin antagonists. Briefly, instead of reacting with benzoylisothiocyanate, the aminoaryl acid can also be reacted with methylisothiocyanate to afford the methyl substituted thiourea. The advantage of this method is that it avoids the debenzoylation step. The N-methyl-S-methylisothiourea upon reaction with 2-hydroxy-1,3-diaminobutane gives the desired 5-hydroxytetra-hydropyrimidinylaminoaryl acid group. Both the N-methyl group and the S-methyl groups are removed during the reaction as volatile by-products.

### Example AA

### 3-hydroxy-5-[(5-hydroxy-1,4,5,6-tetrahydropyrimidin-2-yl)amino]benzoic acid

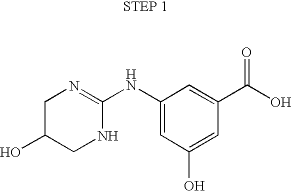

### N-benzoyl)-N′-(5-hydroxy-3-carboxyphenyl)thiourea

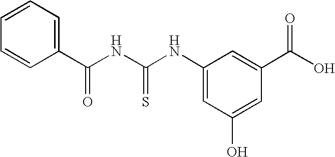

A mixture of 3-amino-5-hydoxybenzoic acid (30.7 g, 200.7 mmol) and benzoylisothiocyanate (26.57 g) in acetonitrile (450 mL) was stirred at room temperature for 1 h. The precipitate was filtered and washed with acetonitrile and dried to afford 57.17 g (90%) of the desired product as a yellow powder. ¹H NMR (CD₃OD) δ 8.01-8.04 (m, 2H), 7.79 (m, 1H), 7.69 (m, 1H), 7.58-7.63 (m, 2H), 7.37 (m, 1H). Anal. Calcd for C₁₅H₁₂N₂SO₄: Mol. Wt, 316.0518. Found. 317.0593 (M+H, HRMS).

Step 2

### N-(5-hydroxy-3-carboxyphenyl)thiourea

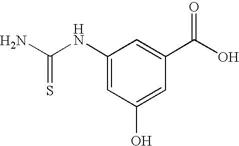

Sodium methoxide (106 mL, 25%) was added slowly to a stirred mixture of N-(benzoyl)-N′-(5-hydroxy-3-carboxyphenyl)thiourea (51.77 g, 163.73 mmol) in anhydrous methanol (250 mL). A clear solution resulted in 10 min. After 1 h stirring at room temperature, methanol was removed in vacuo and the residue was dried in vacuo. The residue was triturated with ether (500 mL) to leave a orange powder. The residue was dissolved in water (150 mL) and acidified to pH 6. The solid formed was filtered and dried. The solid was further washed with ether (100 mL). The residue obtained is the desired product. Yield: 34.6 g, (99.5%). ¹H NMR (CD₃OD) δ 7.42 (m, 1H), 7.28 (m, 1H), 7.11 (m, 1H). Anal. Calcd for C₈H₈N₂SO₃: Mol. Wt, 212.0256. Found. 213.0303 (M+H, HRMS).

Step 3

### N-(5-hydroxy-3-carboxyphenyl)-S-methylisothiourea

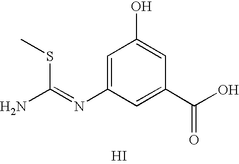

A mixture of N-(5-hydroxy-3-carboxyphenyl)thiourea (32.22 g, 0.164 mol) and iodomethane (23.34 g) in ethanol (200 mL) was heated at reflux for 5 h, the solution turned homogeneous. The solution was concentrated. Yield 56.89 g (100%). 1 H NMR and mass spectra consistent with the structure.This compound has been synthesized previously starting from the isothiourea and 1,3-diamino-2-hydroxy-propane. ¹H NMR (CD₃OD) δ 7.26-7.32 (m, 2H), 6.93 (m, 1H), 2.67 (s, 3H). Anal. Calcd for C₉H₁₀O₃N₂S: Mol. Wt, 226.0412. Found: Mol. W, 227.0462 (M+H, HRMS).

Step 4

### 3-hydroxy-5-[(5-hydroxy-1,4,5,6-tetrahydropyrimidin-2-yl)amino]-benzoic acid

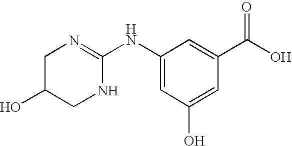

The isothiourea from STEP 3 has been previously converted to the desired 3-N-(5-hydroxytetra hydropyrimidinyl)-5-hydroxybenzoic acid (WO9944996).

### Example BB

### 3-hydroxy-5-(1,4,5,6-tetrahydropyrimidin-2-ylamino)benzoic acid

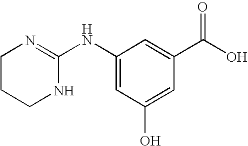

A mixture of N-(5-hydroxy-3-carboxyphenyl)-S-methylisothiourea (28.44 g, 0.084 mol) and diaminopropane (18.66 g, 0.252 mol) was heated at 100 C for 28 hours in DMF (40 mL). The reaction mixture was cooled and filtered, and was washed with ethyl acetate and ether. The solid was dried to afford 27 g. of the crude product. This was added 4N HCl in dioxane and was allowed to stir for 2 h and was concentrated. The residue was washed twice with ether to afford 16.0 g (70%) of the desired product as a powder. ¹H NMR (CD₃OD) δ 7.13-7.21 (m, 2H), 6.86 (m, 1H), 3.26 (m, 4H), 1.83 (m, 2H). Anal. Calcd for C₁₁H₁₃O₃N₃: Mol. Wt, 236.1005 (M+H, HRMS). Found: Mol. W, 236.1035 (M+H, HRMS).

### Example CC

### N-(5-hydroxytetrahydropyrimidinyl)-6-methyl-3-aminobenzoic acid

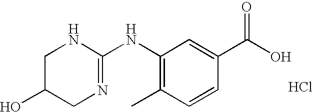 Step 1

### N-(Benzoyl)-N′-3-carboxy-6-methylphenyl)thiourea

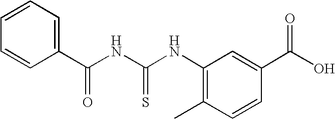

Benzoyl isothiocyanate (25.0 g, 0.153 mol), 3-amino-4-methyl benzoic acid (23.2 g, 0.153 mol) and acetonitrile (200 mL) were stirred at room temperature overnight. The precipitate was filtered and dried under vacuum to afford 44.36 g of the desired product (92%). ¹H NMR (CD₃OD) δ 8.34 (m, 1H), 8.01-8.04 (m, 2H), 7.90 (m, 1H), 7.71 (m, 1H), 7.69 (m, 1 H), 7.58-7.63 (m, 2H), 7.48 (m, 1H), 2.42 (s, 3H). Anal. Calcd for: C₁₆H₁₄N₂O₃S Mol. Wt, 314.0725. Found. 315.0823 (M+H, HRMS)

Step 2

### N-3-carboxy-6-methylphenyl)thiourea

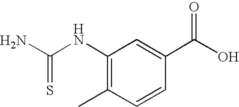

Sodium methoxide (61.12 mL, 0.283 mol) was added to a suspension of N-(benzoyl)-N′-3-carboxy-6-methylphenyl)thiourea (44.36 g, 0.141 mol) and anhydrous methanol (200 mL). The reaction mixture was stirred at room temperature for 45 minutes and concentrated. The residue was triturated with ether three times. The solid was powdered and washed with warm ether, and redissolved in minimum amount of water over 1 hour. The mixture was cooled to 0° C. and acidified with concentrated HCl over 1 h to afford an off-white powder. The product was dried in vacuum overnight. Yield: 29.0 g (98%). ¹H NMR (CD₃OD) δ 7.85-7.88 (m, 2H), 7.42 (m, 1H), 2.35 (s, 3H). Anal. Calcd for: C₉H₁₀N₂O₂S Mol. Wt, 210.0463. Found. 211.0501 (M+H, HRMS)

Step 3

### N-(3-carboxy-6-methylphenyl)-S-methylisothiourea

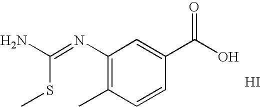

N-(3-carboxy-6-methylphenyl)-thiourea (29.0 g, 0.138 mol) and iodomethane (19.73 g, 8.66 mL, 0.138 mol) was dissolved in ethanol (150 mL) and heated to reflux under a drying tube overnight. The clear reaction mixture was concentrated to afford the desired product. ¹H NMR (CD₃OD) δ 8.01-8.03 (m, 1H), 7.90 (d, 1H, J=1.6 Hz), 7.58 d, 1H, J=7.9 Hz), 2.77 (s, 3H), 2.37 (s, 3H). Anal. Calcd for: C₁₀H₁₂N₂O₂S Mol. Wt, 224.0619. Found. 225.0663 (M+H, HRMS).

Step 4

### 3-hydroxy-5-(5-hydroxy-1,4,5,6-tetrahydropyrimidin-2-ylamino)-6-methylbenzoic acid HCl salt

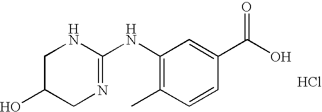

N-(3-Carboxy-6-methylphenyl)-S-methylisothiourea (17.0 g, 0.048 mol) and 1,3-diamino-2-hydroxypropane (12.96 g, 0.144 mol) and DMF (20 mL) were added to 200 mL flask equipped with condenser and drying tube. The solution was heated at 100° C. for 36 h and was cooled and filtered. The solid was washed with ethyl acetate, then ether. The solid was added slowly to stirring 4N HCl in dioxane. The mixture was stirred for 2 h. The reaction mixture became difficult to stir and the solution was concentrated and dried under high vacuum overnight. The solid was washed with ether three times, filtered, and dried. Yield 13.31 g (97%). ¹H NMR (CD₃OD) δ 7.13-7.21 (m, 2H), 6.86 (m, 1H), 3.26 (m, 4H), 1.83 (m, 2H). Anal. Calcd for C₁₁H₁₃O₃N₃: Mol. Wt, 236.1005 (M+H, HRMS). Found: Mol. W, 236.1035 (M+H, HRMS).

### Example DD

### N-(5-hydroxytetrahydropyrimidinyl)-3-aminonicotinic acid

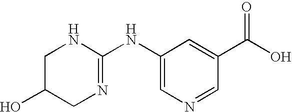 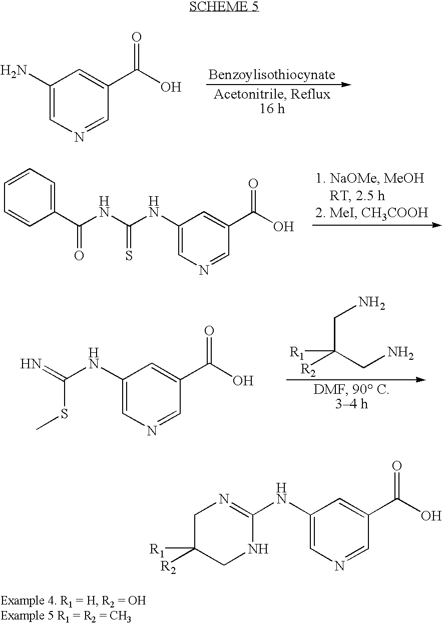 Step 1

### N-benzoyl-N′-(3-carboxy-5-pyridyl)thiourea

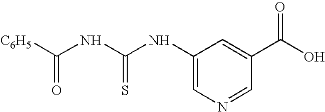

A mixture of 5-aminonicotinc acid (10.0 g, 0.072 mole), benzoylisothiocyanate (11.8 g, 0.072 mole), and DMAP (catalytic amount) in anhydrous acetonitrile (250 mL) was heated to reflux overnight under anhydrous conditions with vigorous stirring. The resulting yellow suspension was cooled and filtered. The residue was washed with water, followed by acetonitrile, and dried in vacuo overnight to yield the desired product as a pale yellow solid (21.4 g, 98%). ¹H NMR (CD₃OD) δ 8.9 (m, 2H), 8.6 (s, 1H), 7.9 (m, 2H), 7.6 (m, 1H), 7.5 (m, 2H). Anal. Calcd for C₁₄H₁₂N₃SO₃: Mol. Wt, Found. 302.1 (M+H, LRMS).

Step 2

### N-benzoyl-N′-(3-carboxy-5-pyridyl)-S-methylisothiourea

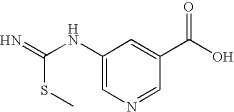

To the suspension of the product from Step 1 (11.1 g, 0.037 mole) in anhydrous MeOH (230 mL), was added NaOMe (25 wt % solution in methanol, 21.1 mL, 0.092 mole), at which point the reactant went into solution to give an orange-brown solution. This solution was stirred at room temperature for 3 h, cooled in an ice bath, and added methyl iodide (3.45 mL, 0.055 mole). The resulting mixture was stirred at 10° C. for 30 minutes and 1.5 h at room temperature. The reaction mixture was then quenched with acetic acid (2 mL), cooled in an ice bath, and filtered. The solids were washed with cold MeOH and dried in vacuo to afford the desired product as beige solid (2.66 g, 37%). ¹H NMR (CD₃OD) δ 8.66 (s, 1H), 8.27 (s, 1H), 7.64 (s, 1H), 2.37 (s, 3H). Anal. Calcd for C₈H₁₁O₂N₃S: Mol. Wt, 212.0493 (M+H, HRMS). Found: Mol. W, 212.0490 (M+H, HRMS).

Step 3

### N-(5-hydroxytetrahydropyrimidinyl)-3-aminonicotinic acid

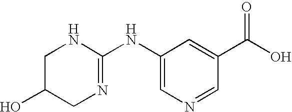

To a solution of the 1,3-diamino-2-hydroxypropane (11.2 g, 0.124 mole) in anhydrous DMF (80 mL), was added the product from STEP 2 (8.7 g, 0.041 mole). This mixture was heated at 85° C. under anhydrous conditions for 3 h. After 1-2 h of heating, the solution became turbid and turbidity increased during the course of heating. The reaction mixture was then cooled in an ice bath and filtered. The solids were washed with acetonitrile, water, acetonitrile, and dried in vacuo to yield the desired product as beige solid (3.7 g, 38%). ¹H NMR (CD₃OD) δ 9.06 (s, 1H), 8.72 (s, 1H), 8.34 (d, 1H), 4.3 (d, 1H), 3.5 (m, 4H). Anal. Calcd for C₁₀H₁₃O₃N₄: Mol. Wt, 237.0987 (M+H, HRMS). Found: Mol. W, 237.0945 (M+H, HRMS).

### Example EE

### N-(5,5-dimethyltetrahydropyrimidinyl)-3-aminonicotinic acid

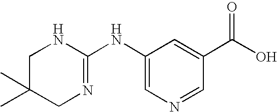

N-(5,5-dimethyltetrahydropyrimidinyl)-3-aminonicotinic acid was synthesized using the methodology described for EXAMPLE D substituting 4 equivalents of 2,2-dimethyl-1,3-propanediamine for 1,3-diamino-2-hydroxypropane in STEP 3, EXAMPLE D. Each of the products from STEP 3 were converted to their respective TFA or HCl salts by stirring 1 hour at 10° C. in a solution of anhydrous THF (10 mL for 1.0 g substrate) and TFA (1 eqv.) or 4N HCl/dioxane (2 eqv.). ¹H NMR (CD₃OD) δ 9.13 (s, 1 H), 8.73 (s, 1 H), 8.31 (d, 1H), 3.14 (m, 4H), 1.14 (s, 6H). Anal. Calcd for C₁₂H₁₆O₂N₄: Mol. Wt, 249.1351 (M+H, HRMS). Found: Mol. Wt, 249.1375 (M+H, HRMS).

### Example FF

### N-(5-fluorotetrahydropyrimidinyl)-3-aminonicotinic acid

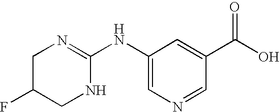 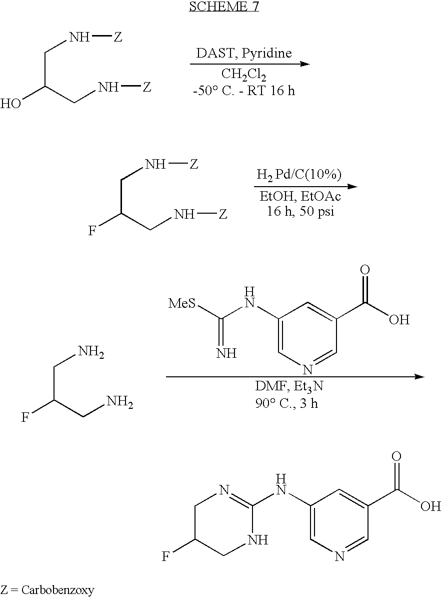 Step 1

### bis-N-benzyloxycarbonyl-2-fluoro-1,3-diaminopropane

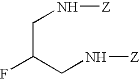

To a stirred suspension of bis-N-benzyloxycarbonyl-2-hydoxy-1,3-diaminopropane (6.0 g, 0.017 mol) in dichloromethane (50 mL) and pyridine (2.7 mL) at −50° C., was added dropwise a solution of DAST (2.5 mL) in dichloromethane (7.5 mL). The reaction mixture was gradually allowed to warm to room temperature over a period of 16 h under an atmosphere of argon, when a clear yellow solution was obtained. It was cooled and poured into a mixture of ice, water (100 mL), and dichloromethane (50 mL). The organic phase was washed with water (2×50 mL), and dried (Na₂SO₄). After removal of the solvent, the residue was purified by silica gel flash chromatography using 30% EtOAc in hexane. The appropriate fractions were combined, concentrated to dryness and the product was crystallized from dichloromethane/hexane to afford the desired fluoro intermediate (2.0 g) as a white fluffy powder. ¹H NMR (CDCl₃) δ 7.33 (m, 10H), 5.21 (br, 2H), 4.60 (d, 2H), 3.41 (m, 4H). Anal. Calcd for C₁₉H₂₂O₄N₂F: Mol. Wt, 361.1588 (M+H, HRMS). Found: Mol. Wt, 361.1543 (M+H, HRMS).

Step 2

### N-(5-fluorotetrahydropyrimidinyl)-3-aminonicotinic acid

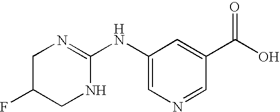

A solution of bis-N-benzyloxy-carbonyl-2-fluoro-1,3-diamino-propane (3.3 g, 0.0092 mol) as obtained from STEP 1, in EtOAc (30 mL), and EtOH (30 mL) was hydrogenated at 50 psi in the presence of Pd/C (10%, 2.7 g) for 16 h at room temperature. Following filtration, the catalyst was stirred with EtOH containing 40% water (50 mL) and filtered again. The filtrate was concentrated to dryness to afford a syrup (0.7 g). This was suspended in DMF (8.0 mL), added the product from step 2 of Example 4 (0.7 g, 0.0033 mol), catalytic amount of DMAP (0.01 g), and heated at 90° C. for 3 h under anhydrous conditions. DMF was distilled in vacuo, the residue was suspended in water (25 mL) and pH was adjusted to 4.5 by the addition of 1N HCl. The resulting mixture was cooled, solid that separated was filtered , and washed thoroughly with water, acetonitrile and dried in a desiccator in vacuo to provide the desired compound (0.24 g) as brown powder. ¹H NMR (CD₃OD) δ 9.0 (s,1H), 8.7 (d, 1H), 8.4 (t, 1H), 5.2 (m, 1H,_{JH}=46 Hz), 3.6 (m, 4H). Anal. Calcd for C₁₀H₁₂O₂N₄F: Mol. Wt, 239.0939 (M+H, HRMS). Found: Mol. W, 239.0984 (M+H, HRMS)

### Example GG

### 5-{[(5S,6S)-5,6-dihydroxy-4,5,6,7-tetrahydro-1H-1,3-diazepin-2-yl]amino}nicotinic acid dihydrochloride

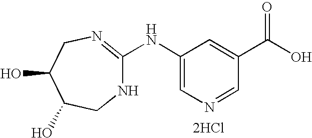

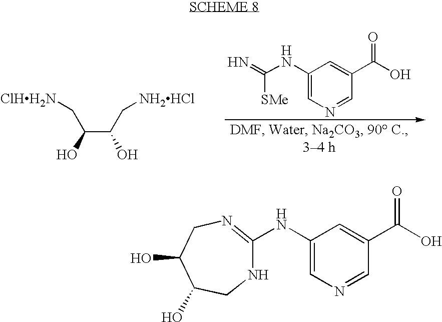

### 5-{[(5S,6S)-5,6-dihydroxy-4,5,6,7-tetrahydro-1H-1,3-diazepin-2-yl]amino}nicotinic acid dihydrochloride

To a solution of 1,4-diamino-2,3-dihydroxybutane dihydrochloride (2.21 g, 0.012 mole, synthesized from dimethyl-L-tartrate as described in J. Carbohydrate Chemistry, 5, (2), 183-197, [1986]), in water (6 mL) and anhydrous DMF (10 mL), was added sodium carbonate (1.83 g, 0.017 mole). To this mixture, the product from STEP 2, Example D (1.21 g, 0.006 mole) was added and the mixture was heated at 85° C. for 3 h. After cooling in an ice bath, DMF was distilled in vacuo, the resulting residue was suspended in water, and the pH was adjusted to 5.6. This solution was lyophilized to afford the desired product (0.907 g, 59% yield). ¹H NMR (CD₃OD) δ 9.01 (d,1H), 8.7 (d, 1H), 8.3 (m, 2H), 3.6 (m, 5H). Anal. Calcd for C₁₁H₁₅O₄N₄F: Mol. Wt, 267.1093 (M+H, HRMS). Found: Mol. W, 267.1084 (M+H, HRMS). This compound was converted to its HCl salt by stirring with 4N HCl/dioxane (2 eq) in THF(10 mL) at 10° C. for 1 h

### Example HH

### 3-N-(5-hydroxytetrahydropyrimidinyl )-5-hydroxybenzoic acid

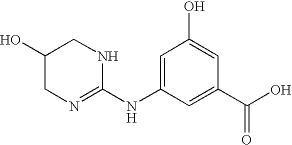 Step 1

### N-methyl-N′-(5-hydroxy-3-carboxyphenyl) thiourea

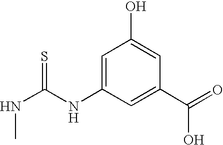

3-Amino-5-hydroxybenzoic acid (2000 g, 13.07 moles) was dissolved in dimethylformamide (8 L) and then methyl isothiocyanate (954 g, 13.07 moles) was added and the reaction mixture was stirred overnight. This gave crude N-methyl-N′-(5-hydroxy-3-carboxyphenyl)thiourea in solution, which was used for the next reaction. ¹H NMR (DMSOd6) δ 2.95 (s, 3H), 7.08 (s, 1H), 7.19 (s, 1H), 7.37 (s,1H), 7.78 (s,1H), 9.63 (s,1H), 9.78 (s,1H), 12.85 (s,1H).

Step 2

### N-methyl-N′-(5-hydroxy-3-carboxyphenyl)-S-methylisothiourea

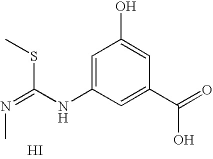

Methyl iodide (2598 g, 18.30 moles) was added to the crude reaction mixture from STEP 1 and stirred over the weekend. The reaction mixture was concentrated reaction mixture to 10 liters. This gave crude desired product in solution, which was used for the next reaction. Assumed 100% yield. ¹H NMR (DMSOd6) δ 2.54 (s, 3H), 3.09 (s, 3H), 6.98 (s, 1H), 7.35 (s, 1H), 7.37 (s, 1H), 9.20 (s, 1H), 10.23 (s, 2H).

Step 3

### 3-hydroxy-5-[(5-hydroxy-1,4,5,6-tetrahydropyrimidin-2-yl)amino]benzoic acid

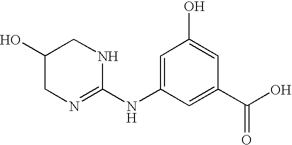

The crude reaction mixture from step 2 was cooled in ice/water to keep temperature <50° C. while adding the 1,3-diamino-2-hydroxy-propane (3529 g, 39.21 moles). Attached a N₂ gas source to the reaction vessel to sweep the gases produced during the reaction into a caustic scrubber. The reaction mixture was slowly heated to 90° C., and held at this temperature for 2.5 hours. The reaction mixture was cooled to ambient temperature, and water (12 L) was added and the pH of the solution was adjusted to 6.0 with concentrated hydrochloric acid. The suspension was stirred overnight. The solid was filtered, washed the cake with water and acetonitrile. This cake was dried on a fluid bed drier to give the title compound as a tan solid (2265.7 g, 69% yield). ¹H NMR (D₂0/DCl) δ 3.24 (dd, J=11.99 and 5.99, 4H), 4.19 (t, J=3.0, 1H), 6.79 (t, J=2.99, 1H), 7.13-7.17 (m, 1H), 7.17-7.21 (m, 1H). HRMS (ES+) M+H, theoretical 252.0984, observed 252.0962.

### Example 11

### 3,5-dichloro-2-hydroxy-β-[[2-[[[3-hydroxy-5-[(1,4,5,6-tetrahydro-5-hydroxypyrimidin-2-yl)amino]pheny]carbonyl]amino]-acetyl]amino]-benzenepropanoic acid, trifluroacetate salt

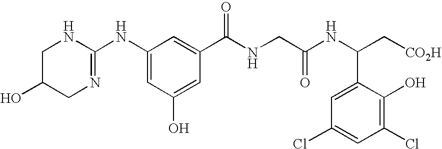

The above compound was prepared according to the methodology of Example 1, by reacting Example A with ethyl N-gly-3-amino-3-(3,5-dichloro-2-hydoxy)phenyl propionate. The yield, after lyophilization was 320 mg of as a white solid.

MS and ¹H NMR were consistent with the desired structure.

### Example JJ

### 3-iodo-5-bromo-2-hydroxy-β-[[2-[[[3-hydroxy-5-[(1,4,5,6-tetrahydro-5-hydroxypyrimidin-2-yl)amino]pheny]carbonyl]amino]-acetyl]amino]-benzenepropanoic acid, trifluroacetate salt

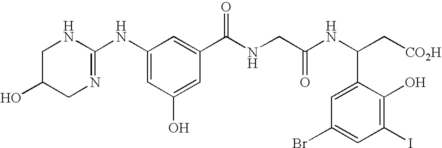

The above compound was prepared according to the methodology of Example 1, by reacting Example A with ethyl N-gly-3-amino-3-(3-iodo-5-bromo-2-hydoxy)phenyl propionate. The yield (after lyophilization) was 180 mg as a white solid.

MS and ¹H NMR were consistent with the desired structure.

### Example KK

### 3-chloro-5-bromo-2-hydroxy-β-[[2-[[[3-hydroxy-5-[(1,4,5,6-tetrahydro-5-hydroxypyrimidin-2-yl)amino]pheny]carbonyl]amino]-acetyl]amino]benzenepropanoic acid, trifluroacetate salt

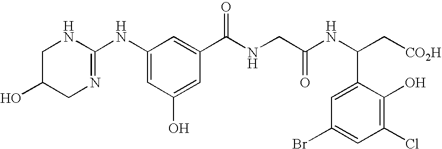

The above compound was prepared according to the methodology of Example 1, by reacting Example A with ethyl N-gly-3-amino-3-(3-chloro-5-bromo-2-hydoxy)phenyl propionate. The yield (after lyophilization) was 180 mg as a white solid.

MS and ¹H NMR were consistent with the desired structure.

### Example LL

### 3-iodo-5-chloro-2-hydroxy-β-[[2-[[[3-hydroxy-5-[(1,4,5,6-tetrahydro-5-hydroxypyrimidin-2-yl)amino]pheny]carbonyl]amino]acetyl]amino]-benzenepropanoic acid, trifluroacetate salt

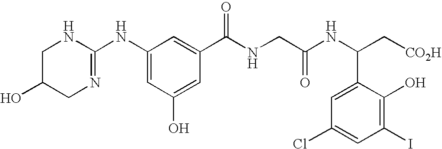

The above compound was prepared according to the methodology of Example 1, by reacting Example A with ethyl N-gly-3-amino-3-(3-iodo-5-chloro-2-hydoxy)phenyl propionate. The yield (after lyophilization) was 250 mg as a white solid.

MS and ¹H NMR were consistent with the desired structure.

### Example MM

### 3,5-dibromo-2-hydroxy-β-[[2-[[[3-hydroxy-5-[(1,4,5,6-tetrahydro-5-hydroxypyrimidin-2-yl)amino]pheny]carbonyl]amino]acetyl]amino]-benzenepropanoic acid, trifluroacetate salt

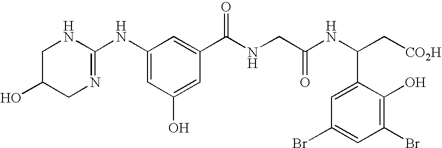

The above compound was prepared according to the methodology of Example 1, by reacting Example A with ethyl N-gly-3-amino-3-(3,5-dibromo-2-hydoxy)phenyl propionate. The yield (after lyophilization) was 220 mg as a white solid.

MS and ¹H NMR were consistent with the desired structure.

### Example NN

### 3 ,5-dichloro-2-hydroxy-β-[[2-[[[5-[(1,4,5,6-tetrahydropyrimidin-2-yl)amino]pyridinyl]carbonyl]amino]acetyl]amino]benzenepropanoic acid, trifluroacetate salt

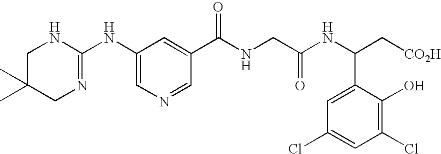

To a suspension of Example E (0.40 g, 0.00125 mole) in anhydrous DMF (10 mL) at −20° C. was added isobutylchioroformate (0.17 g, 0.00125 mole), followed by the dropwise addition of N-methyl-morpholine (0.14 g, 0.00137 mole). After stirring this mixture under argon atmosphere for 20 minutes at −20° C., an additional amount of N-methylmorpholine (0.14 g, 0.00137 mole) was added, followed by the addition of ethyl N-gly-3-amino-3-(3,5-dichloro-2-hydoxy)phenyl propionate (0.46 g, 0.00125 mole). The resulting mixture was stirred at −20° C. for 15 minutes, and then stirred at room temperature for 2 h. DMF was distilled in vacuo and the residue was purified by reverse phase HPLC to yield (after lyophilization) the desired ester as a white solid (0.20 g, 21%). MS (m/z M+H C₂₅H₃₂N₆O₅Cl₂) cal 565.1733, 565.1736 obs. ¹H-NMR (400 MHz, CD₃OD): δ 8.8 (d, 1H), 8.6 (d, 1H), 8.1 (s, 1H), 7.3 (d, 1H), 7.2 (d, 1H) 5.6 (m, 1H), 4.1 (m, 4H), 3.2 (m, 4H), 2.8 (m, 2H), 1.18 (t, 3H), 1.09 (s, 6H).

The ester (0.2 g) was stirred with 1M LiOH (2 mL) for 1 h at room temperature. The pH was adjusted to 2 with trifluoroacetic acid and the product was purified by reverse phase HPLC to provide (after lyophilization) the desired acid as a white solid (0.11 g). MS (m/z M+H C₂₃H₂₇N₆O₅Cl₂) cal 537.1419, 537.1405 obs. ¹H-NMR (400 MHz, CD₃OD): δ 8.8 (d, 1H), 8.6 (d, 1H), 8.1 (s, 1H), 7.3 (d, 1H), 7.2 (d, 1H) 5.56 (m, 1H), 4.1 (m, 2H), 3.2 (m, 4H), 2.8 (m, 2H), 1.09 (s, 6H).

### Example OO

### 3-bromo-5-chloro-2-hydroxy-β-[[2-[[[5-[(1,4,5,6-tetrahydro-5,5-dimethyl pyrimidin)amino]pyridinyl]carbonyl]amino]acetyl]amino]benzenepropanoic acid, trifluroacetate salt

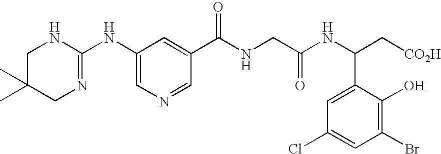

The above compound was prepared according to the procedure described in the Example 7 using ethyl N-gly-3-amino-3-(3-bromo-5-chloro-2-hydoxy)phenyl propionate in the place of ethyl N-gly-3-amino-3-(3,5-dichloro-2-hydoxy)phenyl propionate. The resulting ester (0.19 g, 0.00023 mole) was stirred with 1M LiOH (2 mL) for 1 h at room temperature. The pH was adjusted to 2 with trifluoroacetic acid and the product was purified by reverse phase HPLC to provide (after lyophilization) the desired acid as a white solid (0.13 g, 72%). MS (m/z M+H C₂₃H₂₇N₆O₅ClBr) cal 581.0914, 581.0866 obs. ¹H-NMR (400 MHz, CD₃OD): δ 8.9 (d, 1H), 8.59 (d, 1H), 8.1 (s, 1H), 7.41 (d, 1H), 7.25 (d, 1H) 5.56 (m,1H), 4.1 (m, 2H), 3.2 (m, 4H), 2.8 (m, 2H), 1.09 (s, 6H).

### Example PP

### 3-bromo-5-chloro-2-hydroxy-β-[[2-[[[5-[(1,4,5,6-tetrahydro-1,3-dioxolanepyrimidin-2-yl)amino]pyridinyl]carbonyl]amino]-acetyl]amino]benzenepropanoic acid, trifluroacetate salt

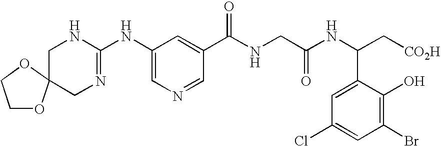

Example F (0.38 g, 0.0014 mol) was suspended in dry THF (5.0 mL), added trifluoroacetic acid (0.1 mL) and stirred at 10° C. under anhydrous conditions. After 30 mins, THF was distilled under reduced pressure and the residue was dried in vacuo for 3 h. This material was dissolved in dry DMF (4.0 mL), cooled to −15° C., and added isobutyl-chloroformate (0.18 mL), followed by the addition of N-methylmorpholine (0.17 mL) and stirred for 30 mins under argon atmosphere. To this mixture was added a solution of the amine generated by the addition of N-methylmorpholine (0.17 mL) to a solution of ethyl N-gly-3-amino-3-(3-bromo-5-chloro-2-hydoxy)phenyl propionate (0.51 g) in DMF (3.0 mL) at 0° C. The resulting mixture was stirred at −15° C. for 30 mins, and at room temperature for 16 h. The solvents were then removed by distillation in vacuo, and the residue was purified by reverse-phase HPLC using 10-90% acetonitrile/water gradient (40 min) at a flow rate of 70 mL/min. The desired fractions were combined and freeze dried to afford the desired ethylester (0.4 g) as a fluffy white powder: ¹H-NMR (300 MHz, CD₃OD): δ 8.91 (d, J=1.5 Hz,1H), 8.59 (1H, J=1.5 Hz, iH), 8.12 (s, 1H), 7.41 (d, J=1.8 Hz, 1H), 7.24 (d, J=1.8 Hz, 1H), 5.51 (m, 1H), 4.1(m, 8H), 3.38(m, 4H), and 2.85 (m, 2H); HRMS: m/z calcd. C₂₅H₂₉N₆O₇ClBr (MH⁺) 639.09696, found 639.0983.

This material was then stirred with lithium hydroxide (1M, 2.0 mL) at room temperature. After 45 mins, the reaction mixture was cooled, diluted with water, acidified with trifluoroacetic acid, and the desired acid (0.25 g, Example 6) was isolated by reverse-phase HPLC using 10-90% acetonitrile/water as described above. ¹H-NMR (300 MHz, CD₃OD): δ 8.91 (d, 1H, J=1.5 Hz), 8.59 (d, 1H, J=1.5 Hz), 8.12 (s, 1H, 7.41 (d, 1H, 1.8 Hz), 7.25 (d, 1H, 1.8 Hz), 5.49 (m 1H), 4.10 (s, 2H), 4.08 (s, 4H), 3.37 (s, 4H), & 2.84 (m, 2H); HRMS: m/z calcd C₂₃H₂₅N₆O₇ClBr (MH⁺) 611.0651, found 611.0685.

### Example QQ

### 3-bromo-5-chloro-2-hydroxy-β-[[2-[[[5-[(1,4,5,6-tetrahydro-5-hydroxy-pyrimidin-2-yl)amino]pyridinyl]carbonyl]amino]-acetyl]amino]-benzenepropanoic acid, trifluroacetate salt

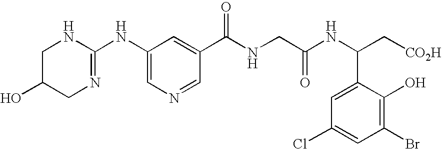

Example G (0.22 g) as obtained above was suspended in dry THF (4.0 mL), added trifluoroacetic acid (0.1 mL), stirred at 10 C for 30 mins, and concentrated under reduced pressure. The residue was dried in a desiccator in vacuo. This material was suspended in dry DMF (5 mL), added isobutylchloroformate (0.12 mL) followed by the addition of N-methylmorpholine (0.11 mL), and stirred at −15° C. under argon atmosphere. After 30 min, added a solution of the amine generated by the addition of N-methylmorpholine (0.095 mL) to a solution of ethyl N-gly-3-amino-3-(3-bromo-5-chloro-2-hydoxy)phenyl propionate (0.37 g) in DMF (3.0 mL). The resulting mixture was stirred at −15° C. for 30 mins, and at room temperature for 16 h. DMF was distilled in vacuo and the residue was purified by reverse-phase HPLC using 10-90% acetonitrile/water. The desired fractions were combined and freeze dried to afford the desired ester product as a pale yellow powder (0.35 g). ¹H-NMR and mass spectral data were consistent with the structure.

The ester (0.3 g) was stirred with 1M LiOH ( 3.0 mL) at room temperature. After 1 h, the solution was diluted with water (3.0 mL), cooled and acidified with trifluoroacetic acid. The resulting mixture was then purified by reverse-phase HPLC using 10-90% acetonitrile/water (30 min gradient) at flow rate of 70 mL/min. The appropriate fractions were combined and freeze dried to provide the desired compound (0.22 g) as a white powder. ¹H-NMR (300 MHz, CD₃OD): δ 8.92 (d, J=1.5 Hz, 1H ), 8.60 (d, J=1.5 Hz, 1H), 8.12(m, 1H), 7.41 (d, J=1.8 Hz, 1H), 7.25 (d, J=1.8 Hz), 5.38 (m, 1H), 5.25 (d, iH), 4.10 (s, 2H), 6.63 (m 4H), & 2.83 (m, 2H); HRMS: m/z calcd C₂₁H₂₁N₆O₅FClBr (MH⁺) 573.0487, found 573.0474.

### Example RR

### 3-bromo-5-chloro-2-hydroxy-β-[[2-[[[5-[((5S,6S)-5,6-dihydroxy-4,5,6,7-tetrahydro-1H-1,3-diazepin-2-yl)amino]pyridinyl]carbonyl]amino]-acetyl]amino]-benzenepropanoic acid, trifluroacetate salt

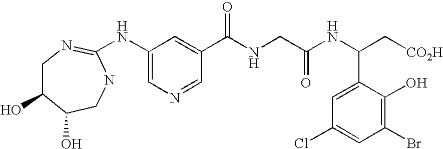

To a suspension of Example H (0.11 g, 0.00023 mole)) in anhydrous DMF (10 mL) at −20° C., was added isobutylchloroformate (0.016 g, 0.00012 mole), followed by the dropwise addition of N-methylmorpholine (0.013 g, 0.00013 mole). After stirring this mixture under argon atmosphere for 20 minutes at −20° C., an additional amount of N-methylmorpholine (0.013 g, 0.00013 mole) was added followed by the addition of ethyl N-gly-3-amino-3-(3-bromo-5-chloro-2-hydoxy)phenyl propionate (0.048 g, 0.00012 mole). The resulting mixture was stirred at −20° C. for 15 minutes. After stirring at room temperature for 2 h, DMF was distilled in vacuo and the residue was purified by reverse-phase HPLC to yield (after lyophilization) the desired ester as a white solid (0.03 g, 33 %). MS (M+H 627 M+H 629) ¹H-NMR (400 MHz, Cd₃Od): δ 8.8 (s, 1H) δ 8.5 (s, 1H), 8.1 (s, 1H), 7.4 (s, 1H), 7.2 (s, 1H) 5.6 (m, 1H), 4.1 (m, 4H), 3.7 (m, 2H), 3.6 (m, 2H), 3.3 (m, 2H), 2.9 (m, 2H), 1.2 (m, 3H)

This ester (0.03 g, 0.000035 mole) was then stirred with 1M LiOH (2 mL). After stirring for 1 h at room temperature, the pH was adjusted to 20 with trifluoroacetic acid, and the product was isolated by reverse-phase HPLC to provide (after lyophilization) the desired acid as a white solid (0.001 g, 3.5 %) MS (M+H 599 M+H 601). ¹H-NMR (400 MHz, CD₃OD): δ 8.8 (s, 1H), 8.5 (s, 1H), 8.0 (s, 1H), 7.4 (s, 1H), 7.2 (s, 1H) 5.6 (m, 1H), 4.1 ( m, 2H), 3.8 (m, 2H), 3.5 (m, 2H), 3.3 (m, 2H), 2.8 (m, 2H)

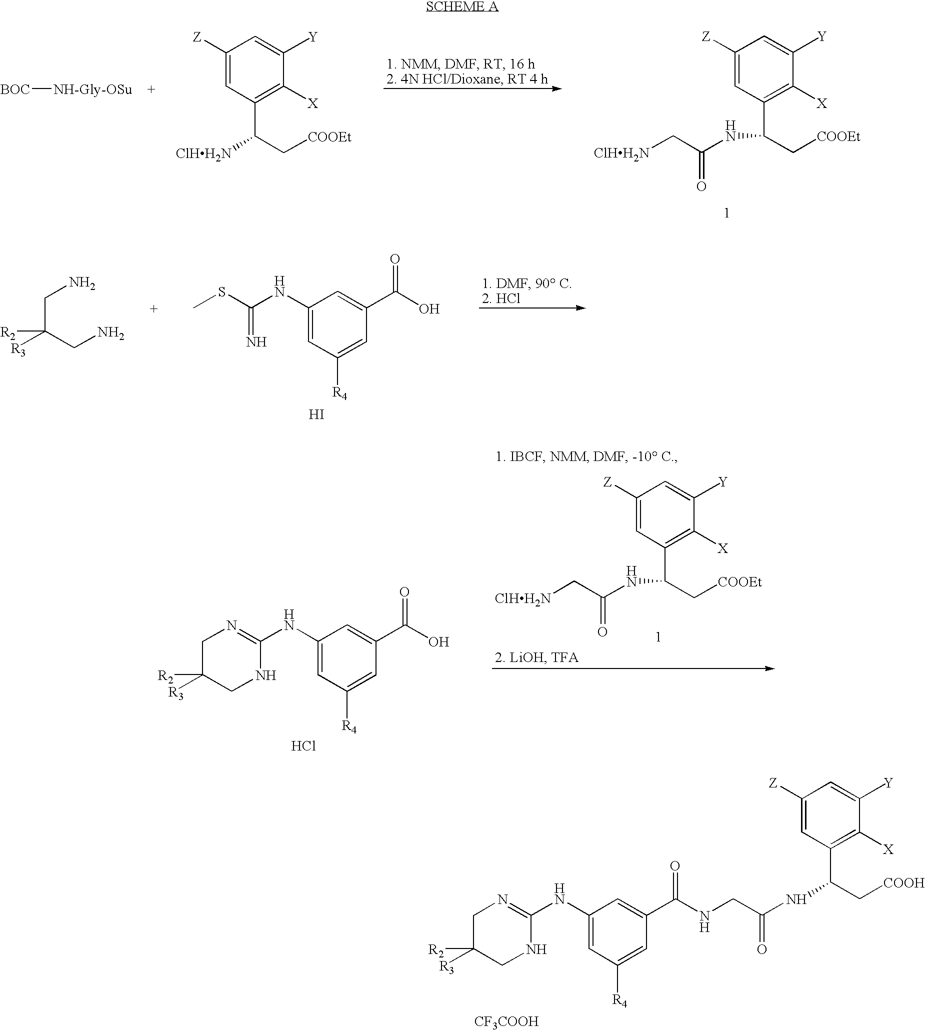

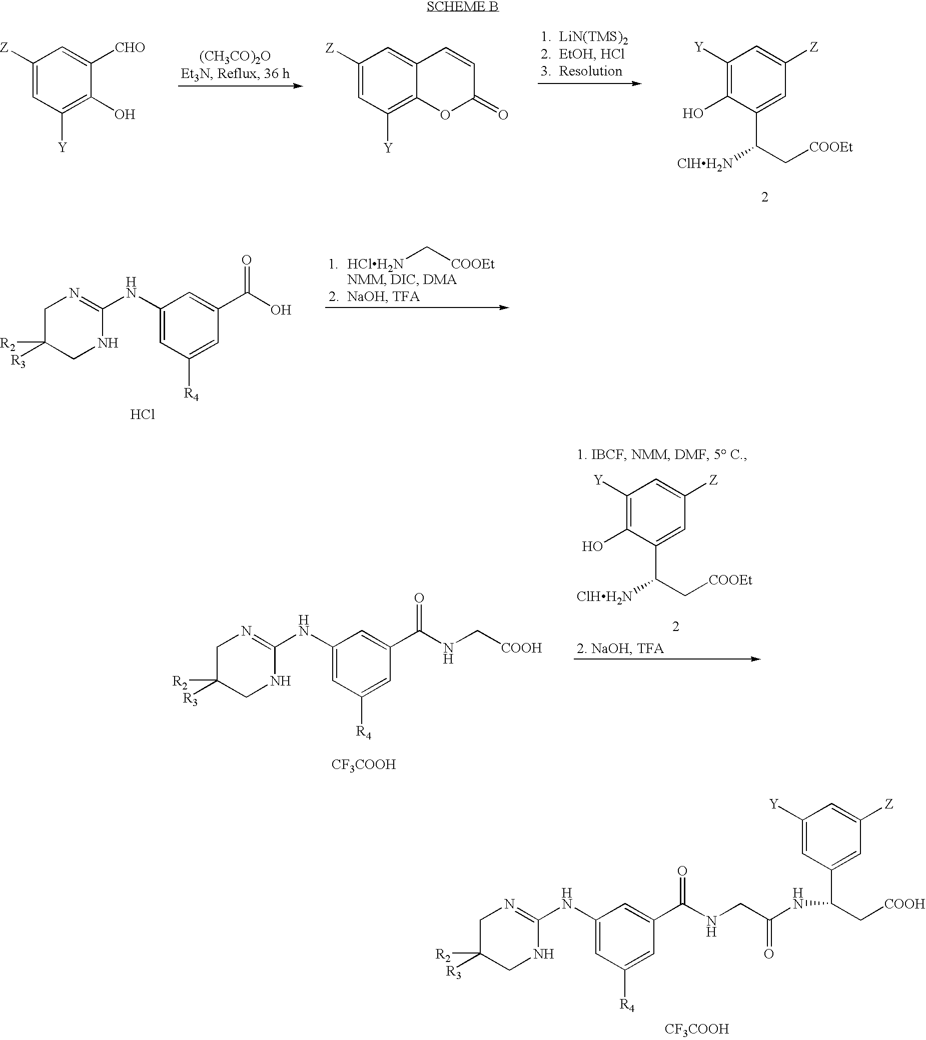

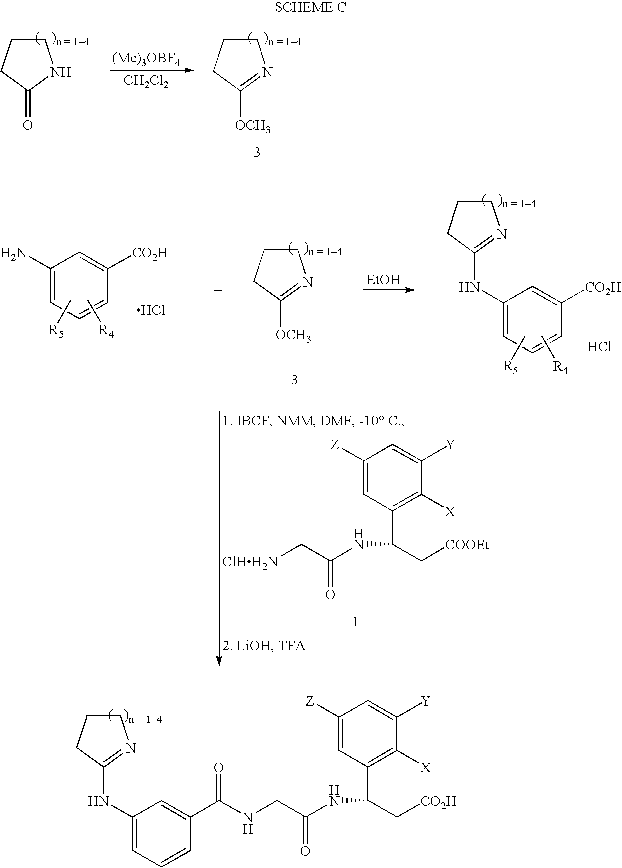

### Example 1

### (3R)-3-(3-bromo-5-chloro-2-hydroxyphenyl)-3-[(N-{3-hydroxy-5-[(5-hydroxy-1,4,5,6-tetrahydropyrimidinyl)amino]benzoyl}glycyl)amino]propanoic acid, monotrifluoroacetate

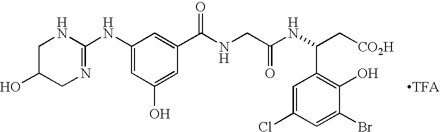 Step 1

### Ethyl (3R)-3-amino-3-(3-bromo-5-chloro-2-hydroxyphenyl)propanoate

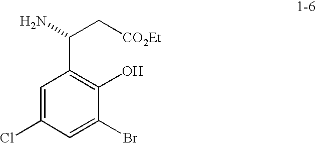

To a solution of the (R)-(CBZ)-β-amino ester (the synthesis of the ester was described in U.S. Pat. No. 6,013,651) (55.3 g, 121.0 mmol) in CH₂Cl₂ (500 mL) was added trimethylsilyl iodide (30.5 g, 152.0 mmol) in CH₂Cl₂ (100 mL) via canula. The reaction solution was stirred at room temperature for 1.5 h. Methanol (25.0 mL, 609.2 mmol) was added dropwise and the solution stirred for 15 minutes. The reaction solution was concentrated in vacuo. The residue was dissolved in MTBE (550 mL) and extracted with 1M HCl (340 mL) and water (1×200 mL, 1×150 mL). The aqueous extracts were back washed with MTBE (150 mL). To the aqueous solution was added solid NaHCO₃ (43.0 g, 512 mmol) in small portions. The basified aqueous mixture was extracted with MTBE (1×1 L, 2×250 mL). The combined organic solution was washed with brine and concentrated in vacuo to give the desired product 1-6 (30.3 g, 76% yield): ¹H NMR (300 MHz, DMSO-d₆) δ 7.41 (d, 1H, J=2 Hz), 6.91 (d, 1H, J=2 Hz), 4.42 (t, 1H, J=6 Hz), 4.05 (q, 2H, J=7 Hz), 2.75 (m, 2H), 1.14 (t, 3H, J=7 Hz). Anal. Calcd for C₁₁H₁₃BrClNO₃+0.5 H₂O: C, 39.84; H, 4.26; N, 4.22. Found: C, 39.49; H, 3.89; N, 4.13.

Step 2

### Ethyl (3R)-3-(3-bromo-5-chloro-2-hydroxyphenyl)-3-{[N-(tert-butoxycarbonyl)-glycyl]amino}propanoate

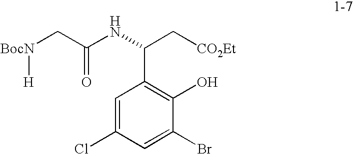

To a solution of ethyl (3R)-3-amino-3-(3-bromo-5-chloro-2-hydroxyphenyl)-propanoate, (29.3 g, 90.7 mmol) in DMF (250 mL) was added N-t-Boc-glycine N-hydroxysuccinimide ester (24.7 g, 90.7 mmol). The reaction mixture was stirred at room temperature for 20 h. The mixture was poured into ethyl acetate (1.2 L) and washed with 1M HCl (2×250 mL), sat. aqueous NaHCO₃ solution (2×250 mL) and brine (2×250 mL). The solution was dried (MgSO₄) and concentrated in vacuo to give the desired product 1-7 (43.8 g, 100% yield). ¹H NMR was consistent with the proposed structure.

Step 3

### Ethyl (3R)-3-(3-bromo-5-chloro-2-hydroxyphenyl)-3-(glycylamino)-propanoate, hydrochloride

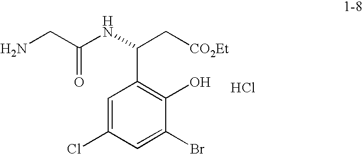

To a solution of 1-7 from STEP 2 (43.5 g, 90.7 mmol) in absolute ethanol (300 mL) was added an ethanolic HCl solution (105 mL of a 4.3M solution, 453.5 mmol). The reaction solution was kept at room temperature for 1 h. The solution was cooled and concentrated in vacuo. The residue was dissolved in ethyl acetate (300 mL) and stirred at 0° C. for 2 h. A white precipitate was collected by filtration and washed with cold ethyl acetate. The solid was dried in vacuo to give the desired product (30.4 g, 81% yield): ¹H NMR (300 MHz, DMSO-d₆) δ 7.55 (d, 1H, J=2 Hz), 7.32 (d, 1H, J=2 Hz), 5.53 (m, 1H), 4.07 (q, 2H, J=7 Hz), 3.61 (m, 2H), 2.73 (m, 2H), 1.14 (t, 3H, J=7 Hz). Anal. Calcd. for C₁₃H₁₆BrClN₂O₄+1.0 HCl+0.5 H₂O: C, 36.73; H, 4.27; N, 6.59. Found: C, 36.68; H, 4.07; N, 6.78.

Step 4

### (3R)-3-(3-bromo-5-chloro-2-hydroxyphenyl)-3-[(N-{3-hydroxy-5-[(5-hydroxy-1,4,5,6-tetrahydropyrimidinyl)amino]benzoyl}glycyl)amino]-propanoic acid, monotrifluoroacetate

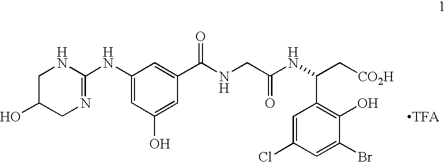

To a solution of 3-N-(5-hydroxytetrahydropyrimidinyl)-5-hydroxybenzoic acid (prepared according to U.S. Pat. No. 6,013,651, Example H, 3.0 g, 10.3 mmol) in DMA (36 mL) at −8° C. was added isobutyichloroformate (1.5 mL, 11.4 mmol) and NMM (1.3 mL, 11.4 mmol). The reaction solution was warmed to 8° C. over 30 min. The solution was cooled to −5° C. and a solution of the product from STEP 3 (4.3 g, 10.3 mmol) in DMA (18 mL) was added followed by NMM (1.3 mL, 11.4 mmol). The reaction mixture was warmed to room temperature and stirred overnight. The mixture was filtered and the filtrate was concentrated in vacuo. The residue was dissolved in 2.5N NaOH (30 mL) and water (30 mL). The reaction solution was kept at room temperature for 1.5 h. The pH was adjusted to 5 with TFA and the product was purified by reverse phase HPLC (95:5 H₂O/TFA:MeCN to 60:40 H₂O/TFA:MeCN) to give the desired product (1.8 g, 22%). Anal. Calcd for C₂₂H₂₃BrClN₅O₇+1.6 TFA: C, 39.45; H, 3.23; N, 9.13. Found: C, 39.36; H, 3.32; N, 9.52. ¹H NMR was consistent with the proposed structure.

### Example 2

### (3R)-3-(3-bromo-5-chloro-2-hydroxyphenyl)-3-{[N-({5-[(5-hydroxy-1,4,5,6-tetra-hydropyrimidin-2-yl)amino]pyridin-3-yl}carbonyl)glycyl]amino}propanoic acid, monotrifluoroacetate

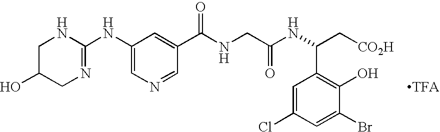 Step 1.

### N-benzoyl-N′-(3-carboxy-5-pyridyl)thiourea.

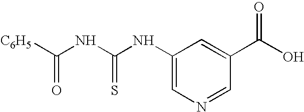

A mixture of 5-aminonicotinc acid (10.0 g, 0.072 mole), benzoylisothiocynate (11.8 g, 0.072 mole), and DMAP (catalytic amount) in anhydrous acetonitrile (250 mL) was heated to reflux overnight under anhydrous conditions with vigorous stirring (Scheme A1). The resulting yellow suspension was cooled and filtered. The residue was washed with water, followed by acetonitrile, and dried in vacuo overnight to yield the desired product as a pale yellow solid (21.4 g, 98%). MS and ¹H-NMR were consistent with the desired structure.

Step 2.

### N-benzoyl-N′-(3-carboxy-5-pyridyl)-S-methylisothiourea.

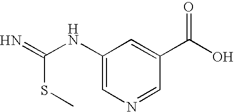

To the suspension of the product from Step 1 (11.1 g, 0.037 mole) in anhydrous MeOH (230 mL), was added NaOMe (25 wt % solution in methanol, 21.1 mL, 0.092 mole), at which point the reactant went into solution to give an orange-brown solution (SCHEME A1). This solution was stirred at room temperature for 3 h, cooled in an ice bath, and added methyliodide (3.45 mL, 0.055 mole). The resulting mixture was stirred at 10° C. for 30 minutes and 1.5 h at room temperature. The reaction mixture was then quenched with acetic acid (2 mL), cooled in an ice bath, and filtered. The solids were washed with cold MeOH and dried in vacuo to afford the desired product as beige solid (2.66 g, 37%). MS and ¹H-NMR were consistent with the desired structure.

Step 3.

### N-(5-hydroxytetrahydropyrimidinyl)-3-aminonicotinic acid.

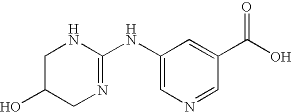

To a solution of the 1,3-diamino-2-hydroxypropane (11.2 g, 0.124 mole) in anhydrous DMF (80 mL), was added the product from Step 2 (8.7 g, 0.041 mole). This mixture was heated at 85° C. under anhydrous conditions for 3 h. After 1-2 h of heating, the solution became turbid and turbidity increased during the course of heating. The reaction mixture was then cooled in an ice bath and filtered. The solids were washed with acetonitrile, water, acetonitrile, and dried in vacuo to yield the desired product as beige solid (3.7 g, 38%). MS and ¹H-NMR were consistent with the desired structure.

Step 4.

### Ethyl (3R)-3-(3-bromo-5-chloro-2-hydroxyphenyl)-3-{[N-({5-[(5-hydroxy-1,4,5,6-tetrahydropyrimidin-2-yl)amino]pyridin-3-yl}carbonyl)glycyl]-amino}propanoate, monotrifluoroacetate

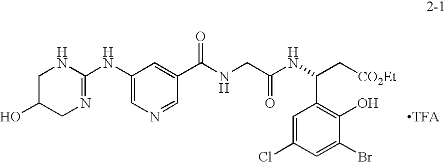

To a solution of N-(5-hydroxytetrahydropyrimidinyl)-3-aminonicotinic acid from Step 3 (1.0 g, 3.2 mmol) in DMF (10 mL) at 0° C. was added isobutylchloroformate (0.42 mL, 3.2 mmol) and NMM (1.1 mL, 9.6 mmol). The reaction solution was kept at 0° C. for 20 minutes. A solution of ethyl (3R)-3-(3-bromo-5-chloro-2-hydroxyphenyl)-3-(glycylamino)-propanoate, hydrochloride prepared as in Example 1, STEP 3 (1.3 g, 10.3 mmol) and NMM (0.36 mL, 3.2 mmol) in DMF (6 mL) was added. The reaction mixture was stirred at 0° C. for 30 minutes and then warmed to room temperature overnight. The mixture was concentrated in vacuo. The product was purified by reverse phase HPLC (90:10 H₂O/TFA:MeCN to 50:50 H₂O/TFA:MeCN) to give the desired product 2-1, along with unreacted amine (600 mg).

Step 5

### (3R)-3-(3-bromo-5-chloro-2-hydroxyphenyl)-3-{[N-({5-[(5-hydroxy-1,4,5,6-tetrahydropyrimidin-2-yl)amino]pyridin-3-yl}carbonyl)glycyl]amino}-propanoic acid, monotrifluoroacetate

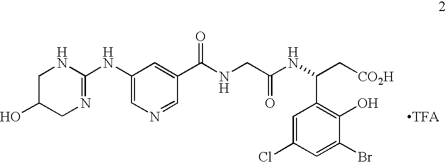

A solution of the crude product from STEP 1 (480 mg) in 1M NaOH solution (6 mL) was kept at room temperature for 4 h. The reaction solution was acidified to pH 4 with TFA. The mixture was purified by reverse phase HPLC (95:5 H₂O/TFA:MeCN to 60:40 H₂O/TFA:MeCN) to give the desired product 2 (160 mg, 9% from STEP 1): ¹H NMR (300 MHz, DMSO-d₆) δ 9.91 (s,1H), 9.75 (br s, 1H), 9.06 (t, 1H, J=6 Hz), 8.90 (d, 1H, J=2 Hz), 8.62 (d, 1H, J=6 Hz), 8.59 (d, 1H, J=2 Hz) 8.45 (br s, 2H), 8.02 (dd, 1H), 7.53 (d,1H, J=2 Hz), 7.28 (d, 1H, J=2 Hz), 5.43 (m, 1H), 4.11 (m, 1H), 3.96 (m, 3H), 3.34 (m, 2H), 3.17 (m, 2H), 2.72 (dd, 1H, J=4 Hz, J=15 Hz), 2.63 (dd, 1H, J=4 Hz, J=15 Hz). Anal. Calcd for C₂₁H₂₂BrClN₆O₆+1.7 TFA: C, 38.38; H, 3.13; N, 11.01. Found: C, 38.20; H, 3.21; N, 11.11.

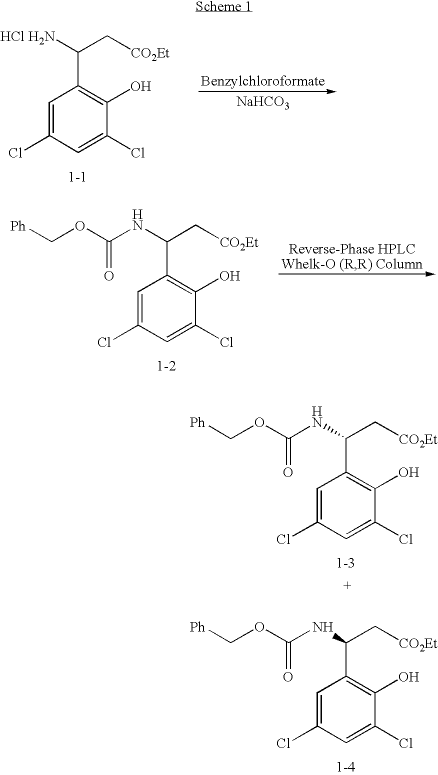

### Example 3

### (3R)-3-[( N-{3-amino-5-[(5-hydroxy-1,4,5,6-tetrahydropyrimidin-2-yl)amino]-benzoyl}glycyl)amino]-3-(3,5-dichloro-2-hydroxyphenyl)propanoic acid, monotrifluoroacetate

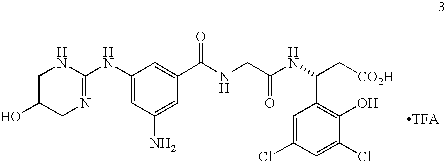 STEP1

### Ethyl (3R)-3-{[(benzyloxy)carbonyl]amino}-3-(3,5-dichloro-2-hydroxyphenyl )-propanoate

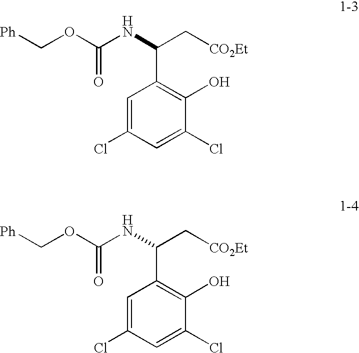

To the racemic amino acid ester hydrochloride 1-1 (procedure to prepare racemic compound was described in U.S. Pat. No 6,013,651) (50.0 g, 158.9 mmol) and NaHCO₃ (38.2 g, 454.5 mmol) was added CH₂Cl₂ (500 mL) and water (380 mL). The mixture was stirred at room temperature for 10 min with vigorous gas evolution. A solution of benzyl chloroformate (43.4 g, 222.8 mmol) in CH₂Cl₂ (435 mL) was added over 20 min with rapid stirring. After 40 min, the reaction mixture was poured into a separatory funnel and the organic solution collected. The aqueous phase was washed with CH₂Cl₂ (170 mL). The combined organic solution was dried (MgSO₄) and concentrated in vacuo. The resulting gummy solid was triturated with hexane and collected by filtration. The tan solid was dried in vacuo to give the desired racemic product, 62.9 g (96%). This material was subjected to reverse phase HPLC on a chiral column Whelk-O (R,R), (10 micron) using a 90:10 heptane:ethanol mobile phase to give pure enantiomers, 1-3 and 1-4. Optical purity was determined to be >98% using analytical hplc with similar solvent and conditions. ¹H NMR spectrum was consistent with proposed structure.

Step 2

### Ethyl (3R)-3-amino-3-(3,5-dichloro-2-hydroxyphenyl)propanoate

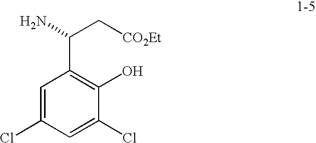

To a solution of the carbamate from STEP 1 (38.5 g, 93.4 mmol) in CH₂Cl₂ (380 mL) was added trimethylsilyl iodide (25.0 g, 125.0 mmol) in CH₂Cl₂ (80 mL) via canula. The orange solution was stirred at room temperature for 1.5 h. Methanol (20.0 mL, 500 mmol) was added dropwise and the solution stirred for 20 min. The reaction solution was concentrated in vacuo to give orange oil. The residue was dissolved in methyl t-butyl ether (450 mL) and extracted with 1M HCl (320 mL) and water (1×200 mL, 1×100 mL). The aqueous extracts were back washed with MTBE (130 mL). To the aqueous solution was added solid NaHCO₃ (40.1 g, 478 mmol) in small portions. The basified aqueous mixture was extracted with MTBE (1×1.0 L, 2×200 mL). The combined organic solution was washed with brine and concentrated in vacuo to give the desired product 1-5, 20.8 g (80%).¹H NMR (300 MHz, DMSO-d₆) δ 7.29 (d, 1H, J=2 Hz), 6.97 (d, 1H, J=2 Hz), 4.42 (t,1H, J=6 Hz), 4.04 (q, 2H, J=7 Hz), 2.71 (m, 2H), 1.13 (t, 3H, J=7 Hz). Anal. Calcd for C₁₁H₁₃Cl₂NO₃: C, 47.50; H, 4.71; N, 5.04. Found: C, 47.11; H, 4.66; N, 4.93.

Step 3.

### Ethyl (3R)-3-(3,5-dichloro-2-hydroxyphenyl)-3-[(N-{3-[(5-hydroxy-1,4,5,6-tetra-hydropyrimidin-2-yl)amino]-5-[(trifluoroacetyl)amino]benzoyl}glycyl)amino]-propanoate

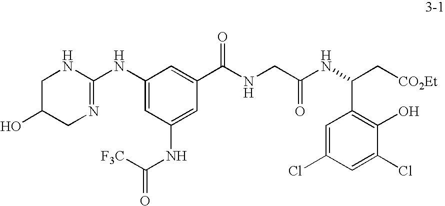

To a solution of the acid (670 mg, 1.75 mmol) in DMA (5 mL) at 0° C. was added isobutylchloroformate (0.25 mL, 1.9 mmol) and NMM (0.21 mL, 1.9 mmol). The reaction solution was kept at 0° C. for 15 minutes. A solution of ethyl (3R)-3-(3-bromo-5-chloro-2-hydroxyphenyl )-3-(glycylamino)-propanoate, hydrochloride (prepared from ethyl (3R)-3-amino-3-(3,5-dichloro-2-hydroxyphenyl)propanoate and BOC-Gly-Osu, using the procedure in Example 1, steps 2 and 3) (650 mg, 1.75 mmol) and NMM (0.21 mL, 1.9 mmol) in DMA (4 mL) was added. The reaction mixture was stirred at 0° C. for 30 minutes and then warmed to room temperature overnight. The mixture was concentrated in vacuo. The product was purified by reverse phase HPLC (90:10 H₂O/TFA:MeCN to 50:50 H₂O/TFA:MeCN) to give the desired product 3-1 (530 mg, 36%): ¹H NMR (300 MHz, DMSO-d₆) δ 9.91 (s, 1H), 9.75 (br s,10H), 9.06 (t, 1H, J=6 Hz), 8.90 (d, 1H, J=2 Hz), 8.62 (d, 1H, J=6 Hz), 8.59 (d, 1H, J=2 Hz), 8.45 (brs, 2H), 8.02 (dd, 1H), 7.53 (d, 1H, J=2 Hz), 7.28 (d, 1H, J=2 Hz), 5.43 (m, 1H), 4.11 (m, 1H), 4.05 (m, 4H), 3.92 (d, 2H, J=6 Hz), 3.34 (m, 2H), 3.17 (m, 2H), 2.75 (dd, 1H, J=4 Hz, J=15 Hz), 2.63 (dd, 1H, J=4 Hz, J=15 Hz), 1.12 (t, 3H, J=7 Hz). Anal. Calcd for C₂₆H₂₇F₃Cl₂N₆O₇+1.3 TFA: C, 42.32; H, 3.63; N, 10.35. Found: C, 41.97; H, 3.63; N, 10.21.

Step 4

### (3R)-3-[(N-{3-amino-5-[(5-hydroxy-1,4,5,6-tetrahydropyrimidin-2-yl)amino]-benzoyl}glycyl)amino]-3-(3,5-dichloro-2-hydroxyphenyl)propanoic acid, monotrifluoroacetate

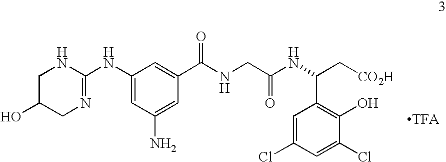

A solution of the product from STEP 1 (500 mg 0.7 mmol) in 1M NaOH solution (7 mL) was kept at room temperature for 3 h. The reaction solution was acidified to pH 5 with TFA. The mixture was purified by reverse phase HPLC (95:5 H₂O/TFA:MeCN to 60:40 H₂O/TFA:MeCN) to give the desired product 3 (325 mg, 57%): ¹H NMR (300 MHz, DMSO-d₆) δ 9.86 (br s,1H), 9.42 (s, 1H), 8.49 (d, 1H, J=6 Hz), 8.45 (t, 1H, J=6 Hz), 7.91 (br s, 2H), 7.37 (d, 1H, J=2 Hz), 7.19 (d, 1H, J=2 Hz), 6.90 (m, 1H), 6.77 (m, 1H), 6.46 (m, 1H), 5.39 (m, 1H), 4.03 (m, 1H), 3.83 (m, 3H), 3.30 (m, 2H), 3.12 (m, 2H), 2.68 (dd, 1H, J=4 Hz, J=15 Hz), 2.58 (dd, 1H, J=4 Hz, J=15 Hz). Anal. Calcd for C₂₂H₂₄Cl₂N₆O₇+2.5 TFA: C, 39.34; H, 3.24; N, 10.19. Found: C, 39.22; H, 3.47; N, 10.51.

### Example 4

### (3R)-3-(3,5-dichloro-2-hydroxyphenyl )-3-[( N-{3-hydroxy-5-[(5-hydroxy-1,4,5,6-tetrahydropyrimidinyl)amino]benzoyl}glycyl)amino]propanoic acid, monotrifluoroacetate

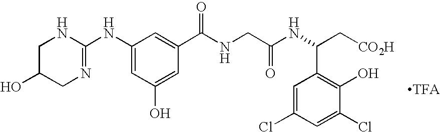 STEP1

### Ethyl (3R)-3-(3,5-dichloro-2-hydroxyphenyl)-3-[(N-{3-hydroxy-5-[(5-hydroxy-1,4,5,6-tetrahydropyrimidin-2yl)amino]benzoyl}glycyl)amino]-propanoate

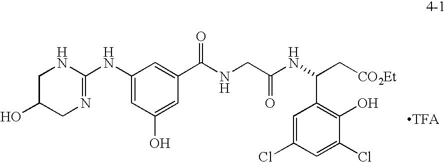

To a solution of the acid, 3-N-(5-hydroxytetrahydropyrimidinyl)-5-hydroxybenzoic acid (prepared according to U.S. Pat. No. 6,013,651, Example H, 772 mg, 2.7 mmol) in DMF (10 mL) at 0° C. was added isobutylchloroformate (0.35 mL, 2.7 mmol) and NMM (0.58 mL, 5.4 mmol). The reaction solution was kept at 0° C. for 15 minutes. A solution of ethyl (3R)-3-(3-bromo-5-chloro-2-hydroxyphenyl)-3-(glycylamino)-propanoate, hydrochloride, prepared as reported in Example 3 (1.0 g, 2.7 mmol) and NMM (0.29 mL, 2.7 mmol) in DMF (5 mL) was added. The reaction mixture was stirred at 0° C. for 15 minutes and then warmed to room temperature overnight. The mixture was concentrated in vacuo. The product was purified by reverse phase HPLC (90:10 H₂O/TFA:MeCN to 50:50 H₂O/TFA:MeCN) to give the desired product (625 mg, 31 %). ¹H NMR (300 MHz, DMSO-d₆) δ 10.07 (br s, 1H), 9.92 (br s, 1H), 9.80 (s, 1H), 8.67 (t, 1H, J=6 Hz), 8.54 (d, 1H, J=6 Hz), 8.22 (brs, 2H), 7.41 (d, 1H, J=2 Hz), 7.28 (d, 1H, J=2 Hz), 7.12 (m, 2H), 6.75 (m, 1H), 5.50 (m, 1H), 4.05 (m, 4H), 3.90 (m, 2H), 3.34 (m, 2H), 3.17 (m, 2H), 2.72 (m, 2H), 1.15 (t, 3H, J=7 Hz). Anal. Calcd for C₂₄H₂₇Cl₂N₅O₇+1.5 TFA: C, 43.86; H, 3.88; N, 9.47. Found: C, 43.87; H, 4.08; N, 9.61.

Step 2

### (β¹R)-3,5-dichloro-p-[[[[3-[(5-hydroxy-1,4,5,6-tetrahydro-2-pyrimidinyl)hydroxy]-benzoyl]amino]acetyl]amino]-2-hydroxybenzenepropanoic acid, monotrifluoroacetate

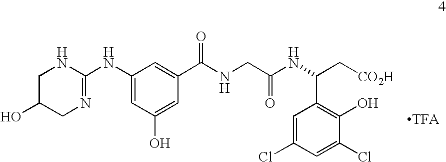

A solution of the product from STEP 1 (550 mg 0.74 mmol) in 1M NaOH solution (7 mL) was kept at room temperature for 2.5 h. The reaction solution was acidified to pH 5 with TFA. The mixture was purified by reverse phase HPLC (95:5 H₂O/TFA:MeCN to 60:40 H₂O/TFA:MeCN) to give the desired product (310 mg, 57%). ¹H NMR (300 MHz, DMSO-d₆) δ 12.37 (br s, 1H), 10.02 (s, 1H), 9.90 (s, 1H), 9.58 (s, 1H), 8.65 (t, 1H, J=6 Hz), 8.54 (d, 1H, J=6 Hz), 8.10 (br s, 2H), 7.41 (d, 1H, J=2 Hz), 7.23 (d, 1H, J=2 Hz), 7.13 (m, 2H), 6.75 (m, 1H), 5.43 (m, 1H), 4.08 (m, 1H), 3.90 (m, 2H), 3.34 (m, 2H), 3.15 (m, 2H), 2.65 (m, 2H). Anal. Calcd for C₂₂H₂₃Cl₂N₅O₇+1.7 TFA: C, 41.55; H, 3.39; N, 9.54. Found: C, 41.47; H, 3.36; N, 9.81.

### Example 5

### (3R)-3-[(N-{3-(aminocarbonyl)-5-[(5-hydroxy-1,4,5,6-tetrahydropyrimidin-2-yl)amino]benzoyl}glycyl)amino]3-(3,5-dichloro-2-hydroxyphenyl)propanoic acid

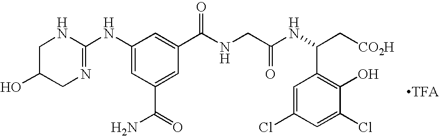 Step 1

### Ethyl (3R)-3-({N-[3-(aminocarbonyl)-5-nitrobenzoyl]glycyl}amino)-3-(3,5-dichloro-2-hydroxyphenyl)propanoate

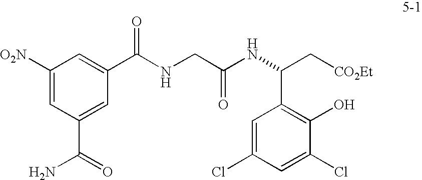

To a solution of 3-carboxamido-5-nitrobenzoic acid (610 mg, 2.9 mmol) in DMF (10 mL) at 0° C. was added isobutylchloroformate (0.38 mL, 2.9 mmol) and NMM (0.32 mL, 2.9 mmol). The reaction solution was kept at 0° C. for 5 minutes. A solution of ethyl (3R)-3-(3-bromo-5-chloro-2-hydroxyphenyl)-3-(glycylamino)-propanoate, hydrochloride, prepared as reported in Example 3 (1.1 g, 2.9 mmol) and NMM (0.32 mL, 2.9 mmol) in DMF (5 mL) was added. The reaction solution was warmed to room temperature overnight. The mixture was concentrated in vacuo. The residue was dissolved in EtOAc (100 mL) and washed with water and brine. The organic solution was concentrated and the product was purified by chromatography (90:9:1 CH₂Cl₂:MeOH:NH₄OH) to give the desired product 5-1 (1.1 g, 72%): ¹H NMR (300 MHz, CD₃OD) δ 8.90 (m, 1H), 8.77 (m, 1H), 7.25 (d, 1H, J=2 Hz), 7.18 (d, 1H, J=2 Hz), 5.59 (m, 1H), 4.10 (m, 4H), 2.86 (m, 2H), 1.19 (t, 3H, J=6 Hz).

Step 2

### Ethyl (3R)-3-({N-[3-amino-5-(aminocarbonyl)benzoyl]glycyl}amino)-3-(3,5-dichloro-2-hydroxyphenyl)propanoate

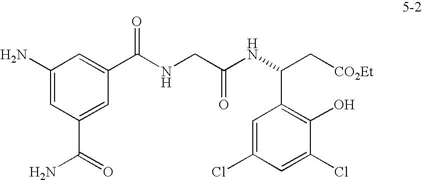

To a solution of the product from STEP 1 (1.1 g, 2.1 mmol) in EtOH with 5% Pt/C was hydrogenated for 4 h at room temperature and atmospheric pressure. The catalyst was removed by filtration and the filtrate was concentrated in vacuo to give the desired product 5-2 (1.1 g).

Step 3

### (β¹R)-3,5-dichloro-β-[[[[3-[(5-hydroxy-1,4,5,6-tetrahydro-2-pyrimidinyl)amino-acetyl]benzoyl]amino]acetyl]amino]-2-hydroxybenzenepropanoic acid, ethyl ester monotrifluoroacetate

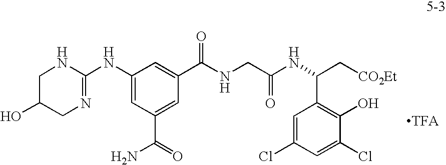

To a solution of the product from STEP 2 (1.1 g, 2.2 mmol) in DMF (12 mL) was added di(tert-butyl) 5-[(tert-butoxycarbonyl)oxy]-2-thioxodihydro-pyrimidine-1,3(2H,4H)-dicarboxylate (1.15 g, 2.6 mmol), triethylamine (0.62 mL, 4.4 mmol) and mercuric chloride (783 mg, 2.8 mmol). The mixture was heated at 70° C. for 3 h. The mixture was cooled to room temperature and filtered through celite. The celite pad was washed with EtOAc. The organic solution was concentrated in vacuo and the residue dissolved in CH₂Cl₂ (8 mL) and TFA (8 mL). The solution was kept at room temperature for 1.5 h and concentrated in vacuo. The residue was purified by reverse phase HPLC (90:10 H₂O/TFA:MeCN to 50:50 H₂O/TFA:MeCN) to give the desired product (180 mg, 14%). ¹H NMR (300 MHz, DMSO-d₆) δ 9.91 (s, 1H), 9.78 (s, 1H), 8.83 (m, 1H), 8.59 (m, 1H), 8.25 (m, 3H), 8.05 (m, 1H), 7.80 (m, 2H), 7.60 (m, 1H), 7.42 (d, 1H, J=2 Hz), 7.28 (d, 1H, J=2 Hz), 5.50 (m, 1H), 4.07 (m, 4H), 3.95 (m, 2H), 3.33 (m, 2H), 3.18 (m, 2H), 2.71 (m, 2H), 1.14 (t, 3H, J=6 Hz).

Step 4

### (β¹R)-3,5-dichloro-β-[[[[3-[(5-hydroxy-1,4,5,6-tetrahydro-2-pyrimidinyl)amino-acetyl]benzoyl]amino]acetyl]amino]-2-hydroxybenzenepropanoic acid, monotrifluoroacetate

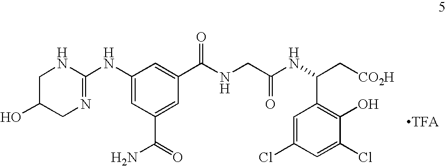

A solution of the product from STEP 3 (180 mg 0.3 mmol) in 1M NaOH solution (2.5 mL) and methanol 4 (mL) was kept at room temperature for 3 h. Volatiles were removed in vacuo and the aqueous solution was acidified to pH 5 with TFA. The mixture was purified by reverse phase HPLC (95:5 H₂O/TFA:MeCN to 60:40 H₂O/TFA:MeCN) to give the desired product (86 mg, 86%): ¹H NMR (300 MHz, DMSO-d₆) δ 12.37 (br s, 1H), 9.90 (s, 1H), 9.72 (s, 1H), 8.82 (t, 1H, J=6 Hz), 8.26 (m, 1H), 8.21 (m, 2H), 8.05 (m, 1H), 7.80 (m, 2H), 7.60 (m, 1H), 7.41 (d, 1H, J=2 Hz), 7.25 (d, 1H, J=2 Hz), 5.47 (m, 1H), 4.11 (m, 1H), 3.95 (m, 2H), 3.33 (m, 2H), 3.18 (m, 2H), 2.67 (m, 2H). Anal. Calcd for C₂₂H₂₃Cl₂N₅O₇+1.5 TFA: C, 42.29; H, 3.48; N, 11.38. Found: C, 42.25; H, 3.11; N, 11.54.

### Example 6

### (3R)-3-(3-bromo-5-chloro-2-hydroxyphenyl)-3-[(N-{3-hydroxy-5-[(5-hydroxy-1,4,5,6-tetrahydropyrimidin-2-yl)amino]benzoyl}-N-methyl-glycyl)amino]propanoic acid, monotrifluoroacetate

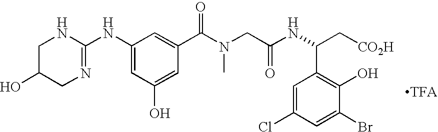 Step 1

### (3R)-3-(3-bromo-5-chloro-2-hydroxyphenyl)-3-[(N-{3-hydroxy-5-[(5-hydroxy-1,4,5,6-tetrahydropyrimidin-2-yl )amino]benzoyl}-N-methyl-glycyl)amino]propanoic acid, ethyl ester mono trifluoroacetate

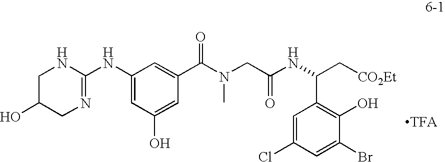

To a solution of 3-N-(5-hydroxytetrahydropyrimidinyl)-5-hydroxybenzoic acid (prepared according to U.S. Pat. No. 6,013,651, Example H, 468 mg, 1.6 mmol) in DMA (7 mL) at 0° C. was added isobutylchloroformate (0.21 mL, 1.6 mmol) and NMM (0.16 mL, 1.6 mmol). The reaction solution was kept at 0° C. for 20 minutes. A solution of ethyl (3R)-3-(3-bromo-5-chloro-2-hydroxyphenyl)-3-(glycylamino)-propanoate, hydrochloride prepared as in Example 1, STEP 3 (700 mg, 1.6 mmol) and NMM (0.16 mL, 1.6 mmol) in DMA (7 mL) was added. The reaction mixture was stirred and warmed to room temperature overnight. The mixture was concentrated in vacuo. The product was purified by reverse phase HPLC (90:10 H₂O/TFA:MeCN to 50:50 H₂O/TFA:MeCN) to give the desired product (85 mg). ¹H NMR was consistent with the proposed structure.

Step 2

### (3R)-3-(3-bromo-5-chloro-2-hydroxyphenyl)-3-[(N-{3-hydroxy-5-[(5hydroxy-1,4,5,6-tetrahydropyrimidin-2-yl)amino]benzoyl}-N-methylglycyl)-amino]propanoic acid, monotrifluoroacetate

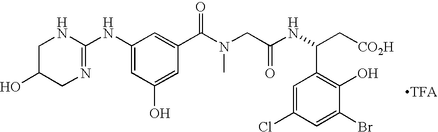

A solution of the product from STEP 1 (85 mg) in 1M NaOH solution (2 mL) was kept at room temperature for 2 h. Volatiles were removed in vacuo and the aqueous solution was acidified to pH 4 with TFA. The mixture was purified by reverse phase HPLC (95:5 H₂O/TFA:MeCN to 60:40 H₂O/TFA:MeCN) to give the desired product (61 mg, 5% for the two steps). Anal. Calcd for C₂₃H₂₅BrClN₅O₇+1.8 TFA: C, 39.73; H, 3.36; N, 8.71. Found: C, 39.52; H, 3.47; N, 9.04. ¹H NMR was consistent with the proposed structure.

### Example 7

### (3R)-3-(3-bromo-5-chloro-2-hydroxyphenyl)-3-{[N-({5-[(5-hydroxy-1,4,5,6-tetrahydropyrimidin-2-yl)amino]pyridin-3-yl}carbonyl)-N-methylglycyl]-amino}propanoic acid, monotrifluoroacetate

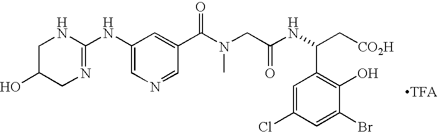 Step 1

### (3R)-3-(3-bromo-5-chloro-2-hydroxyphenyl )-3-{[N-({5-[(5-hydroxy-1,4,5,6-tetra-hydropyrimidin-2-yl)amino]pyridin-3-yl}carbonyl)-N-methylglycyl]amino}-propanoic acid, ethyl ester monotrifluoroacetate

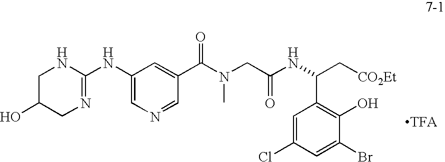

To a solution N-(5-hydroxytetrahydropyrimidinyl)-3-aminonicotinic acid described in Example 2, steps 1-3, (427 mg, 1.2 mmol) in DMA (5 mL) at 0° C. was added isobutylchloroformate (0.16 mL, 1.2 mmol) and NMM (0.14 mL, 1.2 mmol). The reaction solution was kept at 0° C. for 20 min. A solution of ethyl (3R)-3-(3-bromo-5-chloro-2-hydroxyphenyl)-3-(glycylamino)-propanoate, hydrochloride prepared as in Example 1, STEP 3 (525 mg, 1.2 mmol) and NMM (0.14 mL, 1.2 mmol) in DMA (5 mL) was added. The reaction mixture was stirred and warmed to room temperature overnight. The mixture was concentrated in vacuo. The product was purified by reverse phase HPLC (90:10 H₂O/TFA:MeCN to 50:50 H₂O/TFA:MeCN) to give the desired product (110 mg). ¹H NMR was consistent with the proposed structure.

Step 2

### (3R)-3-(3-bromo-5-chloro-2-hydroxyphenyl)-3-{[N-({5-[(5-hydroxy-1,4,5,6-tetrahydropyrimidin-2-yl)amino]pyridin-3-yl}carbonyl)-N-methylglycyl]amino}-propanoic acid, monotrifluoroacetate

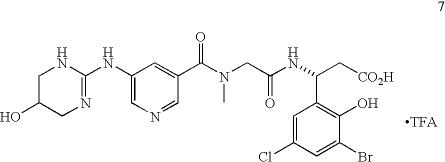

A solution of the product from STEP 1 (110 mg) in 1M NaOH solution (2.5 mL) was kept at room temperature for 2 h. Volatiles were removed in vacuo and the aqueous solution was acidified to pH 4 with TFA. The mixture was purified by reverse phase HPLC (95:5 H₂O/TFA:MeCN to 60:40 H₂O/TFA:MeCN) to give the desired product 7 (70 mg, 7% for the two steps). Anal. Calcd for C₂₂H₂₄BrClN₆O₆+2.5 TFA and 2.0 H₂O: C, 35.84; H, 3.40; N, 9.29. Found: C, 35.90; H, 3.65; N, 9.23. ¹H NMR was consistent with the proposed structure.

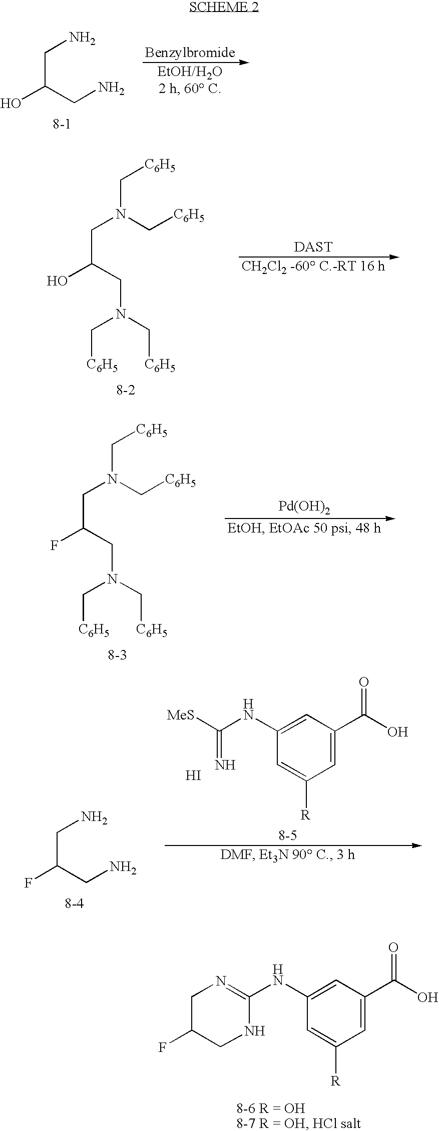

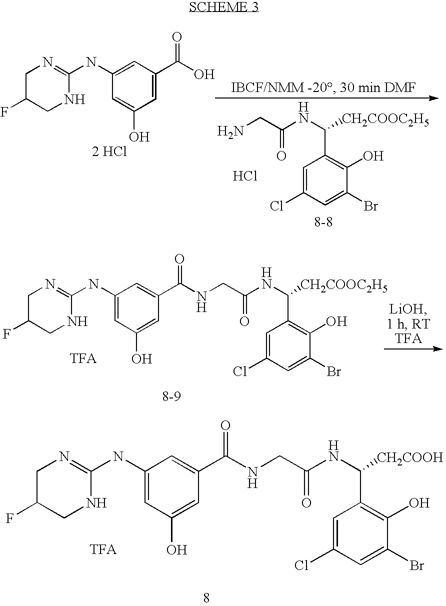

### Example 8

### (β¹R)-3-bromo-5-chloro-β-[[[[3-[(5-fluoro-1,4,5,6-tetrahydro-2-pyrimidinyl)amino]-5-hydroxybenzoyl]amino]acetyl]amino]-2-hydroxybenzenepropanoic acid, monotrifluoroacetate.

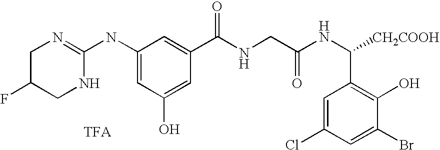 Step 1

### N,N,N,N-Tetrabenzyl-1,3diamino-2-hydroxypropane

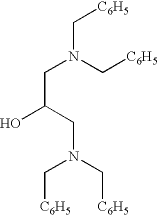

A mixture of 1,3diamino-2-hydroxypropane (2.5 g) in ethanol (45 mL) and water (15 mL), containing potassium carbonate (11.5 g) and benzyl-bromide (14.6 mL) was heated at 60° C. for 2 h with vigorous stirring. Ethanol was removed under reduced pressure, and the residue was partitioned between water (100 mL) and EtOAc (200 mL). The organic phase was washed with water, dried (Na₂SO₄), and concentrated to dryness to give 12.4 g of the desired product 8-2 as a colorless syrup: ¹H-NMR (CDCl₃) δ 7.31 (m, 20H), 3.83 (t, 1H), 3.67 (d, 4H, J=13.5 Hz), 3.5 (d, 4H, J=13.5 Hz), 3.4 (s,1H), 2.45 (d, 4H, J=6.0 Hz); HR-MS (ES) m/z calcd C₃₁H₃₄N₂O (MH⁺) 451.2749. Found 451.2721.

Step 2

### N,N,N,N-Tetrabenzyl-1,3diamino-2-fluoropropane

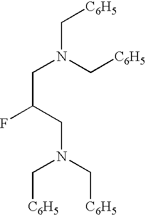

To a solution of N,N,N,N-Tetrabenzyl-1,3diamino-2-hydroxypropane (25.0 g) in dichloromethane (200 mL), at −65° C., was added dropwise a solution of DAST (8.1 mL) in dichloromethane (25 mL) over a period of 15 min. with vigorous stirring under an atmosphere of argon. The reaction mixture was gradually allowed to warm to 15° C. overnight. It was cooled to −40° C. and poured in portions into saturated NaHCO₃ solution containing ice and the products were extracted with dichloromethane (2×200 mL). The combined organic extracts were washed with water, dried (Na₂SO₄), and concentrated to dryness to give orange colored syrup. This was dissolved in EtOAc (150 mL), added activated charcoal (5 g), stirred for 30 min. and filtered through celite. The filtrate was concentrated to dryness and the residue was dried invacuo to afford 23.5 g of 8-3 as a thick orange syrup: ¹H-NMR (CDCl₃) δ 7.28 (m, 20H), 4.92 and 4.75 (m, 1H), 3.67 (d, 4H, J=13.8 Hz), 3.52 (d, 4H, J=13.8 Hz), 2.6 ( m, 4H); HR-MS (ES) m/z calcd for C₃₁H₃₄N₂F (MH⁺) 453.2706. Found 453.2709.

Step 3

### 1,3diamino-2-fluoropropane

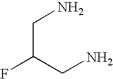

A solution of N,N,N,N-Tetrabenzyl-1,3 diamino-2-fluoropropane ( 20.0 g) in EtOAc (50.0 mL) and MeOH (50.0 mL), was hydrogenated at 50 psi in the presence of 20% Pd(OH)₂ on carbon (10 g) for 16 h. The catalyst was removed by filtration and washed with ethanol. The combined filtrate and washings were again hydrogenated at 50 psi in the presence of 20% Pd(OH)₂ on carbon (10 g) for 24 h. The catalyst was removed by filtration, and it was washed with 10% water in ethanol (100 mL). The filtrate and the washings were combined, and concentrated to dryness under reduced pressure to afford 3.9 g of 8-4 as a colorless syrup: ¹H-NMR (CD₃OD) δ 4.62 and 4.45 (m, 1H), 2.77 (m, 2H), and 2.7 (m, 2H).

Step 4

### 3-[(5-fluoro-1,4,5,6-tetrahydro-2-pyrimidinyl)amino]-5-hydroxybenzoic acid

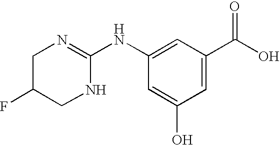

To a solution of 1,3 diamino-2-fluoropropane (8.3 g), in DMF (100 mL), was added triethylamine (10.0 mL), followed by the addition of S-methyl-isothiourea (16 g) and the resulting mixture was stirred at room temperature. After 30 mins of stirring, the reaction mixture was heated to 90° C., under anhydrous conditions for 3 h, when a light brown precipitate was obtained. DMF was distilled in vacuo and the residue was triturated with water, and filtered. The precipitate was washed thoroughlly with water, followed by acetonitrile, and dried in a desiccator in vacuo to afford 8.0 g of product as a light brown powder.

Step 5

### 3-[(5-fluoro-1,4,5,6-tetrahydro-2-pyrimidinyl)amino]-5-hydroxybenzoic acid hydrochloride

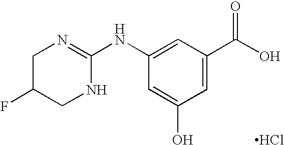

To a chilled suspension of the product of STEP 4 (0.265g, 0.001 mol) in anhydrous THF (5 mL) was added HCl/dioxane (4N, 0.52 mL, 2 equiv) and stirred cold for 1 h. The solvent was removed under reduced pressure to afford the desired hydrochloride salt 8-7 after drying (0.339 g, 99%): ¹H-NMR (CD₃OD) δ 7.38 (m, 1H), 7.33 (m, 1H), 5.15 (m, 1H), 3.63 -3.4 (m, 4H); HR-MS m/z (MH⁺) calcd C₁₁H₁₃N₃FO₃ (MH⁺) 254.0941. Found 254.0944.

Step 6

### (β¹R)-3-bromo-5-chloro-β[[[[3-[(5-fluoro-1,4,5,6-tetrahydro-2-pyrimidinyl)amino]-5-hydroxybenzoyl]amino]acetyl]amino]-2-hydroxybenzenepropanoic acid, ethyl ester monotrifluoroacetate.

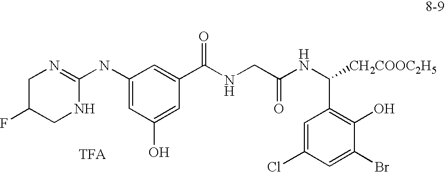

To a suspension of 3-[(5-fluoro-1,4,5,6-tetrahydro-2-pyrimidinyl)amino]-5-hydroxybenzoic acid hydrochloride in DMF (40 mL) at −15° C., was added dropwise, a solution of isobutylchloroformate (4.0 mL) in dichloromethane (10 mL), followed by the addition of a solution of N-methylmorpholine (3.5 mL) in DMF (10 mL). The reaction mixture was stirred at −15 C for for 30 mins, and then added a solution of ethyl (3R)-3-(3-bromo-5-chloro-2-hydroxyphenyl)-3-(glycylamino)-propanoate, hydrochloride prepared as in Example 1, STEP 3, generated by the addition of N-methylmorpholine (2.9 ml) to a solution of the HCl salt (10.9 g) in DMF (25 ml) at 5 C. The resulting mixture was stirred at −15 C for 30 min, and at room temperature for 6 h. The solvents were distilled in vacuo, and residue was purified by reverse-phase HPLC using 10-90% acetonitrile/water at flow rate of 100 mL/min. The appropriate fractions were combined and freeze dried to obtain 11.5 g of the desired ester as a white powder.

Step 7

### (β¹R)-3-bromo-5-chloro-β-[[[[3-[(5-fluoro-1,4,5,6-tetrahydro-2-pyrimidinyl)-amino]-5-hydroxybenzoyl]amino]acetyl]amino]-2-hydroxy-benzene-propanoic acid, monotrifluoroacetate.

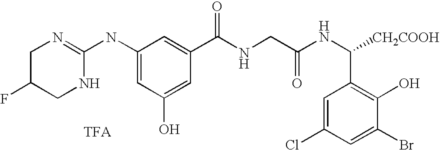

The ester (11.5 g) from STEP 1 was stirred with 1M LiOH (55.0 mL) at room temperature for 1.5 h. The solution was cooled, acidified with trifluoroacetic acid, and the preciptate was purified by reverse-phase HPLC using using 10-90% acetonitrile/water at flow rate of 100 mL/min. The appropriate fractions were combined and freeze dried to obtain 10.2 g of the desired acid 8 as a white powder: ¹H-NMR (CD₃OD) δ 7.41 (s, 1H), 7.24 (d, 1H, J=1.8 Hz), 7.25 (s, 1H) 7.21 (s, 1H), 7.17 (s, 1H), 6.81 (s, 1H), 5.55 (t, 1H, J=6.0Hz), 5.20 (m, 1H, _{JH,}=46.4.0 Hz), 4.06 (s. 2H, ), 3.7-3.41 (m, 4H), 2.85 (ab q, 2H, J₁=5.6 Hz, J₂=9.6 Hz), 1.18 (t, 3H, J=5.4 Hz), MS m/z (MH⁺) calcd C₂₂H₂₃N₅FClBrO₆ (MH⁺) 586.0504. Found 586.0495.

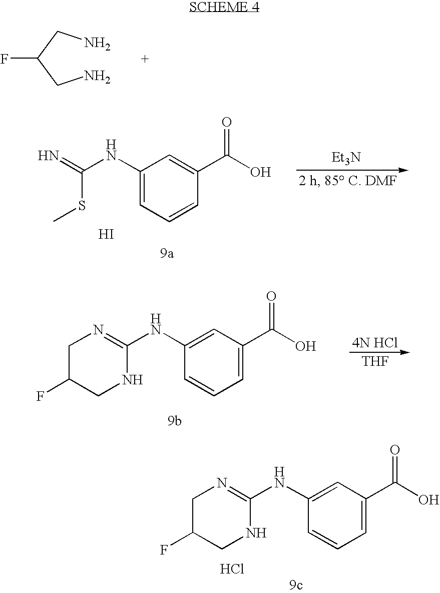

### Example 9

### (β¹R)-3-bromo-5-chloro-β-[[[[3-[(5-fluoro-1,4,5,6-tetrahydro-2-pyrimidinyl)-amino]benzoyl]amino]acetyl]amino]-2-hydroxybenzenepropanoic acid, monotrifluoroacetate

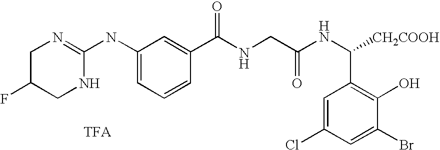 Step 1

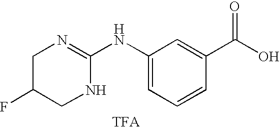

To a solution of 1,3-diamino-2-fluoropropane (2.5 g, 0.0271 mol, 2 equiv) in anhydrous DMF (13.0 mL) containing triethylamine (2.05 g, 0.0203 mol, 1.5 equiv) was added compound (2) (4.6 g, 0.0135 mol, 1 equiv) in one portion. The solution was heated at 85° C. under anhydrous conditions for 2 h. After 1 h heating, the solution became turbid which increased during heating. The solvent was removed in vacuo to afford a light beige residue. The resulting residue was diluted with water, chilled, and filtered. The residue was washed with water, followed by acetonitrile and dried in a desiccator in vacuo to afford the desired product (2.31 g, 72%): ¹H-NMR (CD₃CD, 400 Hz) δ 7.97 (m, 2H), 7.56 (m, 2H), 5.26 (m, 1H, _{JH}=44 Hz), 3.65-3.5 (m, 4H); HR-MS (ES) m/z calcd C11 H₁₃ N₃FO₂ (MH⁺) 238.0992. Observed: 238.0996
Step 2

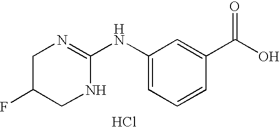

To a chilled suspension of STEP 1 (2.31g, 0.0097 mol) in anhydrous THF (10 mL) was added HCl/dioxane (4N, 4.85 mL, 2 eq) and stirred at 10° C. for 1 h. The solvent was removed under reduced pressure and the resulting residue was triturated with ether (2×10 mL), and filtered, and dried in vacuo to afford the desired product (2.80 g, 93% yield).

Step 3

### (β¹R)-3-bromo-5-chloro-β-[[[[3-[(5-fluoro-1,4,5,6-tetrahydro-2-pyrimidinyl)amino]-benzoyl]amino]acetyl]amino]-2-hydroxybenzenepropanoic acid, ethyl ester monotrifluoroacetate

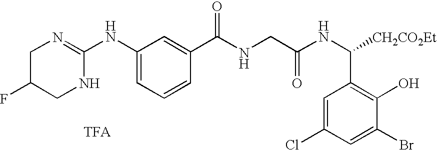

To a solution of 3-[(5-fluoro-1,4,5,6-tetrahydro-2-pyrimidinyl)amino]benzoic acid, monohydrochloride salt 9 c (0.50 g, 0.00161 mol) in anhydrous DMF (5 mL) at −5° C. was added diisopropylethylamine (DIEA, 0.23g, 0.0018 mol) followed by the addition of HBTU (0.64 g, 0.00168 mol). After stirring this mixture under argon atmosphere for 1 hour at −5° C., solution was allowed to warm up to RT and stirred for 1 hour. After activating the ester, a cold solution of ethyl (3R)-3-(3-bromo-5-chloro-2-hydroxyphenyl)-3-(glycylamino)-propanoate, hydrochloride prepared as in Example 1, STEP 3 (0.655 g, 0.00157 mol) in anhydrous DMF (5 mL) and N-methylmorpholine (0.19g, 0.0019 mol) was added. The pH of the resulting mixture was 7 at 1 hour, and was allowed to stir at room temperature overnight.

Solvent was distilled in vacuo and the residue was purified by reverse phase HPLC to yield (after lyophilization) the desired ester as a white powder, 0.7 g (64% yield): ¹H-NMR (CD₃OD, 400 Hz) δ 7.6 (d, 1H), 7.73 (t, 1H), 7.55 (t, 1H), 7.41 (m, 2H), 7.24 (d, 1H), 5.57 (t, 1H), 5.25 (m, 1H, _{JH}=48 Hz), 4.07 (m, 4H), 3.64 (m, 4H), 2.88 (m, 2H), 1.17 (t, 3H); HR-MS (ES) m/z calcd for C₂₄H₂₆N₅FClBrO₅ (MH⁺) 598.0868. Observed: 598.0850.

Step 4

### (β¹R)-3-bromo-5-chloro-β-[[[[3-[(5-fluoro-1,4,5,6-tetrahydro-2-pyrimidinyl)amino]-enzoyl]amino]acetyl]amino]-2-hydroxybenzenepropanoic acid, monotrifluoroacetate

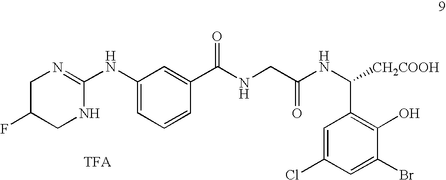

The ester from STEP 3 (0.7 g, 0.00098 mol) was stirred with 1M LiOH (4 mL) for 2 h at room temperature. The pH was adjusted to 2 with trifluoro-acetic acid and the product was purified by reverse phase HPLC to provide (after lyophilization) the desired acid 9 as a white powder (0.4 g, 59%): ¹H-NMR (CD₃OD, 400 Hz) δ 7.79 (d, 1H), 7.73 (t, 1H), 7.55 (t, 1H), 7.42 (m, 2H), 7.24 (d, 1H), 5.55 (t, 1H), 5.26 (m, 1H, _{JH}=46.4 Hz), 4.08 (s, 2H), 3.64 to 3.3 (m, 4H), 2.86 (m, 2H); HR-MS (ES) m/z calcd for C₂₂H₂₂N₅FO₅ClBr (MH⁺) 570.0555. Observed: 570.0550.

### Example 10

### (β¹R)-3,5-dichloro-β-[[[[3-[(5-fluoro-1,4,5,6-tetrahydro-2-pyrimidinyl)amino]-benzoyl]amino]acetyl]amino]-2-hydroxybenzenepropanoic acid, monotrifluoroacetate

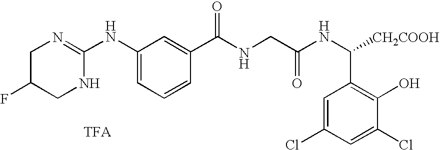 Step 1

### (β¹R)-3,5-dichloro-β-[[[[3-[(5-fluoro-1,4,5,6-tetrahydro-2-pyrimidinyl )amino]-benzoyl]amino]acetyl]amino]-2-hydroxybenzenepropanoic acid, ethyl ester monotrifluoroacetate

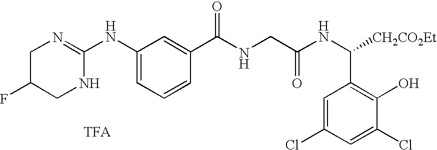

To a solution of 3-[(5-fluoro-1,4,5,6-tetrahydro-2-pyrimidinyl)amino]benzoic acid, monohydrochloride salt, prepared as in Example 9 (0.51 g, 0.00164 mol) in anhydrous DMF (5 mL) at −5° C. was added diisopropylethylamine (DIEA, 0.53 g, 0.0041 mol) followed by the addition of HBTU (0.92 g, 0.00242 mol). After stirring this mixture under argon atmosphere for 1 hour at −5° C., solution was allowed to warm up to RT and stirred for 1 hour. After activating the ester, a cold solution of ethyl (3R)-3-(3,5-dichloro-2-hydroxyphenyl)-3-(glycylamino)-propanoate, hydrochloride prepared as in Example 3 (0.566 g, 0.00152 mol) in anhydrous DMF (10 mL) and N-methylmorpholine (0.20g, 0.0020 mol) was added. The pH of the resulting mixture was 7 at 1 hour, and was allowed to stir at room temperature overnight. Solvent was distilled in vacuo and the residue was purified by reverse phase HPLC to yield (after lyophilization) the desired ester as a white powder, 0.28 g (27% yield): ¹H-NMR (CD₃OD, 400 Hz) δ 7.73 (m, 2H), 7.55 (t, 2H), 7.20 (m, 1H), 7.19 (d, 1H), 5.57 (t, 1H), 5.20 (m, 1H, _{JH}=48 Hz), 4.09 (m, 4H), 3.64 (m, 4H), 2.88 (m, 2H), 1.19 (t, 3H); HR-MS (ES) m/z calcd for C₂₄H₂₅N₅O₅FCl₂ (MH⁺) 554.1373. Observed: 554.1392.

Step 2

### (β¹R)-3,5-dichloro-β-[[[[3-[(5-fluoro- 1,4,5,6-tetrahydro-2-pyrimidinyl)amino)-benzoyl]amino]acetyl]amino]-2-hydroxybenzenepropanoic acid, monotrifluoroacetate

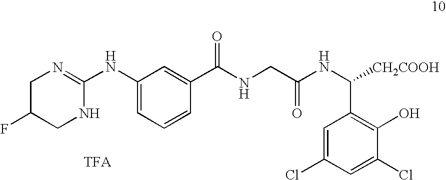

The ester 10-2 (0.28 g, 0.00042 mol) was stirred with 1M LiOH (3 mL) for 3 h at room temperature. The pH was adjusted to 2 with trifluoroacetic acid and the product was purified by reverse phase HPLC to provide (after lyophilization) the desired acid 10 as a white powder, 0.040 g (15%): ¹H-NMR (CD₃OD, 400 Hz) δ 7.73 (m, 1H), 7.55 (t, 1H), 7.40 (m, 1H), 7.21 (d, 1H), 7.20 (d, 1H,), 5.53 (t, 1H), 5.26 (m, 1H, _{JH}=48 Hz), 4.07 (s, 2H), 3.64 (m, 4H), 2.85 (m, 2H); HR-MS (ES) m/z calcd for C₂₂H₂₂N₅O₅FCl₂ (MH⁺) 526.1060. Observed: 526.1054.

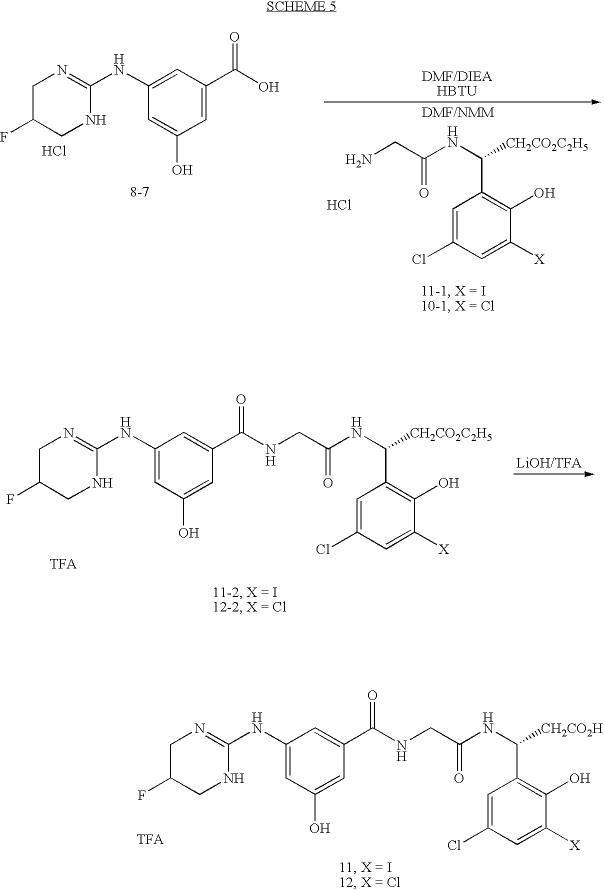

### Example 11

### (β¹R)-3-lodo-5-chloro-β-[[[[3-[(5-fluoro-1,4,5,6-tetrahydro-2-pyrimidinyl)-amino]-5-hydroxybenzoyl]amino]acetyl]amino]-2-hydroxybenzenepropanoic acid, monotrifluoroacetate

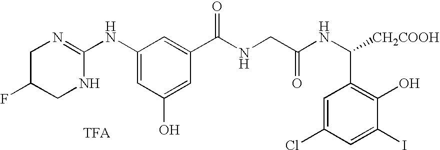 Step 1

### (β¹R)-3-lodo-5-chloro-β-[[[[3-[(5-fluoro-1,4,5,6-tetrahydro-2-pyrimidinyl)-amino]-5-hydroxybenzoyl]amino]acetyl]amino]-2-hydroxybenzene-propanoic acid, ethyl ester monotrifluoroacetate

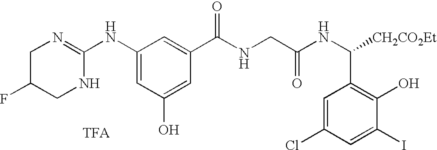

To a solution of 3-[(5-fluoro-1,4,5,6-tetrahydro-2-pyrimidinyl)amino] 5-hydroxy-benzoic acid, monohydrochloride salt prepared as in Example 8 (0.65 g, 0.00225 mol) in anhydrous DMF (5 mL) at −5° C. was added diisopropylethylamine (DIEA, 0.30 g, 0.00235 mol) followed by the addition of HBTU (0.852 g, 0.00225 mol). After stirring this mixture under argon atmosphere for 1 hour at −5° C., solution was allowed to warm up to RT and stirred for 1 hour. After activating the ester, a cold solution of ethyl R-3-(N-gly)-amino-3-(5-chloro-2-hydroxy-3-iodophenyl)propionate hydrochloride, prepared as in Example 60, (0.98 g, 0.00212 mol) in anhydrous DMF (10 mL) and N-methylmorpholine (0.21 g, 0.0021 mol) was added. The pH of the resulting mixture was 7 at 1 hour, and was allowed to stir at room temperature overnight. Solvent was distilled in vacuo and the residue was purified by reverse phase HPLC to yield (after lyophilization) the desired ester 11-2 as a white powder (0.49 g, 30%): ¹H-NMR (CD₃OD, 400 Hz) δ 7.6 (d, 1H), 7.25 (t, 1H), 7.20 (m, 2H), 7.16 (d, 1H), 6.81 (m, 1H), 5.57 (t, 1H), 5.20 (m, 1H, _{JH}=48 Hz), 4.07 (m, 4H), 3.64 to 3.49 (m, 4H), 2.89 (m, 2H), 1.19 (t, 3H); HR-MS (ES) m/z calcd for C₂₄H₂₆N₅IFClO₆ (MH⁺) 662.0676. Observed: 662.0654.

Step 2

### (β¹R)-3-lodo-5-chloro-β-[[[[3-[(5-fluoro-1,4,5,6-tetrahydro-2-pyrimidinyl)-amino]-5-hydroxybenzoyl]amino]acetyl]amino]-2-hydroxybenzene-propanoic acid, mono-trifluoroacetate

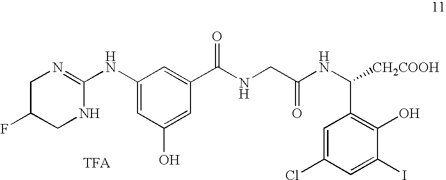

The ester from STEP 1 (0.40 g, 0.0005 mol) was stirred with 1M LiOH (3 mL) for 1 hour at room temperature. The pH was adjusted to 2 with trifluoroacetic acid and the product was purified by reverse phase HPLC to provide (after lyophilization) the desired acid 11 as a white powder (0.193 g, 50%): ¹H-NMR (CD₃OD, 400 Hz) δ 7.6 (d, 1H), 7.27 (t, 1H), 7.20 (t, 1H), 7.18 (t, 1H), 6.81 (t, 1H), 5.53 (t, 1H), 5.26 (m, 1H, _{JH}=48 Hz), 4.04 (s, 2H), 3.64 to 3.3 (m, 4H), 2.85 (m, 2H); HR-MS (ES) m/z calcd for C₂₂H₂₂N₅IFClO₆ (MH⁺) 634.0366. Observed: 634.0343.

### Example 12

### (β¹R)-3,5-dichloro-β-[[[[3-[(5-fluoro-1,4,5,6-tetrahydro-2-pyrimidinyl)amino]-5-hydroxybenzoyl]amino]acetyl]amino]-2-hydroxybenzenepropanoic acid, monotrifluoroacetate

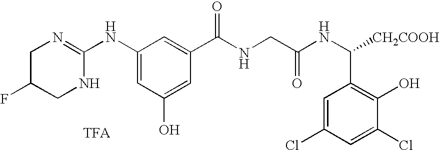 Step 1

### (β¹R)-3,5-dichloro-β-[[[[3-[(5-fluoro-1,4,5,6-tetrahydro-2-pyrimidinyl)amino]-5-hydroxybenzoyl]amino]acetyl]amino]-2-hydroxybenzenepropanoic acid, ethyl ester monotrifluoroacetate

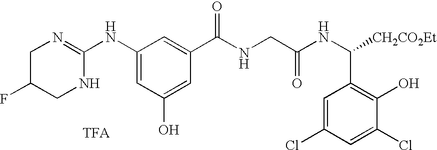

To a solution of 3-[(5-fluoro-1,4,5,6-tetrahydro-2-pyrimidinyl)amino]5-hydroxy-benzoic acid, monohydrochloride salt prepared as in Example 8 (0.49 g, 0.00169 mol) in anhydrous DMF (5 mL) at −5° C. was added diisopropylethylamine (DIEA, 0.25 g, 0.00201 mol) followed by the addition of HBTU (0.768 g, 0.00202 mol). After stirring this mixture under argon atmosphere for 1 hour at −5° C., solution was allowed to warm up to RT and stirred for 1 h. To this, ethyl (3R)-3-(3,5-dichloro-2-hydroxyphenyl)-3-(glycylamino)-propanoate, hydrochloride prepared as in Example 3 (0.62 g, 0.00167 mol) in anhydrous DMF (5 mL) and N-methylmorpholine (0.20 g, 0.0020 mol) was added. The pH of the resulting mixture was 7 at 1 hour, and was allowed to stir at room temperature overnight. The solvent was distilled in vacuo and the residue was purified by reverse phase HPLC to yield (after lyophilization) the desired ester 12-2 as a white powder (0.42 g, 37% yield): ¹H-NMR (CD₃OD, 400 Hz) δ 7.26 (t, 1H), 7.21 (m, 2H), 7.16 (d, 1H), 6.81 (m, 1H), 5.57 (t, 1H), 5.20 (m, 1H, _{JH}=48 Hz), 4.07 (m, 4H), 3.64 to 3.29 (m, 4H), 2.87 (m, 2H), 1.19 (t, 3H); HR-MS (ES) m/z calcd for C₂₄H₂₆N₅FCl₂O₆ (MH⁺) 570.1322. Observed: 570.1317.

Step 2

### (β¹R)-3,5-dichloro-β-[[[[3-[(5-fluoro-1,4,5,6-tetrahydro-2-pyrimidinyl)amino]-5-hydroxybenzoyl]amino]acetyl]amino]-2-hydroxybenzenepropanoic acid, monotrifluoroacetate

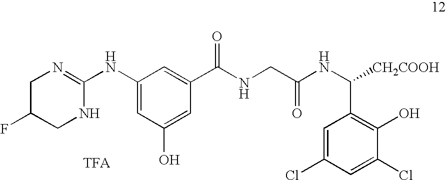

The ester from STEP 1 (0.42 g, 0.0006 mol) was stirred with 1M LiOH (5 mL) for 1 hour at room temperature. The pH was adjusted to 2 with trifluoroacetic acid and the product was purified by reverse phase HPLC to provide (after lyophilization) the desired acid 12 as a white powder (0.201 g, 50%): ¹H-NMR (CD₃OD, 400 Hz) δ 7.25 (t, 1H), 7.21 (t, 2H), 7.19 (t, 1H), 6.80 (t, 1H,), 5.51 (t, 1H), 5.26 (m,1H, _{JH}=48 Hz), 4.05 (s, 2H), 3.64 to 3.48 (m, 4H), 2.85 (m, 2H); HR-MS (ES) m/z calcd for C₂₂H₂₂N₅FCl₂O₆ (MH⁺) 542.1009. Observed: 542.1000.

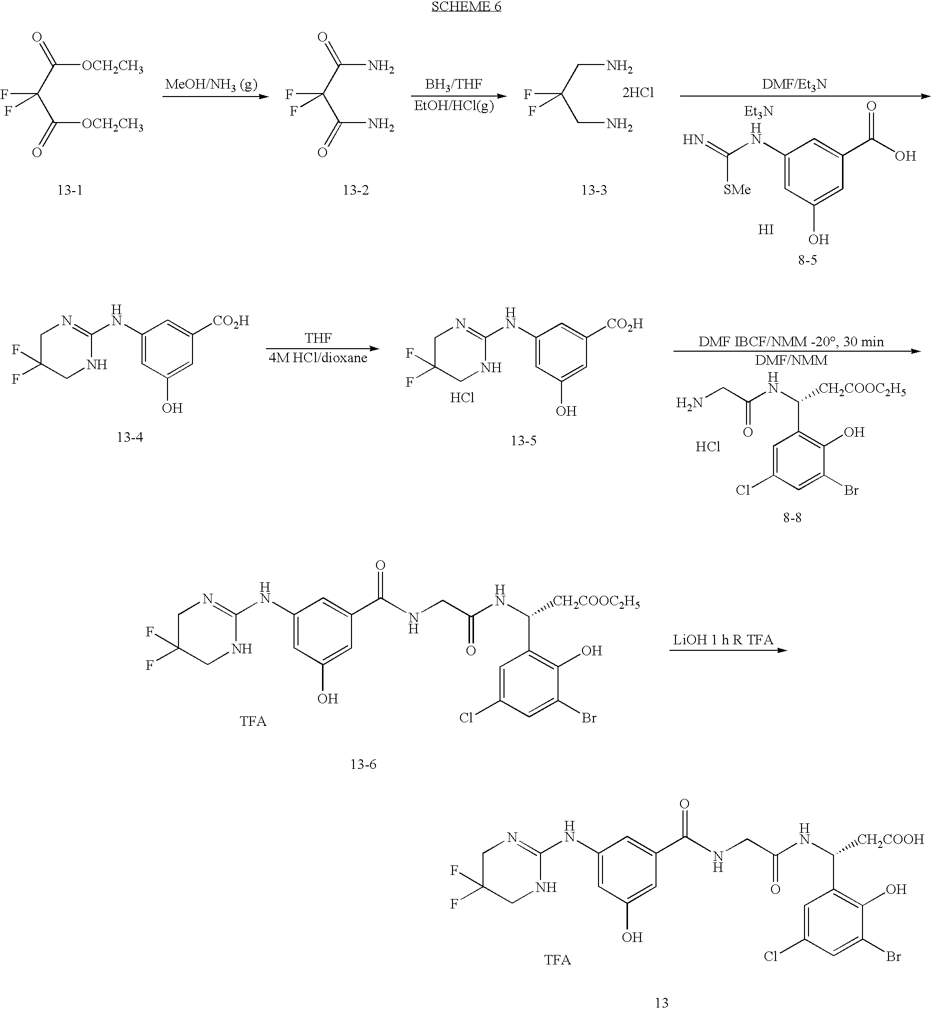

### Example 13

### (β¹R)-3-bromo-5-chloro-β-[[[[3-[(5,5-difluoro-1,4,5,6-tetrahydro-2-pyrimidinyl)-amino]-5-hydroxybenzoyl]amino]acetyl]amino]-2-hydroxybenzenepropanoic acid, monotrifluoroacetate

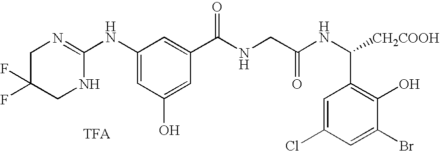 Step 1

### 2,2-difluoromalonamide

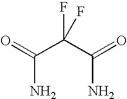

A solution of commercially available diethyl difluoromalonate (10.1145 g, 0.0515 mol) in anhydrous methanol (110 mL) was saturated with ammonia (g) at 0° C. for 45 min. The resulting mixture was stirred in an ice bath for 3 h and monitored by MS (m/z M+H 139). The product was concentrated under reduced pressure to afford desired amide 13-2 as a white powder (7.1027 g, 99%): HR-MS (ES) m/z calcd for C₃H₄N₂F₂O₂ (MH⁺)=139.0314. Observed: 139.0278.

Step 2

### 1,3-diamino-2,2-difluoropropane, dihydrochloride salt

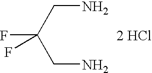

2,2-difluoromalonamide (2.09 g, 0.01514 mol) obtained from STEP 1, was added to cold 1.0 M BH₃-THF (72 mL, 0.072 mol) maintaining bath temperature at 0° to −4° C. The ice bath was removed and mixture was allowed to warm up to room temperature at which time a clear solution was formed. The solution was then heated to reflux (75° C.) overnight. The reaction was chilled in an ice bath and slowly quenched with methanol (25 mL). The solvents were removed under reduced pressure and the residue was co-evaporated with methanol again (3×100 mL) to remove excess boric acid. The residue, a milky white syrup, was dried overnight to remove excess solvent. Anhydrous ethanol (100 mL) was added to the residue and chilled in ice bath. The solution was then saturated with HCl (g) for 45 min. which became very exothermic and precipitated within minutes. Allowed reaction to stir at RT for 2 h. Filtered white precipitate and rinsed with ethanol. The product was washed with fresh ethanol and filtered a second time. The desired product 13-3 was isolated and dried as a white solid (0.4434 g, 34.8%): HR-MS (ES) m/z calcd for C₃H₈N₂F₂ (MH⁺)=111.0734. Observed: 111.0702.

Step 3

### 3-[(5,5-difluoro-1,4,5,6-tetrahydro-2-pyrimidinyl)amino]-5-hydroxybenzoic acid, monohydrochloride salt

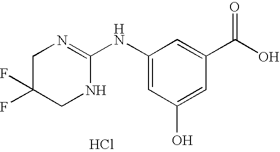

To a cold suspension of 1,3-diamino-2,2-difluoropropane hydrochloride salt as obtained in Step 3, (1.17 g, 0.0064 mol) in anhydrous DMF (20 mL) was added triethylamine (1.29 g, 0.0127 mol) to free the amine. Another portion of triethylamine (3.3 g, 0.0329 mol, 2 eq) was added to free amine suspension, followed by the addition of 3-hydroxy-5-[[imino-(methylthio)-methyl]amino]benzoic acid, monohydroiodide 8-5 (0.910 g, 0.0025 mol) in one portion. The solution was then heated at 85° C. under anhydrous conditions for 3 h. The reaction mixture was concentrated and water was added to the residue. The pH was adjusted to 4 using 2M HCl. The beige precipitate was filtered and washed with cold water followed by acetonitrile. The beige compound was dried in a desiccator in vacuo to afford the desired product 13-4 (0.3291 g, 50.6%).

Step 4

To a chilled suspension of the product 13-4 as obtained in STEP 3 (0.310 g, 0.00114 mol) in anhydrous THF (5 mL) was added HCl/dioxane (4N, 0.57 mL) and stirred cold to RT for 1 h. The solvent was removed under reduced pressure to afford the desired HCl salt 13-5 after drying (0.345 g, 98%): ¹H-NMR (CD₃OD, 400 Hz) δ 7.4 (t, 1H), 7.35 (m, 1H), 6.8 (t, 1H), 3.75 (t, 4H); HR-MS (ES) m/z calcd for C₁₁H₁₁N₃F₂O₃ (MH⁺)=272.0847. Observed: 272.0860.

Step 5

### (β¹R)-3-bromo-5-chloro-β-[[[[3-[(5,5-difluoro-1,4,5,6-tetrahydro-2-pyrimidinyl)amino]-5-hydroxybenzoyl]amino]acetyl]amino]-2-hydroxybenzenepropanoic acid, ethyl ester monotrifluoroacetate

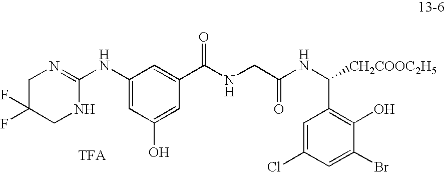

To a solution of the acid 13-5 (0.3295 g, 0.00107 mol) in anhydrous DMF (3 mL) at −10° C. was added isobutylchloroformate (0.136 g, 0.0010 mol) followed by the dropwise addition of N-methylmorpholine (0.110 g, 0.00109 mol). After stirring this mixture under argon atmosphere for 30 minutes at −20° C., a cold solution of ethyl (3R)-3-(3-bromo-5-chloro-2-hydroxyphenyl)-3-(glycylamino)-propanoate, hydrochloride prepared as in Example 1, Step 3 (0.423 g, 0.0010 mol) in anhydrous DMF (3 mL) and N-methylmorpholine (0.101 g, 0.0010 mol) was added. The resulting mixture was stirred at −10° C. for 15 minutes, and then stirred at room temperature overnight. Solvent was distilled in vacuo and the residue was purified by reverse phase HPLC to yield (after lyophilization) the desired ester 13-6 as a white powder, 0.22 g, 30%): ¹H-NMR (CD₃OD, 400 Hz) δ 7.41 (d, 1H), 7.24 (d, 2H), 7.18 (t, 1H), 6.83 (t, 1H), 5.57 (m, 1H), 4.04 (m, 4H), 3.72 (m, 4H), 2.84 (m, 2H), 1.18 (t, 3H); HR-MS (ES) m/z calcd for C₂₄H₂₅N₅F₂ClBrO₆ (MH⁺)=632.0723. Observed: 632.0727.

Step 6

### (β¹R)-3-bromo-5-chloro-β-[[[[3-[(5,5-difluoro-1,4,5,6-tetrahydro-2-pyrimidinyl)-amino]-5-hydroxybenzoyl]amino]acetyl]amino]-2-hydroxybenzenepropanoic acid, monotrifluoroacetate

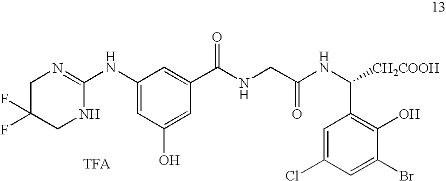

The ester 13-6 as obtained in STEP 5 (0.2 g, 0.00026 mol) was stirred with 1M LiOH (3 mL) for 2 h at room temperature. The pH was adjusted to 2 with trifluoroacetic acid and the product was purified by reverse phase HPLC to provide (after lyophilization) the desired acid 13 as a white powder, 0.09 g (46%): ¹H-NMR (CD₃OD, 400 Hz) δ 7.4 (t, 1H), 7.24 (m, 3H), 6.8 (t, 1H), 5.56 (q, 1H), 4.05 (d, 2H), 3.68 (t, 4H), 2.8 (dd, 2H); HR-MS (ES) m/z calcd for C₂₂H₂₁N₅F₂ClBrO₆ (MH⁺)=604.0410. Observed: 604.0414.

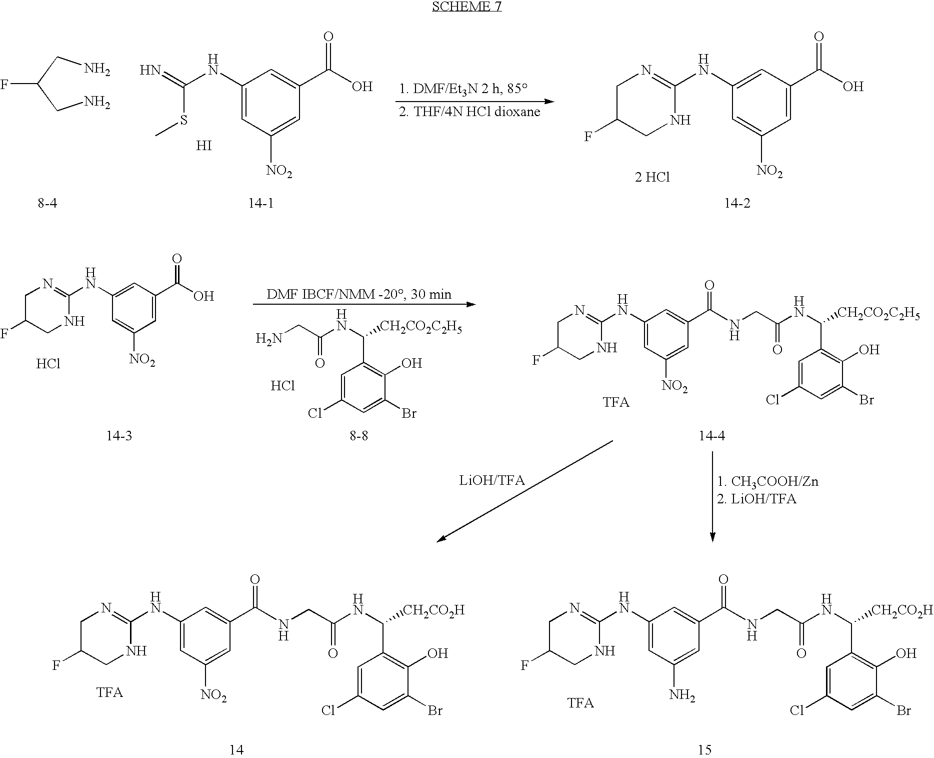

### Example 14

### (β¹R)-3-bromo-5-chloro-β-[[[[3-[(5-fluoro-1,4,5,6-tetrahydro-2-pyrimidinyl)-amino]-5-nitrobenzoyl]amino]acetyl]amino]-2-hydroxybenzenepropanoic acid, monotrifluoroacetate

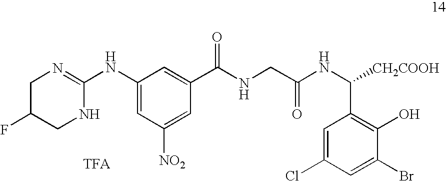 Step 1

### (3) 3-[(5-fluoro-1,4,5,6-tetrahydro-2-pyrimidinyl)amino]5-nitrobenzoic acid, hydrochloride salt

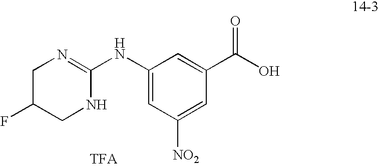

To a solution of 1,3-diamino-2-fluoropropane 8-4 (3.73 g, 0.0405 mol) in anhydrous DMF (50 mL) containing triethylamine (5.08 g, 0.05 mol) was added compound (2) (7.5 g, 0.0195 mol) in one portion. Initial pH was basic. The solution was heated at 85° C. under anhydrous conditions for 3 h. Solvent was removed in vacuo to afford a light beige residue. The residue was diluted with water and was acidic to pH. The residue was chilled, filtered, and washed with water followed by acetonitrile. The beige compound was dried in a desiccator in vacuo to afford 14-2 (1.46 g, 25%): ¹H-NMR (DMSO, 400 Hz) δ 8.65 (m, 1H), 8.30 (t, 1H), 8.20(m, 1H), 5.31 (m,1H, _{JH}=48 Hz), 3.65 (m, 4H); HR-MS (ES) m/z calcd for C₁₁H₁₁N₄FO₄ (MH⁺)=283.0843. Observed: 283.0821.

Step 2

To a chilled suspension of 14-2 (1.404 g, 0.0049 mol) in anhydrous THF (12 mL) was added HCl/dioxane (4N, 2.48 mL) and stirred cold 1 h. The solvent was removed under reduced pressure to afford the desired salt 14-3 after drying (1.8 g, 100%).

Step 3

### (β¹R)-3-bromo-5-chloro-β-[[[[3-[(5-fluoro-1,4,5,6-tetrahydro-2-pyrimidinyl)-amino]-5-nitrobenzoyl]amino]acetyl]amino]-2-hydroxybenzene-propanoic acid, ethyl ester monotrifluoroacetate

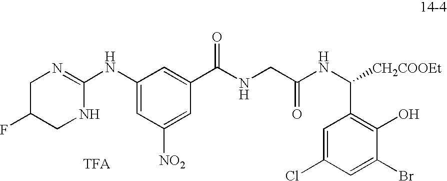

To a solution of 3-[(5-fluoro-1,4,5,6-tetrahydro-2-pyrimidinyl)amino]5-nitrobenzoic acid, hydrochloride salt (0.712 g, 0.0021 mol) in anhydrous DMF (5 mL) at −10° C was added isobutylchloroformate (0.28 g, 0.0020 mol) followed by the dropwise addition of N-methylmorpholine (0.23 g, 0.0022 mol). After stirring this mixture under argon atmosphere for 30 minutes at −20° C., a cold solution of ethyl (3R)-3-(3-bromo-5-chloro-2-hydroxyphenyl)-3-(glycylamino)-propanoate, hydrochloride prepared in Example 1, Step 3 (0.88 g, 0.0021 mol) in anhydrous DMF (5 mL) and N-methylmorpholine (0.21 g, 0.0022 mol) was added. The resulting mixture was stirred at −10° C. for 15 minutes, and then stirred at room temperature overnight. Solvent was distilled in vacuo and the residue was purified by reverse phase HPLC to yield (after lyophilization) the desired ester 14-4 as a white powder, 0.49 g, 30%): ¹H-NMR (CD₃CD, 400 Hz) δ 8.63 (m, 1H), 8.26 (m, 1H), 8.1 (t, 1H) 7.41 (d, 1H), 7.24 (d, 1H) 5.57 (m, 1H), 5.19 (m, 1H, _{JH}=46.8 Hz), 4.09 (m, 4H), 3.64 (m, 4H), 2.84 (m, 2H), 1.18 (t, 3H); HR-MS (ES) m/z calcd for C₂₃H₂₅N₆FClBrO₇ (MH⁺)=643.0719. Observed: 643.0732.

Step 4

### (β¹R)-3-bromo-5-chloro-β-[[[[3-[(5-fluoro-1,4,5,6-tetrahydro-2-pyrimidinyl)-amino]-5-nitrobenzoyl]amino]acetyl]amino]-2-hydroxybenzenepropanoic acid, monotrifluoroacetate.

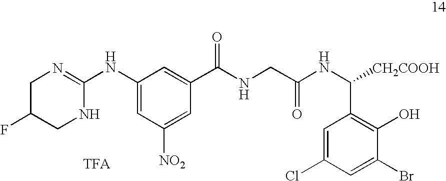

The above ester 14-4 as obtained in STEP 3 (0.307 g, 0.0004 mol) was stirred with 1M LiOH (5 mL) for 2 h at room temperature. The pH was adjusted to 2 with trifluoroacetic acid and the product was purified by reverse phase HPLC to provide (after lyophilization) the desired acid 14 as a white powder, 0.10 g (33%): ¹H-NMR (CD₃0D, 400 Hz) δ 8.63 (t, 1H), 8.25 (t, 1H), 8.1 (t, 1H), 7.41 (d, 1H), 7.25 (d, 1H), 5.56 (q, 1H), 5.26 (m, 1H, _{JH}=44 Hz), 4.1 (d, 2H), 3.68 (m, 4H), 2.8 (dd, 2H); HR-MS (ES) m/z calcd for C₂₂H₂₁N₆FClBrO₇ (M+H)=615.0406. Observed: 615.0417.

### Example 15

### (β¹R)-β-[[[[3-amino-5-[(5-fluoro-1,4,5,6-tetrahydro-2-pyrimidinyl)amino]benzoyl]-amino]acetyl]amino]bromo-5-chloro-2-hydroxybenzenepropanoic acid, monotrifluoroacetate

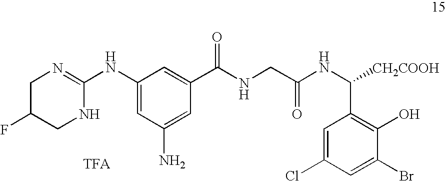

To the ester 14-4 from STEP 3 (0.28g, 0.00036 mol) was added acetic acid (7 mL) and Zn (1.5 g). The reaction mixture was stirred at 0° C. under an atmosphere of nitrogen for 1 h. The reaction was monitored by ES mass spectrometry for completion. The reaction stirred overnight at RT. The mixture was filtered and rinsed with ethanol. The filtrate was concentrated under reduced pressure and the residue was hydrolyzed with 1M LiOH for 2 h. The pH was adjusted to 2 with trifluoroacetic acid and the product was purified by reverse phase HPLC to provide (after lyophilization) the desired acid 15 as a white powder, 0.060 g (22%): ¹H-NMR (CD₃OD, 400 Hz) δ 7.42 (d,1H), 7.24 (d, 1H), 7.1 (t, 1H), 6.94 (t, 1H), 6.68 (t, 1H), 5.56 (q, 1H), 5.26 (m, 1H, _{JH}=44 Hz), 4.04 (s, 2H), 3.68 (m, 4H), 2.8 (dd, 2H); HR-MS (ES) m/z calcd for C₂₂H₂₃N₆O₅FClBr (MH⁺) 585.0664. Observed: 585.0694.

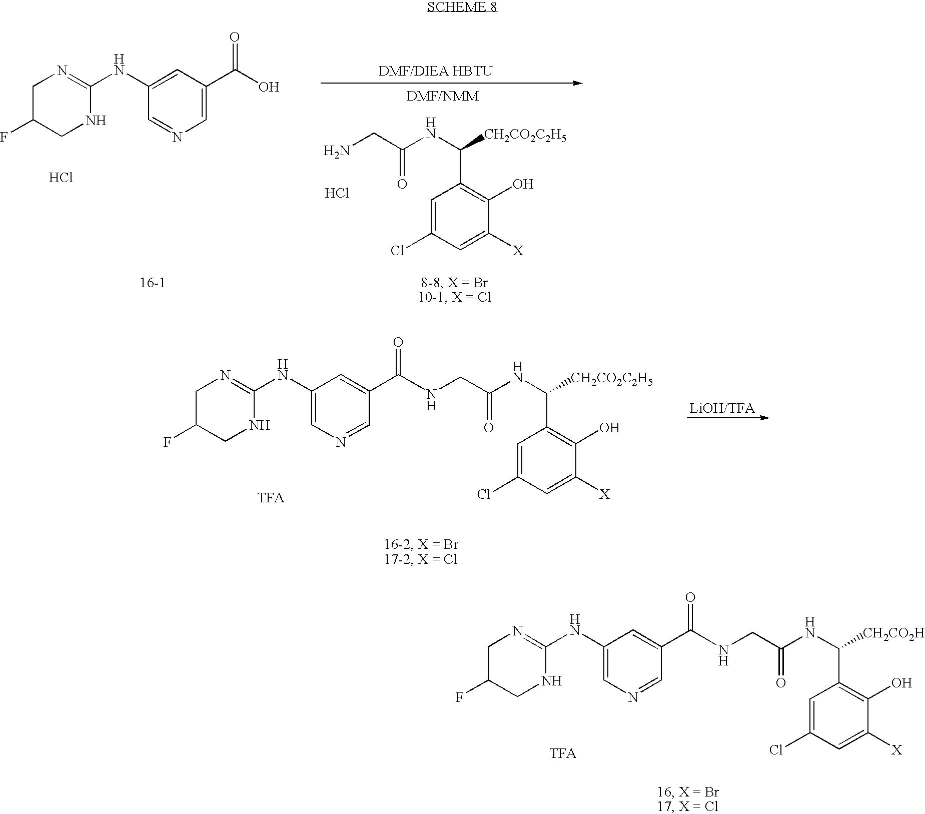

### Example 16

### (β¹R)-3-bromo-5-chloro-β-[[[[[5-[(5-fluoro-1,4,5,6-tetrahydro-2-pyrimidinyl)amino]-3-pyridinyl]carbonyl]amino]acetyl]amino]-2-hydroxybenzenepropanoic acid, monotrifluoroacetate

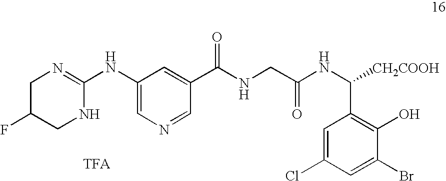 Step 1

### bis-N-benzyloxycarbonyl-2-fluoro-1,3-diaminopropane.

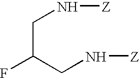

To a stirred suspension of bis-N-benzyloxycarbonyl-2-hydoxy-1,3-diamino-propane (6.0 g, 0.017 mol) in dichloromethane (50 mL) and pyridine (2.7 mL) at −50° C., was added dropwise a solution of DAST (2.5 mL) in dichloromethane (7.5 mL). The reaction mixture was gradually allowed to warm to room temperature over a period of 16 h under an atmosphere of argon, when a clear yellow solution was obtained. It was cooled and poured into a mixture of ice water (100 mL), and dichloromethane (50 mL). The organic phase was washed with water (2×50 mL), and dried (Na₂SO₄). After removal of the solvent, the residue was purified by silicagel flash chromatography using 30% EtOAc in hexane. The appropriate fractions were combined, concentrated to dryness and the product was crystallized from dichloromethane/hexane to afford the desired fluoro intermediate (2.0 g) as a white fluffy powder. ¹H-NMR and mass spectral data were consistent with the structure.

Step 2

### N-(5-fluorotetrahydropyrimidinyl)-3-aminonicotinic acid

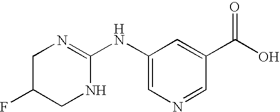

A solution of bis-N-benzyloxycarbonyl-2-fluoro-1,3-diaminopropane (3.3 g, 0.0092 mol) as obtained from step 1, in EtOAc ( 30 mL), and EtOH (30 mL) was hydrogenated at 50 psi in the presence of Pd/C (10%, 2.7 g) for 16 h at room temperature (Scheme 4). Following filtration, the catalyst was stirred with EtOH containing 40% water (50 mL) and filtered again. The filtrate was concentrated to dryness to afford syrup (0.7 g). This was suspended in DMF (8.0 mL), added the product from step 2 of Example 4 (0.7 g, 0.0033 mol), catalytic amount of DMAP (0.01 g), and heated at 90° C. for 3 h under anhydrous conditions. DMF was distilled in vacuo, the residue was suspended in water (25 mL) and pH was adjusted to 4.5 by the addition of 1N HCl. The resulting mixture was cooled, solid that separated was filtered, and washed thoroughly with water, acetonitrile and dried in a desiccator in vacuo to provide the desired compound (0.24 g) as brown powder. ¹H-NMR and mass spectral data were consistent with the structure.

Step 3.

### (β¹R)-3-bromo-5-chloro-β-[[[[[5-[(5-fluoro-1,4,5,6-tetrahydro-2-pyrimidinyl)-amino]-3-pyridinyl]carbonyl]amino]acetyl]amino]-2-hydroxybenzene-propanoic acid, ethyl ester monotrifluoroacetate

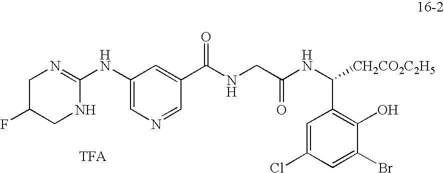

To a solution of 5-[(5-fluoro-1,4,5,6-tetrahydro-2-pyrimidinyl)amino]3-pyridine-carboxylic acid, monohydrochloride 16-1 (0.442 g, 0.0016 mol) in anhydrous DMF (5 mL) at −5° C. was added diisopropylethylamine (DIEA, 0.52 g, 0.004 mol) followed by the addition of HBTU (0.733 g, 0.00193 mol). After stirring this mixture under nitrogen atmosphere for 1 h at −5° C., solution was allowed to warm up to RT and stirred for 1 h. After activating the ester, a cold solution containing ethyl (3R)-3-(3-bromo-5-chloro-2-hydroxyphenyl)-3-(glycylamino)-propanoate, hydrochloride prepared in Example 1, Step 3 (0.567 g, 0.00153 mol) in anhydrous DMF (5 mL) and N-methylmorpholine (0.199 g, 0.00197 mol) was added. The pH of the resulting mixture was 7 at 1 h, and was allowed to stir at room temperature overnight. Solvent was distilled in vacuo and the residue was purified by reverse phase HPLC to yield (after lyophilization) the desired ester 16-2 as a white powder, 0.47 g (46%): ¹H-NMR (CD₃OD, 400 Hz) δ 8.93 (d, 1H), 8.61 (d, 1H), 8.13 (t, 1H), 7.42 (d, 1H), 7.25 (d, 1H,) 5.54 (q, 1H), 5.29 (m, 1H, _{JH}=46 Hz), 4.09 (m, 4H), 3.66 (m, 4H), 2.83 (dd, 2H), 1.18 (t, 3H); HR-MS (ES) m/z calcd for C₂₃H₂₅N₆O₆FClBr (MH⁺) 599.0821. Observed: 599.0779.

Step 4

### (β¹R)-3-bromo-5-chloro-β-[[[[[5-[(5-fluoro-1,4,5,6-tetrahydro-2-pyrimidinyl)-amino]-3-pyridinyl]carbonyl]amino]acetyl]amino]-2-hydroxybenzene-propanoic acid, monotrifluoroacetate.

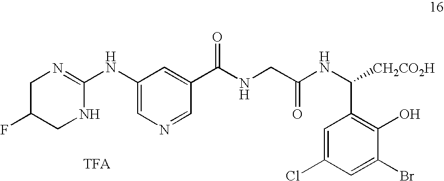

The ester 16-2 (0.45 g, 0.00067 mol) was stirred with 1M LiOH (2 mL) for 1 hour at room temperature. The pH was adjusted to 2 with trifluoroacetic acid and the product was purified by reverse phase HPLC to provide (after lyophilization) the desired acid 16 as a white powder, 0.44 g (97%): ¹H-NMR (CD₃OD, 400 Hz) δ 8.93 (d, 1H), 8.6 (d, 1H), 8.13 (t, 1H), 7.41 (d, 1H), 7.25 (d, 1H,) 5.55 (q, 1H), 5.29 (m, 1H, _{JH}=46 Hz), 4.1 (s, 2H), 3.5 (m, 4H), 2.85 (dd, 2H); HR-MS (ES) m/z calcd for C₂₁H₂₁N₆O₆FClBr (MH⁺) 571.0508. Observed: 571.0512.

### Example 17

### (β¹R)-3-chloro-5-chloro-β-[[[[[5-[(5-fluoro-1,4,5,6-tetrahydro-2-pyrimidinyl)-amino]-3-pyridinyl]carbonyl]amino]acetyl]amino]-2-hydroxybenzenepropanoic acid, monotrifluoroacetate

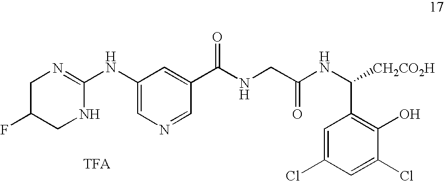 Step 1

### (¹R)-3,5-Dichloro-β-[[[[[5-[(5-fluoro-1,4,5,6-tetrahydro-2-pyrimidinyl)-amino]-3-pyridinyl]carbonyl]amino]acetyl]amino]-2-hydroxybenzene-propanoic acid, ethyl ester monotrifluoroacetat

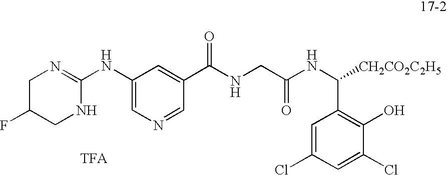

To a solution of 5-[(5-fluoro-1,4,5,6-tetrahydro-2-pyrimidinyl)amino]3-pyridine-carboxylic acid, monohydrochloride prepared as in Example 16, (0.45 g, 0.00164 mol) in anhydrous DMF (5 mL) at −5° C. was added diisopropylethylamine (DIEA, 0.29 g, 0.0023 mol) followed by the addition of HBTU (0.746 g, 0.00196 mol). After stirring this mixture under nitrogen atmosphere for 1 h at −5° C., solution was allowed to warm up to RT and stirred for 1 h. After activating the ester, a cold solution containing ethyl (3R)-3-(3,5-dichloro-2-hydroxyphenyl)-3-(glycylamino)-propanoate, hydrochloride prepared as in Example 3 (0.667 g, 0.00155 mol) in anhydrous DMF (5 mL) and N-methylmorpholine (0.199 g, 0.00197 mol) was added. The pH of the resulting mixture was 7 at 1 h, and was allowed to stir at room temperature overnight. Solvent was distilled in vacuo and the residue was purified by reverse phase HPLC to yield (after lyophilization) the desired ester 17-2 as a white powder, 0.74 g (66%): ¹H-NMR (CD₃OD, 400 Hz) δ 8.93 (d, 1H), 8.61 (d, 1H), 8.13 (t, 1H), 7.26 (d, 1H), 7.20 (d, 1H,) 5.54 (q, 1H), 5.29 (m, 1H, _{JH}=44 Hz), 4.09 (m, 4H), 3.66 (m, 4H), 2.83 (dd, 2H), 1.2 (t, 3H); HR-MS (ES) m/z calcd for C₂₃H₂₅N₆O₆FCl₂ (MH⁺) 555.1326. Observed: 555.1330.

Step 2

### (β¹R)-3,5-Dichloro -β-[[[[[5-[(5-fluoro-1,4,5,6-tetrahydro-2-pyrimidinyl)-amino]-3-pyridinyl]carbonyl]amino]acetyl]amino]-2-hydroxybenzenepropanoic acid, monotrifluoroacetate.

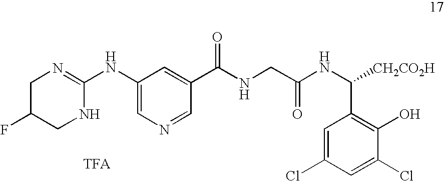

The ester 17-2 (0.72 g, 0.0001 mol) from STEP 1 was stirred with 1M LiOH (3 mL) for 3 h at room temperature. The pH was adjusted to 2 with trifluoroacetic acid and the product was purified by reverse phase HPLC to provide (after lyophilization) the desired acid as a white powder, 0.48 g (69%): ¹H-NMR (CD₃OD, 400 Hz) β 8.93 (d, 1H), 8.61 (d, 1H), 8.13 (t, 1H), 7.25 (d, 1H), 7.21 (d, 1H,) 5.54 (q, 1H), 5.29 (m, 1H, _{JH}=44 Hz), 4.1 (s, 2H), 3.5 (m, 4H), 2.85 (dd, 2H); HR-MS (ES) m/z calcd for C₂₁H₂₁N₆O₆FCl₂ (MH⁺) 527.1013. Observed: 527.1039

### Example 18

### (β¹R)-5-bromo-3-chloro-β-[[[[3-[(5-hydroxy-1,4,5,6-tetrahydro-2-pyrimidinyl )-amino]-5-hydroxybenzoyl]amino]acetyl]amino]-2-hydroxybenzenepropanoic acid, monotrifluoroacetate.

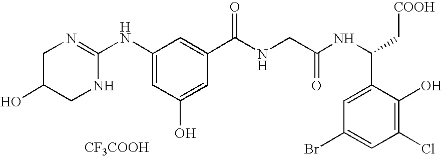 Step 1

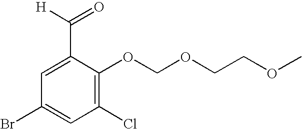

To a mixture of 3-chloro-5-bromosalycil aldehyde (50.0 g, 0.214 mol), and potassium carbonate (29.6 g, 0.214 mol) in DMF (120.0 mL) was added dropwise MEM chloride (27.2 mL) and stirred at 10° C. under an atmosphere of argon. After 30 min, the reaction mixture was allowed to warm to room temperature over a period of 2.5 h. It was then cooled, and poured into a mixture of cold water (500 mL), and dichloromethane ((300 mL). The organic phase was separated, and the aqueous phase was extracted twice with dichloromethane (2×100 mL). The organic extracts were washed with water (3×100 mL), dried (Na₂SO₄), and concentrated under reduced pressure. The resulting residue was washed with hexane and dried to give 54.4 g (835) of the desired product as a light brown solid: ¹H-NMR (CDCl₃) δ 10.27 (s, 1H), 7.85 (d, 1H, J=2.4 Hz), 7.76 (sd, 1H, J=2.4 Hz), 5.23 (s, 2H), 3.9 (m, 2H), 3.53 (m, 2H), and 3.35 (s, 3H); ES-MS m/z=340 (M+NH₄); HRMS: calcd C₁₃H₁₂O₄BrCl NH₄ 339.9997. Found 339.9951.
Step 2

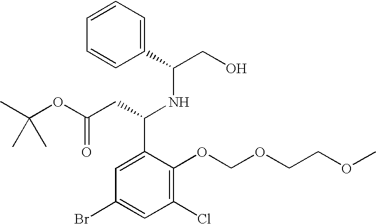

To a degassed solution of the MEM-protected aldehyde (50.0 g, 0.163 mol) in THF (200 mL), was added R-phenylglycinol (24.7 g, 0.18 mol). After stirring for 30 min at room temperature, added anhydrous MgSO₄ (6.0 g) and stirred for an additional 1.5 h and filterd. The filtrate was concentrated under reduced pressure and the resulting residue was dried in vacuo for 45 min. This material was dissolved in N-methylpyrrolidinone (200 mL) and added dropwise to degassed solution of the zinc-t-butylbromoacetate (120.0 g) in N-methyl-pyrrolidinone at −5° C. The resulting mixture was stirred for 1 h, when TLC (EtOAc/Hexane 1:3 v/v) revealed completion of the reaction. It was then poured into a stirred mixture of cold (10° C.) mixture of conc. HCl (11.0 mL), saturated ammonium chloride (250 mL), and EtOAc (300 mL). The aqueous phase was extracted twice with EtOAc (2×100 mL). The combined organic extracts were washed with water, (3×100 mL), dried (Na₂SO₄), and concentrated under reduced pressure to give 86.0 g (94%) of an orange syrup of the desired adduct. This substance was used as such in the following step: ¹H-NMR (CDCl₃) δ 7.32 (d, 1H, J=2.4 Hz), 7.19 (m, 6H), 5.13 (m 2H), 4.61 (m, 1H), 3.92 (m, 2H), 3.82 (m, 1H), 3.58 (m, 4H), 3.05 (m, 1H), 2.55 (m, 2H), 1.43 (s, 9H); ES-MS m/z=558 (M+H).
Step 3

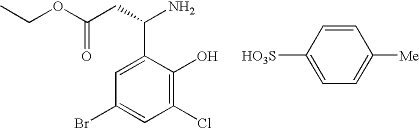

The adduct (43.6 g, 0.079 mol) from STEP 2, was dissloved in EtOH (500.0 mL), cooled to 0° C., added in portions leadtetraacetate (38.2 g) over a period of 15 min, and stirred under an argon atmosphere. After 2 h the reaction was quenched with 15% NaOH (70 mL) and concentrated under reduced pressure to half the volume. Then added an additional cold 15% NaOH (280 mL) and EtOAc (500 mL). The resulting suspension was filtered through a celite pad. The organic phase was washed with brine (3×150 mL), dried Na₂SO₄), and concentrated under reduced pressure. The resulting residue (39.0 g) was dissolved in dry ethanol (150 mL), added p-toluenesulfonic acid mono hydrate (17.0 g, 0.09 mol), and refluxed for 8 h. The resulting dark colored solution was concentrated to dryness and the residue was triturated with ether (100 mL) and filtered the solid. The solids were washed with a solvent mixture containing THF/ EtOH (1:1 v/v, 200 mL), and dried in vacuo afford the desired beta-amino ester (19.1 g, 50%) as its tosylate salt: ¹H-NMR (CD₃OD) δ 7.68 (d, 2H, J=6.0 Hz), 7.57 (s, 1H), 7.42 (s, 1H), 7.2 (d, 2H, J=6.0 Hz), 4.18 (q, 2H), 3.15 (dd 2H), 2.34 (s, 3H), 1.2 (t, 3H, J=6.6 Hz); ES-MS m/z=322 (M+H); HRMS: calcd C₁₁H₁₄NO₃BrCl 321.9846. Found 321.9877.
Step 4

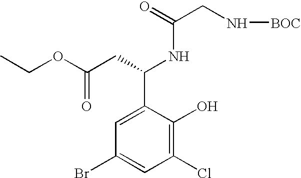

To a cold (10° C.) solution of the tosylate salt (18.0 g, 0.0365 mol) in DMA (30.0 mL) and dichloromethane (30.0 mL), was added N-methyl-morpholine (4.4 mL), and BOC-Gly-OSu (10.0 g, 0.0368 mol), and stirred at room temperature for 16 h. The reaction mixture was partitioned between 10% citric acid (100 mL) and dichloromethane (200 mL). The organic phase was washed with brine (2×100 mL), dried (Na₂SO₄), and concentrated under reduced pressure to give 18.0 g of the crude product. This material was used without purification in the following step.
Step 5

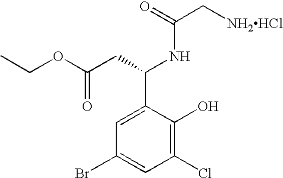

HCl gas was bubbled into cold (5° C.) ethanol (30.0 mL). After 30 min, 1.0 g of the product obtained in step D was added, and stirred at room temperature for 2 h. The solution was concentrated and the residue was triturated with EtOAc, and filtered the solid. The solid was washed with ethyl acetate and dried to give 0.59 g the desired product as its HCl salt: ¹H-NMR (CD₃OD, 400 Mz) δ 7.41 (d, 1H, J=2.4 Hz), 7.30 (d, 1H, J=2.4 Hz), 5.55 (m, 1H), 4.06 (t, 2H, J=7.2 Hz), 3.3 (s, 2H), 2.84 (m 2H), and 1.82 (t, 3H, J=7.2 Hz); ES-MS m/z=379 (M+); HRMS: calcd C₁₃H₁₇N₂O₄ClBr: 379.0060. Found 379.0061.
Step 6

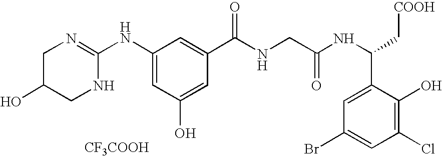

A solution 3-N-(5-hydroxytetrahydropyrimidinyl)-5-hydroxybenzoic acid (prepared according to U.S. Pat. No. 6,013,651, Example H, 0.35 g, 0.0012 mol) in DMF (4.0 mL) was cooled to −10° C., added dropwise isobutylchloroformate (0.16 mL), and N-methylmorpholine (0.15 mL). After stirring for 20 min, added another 0.15 ml of N-methylmorpholine, followed by the addition of amino ester hydrochloride (0.5 g, 0.0012 mol). The resulting mixture was allowed to stir at room temperature for 16 h. DMF was distilled in vacuo and the residue was purified by reverse-phase HPLC using 10-90% acetonitrile/-water gradient (40 in) at a flow rate of 70 mL/min. The appropriate fractions were combined and freeze dried to 0.25 g of the desired ester as a white powder: ¹H-NMR (CD₃OD) δ 7.39 (d, 1H, J=1.8 Hz), 7.31 (d, 2H, J=1.8 Hz), 7.17 (m, 2H), 6.81 (m, 1H), 5.8 (m, 1H), 4.21 (m, 1H), 4.08 (q, 2H), 4.05 (s, 2H), 3.32 (m, 2H), 2.85 (m, 2H), 1.18 (t, 3H, J=5.4 Hz); ES-MS m/z=612 (M+H); HRMS: calcd C₂₄H₂₈N₅O₇ClBr: 612.0861. Found, 612.0824.

Step 7

The ester (0.4 g) as obtained in STEP 6, was stirred with 1M LiOH (3.0 mL) and acetonitrile (1.0 mL). After 1 h, the solution was diluted with water (2 mL) acidified with trifluroacetic acid and the product was isolated by reverse-phase HPLC using 10-90% acetonitrile/water gradient (40 in) at a flow rate of 70 mL/min. The appropriate fractions were combined and freeze dried to 0.26 g of the desired acid as its trifluroacetate salt: ¹H-NMR (CD₃OD) □ 7.38 (d, 1H J=1.8 Hz), 7.32 (d, 1H, J=1.8 Hz), 7.19 (m, 2H), 6.81 (m, 1H), 5.45 (m, 1H), 4.21 (m 1H), 4.055 (s, 2H), 3.35 (m, 2H), and 2.85 (m 2H); ES-MS m/z=612 (M+H); HRMS: calcd C₂₂H₂₄N₅O₇ClBr: 584.0548. Found: 584.0500.

### Example 19

### (β¹R)-5-bromo-3-chloro-β-[[[[[5-[(5-hydroxy -1,4,5,6-tetrahydro-2-pyrimidinyl)-amino]-3-pyridinyl]carbonyl]amino]acetyl]amino]-2-hydroxybenzenepropanoic acid, monotrifluoroacetate

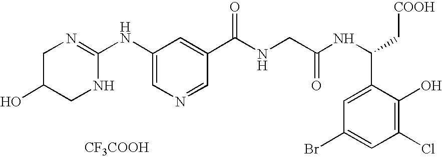

To a cold suspension of 5-[(5-hydroxy -1,4,5,6-tetrahydro-2-pyrimidinyl)-amino]-]nicotinic acid hydrochloride, prepared as in Example 2, (0.62 g, 0.002 mol) in DMF (10.00 mL) was added isobutylchloroformate (0.28 mL), followed by the dropwise addition of N-methyl-morpholine (0.22 mL) and stirred the mixture at −10° C. under an atmosphere of argon. After 25 min, a solution of the amine generated by the addition of N-methylmorpholine (0.2 mL) to a solution ethyl-3-chloro-5-bromo-2-hydroxyphenyl)-3-(glycylamino)-propanoate, hydrochloride prepared as in Example 18 (0.75 g, 0.0018 mol) in DMF (5.00 mL) was added and the resulting mixture was stirred at room temperature for 30 min, and left in the refrigerator overnight. The solvents were distilled in vacuo, and the residue was purified by reverse-phase HPLC using 10-90% acetonitrile/-water gradient (40 in) at a flow rate of 70 mL/min. The appropriate fractions were combined and freeze dried to 0.3 g of the desired ester (m/z=599) as a white powder. This material was stirred with 1M LiOH (2.0 mL) for 1 h, cooled, acidified with trifluroacetic acid and the product was isolated by reverse-phase HPLC using 10-90% acetonitrile/-water gradient (40 min) at a flow rate of 70 mL/min. The appropriate fractions (m/z=570) were combined and freeze dried to afford the 0.16 g of the title compound as its trifluroacetate salt: ¹H-NMR (300 Mz, CD₃OD) δ 8.91 (d, 1H, J=1.2 Hz), 8.60 (d, 1H, J=1.2 Hz), 8.13 (m, 1H), 7.39 (d, 1H, J=1.5 Hz), 7.33 (d, 1H, J=1.5 Hz), 5.48 (m 1H), 4.25 (1H, t, J=2.4 Hz), 4.10 (d, 2H, J=1.5 Hz), 3.46 (dd, 2H), 3.29 (dd, 2H), 2.88 (dd, 1H), 2.78 (dd, 2H); HRMS, m/z: Calcd for C₂₁H₂₃N₆O₆ClBr: 569.0551. Found: 569.0584.

### Example 20

### (β¹R)-3-bromo-5-chloro-β-[[[[[5-[(5,5-dimethyl-1,4,5,6-tetrahydro-2-pyrimidinyl)-amino]-3-pyridinyl]carbonyl]amino]acetyl]amino]-2-hydroxybenzenepropanoic acid, monotrifluoroacetate

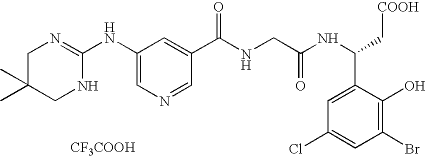 Step 1

### N-(5,5-dimethyltetrahydropyrimidinyl)-3-aminonicotinic acid

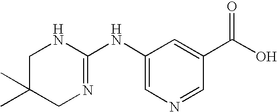

The compound was synthesized using the methodology described for Example 2 Step 3 substituting 4 equivalents 2,2-dimethyl-1,3-propanediamine for 1,3-diamino, 2-hydroxypropane.

A solution of N-(5,5-dimethyltetrahydropyrimidinyl)-3-aminonicotinic acid (0.78 g, 0.002 mol) in DMF was cooled to −10° C., and added isobutyichloroformate (0.3 mL), followed by the dropwise addition of N-methylmorpholine (0.3 mL). After 30 min, added a solution of the of the amine generated by the addition of N-methylmorpholine (0.3 mL) to a solution of solution ethyl-3-bromo-5-chloro-2-hydroxyphenyl)-3-(glycylamino)-propanoate, hydrochloride prepared as in Example 1 (0.75 g, 0.0018 mol) in DMF (5.00 mL), and resulting mixture was stirred at room temperature for 16 h under an atmosphere of argon. The solvents were distilled in vacuo and the residue was purified by reverse-phase HPLC using 10-90% acetonitrile/water gradient (40 in) at a flow rate of 70 mL/min. The appropriate fractions were combined and freeze dried to 0.5 g of the desired ester as a white powder: ¹H-NMR (300 Mz, CD₃OD) δ 8.91 (d, 1H, J=1.5 Hz), 8.59 (d, 1H, J=1.5 Hz), 8.10 (m, 1H), 7.41 (d, 1H, J=2.1 Hz), 7.24 (d, 1H, J=2.1 Hz), 5.62 (m 1H), 4.09 (m, 4H), 3.05 (s, 4H), 2.83 (m, 2H), 1.18 (t, 3H, J=5.4 Hz), 1.1 (3, 6H); HRMS, m/z (M+H): calcd C₂₅H₃₁N₆O₅ClBr: 609.1228. Found 609.1225.

Step 2

The ester (0.35 g, 0.048 mmol) was stirred with 1M LiOH (2.0 mL) at room temperature for 1 h. The solution was diluted with water (3.0 mL), cooled, acidified with trifluoroacetic acid and the product was isolated by reverse-phase HPLC using 10-90% acetonitrile/water gradient (40 min) at a flow rate of 70 mL/min. The appropriate fractions (m/z=570) were combined and freeze dried to afford the 0.25 g of the title compound as its trifluoro-acetate salt: ¹H-NMR (300 Mz, CD₃OD) δ 8.91 (d, 1H, J=1.2 Hz), 8.59 (d, 1H, J=1.2 Hz), 8.11 (s, 1H), 7.4 (d, 1H, J=2.1 Hz), 7.25 (d, 1H, J=2.1 Hz), 5.58 (m 1H), 4.09 (d, 2H, J=1.8 Hz), 3.09 (s, 4H), 2.84 (m 2H), and 1.1 (s, 6H); HRMS, m/z (M+H): calcd C₂₅H₂₇N₆O₅ClBr: 583.0895. Found 583.0823.

### Example 21

### R-β-[[2-[[[3-hydroxy-5-[4,5-(dihydro-1H-imidazol-2yl)amino]-phenyl]carbonyl]-amino]acetyl]amino]3-bromo-5-chloro-2-hydroxybenzenepropanoic acid, trifluoroacetate salt

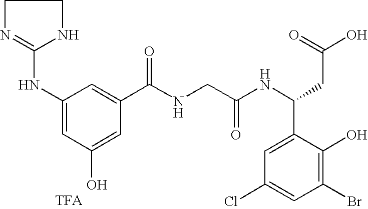 Step 1.

### 3-(4,5-dihydro-1H-imidazol-2-ylamino)-5-hydroxybenzoic acid.

To a solution of 3-hydroxy-5-{[imino(methylthio)methyl]amino}benzoic acid hydroiodide (WO9944996) (10 g, 27 mmol) in DMF (25 mL) was added ethylenediamine (4.9 g, 81 mmol). The reaction mixture was heated at 75° C. overnight then cooled to room temperature. Solid was filtered and washed with excess DMF and ether. Dried to give 3 g (50%) solid. Sample was used without further purification. ¹H NMR (DMSO) δ 8.37 (bs, 2H), 7.90 (s, 1H), 7.23-7.21 (m, 2H), 6.84 (s, 1H), 3.62 (bs, 4H).
Step 2

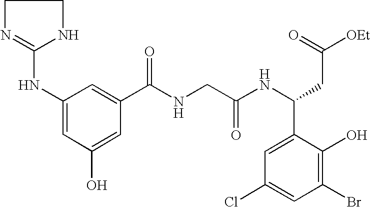

To a solution of 3-hydroxy-5-[4,5-(dihydro-1H-imidazol-2yl)benzoic acid (0.3 g, 1.34 mmol) in DMF (10 mL) was added TFA (0.1 mL, 1.34 mmol). The reaction mixture was stirred at room temperature for 10 min. EDC (0.26 g, 1.35 mmol) and HOBT (0.18 g, 1.35 mmol) were added and the reaction mixture was stirred at room temperature for 30 min. Ethyl R-ethyl 3-(N-gly)-amino-3-(3-bromo-5-chloro-2-hydroxy)phenyl propionate hydrochloride prepared as in Example 1 (0.5 g; 1.22 mmol), followed by triethylamine (0.14 g; 1.34 mmol) were added to the reaction mixture and stirred for 18 h. The reaction mixture was then concentrated in vacuo and purified by reversed phase HPLC to afford 0.26 g (31%) white solid: MS (ES) m/z 584.26 (M+H)⁺; ¹H-NMR (400 MHz, CD₃OD) δ 1.18 (t, 3H, J=7.12 Hz), 2.78-3.01 (m, 2H), 3.37 (s, 4H), 4.05 (s, 2H), 4.06-4.11 (m, 2H), 5.56-5.60 (m, 1H), 6.82 (s, 1H), 7.18-7.24 (m, 3H), 7.41-7.42 (m, 1H).
Step 3

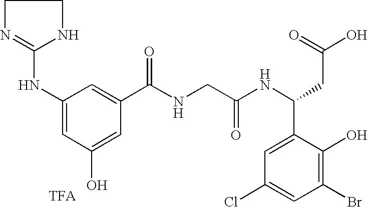

To a solution of ethyl ester obtained from step A (0.2 g, 0.29 mmol) in 50% acetonitrile and water (4 mL), was added LiOH (50 mg). The reaction mixture was stirred at room temperature for 3 h, and purified on reverse phase HPLC to give the title compound as its TFA salt (0.16 g, 84%); ¹H- NMR (400 MHz, CD₃OD) δ 2.76-2.89 (m, 2H), 3.76 (s, 4H), 4.05 (s, 2H), 5.23-5.56 (m, 1H), 6.80-6.81 (m, 1H), 7.19-7.25 (m, 3H), 7.40-7.41 (m, 1H). HRMS (M+H) calculated C₂₁H₂₁N₅O₆ClBr 556.0416. Found 556.0416.

### Example 22

### (β¹R)-3, 5-dimethyl-β-[[[[3-[(5-fluoro-1,4,5,6-tetrahydro-2-pyrimidinyl)-amino]-5-nitrobenzoyl]amino]acetyl]amino]-2-hydroxybenzenepropanoic acid, monotrifluoroacetate

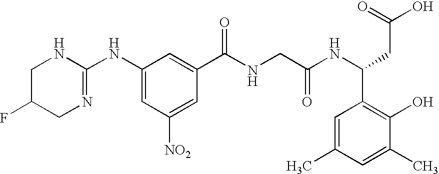 Step 1

### 2-Hydroxy-3,5-dimethylbenzaldehyde

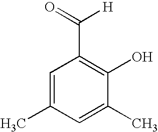

To the solution of ethylmagnesium bromide (400 mL, 1.0 M in THF) was added 2.4-dimethylphenol (49 g, 0.4 mol) in 40 mL toluene slowly at 4° C. To the above solution was added tetramethylethylenediamine (45 g, 0.4 mol) followed by adding paraformaldehyde (30 g) then HMPA (72.1 g, 0.4 mol) at room temperature. The reaction mixture was refluxed 4 hours then stirred at room temperature for 48 hours. The reaction mixture was quenched with 50% HCl (450 ml). The aqueous solution was extracted with ethyl acetate (4×250ml). Combined organic solution was washed with brine, dried over MgSO₄ and concentrated under vacuum. Concentrated residue was chromatographed on silica gel (5% ethyl acetate in hexane) to give 32.8 g (55%) oil. ¹H NMR (400 MHz, DMSO) δ 2.13 ~(s, 3H), 2.22 (s, 3H), 7.28-7.34 (m, 2H), 9.95 (s, 1H), 10.75(s, 1H). MS m/z 150.0681.

Step 2

### Tert-butyl(2E)-3-(2-hydroxy-3,5-dimethylphenyl)prop-2-enoate

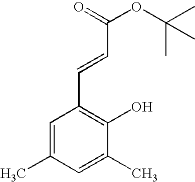

To the solution of 2-hydroxy-3,5-dimethylbenzaldehyde ( 20 g, 0.13 mol) in THF (200 ml) was added tert-butyl(triphenylphosphoranyl)acetate (50 g, 0.13 mol) followed by adding DBU (0.3 ml). The reaction mixture was stirred at room temperature for 48 hours. The reaction mixture was concentrated under vacuum. The concentrated residue was chromatographed on silica gel (10% ethyl acetate in hexane) to give 24 g (73%) white solid: ¹H NMR (400 MHz, CDCl₃) δ 1.52 ~(s, 9H), 2.22 (s, 6H), 5.05 (s, 1H), 6.4 (d, 1H, J=15.8 Hz), 6.93 (s, 1H), 7.12 (s, 1H), 7.90 (d, 1H). HRMS calculated for C₁₅H₂₀O₃ (M+H): 271.1310, found 271.1316.

Step 3

### Tert-butyl 3-(hydroxyamino)-3-(2-hydroxy-3,5-dimethylphenyl)propanoate

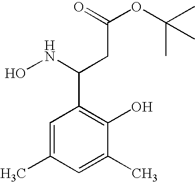

To the solution of tert-butyl(2E)-3-(2-hydroxy-3,5-dimethylphenyl)prop-2-enoate (23.5 g, 94 mmol) in deoxane (60 ml) was added 50% hydroxylamine (20 ml) followed by adding tetrabutylammonium sulfate (0.1 g). The reaction mixture was stirred at room temperature for 48 hours. The reaction mixture was extracted with ethyl acetate (200 ml). Organic layer was separated and washed with water, brine, dried over MgSO₄ and concentrated. Dried to give 26.4 g (99%) oil. The crude material was used for next reaction without further purfication. ¹H NMR (400 MHz, DMSO) δ 1.18 ~(s, 9H), 2.08 (s, 3H), 2.10 (s, 3H), 2.8-2.9 (m, 1H), 3.0 (m, 1H), 4.9 (m, 1H), 6.8 (m, 2H). M+H=282.2

Step 4

### Tert-butyl 3-amino-3-(2-hydroxy-3,5-dimethylphenyl)propanoate

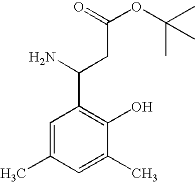

To the solution of tert-butyl 3-(hydroxyamino)-3-(2-hydroxy-3,5-dimethylphenyl)-propanoate (8 g, 28.5 mmol) in acetic acid (60 ml) was added zinc dust (10 g) at 0° C. The reaction was stirred at 0° C. for 30 min. and 6 hours at room temperature. Zinc dust was filter out through celite. The filtrated was concentrated and purified on reverse phase HPLC to give 5.4 g (50%) TFA salt of title compound. ¹H NMR (300 MHz, DMSO) δ 1.26 ~(s, 9H), 2.13 (s, 3H), 2.14 (s, 3H), 2.77-2.95 (m, 2H), 4.71 (m, 1H), 6.88 (s,1H), 6.89 (s, 1H), 8.08 (bs, 3H). M+H=266.2

Step 5

### Ethyl 3-amino-3-(2-hydroxy-3,5-dimethylphenyl)propanoate hydrochloride

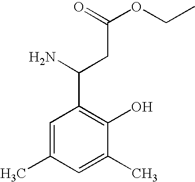

Tert-butyl 3-amino-3-(2-hydroxy-3,5-dimethylphenyl )propanoate trifluoroacetate (19.5 g, 73.5 mmol) was stirred in saturated HCl in ethanol (20 ml) under nitrogen for 4 hours. The reaction mixture was concentrated under vacuum. To the concentrated residue was added ether (100 ml) and the solution was stirred for one hour. Solid was formed and filtered. Dried to give 13.5 g (96%) HCl salt of the title compound. ¹H NMR (400 MHz, DMSO) δ 1.07 ~(t, 3H, J=7.11 Hz), 2.12 (s, 6H), 2.86-3.00 (m, 2H), 4.00 (q, 2H, J=6.78 Hz), 4.74-4.79 (m, 1 H), 6.87 (s, 1H), 6.92 (s, 1H), 8.18 (bs, 3H). C₁₃H₁₉NO₃ (M+H): 238.1443, found 238.1446.

Step 6

### Ethyl (3R)-3-amino-3-(2-hydroxy-3,5-dimethylphenyl)propanoate hydrochloride

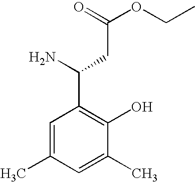

The R isomer of ethyl (3R)-3-amino-3-(2-hydroxy-3,5-dimethylphenyl)propanoate was resolved by enzyme resolution.

Step 7

### Ethyl (3R)-3-[(N-{3-[(5-fluoro-1,4,5,6-tetrahydropyrimidin-2-yl)amino]-5-nitro-benzoyl}glycyl)amino]-3-(2-hydroxy-3,5-dimethylphenyl)propanoate

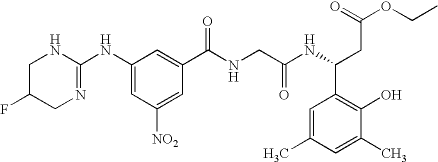

To a solution of 3-[(5-fluoro-1,4,5,6-tetrahydropyrimidin-2-yl)amino]-5-nitrobenzoic acid (0.52 g, 1.8 mmol) in DMF (10 ml) was added TFA (0.15 ml, 1.8 mmol). The reaction mixture was stirred at room temperature for 10 minutes. EDC (0.38 g, 1.35 mmol) was added followed by adding HOBT (0.27 g, 1.35 mmol). The reaction mixture was stirred at room temperature for 30 minutes. Ethyl (3R)-3-(glycylamino)-3-(2-hydroxy-3,5-dimethylphenyl )propanoate hydrochloride, (prepared by treating the product from step 6 and BOC-gly-OSU and treating the resulting product with Ethanolic HCl) (0.75 g; 1.8 mmol) was added to above solution followed by adding triethylamine (0.3 ml, 1.8 mmol). The reaction mixture was stirred for 18 hours. The reaction mixture was concentrated in vacuum and purified on reversed phase HPLC to afford the TFA salt of the title compound (0.54 g, 45%) as white solid. ¹H NMR (400 MHz, CD₃OD) \tilde{□}||~(t, 3H, J=7.21 Hz), 2.15 (s, 3H), 2.18 (s, 3H), 2.87 (d, 2H, J=7.25 Hz). 3.53-3.72 (m, 4H), 4.04-4.12 (m, 4H), 5.19-5.31 (m, 1H), 5.53-5.59 (m, 1H), 6.81 (s, 1H), 6.85 (s, 1H), 8.09-8.10 (m, 1H), 8.26-8.29 (m, 1H), 8.52-8.54 (m, 1H), 8.62-8.63 (m, 1H). HRMS calculated for C₂₆H₃₁N₆O₇F (M+H): 559.2311. Found 559.2300.

Step 8

### (3R)-3-[(N-{3-[(5-Fluoro-1,4,5,6-tetrahydropyrimidin-2-yl)amino]-5-nitrobenzoyl}-glycyl)amino]-3-(2-hydroxy-3,5-dimethylphenyl)propanoic acid trifluoroacetate

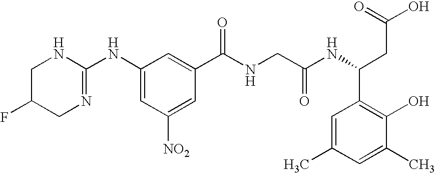

To a solution of above ethyl ester (0.13 g, 0.19 mmol) in 50% acetonitrile in water (1 ml) was added LiOH (45 mg). The reaction mixture was stirred at room temperature for 3 hours then purified on reverse phase HPLC to give the TFA salt of the title compound (82 mg, 68%). ¹H NMR (400 MHz, CD₃OD) δ 2.15 ~(s, 3H), 2.18 (s, 3H), 2.86 (d, 2H, J=7.18), 3.53-3.72 (m, 4H), 4.09 (s, 2H), 5.19-5.30 (m, 1H), 5.54 (t, 1H, J=7.05 Hz), 6.81 (m, 1H), 6.87 (s, 1H), 8.09 (t, 1H, J=1.75), 8.26 (t, 1H, J=2.01 Hz), 8.63 (t, 1H, J=1.74 Hz). ¹⁹F NMR (400 MHz, CD₃OD) δ −190.288 to −189.965. HRMS (M+H) calculated for C₂₄H₂₇N₆O₇F 531.1998. Found: 531.2008.

### Example 23

### (β¹R)-3,5-dimethyl-β-[[[[3-[(5-fluoro-1,4,5,6-tetrahydro-2-pyrimidinyl)-amino]-5-aminobenzoyl]amino]acetyl]amino]-2-hydroxybenzenepropanoic acid, monotrifluoroacetate

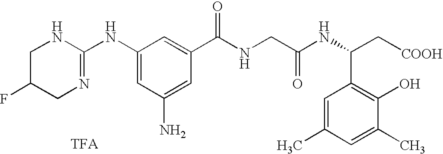

To a solution of the nitro ester obtained from Example 22 (0.49 g, 0.73 mmol) in acetic acid (10 mL) was added zinc dust (0.5 g) and the reaction mixture was stirred for 4 h at room temperature. Zinc dust was removed by filteration through a pad of celite. The filtrate was concentrated and the residue was dissolved in 50% water in acetonitrile (6 mL). To this solution was added LiOH (0.2 g), stirred for 1 h at room temperature, and acidified to pH 4 by adding TFA. The resulting mixture was purified by reverse phase HPLC to give the title compound (0.15 g, 33%): ¹H-NMR (400 MHz, CD₃OD) δ 2.17 (s, 3H), 2.20 (s, 3H), 2.89 (d, 2H, J=6.98), 3.31-3.69 (m, 4H), 4.04 (s, 2H), 5.15-5.27 (m, 1H), 5.55 (t, 1H, J=6.92 Hz), 6.69-6.7 (m, 1H), 6.83-6.87 (m, 1H), 6.94-6.95 (m, 1H), 6.95 (s, 1H), 7.01 -7.08 (m, 1H); ¹⁹F NMR (400 MHz, CD₃OD) δ −190.56 to −190.16 (m, 1F). HRMS (M+H) calcd C₂₄H₂₉N₆O₅F 501.2256. Found 501.2254

### Example 24

### (R)-3-Bromo-5-chloro-2-hydroxy-β-[[2-[[[3-hydroxy-5-[(1,4,5,6-tetrahydro-pyrimidin-2-yl)amino]phenyl]carbonyl]amino]acetyl]amino]benzenepropanoic acid, trifluroacetate salt

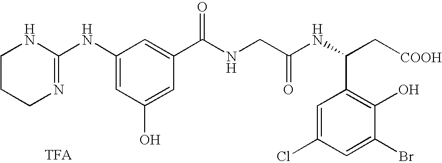 Step 1

### N-benzoyl-N′-(5-hydroxy-3-carboxyphenyl)thiourea

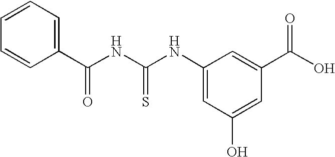

A mixture of 3-amino-5-hydoxybenzoic acid (30.7 g, 200.7 mmol) and benzoyl isothiocyanate (26.57 g) in acetonitrile (450 mL) was stirred at room temperature for 1 h. The precipitate was filtered and washed with acetonitrile and dried to afford 57.17 g (90%) of the desired product as a yellow powder. ¹H NMR (CD₃OD) δ 8.01-8.04 (m, 2H), 7.79 (m, 1H), 7.69 (m, 1H), 7.58-7.63 (m, 2H), 7.37 (m, 1H). Anal. Calcd for C₁₅H₁₂N₂SO₄: Mol. Wt, 316.0518. Found: 317.0593 (M+H, HRMS).

Step 2

### N-(5-hydroxy-3-carboxyphenyl)thiourea

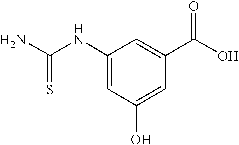

Sodium methoxide (106 mL, 25%) was added slowly to a stirred mixture of N-(benzoyl)-N′-(5-hydroxy-3-carboxyphenyl)thiourea (51.77 g, 163.73 mmol) in anhydrous methanol (250 mL). A clear solution resulted in 10 min. After 1 h stirring at rt, methanol was removed in vacuo and the residue was dried in vacuo. The residue was triturated with ether (500 mL) to leave a orange powder. The residue was dissolved in water (150 mL) and acidified to pH 6. The solid formed was filtered and dried. The solid was further washed with ether (100 mL). The residue obtained is the desired product. Yield: 34.6 g, (99.5%). ¹H NMR (CD₃OD) δ 7.42 (m, 1H), 7.28 (m, 1H), 7.11 (m, 1H). Anal. Calcd for C₈H₈N₂SO₃: Mol. Wt, 212.0256. Found: 213.0303 (M+H, HRMS).

Step 3

### N-(5-hydroxy-3-carboxyphenyl)-S-methylisothiourea

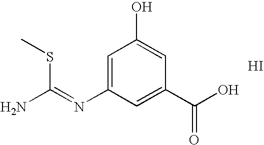

A mixture of N-(5-hydroxy-3-carboxyphenyl)thiourea (32.22 g, 0.164 mol) and iodomethane (23.34 g) in ethanol (200 mL) was heated at reflux for 5 h, the solution turned homogeneous. The solution was concentrated. Yield 56.89 g: (100%). This compound has been synthesized previously starting from the isothiourea and 1,3-diamino-2-hydroxypropane. ¹H NMR (CD₃OD) δ 7.26-7.32 (m, 2H), 6.93 (m, 1H), 2.67 (s, 3H). Anal. Calcd for C₉H₁₀O₃N₂S: Mol. Wt, 226.0412. Found: Mol. W, 227.0462 (M+H, HRMS).

Step 4

### 3-N-(tetrahydropyrimidinyl)-5-hydroxybenzoic acid

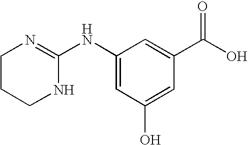

A mixture of N-(5-hydroxy-3-carboxyphenyl)-S-methylisothiourea (28.44 g, 0.084 mol) and diaminopropane (18.66 g, 0.252 mol) was heated at 100 C for 28 hours in DMF (40 mL). The reaction mixture was cooled and filtered, and was washed with ethyl acetate and ether. The solid was dried to afford 27 g. of the crude product. This was added 4N HCl in dioxane and was allowed to stir for 2 h and was concentrated. The residue was washed twice with ether to afford 16.0 g (70%) of the desired product as a powder. ¹H NMR (CD₃OD) δ 7.13-7.21 (m, 2H), 6.86 (m, 1H), 3.26 (m, 4H), 1.83 (m, 2H). Anal. Calcd for C₁₁H₁₃O₃N₃: Mol. Wt, 236.1005 (M+H, HRMS). Found: Mol. W, 236.1035 (M+H, HRMS).
Step 5.

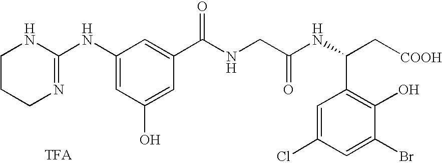

To a solution of 3-hydroxy-5-[(1,4,5,6-tetrahydro-2-pyrimidinyl)amino]benzoic acid hydrochloride (0.3 g; 1.3 mmol) in DMF (7 mL), 1-(3-dimethylamino-propyl)-3-ethylcarbodiimide hydrochloride (0.28 g; 1.5 mmol) and 1-hydroxy-benzo-triazole hydrate, HOBt (0.2 g; 1.5 mmol) were added. After stirring the reaction mixture at room temperature for 30 min, ethyl R-3-(N-gly)-amino-3-(3-bromo-5-chloro-2-hydroxyphenyl)propionate hydroxyphenyl)propionate hydrochloride, prepared as in Example 1 (0.56 g; 1.3 mmol) and triethylamine (0.15 g; 1.5 mmol) were added and the resulting mixture was stirred at room temperature for 18 h. It was concentrated in vacuo and the residue was purified by reversed phase HPLC to afford the ethyl ester of the title compound (0.38 g, 40%) as white solid. This product was dissolved in acetonitrile: water/1:1 (8 mL), added lithium hydroxide (0.16 g), and stirred at room temperature for 3 h. The product was purified by HPLC to afford the title compound (0.3 g): ¹H-NMR (CD₃OD) δ 1.95-2.01 m, 2H), 2.90-2.77 (m, 2H), 3.37 (t, 4H, J=5.91 Hz), 4.05 (s, 2H), 5.5 (d, 1H, J =5.5 Hz), 6.78 (m, 1H), 7.14 (s, 1H), 7.18 (s, 1H), 7.24 (d, 1H, J=2.42 Hz), 7.41 (d, 1H, J=2.42). MS (ES) m/z Calcd for C₂₂H₂₃N₅O₆ 570.0578. Found: 570.0534 (M+H).

### Example 25

### (R)-5-Chloro-3-bromo-2-hydroxy-β-[[2-[[5[(1,4,5,6-tetrahydro-5-hydroxy-pyrimidin-2-yl)amino]phenyl]carbonyl]amino]acetyl]amino]benzenpropanoic acid, trifluroacetate salt

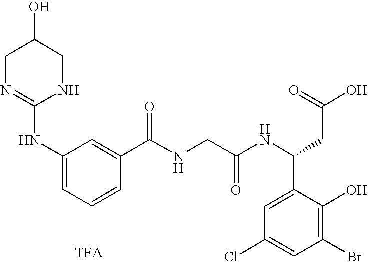

To a solution of 3-(5-hydroxytetrahydropyrimidino)-benzoic acid prepared using similar procedure according to U.S. Pat. No. 6,028,223 Example 415 (0.33 g, 1.2 mmol) in DMF (5 mL), was added EDC (0.25 g, 1.3 mmol) and HOBt (0.18 g, 1.3 mmol). The reaction mixture was stirred at room temperature for 15 min. and then added a solution of ethyl R-3-(N-gly)-amino-3-(3-bromo-5-chloro-2-hydroxy-phenyl)propionate hydrochloride, prepared as in Example 1 (0.5 g, 1.2 mmol) in DMF, followed by the addition of triethylamine (0.15 g, 1.4 mmol). The resulting mixture was stirred 18 h at room temperature and the product was purified by HPLC to afford the ethyl ester of the title compound (0.26 g, 30.6%). This ester (0.2 g, 0.28 mmol) was dissolved in 50% acetonitrile in water (5 mL), added lithium hydroxide (50 mg) and stirred at room temperature for 3 h. The product was purified by HPLC to afford the title compound (0.12 g, 79%). ¹H-NMR (CD₃OD) δ 2.77-2.9 (m, 2H), 3.31-3.46 (m, 4H), 4.08 (d, 2H, J=1.07 Hz), 4.21-4.24 (m, 1H), 5.53-5.56 (m, 1H), 7.24-7.25 (d, 1H, J=2.55 Hz), 7.40-7.42 (m, 2H), 7.53 (t, 1H, J=7.85 Hz), 7.73-7.74 (m, 1H), 7.77-7.79 (m, 1H). MS (ES) m/z calcd C₂₂H₂₃N₅O₆ClBr 570.0578. Found 570.0582(M+H)⁺.

### Example 26

### (β¹R)-3-methyl-5-chloro-β-[[[[3-[(5-hydroxy-1,4,5,6-tetrahydro-2-pyrimidinyl)-amino]-5-hydroxybenzoyl]amino]acetyl]amino]-2-hydroxybenzenepropanoic acid, monotrifluoroacetate

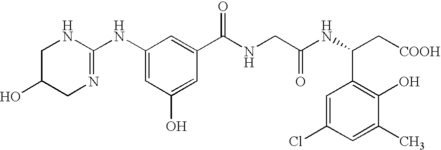

To a solution of 3-hydroxy-5-[(5-hydroxy-1,4,5,6-tetrahydropyrimidin-2-yl)amino]-benzoic acid hydrochloride (prepared according to U.S. Pat. No. 6,013,651, Example H, 0.3 g; 0.97 mmol) in DMF (10 mL), EDC (0.19 g ; 0.97 mmol) and HOBT (0.13 g; 0.97 mmol) were added and the mixture was stirred at room temperature. After 30 min, ethyl R-3-(N-gly)-amino-3-(5-chloro-3-methyl-2-hydroxyphenyl)propionate hydrochloride, prepared as in Example 29, (0.28 g; 0.97 mmol) was added followed by the addition of triethylamine (0.15 mL, 0.97 mmol). The resulting mixture was stirred at room temperature for 18 h and concentrated in vacuo. The residue was purified by reversed phase HPLC to afford the ethyl ester of the title compound (0.38 g, 59%) as white solid. This material was dissolved in acetonitrile: water/1:1 (8 mL), added lithium hydroxide (0.1 g) and the mixture was stirred at room temperature for 3 h. The desired product was purified by HPLC to afford the title compound (0.12 g, 36%). ¹H-NMR (CD₃OD) δ 2.18 (s, 3H), 2.79-2.88 (m, 2H), 3.31-3.45 (m, 4H), 4.04 (s, 2H), 4.21-4.22 (m, 1H), 6.81 (t, 1H, J=2.08 Hz), 6.99 (d, 1H, J=2.01 Hz), 7.05 (d, 1H, J=2.55 Hz), 7.16-7.19 (m, 2H). HRMS (ES) m/z calcd for C₂₃H₂₆N₅O₇Cl 520.1594. Found 520.1571 (M+H)⁺.

### Example 27

### (β¹R)-3,5-dimethyl-β-[[[[3-[(5-hydroxy-1,4,5,6-tetrahydro-2-pyrimidinyl)-amino]-5-hydroxybenzoyl]amino]acetyl]amino]-2-hydroxybenzenepropanoic acid, monotrifluoroacetate

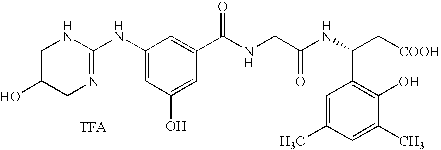

To a solution of N-[3-hydroxy-5-[(5-hydroxy-1,4,5,6-tetrahydropyrimidin-2-yl)amino]benzoyl]glycine (prepared according to U.S. Pat. No. 6,013,651, Example H, 0.3 g; 0.97 mmol) in DMF (10 mL) was added TFA (0.11 g, 0.97 mmol) and stirred for 15 min at room temperature. To this solution, EDC (0.19 g, 0.97 mmol) and HOBT (0.13 g; 0.97 mmol) were added and the mixture was stirred at room temperature for 30 min. Then added ethyl [3R]-3-amino-3-(2-hydroxy-3,5-dimethylphenyl)propionate hydrochloride prepared as in Example 22 (0.22 g; 0.97 mmol), followed by the addition of triethylamine (0.13 mL, 0.97 mmol). The resulting mixture was stirred at room temperatue for 18 h, and concentrated under reduced pressure. The residue was dissolved in acetonitrile: water/1:1 (8 mL), added lithium hydroxide (40 mg), and the reaction mixture was stirred at room temperature for 3 h. The desired product was isolated by HPLC to afford the title compound (0.1 g, 16%). ¹H-NMR (CD₃OD) δ 2.15 (s, 3H), 2.18 (s, 3H), 2.86 (d, 2H, J=7.12 Hz), 3.31-3.45 (m, 4H), 4.03 (s, 2H), 4.20-4.22 (m, 1H), 5.53 (t, 1H, J=7.05 Hz), 6.80-6.82 (m, 1H), 6.85 (bs, 1H), 7.15-7.16 (m, 1H), 7.17-7.18 (m, 1H). HRMS calculated for C₂₄H₂₉N₅O₇ (M+H): 500.2140. Found: 500.2148.

### Example 28

### (R)-β-[[2-[[[3-hydroxy-5-[4,5-(dihydro-1H-imidazol-2yl)amino]-phenyl]carbonyl]-amino]acetyl]amino]3,5-dichloro-2-hydroxybenzenepropanoic acid, trifluoroacetate salt

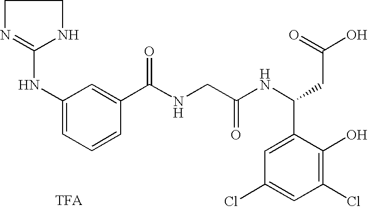

To a solution of 3-[4,5-(dihydro-1H-imidazol-2yl)-benzoic acid monohydro-chloride, prepared according to procedure in U.S. Pat. No. 6,028,223, Example 238 Step A, (0.64 g, 3.0 mmol) in DMF (10 mL), EDC (0.58 g, 0.3 mmol) and HOBT (0.18 g, 1.35 mmol) were added and the mixture was stirred at room temperature for 30 min. Then, ethyl R-ethyl 3-(N-gly)-amino-3-(3,5-dichloro-2-hydroxy)phenyl propionate hydrochloride prepared as in Example 3 (1.12 g, 3.0 mmol) was added, followed by the addition of triethylamine (0.31 g; 3.0 mmol). The resulting mixture was stirred at room temperature for 18 h, and the product was isolated by reversed phase HPLC to afford the title compound (0.36 g, 18%) as white solid. This material was dissolved in 50% acetonitrile in water (6 mL), added LiOH (75 mg), and the mixture was stirred at room temperature for 3 h. The product was isolated by reverse phase HPLC to give the title compound (79%). ¹H NMR (400 MHz, CD₃OD) δ 2.75-2.89 (m, 2H), 3.77 (s, 4H), 4.04-4.13 (m, 2H), 5.53-5.57 (m, 1H), 7.21 (d, 1H, J=2.24 Hz), 7.26 (d, 1H, J=2.55 Hz), 7.41-7.43 (m, 1H), 7.53-7.57 (m, 1 H), 7.76-7.81 (m, 2 H). HRMS (M+H), m/z calcd C₂₁H₂₁N₅O₅Cl₂ 494.093. Found 494.1011.

### Example 29

### (R) 5-chloro-3-methyl-2-hydroxy-β-[[2-[[[3-hydroxy-5-[imidazolidine-2-amino]-phenyl]carbonyl]amino]acetyl]amino]benzenepropanoic acid, trifluroacetate salt

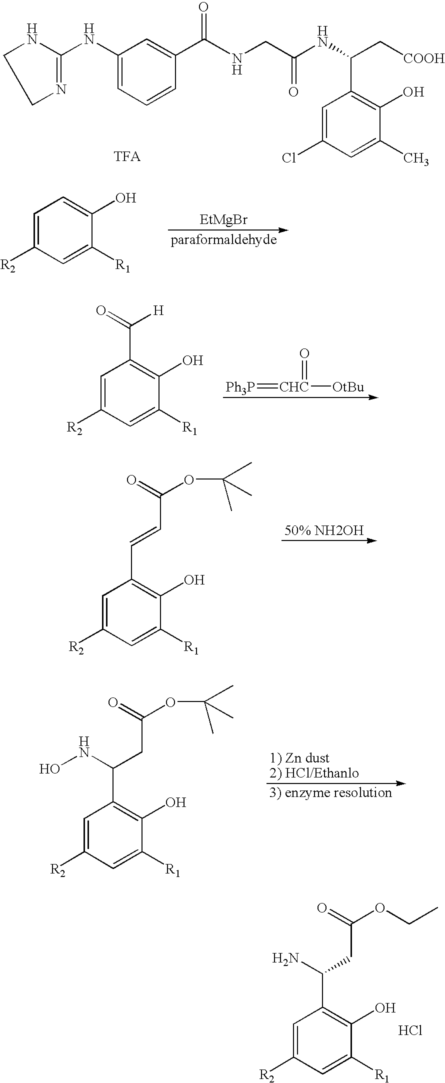 Step 1

### 5-chloro-2-hydroxy-3-methylbenzaldehyde

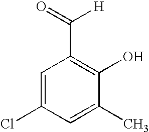

To the solution of ethylmagnesium bromide (400 mL, 1.0 M in THF) was added 4-chloro-2-methylphenol (57 g, 0.4 mol) in 75 mL toluene slowly at 4° C. To the above solution was added tetramethylethylenediamine (45 g, 0.39 mol) followed by adding paraformaldehyde (30 g) then HMPA (72.1 g, 0.4 mol) at room temperature. The reaction mixture was refluxed 4 hours then stirred at room temperature for 48 hours. The reaction mixture was quenched with 50% HCl (450 ml). The aqueous solution was extracted with ethyl acetate (4×250 ml). Combined organic solution was washed with brine, dried over MgSO₄ and concentrated under vacuum. Concentrated residue was chromatographed on silica gel (5% ethyl acetate in hexane) to give 40.8 g (60%) oil. ¹H NMR (400 MHz, CDCl₃) δ 2.25 (s, 3H), 7.36 (m, 2H), 9.8 (s, 1H), 11.16(s, 1H).

Step 2

### tert-butyl (2E)-3-(5-chloro-2-hydroxy-3-methylphenyl)prop-2-enoate

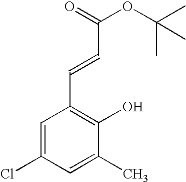

To the solution of 5-chloro-2-hydroxy-3-methylbenzaldehyde (22.6 g, 0.13 mol) in THF (200 ml) was added tert-butyl (triphenylphosphoranyl)acetate (50 g, 0.13 mol) followed by adding DBU (0.3 ml). The reaction mixture was stirred at room temperature for 48 hours. The reaction mixture was concentrated under vacuum. To the concentrated residue was added 10% ethyl acetate in hexane (100 ml). Solid was formed in the solution and filtered out. The filtrated was concentrated and chromatographed on silica gel (10% ethyl acetate in hexane) to give 33.4 g (88%) white solid. ¹H NMR (300 MHz, CDCl₃) δ 1.56 (s, 9H), 2.32 (s, 3H), 6.20 (s, 1H), 6.44 (d, 1H, J=16.11 Hz), 7.13 (m, 1H), 7.35 (m, 1H), 8.01 (d, 1H, J=16.11).

Step 3

### tert-butyl 3-(5-chloro-2-hydroxy-3-methylphenyl)-3-(hydroxyamino)propanoate

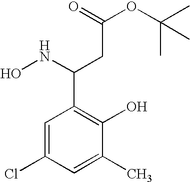

To the solution of tert-butyl (2E)-3-(5-chloro-2-hydroxy-3-methylphenyl)prop-2-enoate (9 g, 33.5 mmol) in deoxane (21 ml) was added 50% hydroxylamine (8 ml) followed by adding tetrabutylammonium sulfate (0.1 g). The reaction mixture was stirred at room temperature for 48 hours. The reaction mixture was extracted with ethyl acetate (200 ml). Organic layer was separated and washed with water, brine, dried over MgSO4 and concentrated. Dried to give 9.7 g (96%) oil. ¹H NMR (300 MHz, DMSO) δ 1.24 (s, 9H), 2.19 (s, 3H), 3.06 (m, 2H), 5.1 (m, 1H), 7.2 (m, 2H). M+H=302.1

Step 4

### tert-butyl 3-amino-3-(5-chloro-2-hydroxy-3-methylphenyl)propanoate trifluoroacetate

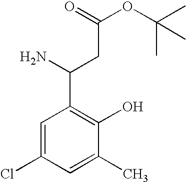

To the solution of tert-butyl 3-(5-chloro-2-hydroxy-3-methylphenyl)-3-(hydroxyamino)propanoate (9.5 g, 31.5 mmol) in acetic acid (65 ml) was added zinc dust (9 g) at 0° C. The reaction was stirred at 0° C. for 30 min. and 6 hours at room temperature. Zinc dust was filter out through celite. The filtrated was concentrated and purified on reverse phase HPLC to give 8 g (65.5%) TFA salt of title compound: ¹H NMR (400 MHz, CD₃OH) δ 1.39 (s, 9H), 2.90-3.03 (m, 2H), 4.76 (t, 1H, J=7.25), 7.11 (m, 1H), 7.17 (m, 1H).

Step 5

### Ethyl 3-amino-3-(5-chloro-2-hydroxy-3-methylphenyl)propanoate hydrochloride

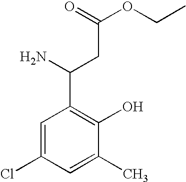

Tert-butyl 3-amino-3-(5-chloro-2-hydroxy-3-methylphenyl)propanoate trifluoroacetate (4 g, 10 mmol) was stirred in saturated HCl in ethanol (20 ml) under nitrogen for 4 hours. The reaction mixture was concentrated under vacuum. To the concentrated residue was added ether (100 ml) and the solution was stirred for one hour. Solid was formed and filtered. Dried to give 2.8 g (93%) HCl salt of the title compound: ¹H NMR (400 MHz, CD₃OH) δ 1.21 (t, 3H), 2.24 (s, 3H), 2.98-3.15 (m, 2H), 4.16 (m, 1H), 7.13 (m, 1H), 7.17 (m, 1H).

Step 6

### Ethyl (3R)-3-amino-3-(5-chloro-2-hydroxy-3-methylphenyl)propanoate hydrochloride

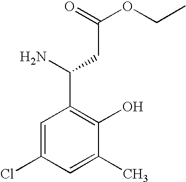

The R isomer of ethyl 3-amino-3-(5-chloro-2-hydroxy-3-methylphenyl)propanoate was resolved by enzyme resolution.

Step 7

### (3R)-3-(5-chloro-2-hydroxy-3-methylphenyl)-3-({N-[3-(4,5-dihydro-1H-imidazol-2ylamino)benzoyl]glycyl}amino)propanoic acid trifluoroacetate

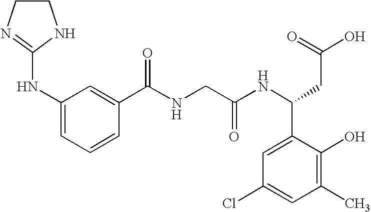

To the solution of N-[3-(4,5-dihydro-1H-imidazol-2-ylamino)benzoyl]glycine hydrochloride (0.3 g, 1 mmol) in 7 mL DMF was added EDC (0.21 g, 1.1 mmol), HOBT (0.15 g, 1.1 mmol). The reaction mixture was stirred at room temperature for 30 min. To the above solution was added ethyl (3R)-3-amino-3-(5-chloro-2-hydroxy-3-methylphenyl)propanoate hydrochloride (0.29 g, 1 mmol) followed by triethyl amine (0.11 g, 1.1 mmol). The reaction mixture was stirred at room temperature overnight. The reaction mixture was concentrated in vacuum and purified on reversed phase HPLC to afford 0.35 g (56%) white solid. The ethyl ester was dissolved in 8 mL 50% acetonitrile in water and treated with lithium hydroxide (0.16 g). The reaction mixture was stirred at room temperature for 3 h and was purified on HPLC to afford quantitative yield of the desired product as its TFA salt. ¹HNMR (CD₃OD) δ 2.24. (s, 3H), 2.88-2.85 (m, 2H), 3.79 (s, 4H), 4.09 (s, 2H), 5.56 (t, 1H, J=6.88 Hz), 7.01 (s, 1H), 7.07 (d, 1H, J=2.20 Hz), 7.4 (m, 1H), 7.57 (t, 1H, J=7.9 Hz), 7.78 (d, 1H, J=9.52 Hz), 7.82 (s, 1H). MS (ES) m/e 474.05 (M+H). HRMS calculated for C₂₁H₂₁N₅O₆ClBr (M+H): 556.0416. Found 556.0487.

### Example 30

### (R) 3,5-Dichloro-2-hydroxy-β-[[2-[[5[(1,4,5,6-tetrahydro-5-hydroxypyrimidin-2-yl)-amino]phenyl]carbonyl]amino]acetyl]amino]benzenepropanoic acid, trifluroacetate salt

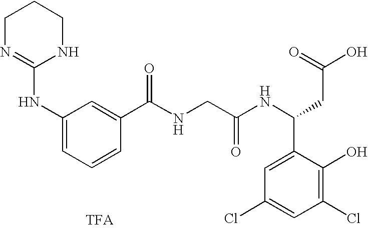 Step 1

### N-(3-Carboxyphenyl)-S-methylisothiourea

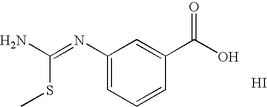

The thiourea (28.0 g, 0.1427 mol) and iodomethane (20.25 g, 8.9 mL, 0.1427 mol) was dissolved in ethanol (280 mL) and heated to reflux under a drying tube overnight. The clear reaction mixture was concentrated to afford 48.2 g (94%) of the desired product. ¹H NMR (CD₃OD) δ 11.3 (br 1H), 9.39 (br, 2H), 7.93 (d, 1H, J=7.25 Hz), 7.85 (s, 1H), 7.54-7.62 (m, 2H), 2.66 (s, 3H). Anal. Calcd for: C₉H₁₂N₂O₂S, Mol. Wt, 210.0463. Found: 211.0498 (M+H, HRMS).

Step 2

### N-(Tetrahydropyrimidinyl)-3-amniobenzoic acid

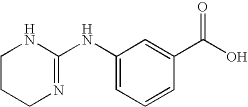

N-(3-Carboxyphenyl)-S-methylisothiourea (11.09 g, 0.0328 mol) and 1,3-diaminopropane (7.3 g, 0.098 mol) and DMF (25 mL) were added to 200 mL flask equipped with condenser and drying tube. The solution was heated at 80° C. for 18 h and was cooled and filtered. The solid was washed with ethyl acetate, then ether. Yield 5.3 g. (74%). ¹H NMR (CD₃OD) δ 9.58 (s, 1H), 8.16 (s, 2H), 7.77 (d, 1H, J=6.3 Hz), 7.72 (m, 1H), 7.47 (t, 1H, J=7.9 Hz), 7.40-7.41 (m, 1H), 3.24-3.25 (m, 4H), 1.83-1.85 (m, 2H). Anal. Calcd for C₁₁H₁₃O₂N₃: Mol. Wt, 219.1008. Found: Mol. W, 220.1048 (M+H, HRMS).
Step 3

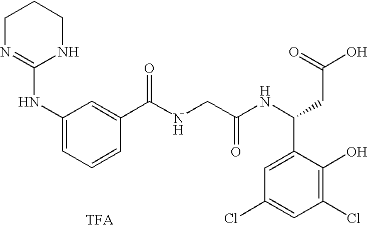

Trifluoroacetic acid (0.11 mL) was added to 3-[(1,4,5,6-tetrahydro-2-pyrimidinyl)-amino]benzoic acid (also reported in U.S. Pat. No. 6,028,223, Example 236), (0.3 g; 1.37 mmol) in 4 mL DMF and was stirred for 15 min. EDC (0.29 g; 1.5 mmol) followed by HOBt (0.2 g; 1.5 mmol) were added to the solution and the reaction mixture was stirred at room temperature for 30 minutes. Ethyl R-3-(N-gly)-amino-3-(3,5-dichloro-2-hydroxyphenyl)propionate hydrochloride, prepared as in Example 3, (0.5 g; 1.37 mmol) followed by triethylamine (0.16 g; 1.6 mmol) was added to the reaction mixture and was stirred for 18 h. The reaction mixture was concentrated in vacuo and purified by reversed phase HPLC and dried by lyophilization. The solid from lyophilization was dissolved in 50% acetonitrile in water (8 mL) and treated with lithium hydroxide (0.3 g). The reaction mixture was stirred at room temperature for 3 h and was purified by HPLC to afford 0.5 g (58.8%) of the desired product as its TFA salt. NMR (CD₃OD) δ 1.97-2.04 (m, 2H), 2.76-2.94 (m, 2H), 3.39 (t, 4H, J=5.78 Hz), 4.09 (d, 2H, J=1.17 Hz), 5.54-5.59 (m, 1H), 7.22 (d, 1H, J=2.49 Hz), 7.27 (d, 1H, J=2.49 Hz), 7.40 (d, 1H, J=7.9 Hz), 7.54 (t, 1H, J=7.83 Hz), 7.73 (d, 1H, J=1.9 Hz), 7.78 (d, 1H, J=7.9 Hz). HRMS (M+H) calcd C₂₂H₂₃N₅O₅Cl₂, 508.1154, found 508.1136.

### Example 31

### (R) 3-Bromo-5-chloro-2-hydroxy-β-[[2-[[[3-hydroxy-5-[(1,4,5,6-tetrahydro-pyrimidin-2-yl)amino]phenyl]carbonyl]amino]acetyl]amino]benzenepropanoic acid, trifluroacetate salt

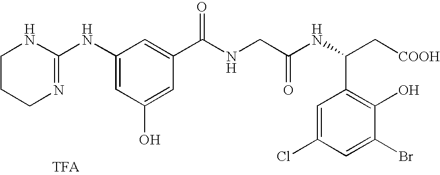

To a solution of 3-hydroxy-5-[(1,4,5,6-tetrahydro-2-pyrimidinyl)amino]-benzoic acid hydrochloride prepared as in Example 24, (0.3 g; 1.3 mmol) in DMF (7 mL) 1-(3-Dimethyl-aminopropyl)-3-ethylcarbodiimide hydrochloride (0.28 g; 1.5 mmol), and 1-hydroxybenzotriazole hydrate (0.2 g; 1.5 mmol) were added. The rection mixture stirred at room temperature. After 30 min., ethyl R-3-(N-gly)-amino-3-(3-bromo-5-chloro-2-hydroxyphenyl)propionate hydrochloride prepared as in Example 1, (0.56 g; 1.3 mmol) and triethylamine (0.15 g; 1.5 mmol) were added and the mixture was stirred at room temperature for 18 h. The reaction mixture was concentrated in vacuo and the product was isolated by reversed phase HPLC to afford 0.38 g (40%) white solid. This material was dissolved in 20 mL acetonitrile: water/1:1 (8.0 mL), added lithium hydroxide (0.16 g), and the mixture was stirred at room temperature for 3 h. The desired product was isolated by reverse-HPLC to afford 0.3 g of the title compound: ¹H-NMR (CD₃OD) δ 1.95-2.01 (m, 2H), 2.90-2.77 (m, 2H), 3.37 (t, 4H, J=5.91 Hz), 4.05 (s, 2H),5.5 (dd, 1H, J=5.5 Hz), 6.78 (m, 1H), 7.14 (s, 1H), 7.18 (s, 1H), 7.24 (d, 1H, J=2.42 Hz), 7.41 (d, 1H, J=2.42 Hz). HR MS (ES) m/z calcd for C₂₂H₂₄N₅O₆ClBr: 570.0578. Found 570.0534 (M+H)+.

### Example 32

### (β¹R)-3,5-dibromo-β-[[[[3-[(5-fluoro-1,4,5,6-tetrahydro-2-pyrimidinyl)-amino]-5-nitrobenzoyl]amino]acetyl]amino]benzenepropanoic acid, monotrifluoroacetate

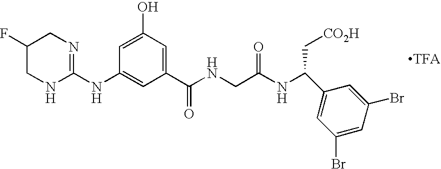 Step 1

### N-{3-[(5-fluoro-1,4,5,6-tetrahydropyrimidin-2-yl)amino]-5-hydroxybenzoyl}-glycine, trifluoroacetate

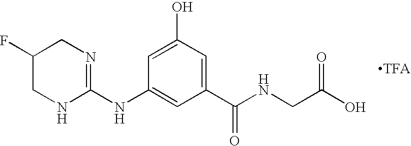

To a solution of 3-hydroxy-5-[(1,4,5,6-tetrahydro-5-fluoro-2-pyrimidyl)-amino]-benzoic acid hydrochloride prepared as in Example 8, (2.0 g (0.0069 mole), 0.7 g (0.0069 mole) of N-methylmorpholine (NMM), and 0.96 g (0.0069 mole) of ethyl glycinate hydrochloride in 18 mL of anhydrous N,N-dimethylacetamide (DMA) were added, followed by the addition of 1.05 g (0.0083 mole) of diisopropylcarbodiimide (DIC). at ice bath temperature. The reaction mixture was stirred overnight at room temperature. The precipitate was filtered off, and DMA was removed under vacuum at 50° C. 50-60 mL of water was added to the residue followed by 4.3 g (0.11 mole) NaOH. This mixture was stirred at room temperature for 3 h and filtered. The filtrate was neutralized with TFA, concentrated, and the residue was purified by reverse phase preparative HPLC to yield (after lyopholization) the title compound (850 mg) as a white solid: ¹H NMR (D₂O, 400 MHz) δ 7.08 (m, aromatic, 2H), 6.83 (m, aromatic, 1H), 5.19 (dm, J=32 Hz, 1H), 4.03 (s, 2H), 3.33-3.64 (m, 4H); HRMS [M+H]⁺ m/z calcd for C₁₃H₁₆FN₄O₄: 311.1156. Found: 311.1182.
Step 2

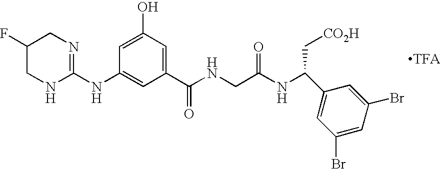

To N-{3-[(5-fluoro-1,4,5,6-tetrahydropyrimidin-2-yl)amino]-5-hydroxybenzoyl}-glycine, trifluoroacetate, Step 1 (0.25 g, 0.00059 mole), in 2 mL of anhydrous DMA in a flame dried flask, was added 81 mg (0.00059 mole) of isobutyl chloroformate (IBCF) at ice bath temp, followed by 60 mg (0.00059 mole) of NMM and stirred at 5° C. for 5 min, under nitrogen atmosphere. After stirring at room temperature for 10 min, ethyl-(R)-3-amino-3-(3,5-dibromo-phenyl)proprionate hydrochloride (0.193 g, 0.0005 mole; the synthesis of the racemate was described in U.S. Pat. No. 6,028,223) was added, followed by the addition of 50 mg (0.0005 mole) of NMM. The reaction was then stirred overnight at room temperature, water (6.0 mL) and acetonitrile (2.0 mL) were added, followed by the addition of 600 mg of NaOH. This mixture was stirred at room temperature for 3 h, acidified with TFA, and the product was isolated by reverse phase preparative HPLC to yield (after lyophilization) the title compound (120 mg) as a white solid: ¹H NMR (D₂O, 300 MHz) δ 7.56 (m, aromatic, 1H), 7.39 (m, aromatic, 2H), 7.06 (m, aromatic, 2H), 6.82 (m, aromatic, 1H), 5.06-5.29 (m, 2H), 3.95 (s, 2H), 3.31-3.64 (m, 4H), 2.73-2.82 (m, 2H); HRMS [M+H]⁺ m/z calcd for C₂₂H₂₃Br₂FN₅O₅: 616.0031. Found: 615.9999.

### Example 33

### (β¹R)-3,5-dimethyl-β-[[[[3-[(5-fluoro-1,4,5,6-tetrahydro-2-pyrimidinyl)amino]-5-nitrobenzoyl]amino]acetyl]amino]benzenepropanoic acid, monotrifluoroacetate

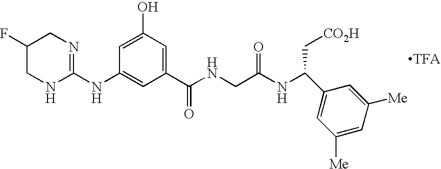

To N-{3-[(5-fluoro-1,4,5,6-tetrahydropyrimidin-2-yl)amino]-5-hydroxybenzoyl}-glycine, trifluoroacetate, prepared as in Example 32, Step 1, 250 mg (0.00059 mole), in 2 mL of anhydrous DMA in a flame dried flask under nitrogen, was added 81 mg (0.00059 mole) of isobutyl chloroformate (IBCF) at ice bath temperature, followed by the addition of 60 mg (0.00059 mole) of NMM. This mixture was stirred at 5° C. for 5 min. After stirring at room temperature for an additional 10 min, ethyl-(R)-3-amino-3-(3,5-dimethylphenyl)propionate hydrochloride 129 mg (0.0005 mole) (synthesis of the racemate described in U.S. Pat. No. 6,028,223; the R-enantiomer was then isolated via enzymatic resolution) was then added at 5° C., followed by the addition of 50 mg (0.0005 mole) of NMM. The resulting mixture was then stirred overnight at room temperature, 6 mL of water and 2 mL of acetonitrile were then added, followed by 600 mg of NaOH. This mixture was stirred at room temperature for 3 h, acidified with TFA, and the product was isolated by reverse phase preparative HPLC to yield (after lyopholization) the title compound (110 mg) as a white solid: ¹H NMR (D₂O, 300 MHz) δ 7.06 (m, aromatic, 2H), 6.89 (m, aromatic, 3H), 6.83 (m, aromatic, 1H), 5.06-5.28 (m, 2H), 3.94 (s, 2H), 3.31-3.63 (m, 4H), 2.71-2.77 (m, 2H), 2.15 (s, 6H); HRMS [M+H]⁺ m/z calcd for C₂₄H₂₉FN₅O₅: 486.2153. Found: 486.2172.

### Example 34

### (β¹R)-3-bromo-5-chloro-β-[[[[3-[(5-fluoro-1,4,5,6-tetrahydro-2-pyrimidinyl)-amino]-5-nitrobenzoyl]amino]acetyl]amino]benzenepropanoic acid, monotrifluoroacetate

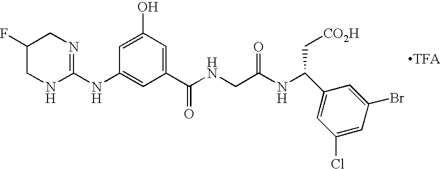

To N-{3-[(5-fluoro-1,4,5,6-tetrahydropyrimidin-2-yl)amino]-5-hydroxybenzoyl}-glycine, trifluoroacetate, prepared as in Example 32, Step 1, 183 mg (0.00043 mole), in 2 mL of anhydrous DMA in a flame dried flask under nitrogen was added 59 mg (0.00043 mole) of isobutyl chloroformate (IBCF) at ice bath temp, followed by the addition of 43 mg (0.00043 mole) of NMM. This mixture was stirred at 5° C. for 5 min. After stirring for an additional 10 min at room temperature, ethyl-(R)-3-amino-3-(3-bromo-5-chlorophenyl)propionate hydrochloride 132 mg (0.00037 mole; synthesis of the racemate was described in U.S. Pat No. 6,028,223; the R-enantiomer was then isolated via enzymatic resolution) was then added followed by the addition of 38 mg (0.00037 mole) of NMM. The reaction was then stirred overnight at room temperature, water (6.0 mL) and acetonitrile (2.0 mL) were then added followed by 600 mg of NaOH. This mixture was stirred at room temperature for 3 h, acidified with TFA, and the product was isolatedisolated by reverse phase preparative HPLC to yield (after lyopholization) the title compound (100 mg) as a white solid: ¹H NMR (D₂O, 300 MHz) δ 7.40 (m, aromatic, 1H), 7.33 (m, aromatic, 1H), 7.23 (m, aromatic, 1H), 7.04 (m, aromatic, 2H), 6.80 (m, aromatic, 1H), 5.07-5.24 (m, 2H), 3.94 (m, 2H), 3.32-3.64 (m, 4H), 2.72-2.77 (m, 2H); HRMS [M+H]⁺ m/z calcd for C₂₂H₂₃ClBrFN₅O₅: 572.0535. Found: 572.0538

### Example 35

### (β¹R)-3,5-dichloro-β-[[[[3-[(5-fluoro-1,4,5,6-tetrahydro-2-pyrimidinyl)amino]-5-nitrobenzoyl]amino]acetyl]amino]benzenepropanoic acid, monotrifluoroacetate

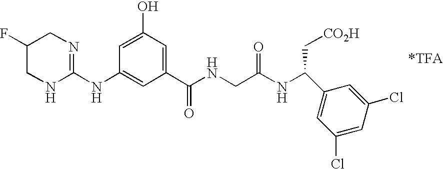

To N-{3-[(5-fluoro-1,4,5,6-tetrahydropyrimidin-2-yl)amino]-5-hydroxybenzoyl}-glycine, trifluoroacetate, prepared as in Example 32, Step 1, (250 mg, 0.00059 mole), in 2 mL of anhydrous DMA in a flame dried flask under nitrogen at 5° C., was added 81 mg (0.00059 mole) of isobutyl chloroformate (IBCF), followed by the addition of 60 mg (0.00059 mole) of NMM. This mixture was stirred at 5° C. for 5 min. After stirring the reaction mixture for an additional 10 min, ethyl-(R)-3-amino-3-(3,5-dichlorophenyl)proprionate hydrochloride (150 mg, 0.0005 mole) of (synthesis of the racemate described in U.S. Pat. No. 6,028,223; the R-enantiomer was isolated via enzymatic resolution) was then added at 5° C. followed by the addition of 50 mg (0.0005 mole) of NMM. The reaction was then stirred overnight at room temperature, water (6 mL) and acetonitrile (2 mL) were then added followed by 600 mg of NaOH. The resulting mixture was stirred at room temperature for 3 h, acidified with TFA, and the product was isolated by reverse phase preparative HPLC to yield (after lyopholization) the title compound (180 mg) as a white solid: ¹H NMR (D₂O, 300 MHz) δ 7.23 (m, aromatic, 1H), 7.19 (m, aromatic, 2H), 7.03 (m, aromatic, 2H), 6.79 (m, aromatic, 1H), 5.06-5.29 (m, 2H), 3.95 (s, 2H), 3.30-3.64 (m, 4H), 2.72-2.81 (m, 2H); HRMS [M+H]⁺ m/z calcd for C₂₂H₂₃Cl₂FN₅O₅: 526.1060. Found: 526.1063.

### Example 36

### (β¹R)-3,iodo-5-bromo-β-[[[[3-[(5-fluoro-1,4,5,6-tetrahydro-2-pyrimidinyl)-amino]-5-nitrobenzoyl]amino]acetyl]amino]benzenepropanoic acid, monotrifluoroacetate

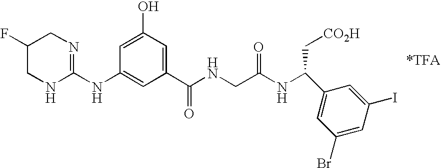

To N-{3-[(5-fluoro-1,4,5,6-tetrahydropyrimidin-2-yl)amino]-5-hydroxybenzoyl}-glycine, trifluoroacetate, prepared as in Example 32, Step 1, (183 mg, 0.00043 mole), in 2 mL of anhydrous DMA in a flame dried flask under nitrogen was added 59 mg (0.00043 mole) of isobutyl chloroformate (IBCF) at 5° C., followed by 43 mg (0.00043 mole) of NMM. This mixture was stirred at 5° C. for 5 min. After stirring at room temperature for an additional 10 min, ethyl-(R)-3-amino-3-(3-bromo-5-iodophenyl)proprionate hydrochloride (160 mg, 0.00037 mole; the synthesis of the racemate was described in U.S. Pat. No. 6,028,223; the R-enantiomer was isolated via enzymatic resolution) was then added 5° C., followed by the addition of 38 mg (0.00037 mole) of NMM. The resulting mixture was then stirred overnight at room temperature, water (6 mL) and acetonitrile (2 mL) were then added followed by the addition of 600 mg of NaOH. This mixture was stirred at room temperature for 3 h, acidified with TFA the product, and the product was isolated by reverse phase preparative HPLC to yield (after lyopholization) the title compound (90 mg) as a white solid: ¹H NMR (D₂O, 300 MHz) δ 7.74 (m, aromatic, 1H), 7.56 (m, aromatic, 1H), 7.40 (m, aromatic, 1H), 7.05 (m, aromatic, 2H), 6.80 (m, aromatic, 1H), 5.04-5.26 (m, 2H), 3.93 (m, 2H), 3.32-3.61 (m, 4H), 2.72-2.79 (m, 2H); HRMS [M+H]⁺ m/z calcd for C₂₂H₂₃BrIFN₅O₅: 663.9894. Found: 663.9837.

### Example 37

### (R) 5-Chloro-3-iodo-2-hydroxy-β-[[2-[[[3-hydroxy-5-[(1,4,5,6-tetrahydro-5-hydroxy-pyrimidin-2-yl)amino]phenyl]carbonyl]amino]acetyl]amino]-benzenepropanoic acid, trifluroacetate salt

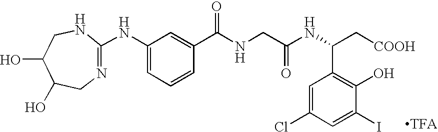 Step 1

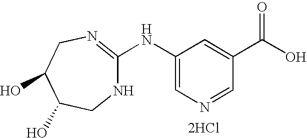

To a solution of 1,4-diamino-2,3-dihydroxybutane dihydrochloride (2.21 g, 0.012 mole, synthesized from dimethyl-L-tartrate as described in J. Carbohydrate Chemistry, 5, (2), 183-197, [1986]), in water (6 mL) and anhydrous DMF (10 mL), was added sodium carbonate (1.83 g, 0.017 mole). To this mixture, the isothiourea from Example 2 (1.21 g, 0.006 mole) was added and the mixture was heated at 85° C. for 3 h. After cooling in an ice bath, DMF was distilled in vacuo, the resulting residue was suspended in water, and the pH was adjusted to 5.6. This solution was lyophilized to afford the desired product (0.907 g, 59% yield). MS was consistent with the desired structure M+H 267. This compound was converted to its HCl salt by stirring with 4N HCl/dioxane (2 eq) in THF (10 mL) at 10° C. for 1 h.

Step 2

A solution of 3-N-(5,6-dihydroxytetrahydrodiazipino)amino-5-hydroxy-benzoic acid hydrochloride (1.65 g g, 5.94 mmol) in dimethylacetamide (25 mL) was heated until all the material had dissolved. This was then cooled to 0° C. and isobutylchloroformate (1.20 mL) was added in one portion followed by N-methyl-morpholine (1.0 mL). After 10 min, ethyl R-3-(N-gly)-amino-3-(3-iodo-5-chloro-2-hydroxphenyl)propionate hydrochloride, prepared as in Example 60, (2.5 g, 5.40 mmol) was added in one portion followed by N-methylmorpholine (0.6 mL). The reaction mixture was stirred for 18 h at room temperature. The reaction mixture was concentrated and the residue was dissolved in ethanol/water (1:1, 20 mL) and was chromatograph-ed (reverse phase, 95:5 water: acetonitrile over 60 min to 30:70 water: acetonitrile containing 0.1% TFA). The combined fractions were concentrated. The residue was dissolved in ethanol/water and was added sodium hydroxide until basic and was stirred for 2 h. The reaction mixture was concentrated and was purified as above by hplc to afford 0.84 g (19%) of the desired acid as the TFA salt. ¹H NMR (CD₃OD) δ 7.6-7.8 (m, 2H), 7.61 (d, 1H, J=3.5 Hz), 7.53 (m, 1H), 7.38 (m, 1H), 7.27 (d, 1H, J=3.5 Hz), 5.53 (m, 1H), 4.08 (s, 2H), 3.5-3.7 (m, 2H), 3.3 (m, 4H), 2.85 (m, 2H). Anal. Calcd for C₂₃H₂₅ClIN₅O₇: Mol. wt, 645.0487. Found: Mol. Wt, 646.0563(M+H, HRMS).

### Example 38

### (R) 3,5-Dichloro-2-hydroxy-β-[[2-[[[3-[(1,4,5,6-tetrahydro-5-hydroxy-pyrimidin-2-yl)amino]phenyl]carbonyl]amino]acetyl]amino]benzenepropanoic acid, trifluroacetate salt

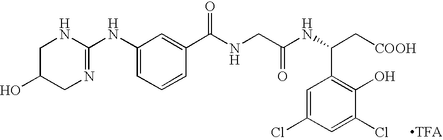

A solution of 3-N-(5-hydroxytetrahydropyrimidino)amino-5-hydroxybenzoic acid hydrochloride, prepared as in Example 25, (1.60 g, 5.92 mmol) in dimethyl-acetamide (16 mL) was heated until all the material had dissolved. This was then cooled to 0° C. and isobutylchloroformate (1.04 mL) was added in one portion followed by N-methyl-morpholine (0.872 mL). After 10 min, ethyl R-3-(N-gly)-amino-3-(3,5-dichloro-2-hydroxy-phenyl)propionate hydrochloride, prepared as in Example 3, (2.0 g, 6.512 mmol) was added in one portion followed by N-methylmorpholine (0.58 mL). The reaction mixture was stirred for 18 h at room temperature. The reaction mixture was concentrated and the residue was dissolved in ethanol/water (1:1, 20 mL) and was added sodium hydroxide until basic and was stirred for 2 h. The reaction mixture was concentrated and was purified as above by hplc to afford 0.984 g (22%) of the desired acid as the TFA salt. ¹H NMR (CD₃OD) δ 7.76-7.79 (m, 2H), 7.53 (m, 1H), 7.40 (m, 1H), 7.27 (d, 1H, J=3.2 Hz), 7.22 (d, 1H, J=3.2 Hz), 5.56 (m, 1H), 4.23 (m, 1H), 4.09 (s, 2H), 3.26-3.48 (m, 4H), 2.90 (m, 2H). Anal. Calcd for C₂₂H₂₃Cl₂N₅O₆: Mol. wt, 523.1025. Found: Mol. Wt, 524.1106 (M+H, HRMS).

### Example 39

### (R) 3,5-Dichloro-2-hydroxy-β-[[2-[[[3-hydroxy-5-[(1,4,5,6-tetrahydro-5-hydroxy-pyrimidin-2-yl)amino]phenyl]carbonyl]amino]acetyl]amino]benzenepropanoic acid, trifluroacetate salt

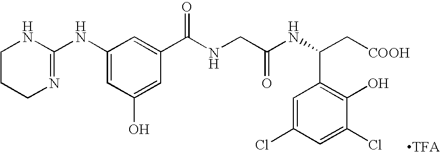

A solution of 3-N-(tetrahydropyrimidino)-amino-5-hydroxybenzoic acid hydro-chloride prepared as in EXAMPLE 24, (2.0 g, 8.26 mmol) in dimethylacetamide (25 mL) was heated until all the material had dissolved. This was then cooled to 0° C. and iso-butylchloroformate (1.45 mL) was added in one portion followed by N-methylmorpholine (1.22 mL). After 10 min, ethyl R-3-(N-gly)-amino-3-(3,5-dichloro-2-hydroxyphenyl)propionate hydrochloride, prepared as in Example 3, (3.34 g, 9.1 mmol) was added in one portion followed by N-methyl-morpholine (0.80 mL). The reaction mixture was stirred for 18 h at room temperature. The reaction mixture was concentrated and the residue was dissolved in ethanol/water (1:1, 20 mL) and was added sodium hydroxide until basic and was stirred for 2 h. The reaction mixture was concentrated and was purified as above by hplc to afford 1.2 g (21%) of the desired acid as the TFA salt. ¹H NMR (CD₃OD) δ 7.27 (m, 1H), 7.18-7.22 (m, 2H), 7.16 (m, 1H), 6.80 (m, 1H), 5.53 (m, 1H), 4.07 (s, 2H), 3.29-3.40 (m, 4H), 2.83 (m, 2H) 1.99 (m, 2H). Anal. Calcd for C₂₂H₂₃C₂N₅O₆: Mol. wt, 523.1025. Found: Mol. Wt, 524.1121 (M+H, HRMS).

### Example 40

### (R) 3-Bromo-5-chloro 2-hydroxy-β-[[2-[[[5-[(1,4,5,6-tetrahydropyrimidin-2-yl)-amino]phenyl]carbonyl]amino]acetyl]amino]benzenepropanoic acid, trifluroacetate salt

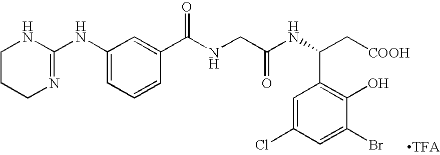

Trifluoroacetic acid (0.264 mL) was added to 3-N-(tetrahydropyrimidino)-aminobenzoic acid, prepared as in Example 30, (0.75 g, 3.43 mmol) in DMF (15 mL) and was stirred for 15 min. EDC (0.60 g, 3.43 mmol) followed by HOBt (0.463 g, 3.43 mmol) and the reaction mixture was stirred for 30 min. Ethyl R-3-(N-gly)-amino-3-(3-bromo-5-chloro-2-hydroxyphenyl)propionate hydrochloride, prepared as in Example 1, (1.59 g, 3.43 mmol) followed by N-methylmorpholine (0.367 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 1.07 g (47%) of the desired product as its TFA salt. ¹H NMR (CD₃OD) δ 7.88 (m, 1H), 7.82 (m, 1H), 7.64 (t, 1H, J=7.9 Hz), 7.48-7.52 (, 2H), 7.36 (d, 1H, J=1.7 Hz), 5.66 (m, 1H), (m, 1H), 4.19 (s, 2H), 3.40-3.5 (m, 4H), 2.97 (m, 2H), 2.10 (m, 2H). Anal. Calcd for C₂₂H₂₃BrClN₅O₅: Mol. wt, 551.0571. Found: Mol. Wt, 552.0624 (M+H, HRMS).

### Example 41

### (R) 3-Bromo-5-chloro-2-hydroxy-β-[[2-[[[5-(5-hydroxy-1,4,5,6-tetrahydro-pyrimidin-2-yl)amino]-3-nitrophenyl]carbonylamino]acetyl]amino]benzene-propanoic acid, trifluroacetate salt

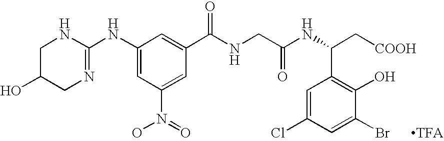 Step 1

### N-(3-Nitro-4-carboxyphenyl)-S-methylisothiourea

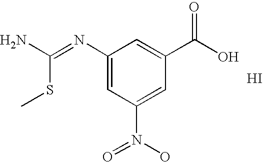

A mixture of 3-amino-5-nitrobenzoic acid (80 g, 0.4 Mol), ammonium isothio-cyanate(100 g), water (200 mL) and concentrated hydrochloric acid (40 mL) was heated at relux for 24 h. The solid formed was filtered, ashed with water and dried to afford 99 g of the thiourea. A mixture of the thiourea (90 g) and iodomethane (53 g) in ethanol (500 mL) was heated at reflux for 24 h. The reaction mixture was concentrated and dried to afford 98 g (79%) of the desired product. ¹H NMR (CD₃OD) δ 8.77 (m, 1H), 8.67 (m, 1H), 8.48 (m, 1H), 2.78 (s, 3H).

Step 2

### 3-N-(5-Hydroxytetrahydropyrimidino)amino-5-nitrobenzoic acid

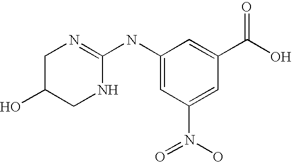

A mixture of N-(3-nitro-4-carboxyphenyl)-S-methylisothiourea (15 g) and 1,3-diamino-2-hydroxypropane (10.6 g) in dimethylacetamide (100 mL) was heated at 80° C. for 18 h and cooled. The solid formed was filtered, washed with acetonitrile to afford 3.4 g of the desired product. ¹H NMR (CD₃OD) δ 8.61 (m, 1H), 8.36 (m, 1H), 8.23 (m, 1H), 4.28 (m, 1H), 3.29-3.67 (m, 4H).

Step 3

(R) 3-Bromo-5-chloro-2-hydroxy-β-[[2-[[[5-(5-hydroxy-1,4,5,6-tetrahydro-pyrimidin-2-yl )amino]-3-nitrophenyl]carbonylamino]acetyl]amino]benzene-propanoic acid, trifluroacetate salt.

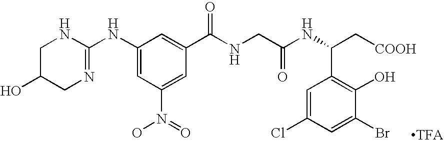

5-N-(5-hydroxytetrahydropyrimidino)-3-nitroaminobenzoic acid hydrochloride (0.7025 g, 2.5 mmol) in DMF (15 mL) was added TFA (0.285 g) and was stirred for 15 min. EDC (0.480 g, 2.5 mmol) followed by HOBt (0.338 g, 2.5 mmol) and the reaction mixture was stirred for 30 min. Ethyl 3-R—(N-gly)-amino-3-(3,5-dibromo-2-hydroxyphenyl)propionate hydrochloride, prepared as in Example 1, (1.04 g, 2.5 mmol) followed by triethylamine (0.35 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 1.20 g (66%) of the desired product as its TFA salt. ¹H NMR (CD₃OD) δ 8.60 (m, 1H), 8.27 (m, 1H), 8.09 (m, 1H), 7.40 (d, 1H, J=2.6 Hz), 7.25 (d, 1H, J=2.6 Hz), 5.56 (m, 1H), 4.26 (t, 1H, J=3.0 Hz), 4.11 (s, 2H), 3.29-3.51 (m, 4H), 2.85 (m, 2H). Anal. Calcd. for C₂₂H₂₂BrClN₆O₈: Mol. wt, 612.0371 Found: Mol. Wt, 613.0463 (M+H, HRMS).

### Example 42

### (R) 3-Bromo-5-chloro -2-hydroxy-β-[[2-[[[5-(5-hydroxy-1,4,5,6-tetrahydro-pyrimidin-2-yl)amino]-3-aminophenyl]carbonyl]amino]acetyl]amino]-benzenepropanoic acid, trifluroacetate salt

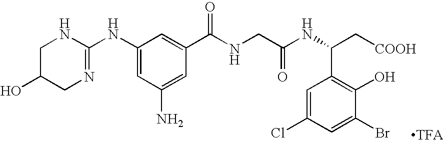

(R) 3-Bromo-5-chloro -2-hydroxy-β-[[2-[[[5-(5-hydroxy-1,4,5,6-tetrahydro-pyrimidin-2-yl)amino]-3-nitrophenyl]carbonyl]amino]acetyl]amino]benzene-propanoic acid, trifluroacetate salt (1.0 g) in acetic acid (25 mL) was added zinc powder (1.80 g) and was stirred for 2 h. After the reaction has been complete, the reaction mixture was filtered and the filtrate was concentrated the residue was purified by hplc to afford 0.50 g (52%) of the desired product as its TFA salt. ¹H NMR (CD₃OD) δ 7.41 (d, 1H, J=2.6 Hz), 7.24 (d, 1H, J=2.6 Hz), 7.07 (m, 1H), 6.98 (m, 1H), 6.72 (m. 1H), 5.55 (m, 1H), 4.22 (m, 1H), 4.05 (s, 2H), 3.28-3.44 (m, 4H), 2.85 (m, 2H). Anal. Calcd. for C₂₂H₂₄BrClN₆O₆: Mol. wt, 582.0629 Found: Mol. Wt, 583.0713 (M+H, HRMS).

### Example 43

### (R) 5-Bromo-3-chloro 2-hydroxy-β-[[2-[[[5-[(5-hydroxy-1,4,5,6-tetrahydro-pyrimidin-2-yl)amino]phenyl]carbonyl]amino]acetyl]amino]benzenepropanoic acid, trifluroacetate salt

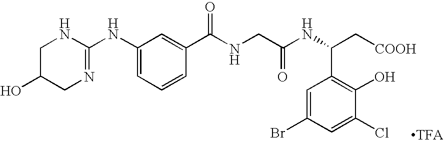 Step 1

5-Bromo-3-chlorosalicylaldehyde. This compound was prepared as described in the U.S. Pat. No. 6,100,423.

Step 2

### 5-Bromo-8-chlorocoumarin

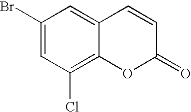

This compound was prepared as described in the U.S. Pat. No. 6,100,423.

Step 3

### Ethyl 3-amino-3-(5-bromo-2-hydroxy-3-chlorophenyl)propionate hydrochloride

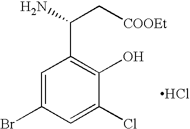

Lithium hexamethyldisilazane (106 mL, 1M, 106 mmol) was added to a solution of 5-bromo-8-chlorocoumarin (27.4 g, 105.8 mmol) in tetrahydro-furan (250 mL) at −78° C. The reaction mixture was stirred at this temperature for 30 min, then at 0° C. for 1 h. Acetic acid (6.36 g, 106 mmol) was added to the reaction mixture. The reaction mixture was poured in to ethyl acetate (300 mL) and saturated sodium carbonate (200 mL) solution. The organic layer was separated, washed with brine (200 mL), dried (MgSO4), and was concentrated to afford a residue. This was added anhydrous ether (200 mL) followed by dioxane/HCl (4N, 30 mL) at 0° C. The reaction mixture was stirred for 1 h at room temperature, filtered, and was dried in vacuo to afford 25 g (76%) of the desired product as a powder. Saturated ethanolic HCl (250 mL) was added to the solid and the reaction mixture was heated at reflux for 6 h. Most of the solvent was removed by distillation. The cooled residue was added anhydrous ether and was stirred for 2 h. The gum that formed initially turned in to a crystalline material. The crystalline product was filtered and was dried to afford 25 g (87%) of the desired product as a off-white crystalline powder. This was resolved enzymatically to afford 8.5 g of the pure R-isomer. ¹H NMR (CD₃OD) δ 7.57 (d, 1H, J=2.3 Hz), 7.44 (d, 1H, J=2.3 Hz), 4.8(m, 1H), 4.15 (q, 2H, 7.1 Hz), 3.3.09 (m, 2H), 1.21 (t, 3H, J=7.1 Hz). Anal. Calcd for C₁₁H₁₃BrClNO₃: Mol. Wt, 320.9846. Found Mol. Wt, 321.9858 (M+H, HRMS).

Step 4

### Ethyl 3-R—(N—BOC-gly)-amino-3-(5-bromo-2-hydroxy-3-chlorophenyl)-propionate

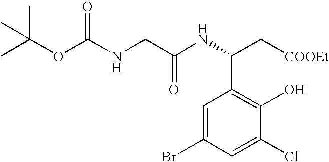

A mixture of BOC-gly-Osu (6.29 g, 23.12 mmol) ethyl 3-amino-3-(5-bromo-2-hydroxy-3-chlorophenyl)propionate hydrochloride (8.30 g, 23.12 mmol) and triethylamine (3.3 mL) in DMF (100 mL) was stirred for 18 h at room temperature. DMF was removed in vacuo and the residue was partitioned between ethyl acetate (300 mL) and sodium bicarbonate (200 mL). The organic layer was washed with hydrochloric acid (1N, 100 mL), brine (200 mL), dried (MgSO₄) and was concentrated to afford 11.0 g (99%) of the desired product as a solid. ¹H NMR (CD₃OD) δ 7.38 (m, 1H), 7.29 (d, 1H, J=2.4 Hz), 5.54 (m, 1H), 4.07 (q, 2H, 7.12 Hz), 3.69 (s, 2H), 2.84 (m, 2H), 1.44 (s, 9H), 1.21 (t, 3H, J=7.1 Hz). Anal. Calcd for C₁₈H₂₄BrClN₂O₆: Mol. Wt, 478.0506. Found Mol. Wt, 479.0610 (M+H, HRMS).

Step 5

### Ethyl 3-R—(N-gly)-amino-3-(5-bromo-2-hydroxy-3-chlorophenyl)propionate hydrochloride

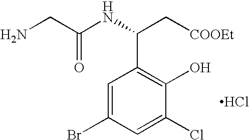

Ethanolic HCl (saturated, 250 mL) was added to ethyl 3-R—(N—BOC-gly)-amino-3-(5-bromo-2-hydroxy-3-chlorophenyl)propionate (10.8 g, 22.53 mmol) at rt and was stirred and heated at reflux for 6 h. The reaction mixture was concentrated, and concentrated once more after addition of toluene (100 mL). The residue obtained was suspended in ether and was filtered and dried to afford 9.0 g (96%) of the desired product as a crystalline powder. ¹H NMR (CD₃OD) δ 7.41 (d, 1H, J=2.4 Hz), 7.30 (d, 1H, J=2.4 Hz), 5.58 (m, 1H), 4.10 (q, 2H, 7.1 Hz), 3.69 (s, 2H), 2.88 (m, 2H), 1.19 (t, 3H, J=7.1 Hz). Anal. Calcd for C₁₃H₁₆BrClN₂O₄: Mol. Wt, 377.9982. Found Mol. Wt, 379.0067 (M+H, HRMS).

Step 6

### R 5-Bromo-3-chloro 2-hydroxy-β-[[2-[[[5-[(5-hydroxy-1,4,5,6-tetrahydro-pyrimidin-2-yl)amino]phenyl]carbonyl]amino]acetyl]amino]benzene-propanoic acid, trifluroacetate salt

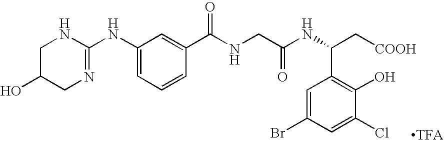

3-(5-hydroxytetrahydropyrimidino)benzoic acid prepared using similar procedure according to U.S. Pat. No. 6,028,223 Example 415 (0.815 g, 3.0 mmol) in DMF (15 mL) and was stirred for 15 min. EDC (0.575 g, 3.0 mmol) followed by HOBt (0.405 g, 3.0 mmol) and the reaction mixture was stirred for 30 min. Ethyl 3-R—(N-gly)-amino-3-(5-bromo-3-chloro-2-hydroxyphenyl)propionate hydrochloride (1.25 g, 3.0 mmol) followed by N-methylmorpholine (0.303 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.80 g (39%) of the desired product as its TFA salt. ¹H NMR (CD₃OD) δ 7.74-7.78 (m, 2H), 7.53-7.54 (m, 1H), 7.33-7.41 (m, H), 5.55 (m, 1H), 4.22 (m, 1H), 4.07 (m, 2H), 4.07 (s, 2H), 3.29 (m, 4H), 2.85 (m, 2H). Anal. Calcd for C₂₂H₂₃BrClN₅O₆: Mol. wt, 567.0520. Found: Mol. Wt, 568.0566 (M+H, HRMS).

### Example 44

### (R) 5-Bromo-3-chloro 2-hydroxy-β-[[2-[[[5-[(1,4,5,6-tetrahydropyrimidin-2-yl)amino]-3-hydroxyphenyl]carbonyl]amino]acetyl]amino]benzenepropanoic acid, trifluroacetate salt

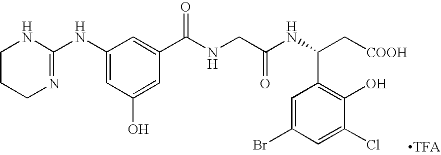

3-Hydroxy-5-N-(tetrahydropyrimidino)aminobenzoic acid hydrochloride, Example 24, (0.815 g, 3.0 mmol) in DMF (15 mL) and was stirred for 15 min. EDC (0.575 g, 3.0 mmol) followed by HOBt (0.405 g, 3.0 mmol) and the reaction mixture was stirred for 30 min. Ethyl 3-R—(N-gly)-amino-3-(5-bromo-3-chloro-2-hydroxphenyl)propionate hydrochloride, prepared as in Example 43, (1.25 g, 3.0 mmol) followed by N-methylmorpholine (0.303 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.72 g (35%) of the desired product as its TFA salt. ¹H NMR (CD₃OD) δ 7.39 (d, 1H, J=2.3 Hz), 7.33 (d, 1H, J=2.3 Hz), 7.18 (m, 1H), 7.15 (m, 1H), 6.79 (t, 1H, J=2 Hz), 5.55 (m, 1H), 4.05 (s, 2H), 3.35-3.38 (m, 4H), 2.85 (m, 2H), 1.97 (m, 2H). Anal. Calcd for C₂₂H₂₃BrClN₅O₆: Mol. wt, 567.0520. Found: Mol. Wt, 568.0574 (M+H, HRMS).

### Example 45

### (R) 5-Bromo-3-chloro 2-hydroxy-β-[[2-[[[5-[(1,4,5,6-tetrahydropyrimidin-2yl)amino]-phenyl]carbonyl]amino]acetyl]amino]benzenepropanoic acid, trifluroacetate salt

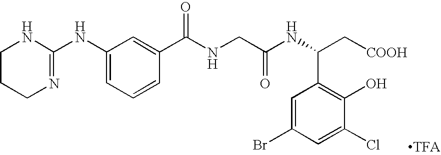

Trifluoroacetic acid (0.23 mL) was added to 3-N-(tetrahydropyrimidino)-amino-benzoic acid, prepared as in Example 30 (0.66 g, 3.0 mmol) in DMF (15 mL) and was stirred for 15 min. EDC (0.575 g, 3.0 mmol) followed by HOBt (0.405 g, 3.0 mmol) and the reaction mixture was stirred for 30 min. Ethyl 3-R—(N-gly)-amino-3-(5-bromo-3-chloro-2-hydroxyphenyl)propionate hydrochloride, prepared as in Example 43, (1.25 g, 3.0 mmol) followed by N-methylmorpholine (0.4 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.60 g (30%) of the desired product as its TFA salt. ¹H NMR (CD₃OD) δ 7.69-7.77 (m, 2H), 7.52 (t, 1H, J=7.6 Hz), 7.37-7.39 (m, 2H), 7.33 (d, 1H, J=2.3 Hz), 5.55 (m, 1H), 4.07 (s, 2H), 3.35-3.38 (m, 4H), 2.90 (m, 2H), 1.97 (m, 2H). Anal. Calcd for C₂₂H₂₃BrClN₅O₅: Mol. wt, 551.0571. Found: Mol. Wt, 552.0623 (M+H, HRMS).

### Example 46

### (R) 5-Bromo-3-chloro 2-hydroxy-β-[[2-[[[5-[5-fluoro-(1,4,5,6-tetrahydro-pyrimidin-2-yl)amino]phenyl]carbonyl]amino]acetyl]amino]benzenepropanoic acid, trifluroacetate salt

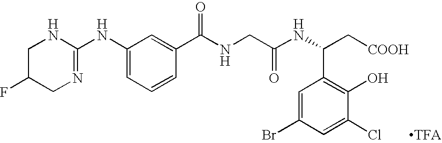

3-N-(5-Fluorotetrahydropyrimidino)aminobenzoic acid hydrochloride, Example 9, (0.514 g, 1.87 mmol) in DMF (15 mL) and was stirred for 15 min. EDC (0.359 g, 1.87 mmol) followed by HOBt (0.253 g, 1.87 mmol) and the reaction mixture was stirred for 30 min. Ethyl 3-R—(N-gly)-amino-3-(5-bromo-3-chloro-2-hydroxy-phenyl)propionate hydrochloride, prepared as in Example 43, (0.779 g, 1.87 mmol) followed by N-methyl-morpholine (0.189 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.72 g (35%) of the desired product as its TFA salt. ¹H NMR (CD₃OD) δ 7.80 (m, 1H), 7.74 (m, 1H), 7.55 (t, 1H, J=7.8 Hz), 7.40-7.42 (m, 1H), 7.39 (d, 1H, J=2.3 Hz), 7.33 (d, 1H, J=2.3 Hz), 5.55 (m, 1H), 4.05 (s, 2H), 3.45-3.7 (m, 4H), 2.85 (m, 2H). Anal. Calcd for C₂₂H₂₂BrClFN₅O₆: Mol. wt, 569.05. Found: Mol. Wt, 570.0613 (M+H, HRMS).

### Example 47

### (R) 5-Bromo-3-chloro-2-hydroxy-β-[[2-[[[3-hydroxy-5-[5-fluoro-(1,4,5,6-tetra-hydro-pyrimidin-2-yl)amino]phenyl]carbonyl]amino]acetyl]amino]benzene-propanoic acid, trifluroacetate salt

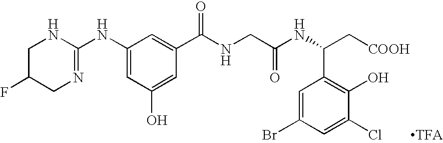

3-Hydroxy-5-N-(5-fluorotetrahydropyrimidino)aminobenzoic acid hydrochloride, Example 8, (0.585 g, 2.02 mmol) in DMF (15 mL) and was stirred for 15 min. EDC (0.387 g, 2.02 mmol) followed by HOBt (0.273 g, 2.02 mmol) and the reaction mixture was stirred for 30 min. Ethyl 3-R—(N-gly)-amino-3-(5-bromo-3-chloro-2-hydroxy-phenyl)-propionate hydrochloride, prepared as in Example 43, (0.841 g, 2.02 mmol) followed by N-methyl-morpholine (0.24 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.44 g (37%) of the desired product as its TFA salt. ¹H NMR (CD₃OD) δ 7.39 (m, 1H), 7.33 (d, 1H, J=2.3 Hz), 7.21(m, 1H), 7.17 (m, 1H), 6.81 (m, 1H), 5.55 (m, 1H), 4.05 (s, 2H), 3.45-3.67 (m, 4H), 2.85 (m, 2H). Anal. Calcd. for C₂₂H₂₂BrClFN₅O₆: Mol. wt, 585.04. Found: Mol. Wt, 586.0503 (M+H, HRMS).

### Example 48

### (R) 5-Bromo-3-chloro 2-hydroxy-β-[[2-[[[5-[5-hydroxy-(1,4,5,6-tetrahydro-pyrimidin-2-yl)amino]-4-methylphenyl]carbonyl]amino]acetyl]amino]-benzenepropanoic acid, trifluroacetate salt

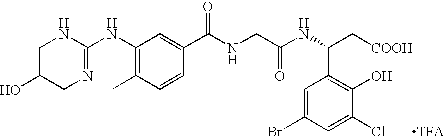 Step A

### N-(Benzoyl)-N′-3-carboxy-6-methylphenyl)thiourea

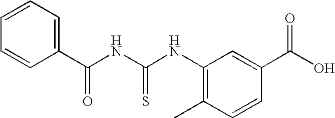

Benzoyl isothiocyanate (25.0 g, 0.153 mol), 3-amino-4-methyl benzoic acid (23.2 g, 0.153 mol) and acetonitrile (200 mL) were stirred at room temperature overnight. The precipitate was filtered and dried under vacuum to afford 44.36 g of the desired product (92%). ¹H NMR (CD₃OD) δ 8.34 (m, 1H), 8.01-8.04 (m, 2H), 7.90 (m, 1H), 7.71 (m, 1H), 7.69 (m, 1H), 7.58-7.63 (m, 2H), 7.48 (m, 1H), 2.42 (s, 3H). Anal. Calcd for: C₁₆H₁₄N₂O₃S Mol. Wt, 314.0725. Found: 315.0823 (M+H, HRMS).

Step 2

### N-3-carboxy-6-methylphenyl)thiourea

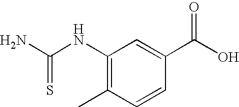

Sodium methoxide (61.12 mL, 0.283 mol) was added to a suspension of N-(benzoyl)-N′3-carboxy-6-methylphenyl)thiourea (44.36 g, 0.141 mol) and anhydrous methanol (200 mL). The reaction mixture was stirred at room temperature for 45 minutes and concentrated. The residue was triturated with ether three times. The solid was powdered and washed with warm ether. Dissolved in minimum amount of water over 1 hour. Cooled to 0° C. and acidified with concentrated HCl over 1 h to afford an off-white powder. Dried in vacuum overnight. Yield: 29.0 g (98%). ¹H NMR (CD₃OD) δ 7.85-7.88 (m, 2H), 7.42 (m, 1H), 2.35 (s, 3H). Anal. Calcd for: C₉H₁₀N₂O₂S Mol. Wt, 210.0463. Found: 211.0501 (M+H, HRMS).

Step 3

### N-(3-carboxy-6-methylphenyl)-S-methylisothiourea

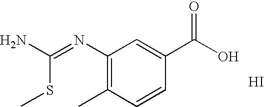

N-(3-carboxy-6-methylphenyl)thiourea (29.0 g, 0.138 mol) and iodo-methane (19.73 g, 8.66 mL, 0.138 mol) was dissolved in ethanol (150 mL) and heated to reflux under a drying tube overnight. The clear reaction mixture was concentrated to afford the desired product. ¹H NMR (CD₃OD) δ 8.01-8.03 (m, 1H), 7.90 (d,1H, J=1.6 Hz), 7.58 d, 1H, J=7.9 Hz), 2.77 (s, 3H), 2.37 (s, 3H). Anal. Calcd for: C₁₀H₁₂N₂O₂S Mol. Wt, 224.0619. Found: 225.0663 (M+H, HRMS).

Step 4

### N-(5-Hydroxytetrahydropyrimidinyl)-6-methyl-3-aminobenzoic acid

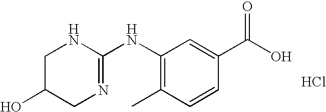

N-(3-Carboxy-6-methylphenyl)-S-methylisothiourea (17.0 g, 0.048 mol) and 1,3-diamino-2-hydroxypropane (12.96 g, 0.144 mol) and DMF (20 mL) were added to 200 mL flask equipped with condenser and drying tube. The solution was heated at 100° C. for 36 h and was cooled and filtered. The solid was washed with ethyl acetate, then ether. The solid was added slowly to stirring 4N HCl in dioxane. The mixture was stirred for 2 h. The reaction mixture became difficult to stir and the solution was concentrated and dried under high vacuum overnight. The solid was washed with ether three times, filtered, and dried. Yield 13.31 g (97%). ¹H NMR (CD₃OD) δ 7.13-7.21 (m, 2H), 6.86 (m, 1H), 3.26 (m, 4H), 1.83 (m, 2H). Anal. Calcd for C₁₁H₁₃O₃N₃: Mol. Wt, 236.1005 (M+H, HRMS). Found: Mol. W, 15 236.1035 (M+H, HRMS).

Step 5

### R 5-Bromo-3-chloro 2-hydroxy-β-[[2-[[[5-[5-hydroxy-(1,4,5,6-tetrahydro-pyrimidin-2-yl)amino]-4-methylphenyl]carbonyl]amino]acetyl]amino]-benzenepropanoic acid, trifluroacetate salt

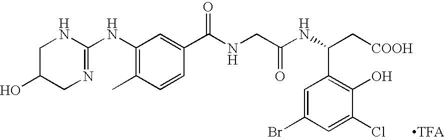

3-N-(5-Hydroxytetrahydropyrimidino)-4-methylamino-benzoic acid hydrochloride (0.753 g, 3.0 mmol) in DMF (15 mL) and was stirred for 15 min. EDC (0.575 g, 3.0 mmol) followed by HOBt (0.405 g, 3.0 mmol) and the reaction mixture was stirred for 30 min. Ethyl 3-R—(N-gly)-amino-3-(5-bromo-3-chloro-2-hydroxy-phenyl)propionate hydrochloride, prepared as in Example 43, (1.25 g, 3.0 mmol) followed by N-methyl-morpholine (0.303 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.38 g (18%) of the desired product as its TFA salt. ¹H NMR (CD₃OD) δ 7.78 (m, 1H), 7.74 (m, 1H), 7.44 (m, 1H), 7.38 (d, 1H, J=2.3 Hz), 7.33 (d, 1H, J=2.3 Hz), 5.54 (m, 1H), 4.21 (t, 1H, J=3.1 Hz), 4.07 (s, 2H), 3.27-3.44 (m, 4H), 2.85 (m, 2H). Anal. Calcd. for C₂₃H₂₅BrClN₅O₆: Mol. wt, 581.07. Found: Mol. Wt, 582.0802 (M+H, HRMS).

### Example 49

### (R) 5-Bromo-3-chloro-2-hydroxy-β-[[2-[[[5-(5-hydroxy-1,4,5,6-tetrahydro-pyrimidin-2-yl)amino]-3-nitrophenyl]carbonylamino]acetyl]amino]-benzenepropanoic acid, trifluroacetate salt

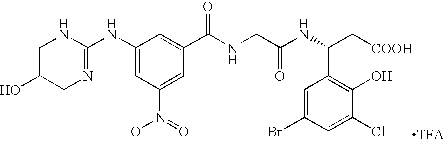

5-N-(5-hydroxytetrahydropyrimidino)-3-nitroaminobenzoic acid hydrochloride prepared asin Example 41, (0.7025 g, 2.5 mmol) in DMF (15 mL) was added TFA (0.285 g) and was stirred for 15 min. EDC (0.480 g, 2.5 mmol) followed by HOBt (0.338 g, 2.5 mmol) and the reaction mixture was stirred for 30 min. Ethyl 3-R—(N-gly)-amino-3-(3,5-dibromo-2-hydroxyphenyl)propionate hydrochloride, prepared as in Example 43, (1.04 g, 2.5 mmol) followed by triethylamine (0.35 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.80 g (44%) of the desired product as its TFA salt. ¹H NMR (CD₃OD) δ 8.60 (m, 1H), 8.27 (m, 1H), 8.09 (m, 1H), 7.39 (d, 1H, J=2.2 Hz), 7.34 (d, 1H, J=2.2 Hz), 5.55 (m, 1H), 4.26 (t, 1H, J=3.0 Hz), 4.11 (s, 2H), 3.28-3.51 (m, 4H), 2.85 (m, 2H). Anal. Calcd. for C₂₂H₂₂BrClN₆O₈: Mol. wt, 612.0371 Found: Mol. Wt, 613.0463 (M+H, HRMS).

### Example 50

### (R) 5-Bromo-3-chloro-2-hydroxy-β-[[2-[[[5-(5-hydroxy-1,4,5,6-tetrahydro-pyrimidin-2-yl)amino]3-aminophenyl]carbonyl]amino]acetyl]amino]-benzenepropanoic acid, trifluroacetate salt

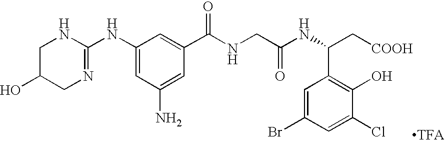

R 5-Bromo-3-chloro -2-hydroxy-β-[[2-[[[5-(5-hydroxy-1,4,5,6-tetrahydro-pyrimidin-2-yl)amino]-3-nitrophenyl]carbonyl]amino]acetyl]amino]-benzenepropanoic acid, trifluroacetate salt (0.60 g) in acetic acid (25 mL) was added zinc powder (1.80 g) and was stirred for 2 h. After the reaction has been complete, the reaction mixture was filtered and the filtrate was concentrated the residue was purified by hplc to afford 0.350 g (60%) of the desired product as its TFA salt. ¹H NMR (CD₃OD) δ 7.39 (d, 1H, J=2.3 Hz), 7.32 (d, 1H, J=2.6 Hz), 7.07 (m, 1H), 6.98 (m, 1H), 6.71 (m. 1H), 5.55 (m, 1H), 4.21 (m, 1H), 4.05 (s, 2H), 3.28-3.44 (m, 4H), 2.85 (m, 2H). Anal. Calcd. for C₂₂H₂₄BrClN₆O₆: Mol. wt, 582.0629 Found: Mol. Wt, 583.0719 (M+H, HRMS).

### Example 51

### (R) 3,5-dibromo-2-hydroxy-β-[[2-[[[3-hydroxy-5-[5-hydroxy-(1,4,5,6-tetrahydro-pyrimidin-2-yl)amino]phenyl]carbonyl]amino]acetyl]amino]benzenepropanoic acid, trifluroacetate salt

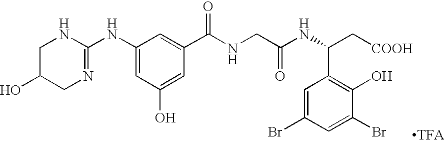 Step 1

### 6,8-dibromocoumarin

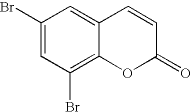

A mixture of 3,5-dibromosalicylaldehyde (100 g, 0.357 mole), acetic anhydride (165 mL) and triethylamine (45 mL) was heated at reflux for 36 h. Upon cooling, the desired coumarin precipitated as a dark brown crystalline material. This was filtered, washed with hexane and saturated sodium bicarbonate and was air-dried. Yield: 68 g (63%). Additional quantities of the desired product (10 g, 9%) may be obtained from the filtrate, upon storage. 1H NMR (DMSO-d6) δ 8.12 (d, 1H, J=2.2 Hz), 8.01 (d, 1H, J=9.7 Hz), 7.99 (d, 1H, J=2.2 Hz), (6.63, d, 1H, J=9.7 Hz). Anal. Calcd for C₉H₄Br₂O₂: Mol. Wt, 301.8578. Found: Mol. Wt, 301.8550 (M+H, HRMS).

Step 2

### Ethyl 3-amino-3-(5,8-dibromo-2-hydroxyphenyl)-propionate hydrochloride

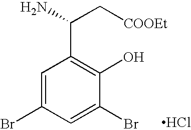

Lithium hexamethyldisilazane (165 mL,1M, 165 mmol) was added to a solution of 6,8-dibromocoumarin (50 g, 165 mmol) in tetrahydrofuran (300 mL) at −78° C. The reaction mixture was stirred at this temperature for 30 min, then at 0° C. for 1 h. Acetic acid (10 g, 165 mmol) was added to the reaction mixture. The reaction mixture was poured in to ethyl acetate (300 mL) and saturated sodium bicarbonate (200 mL) solution. The organic layer was separated, washed with brine (200 mL), dried (MgSO4), and was concentrated to afford a residue. This was added anhydrous ether (200 mL) followed by dioxane/HCl (4N, 100 mL) at 0° C. The reaction mixture was stirred for 1 h at room temperature, filtered, and was dried in vacuo to afford 54 g (92%) of the desired product as a powder. Ethanolic HCl (500 mL) was added to a solution of 4-amino-3,4-dihydro-6,8-dibromocoumarin hydrochloride (51.5.0 g, 144.2 mmol). After 6 h at reflux, most of the solvent was removed by distillation. The cooled residue was added anhydrous ether and was stirred for 2 h. The initial gum turned in to a crystalline material. The crystalline product was filtered and was dried to afford 50 g (86%) of the desired product as off-white crystalline powder. Enzymatic resoultion of this compound afforded 24 g of the desired R-Isomer. ¹H NMR (CD₃OD) δ 7.72 (d, 1H, J=2.3 Hz), 7.49 (d, 1H, J=2.3 Hz), 4.9 (m, 1H), 4.15 (m, 2H), 3.09 (m, 2H), 1.21 (t, 3H, J=7.1 Hz). Anal. Calcd for C₁₁H₁₃Br₂NO₃: Mol. Wt, 364.9262. Found Mol. Wt, 365.9345 (M+H, HRMS).

Step 3

### Ethyl 3-(N—BOC-gly)-amino-3-(5,8-dibromo-2-hydroxyphenyl)propionate

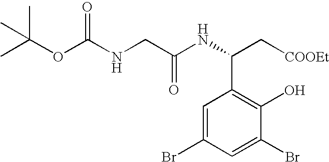

A mixture of BOC-gly-Osu (8.1 g, 29.74 mmol), ethyl 3-amino-3-(5,8-dibromo-2-hydroxphenyl)propionate hydrochloride (12.0 g, 29.74 mmol) and triethylamine (4.2 mL) in DMF (200 mL) was stirred at rt for 18 h. The reaction mixture was stirred for 18 h at room temperature. DMF was removed in vacuo and the residue was partitioned between ethyl acetate (500 mL) and sodium bicarbonate (200 mL). The organic layer was washed with hydrochloric acid (1N, 100 mL), brine (200 mL), dried (MgSO4) and was concentrated to afford 14.9 g (96%) of the desired product as a solid. ¹H NMR (CD₃OD) δ 7.54 (d, 1H, J=1.95 Hz), 7.33 (d, 1H, J=1.96 Hz), 5.54 (m, 1H), 4.07 (q, 2H, 7.4 Hz), 3.69 (s, 2H), 2.85 (m, 2H), 1.44 (s, 9H), 1.16 (t, 3H, J=7.1 Hz). Anal. Calcd for C₁₈H₂₄Br₂N₂O₆: Mol. Wt, 522.0001. Found Mol. Wt, 523.0074 (M+H, HRMS).

Step 4

### Ethyl 3-(N-gly)-amino-3-(5,8-dibromo-2-hydroxyphenyl)propionate hydrochloride

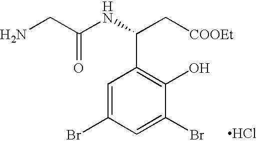

Ethanolic HCl (250 mL) was added to ethyl 3-(N—BOC-gly)-amino-3-(3,5-dibromo-2-hydroxy-phenyl)propionate (12.5, 28.1 mmol g) at 0° C. and was stirred at room temperature for 3 h. The reaction mixture was concentrated. The residue obtained was suspended in ether and was filtered and dried to afford 12.5 g (97%) of the desired product as a crystalline powder. ¹H NMR (CD₃OD) δ 7.56 (d, 1H, J=2.3 Hz), 7.34 (d, 1H, J=2.4 Hz), 5.57 (m, 1H), 4.09 (q, 2H, 7.1 Hz), 3.69 (s, 2H), 2.88 (m, 2H), 1.19 (t, 3H, J=7.1 Hz). Anal. Calcd for C₁₃H₁₆Br₂N₂O₄: Mol. Wt, 421.9477. Found Mol. Wt, 422.9576 (M+H, HRMS).

Step 5

### R 3,5-dibromo-2-hydroxy-β-[[2-[[[3-hydroxy-5-[5-hydroxy-(1,4,5,6-tetrahydro-pyrimidin-2-yl)amino]phenyl]carbonyl]amino]-acetyl]amino]benzenepropanoic acid, trifluroacetate salt

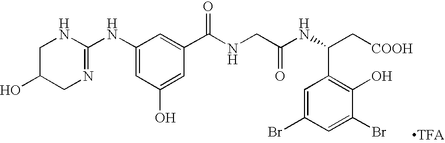

3-N-(5-Hydroxytetrahydro-pyrimidino)-5-hydroxyaminobenzoic acid (prepared according to U.S. Pat. No. 6,013,651, Example H, 0.109 g, 0.434 mmol) in DMF (15 mL) was added TFA (0.033 mL) and was stirred for 15 min. EDC (0.083 g, 0.434 mmol) followed by HOBt (0.059 g, 0.434 mmol) and the reaction mixture was stirred for 30 min. Ethyl 3-R—(N-gly)-amino-3-(3,5-dibromo-2-hydroxyphenyl)-propionate hydro-chloride (0.20 g, 0.434 mmol) followed by N-methylmorpholine (0.044 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.1 g (32%) of the desired product as its TFA salt. ¹H NMR (CD₃OD) δ 7.54 (d, 1H, J=3.0 Hz), 7.38 (d, 1H, J=3.0 Hz), 7.19 (m, 2H), 6.83 (m, 1H), 5.55 (m, 1H), 4.23 (t, 1H, J=4.3 Hz), 4.07 (s, 2H), 3.29-3.47 (m, 4H), 2.85 (m, 2H). Anal. Calcd. for C₂₂H₂₃Br₂N₅O₇: Mol. wt, 627.00. Found: Mol. Wt, 628.0078 (M+H, HRMS).

### Example 52

### (R) 3,5-Dibromo-2-hydroxy-β-[[2-[[[5-[5-hydroxy-(1,4,5,6-tetrahydro-pyrimidin-2-yl)amino]phenyl]carbonyl]amino]acetyl]amino]benzenepropanoic acid, trifluroacetate salt

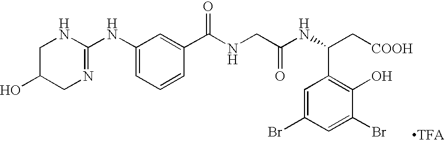

3-N-(5-Hydroxytetrahydropyrimidino)aminobenzoic acid hydrochloride, prepared as in Example 25 (0.102 g, 0.434 mmol) in DMF (15 mL) and was stirred for 15 min. EDC (0.083 g, 0.434 mmol) followed by HOBt (0.059 g, 0.434 mmol) and the reaction mixture was stirred for 30 min. Ethyl 3-R—(N-gly)-amino-3-(3,5-dibromo-2-hydroxyphenyl)-propionate hydrochloride, prepared as in Example 51, (0.20 g, 0.434 mmol) followed by N-methylmorpholine (0.044 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.105 g (32%) of the desired product as its TFA salt. ¹H NMR (CD₃OD) δ 7.54 (d, 1H, J=3.0 Hz), 7.38 (d, 1H, J=3.0 Hz), 7.19-7.15 (m, 2H), 6.79 (m, 1H), 5.55 (m, 1H), 4.07 (s, 2H), 3.29-3.40 (m, 4H), 2.85 (m, 2H). Anal. Calcd. for C₂₂H₂₃Br₂N₅O₆: Mol. wt, 611.00. Found: Mol. Wt, 612.0091 (M+H, HRMS).

### Example 53

### (R) 3,5-Dibromo-2-hydroxy-β-[[2-[[[5-(1,4,5,6-tetrahydropyrimidin-2-yl)-amino]-3-hydroxy]phenyl]carbonyl]amino]acetyl]amino]benzenepropanoic acid, trifluroacetate salt

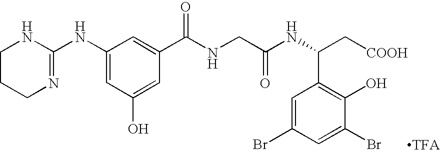

5-N-(Tetrahydropyrimidino)-3-hydroxylaminobenzoic acid hydrochloride, prepared as in Example 24, (0.102 g, 0.434 mmol) in DMF (15 mL) was added TFA (0.033 mL) and was stirred for 15 min. EDC (0.083 g, 0.434 mmol) followed by HOBt (0.059 g, 0.434 mmol) and the reaction mixture was stirred for 30 min. Ethyl 3-R—(N-gly)-amino-3-(3,5-dibromo-2-hydroxyphenyl)propionate hydrochloride, prepared as in Example 51, (0.20 g, 0.434 mmol) followed by N-methylmorpholine (0.044 g) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.125 g (40%) of the desired product as its TFA salt. ¹H NMR (CD₃OD) δ 7.75-7.81 (m, 2H), 7.52-7.57 (m, 2H), 7.19 (m, 2H), 7.38-7.44 (m, 2H), 5.56 (m, 1H), 4.23 (t, 1H, J=4.3 Hz), 4.09 (s, 2H), 3.29-3.48 (m, 4H), 2.85 (m, 2H). Anal. Calcd. for C₂₂H₂₃Br₂N₅O₇: Mol. wt, 627.00 Found: Mol. Wt, 628.0078 (M+H, HRMS).

### Example 54

### (R) 3,5-Dibromo-2-hydroxy-β-[[2-[[[3-(5-hydroxy-1,4,5,6-tetrahydropyrimidin-2-yl)amino]pyridyl]5-carbonyl]amino]acetyl]amino]benzenepropanoic acid, trifluroacetate salt

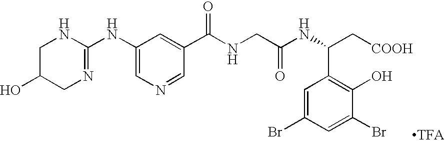

3-N-(Tetrahydropyrimidino)-5-nicotinic acid TFA salt, prepared as in Example 2, (0.875 g, 2.5 mmol) in DMF (20 mL) was added EDC (0.480 g, 2.5 mmol) followed by HOBt (0.338 g, 2.5 mmol) and the reaction mixture was stirred for 30 min. Ethyl 3-R—(N-gly)-amino-3-(3,5-dibromo-2-hydroxyphenyl)propionate hydro-chloride, prepared as in Example 51, (1.15 g, 2.5 mmol) followed by triethylamine (0.35 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.85 g (47%) of the desired product as its TFA salt. ¹H NMR (CD₃OD) δ 8.89 (s, 1H), 8.60 (s, 1H), 8.14 (m, 1H), 7.53 (m, 1H), 7.37 (m, 1H), 5.56 (m, 1H), 4.24 (m, 1H), 4.1 (s, 2H), 3.29-3.48 (m, 4H), 2.86 (m, 2H). Anal. Calcd. for C₂₁H₂₂Br₂N₆O₆: Mol. wt, 611.9968 Found: Mol. Wt, 613.0046 (M+H, HRMS).

### Example 55

### (R) 3,5-Dibromo-2-hydroxy-β-[[2-[[[5-(5-fluoro-1,4,5,6-tetrahydropyrimidin-2-yl)amino]-3-hydroxy]phenyl]carbonyl]amino]acetyl]amino]benzenepropanoic acid, trifluroacetate salt

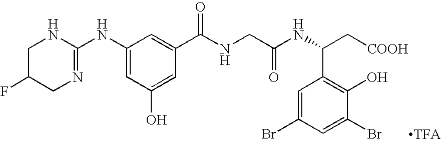

5-N-(5-fluorotetrahydropyrimidino)-3-hydroxylaminobenzoic acid hydrochloride prepared as in Example 8, (0.4946 g, 1.71 mmol) in DMF (25 mL) was added EDC (0.328 g, 1.71 mmol) followed by HOBt (0.23 g, 1.71 mmol) and the reaction mixture was stirred for 30 min. Ethyl 3-R—(N-gly)-amino-3-(3,5-dibromo-2-hydroxphenyl)propionate hydrochloride, prepared as in Example 51, (0.788 g, 1.71 mmol) followed by triethylamine (0.24 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.80 g (63%) of the desired product as its TFA salt. ¹H NMR (CD₃OD) δ 7.55 (m, 1H), 7.38 (m, 1H), 7.18-7.22 (m, 2H), 7.83 (m, 1H), 5.56 (m, 1H), 4.07 (s, 2H), 3.47-3.69 (m, 4H), 2.85 (m, 2H). Anal. Calcd. for C₂₂H₂₂Br₂FN₅O₆: Mol. wt, 628.9921 Found: Mol. Wt, 629.9999 (M+H, HRMS).

### Example 56

### (R) 3,5-Dibromo-2-hydroxy-β-[[2-[[[5-(1,4,5,6-tetrahydropyrimidin-2-yl)amino]-phenyl]carbonyl]amino]acetyl]amino]benzenepropanoic acid, trifluroacetate salt

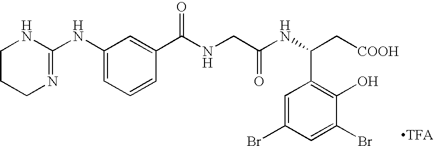

5-N-(Tetrahydropyrimidino)aminobenzoic acid hydrochloride prepared as in Example 30, (0.66 g, 3.0 mmol) in DMF (15 mL) was added TFA (0.23 g) and was stirred for 15 min. EDC (0.575 g, 3.0 mmol) followed by HOBt (0.405 g, 3.0 mmol) and the reaction mixture was stirred for 30 min. Ethyl 3-R—(N-gly)-amino-3-(3,5-dibromo-2-hydroxyphenyl)propionate hydrochloride, prepared as in Example 51, (1.38 g, 3.0 mmol) followed by triethylamine (0.42 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 1.20 g (56%) of the desired product as its TFA salt. ¹H NMR (CD₃OD) δ 7.7-7.76 (m, 2H), 7.36-7.39 (m, 2H), 5.55 (m, 1H), 4.08 (s, 2H), 3.29-3.38 (m, 4H), 2.85 (m, 2H). Anal. Calcd. for C₂₂H₂₃Br₂N₅O₅: Mol. wt, 595.0066 Found: Mol. Wt, 596.0144 (M+H, HRMS).

### Example 57

### (R) 3,5-Dibromo-2-hydroxy-β-[[2-[[[5-(5-hydroxy-1,4,5,6-tetrahydropyrimidin-2-yl)-amino]-4-methyl]phenyl]carbonylamino]acetyl]amino]benzenepropanoic acid, trifluroacetate salt

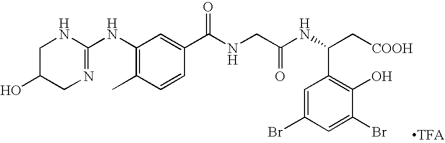

5-N-(5-hydroxy-tetrahydropyrimidino)-4-methylaminobenzoic acid hydrochloride, prepared as in Example 48, (0.747 g, 3.0 mmol) in DMF (15 mL) was added EDC (0.575 g, 3.0 mmol) followed by HOBt (0.405 g, 3.0 mmol) and the reaction mixture was stirred for 30 min. Ethyl 3-R—(N-gly)-amino-3-(3,5-dibromo-2-hydroxphenyl)propionate hydrochloride, prepared as in Example 51, (1.38 g, 3.0 mmol) followed by triethylamine (0.42 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 1.05 g (47%) of the desired product as its TFA salt. ¹H NMR (CD₃OD) δ 7.76-7.79 (m, 1H), 7.72 (m, 1H), 7.53 (d, 1H, J=2.4 Hz), 7.42-7.44 (m, 1H), 7.37 (d, 1H, J=2.4 Hz), 5.55 (m, 1H), 4.20 (t, 1H, J=3.1 Hz), 4.06 (s, 2H), 3.26-3.43 (m, 4H), 2.85 (m, 2H). Anal. Calcd. for C₂₃H₂₅Br₂N₅O₆: Mol. wt, 625.0172 Found: Mol. Wt, 626.0232 (M+H, HRMS).

### Example 58

### (R) 3,5-Dibromo-2-hydroxy-β-[[2-[[[5-(5-hydroxy-1,4,5,6-tetrahydropyrimidin-2-yl)amino]-3-nitro]phenyl]carbonyl]amino]acetyl]amino]benzenepropanoic acid, trifluroacetate salt

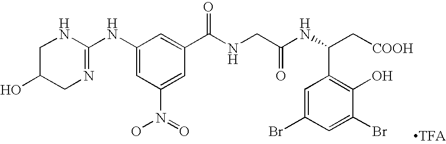

5-N-(5-hydroxytetrahydropyrimidino)-3-nitroaminobenzoic acid hydrochloride, prepared as in Example 41, (0.703 g, 2.5 mmol) in DMF (20 mL) was added TFA (0.285 g) and was stirred for 15 min. EDC (0.480 g, 2.5 mmol) followed by HOBt (0.338 g, 2.5 mmol) and the reaction mixture was stirred for 30 min. Ethyl 3-R—(N-gly)-amino-3-(3,5-dibromo-2-hydroxyphenyl)propionate hydrochloride, prepared as in Example 51, (1.15 g, 2.5 mmol) followed by triethylamine (0.35 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.87 g (45%) of the desired product as its TFA salt. ¹H NMR (CD₃OD) δ 8.59 (br, 1H), 8.27 (m, 1H), 8.09 (m, 1H), 7.53 (m, 1H), 7.37 (m, 1H), 5.55 (m, 1H), 4.26 (m, 1H), 4.10 (s, 2H), 3.28-3.51 (m, 4H), 2.85 (m, 2H). Anal. Calcd. for C₂₂H₂₂Br₂N₆O₈: Mol. wt, 655.9866 Found: Mol. Wt, 656.9944 (M+H, HRMS).

### Example 59

### (R) 3,5-Dibromo-2-hydroxy-β-[[2-[[[5-(5-hydroxy-1,4,5,6-tetrahydropyrimidin-2-yl)-amino]-3-aminophenyl]carbonyl]amino]acetyl]amino]benzenepropanoic acid, trifluroacetate salt

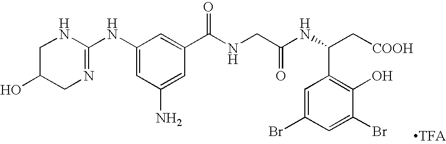

R 3,5-Dibromo-2-hydroxy-β-[[2-[[[5-(5-hydroxy-1,4,5,6-tetrahydropyrimidin-2-yl)amino]-3-nitrophenyl]carbonyl]amino]-acetyl]amino]benzenepropanoic acid, trifluroacetate salt (0.77 g, 0.434 mmol) in acetic acid (25 mL) was added zinc powder (1.80 g) and was stirred for 2 h. After the reaction has been complete, the reaction mixture was filtered and the filtrate was concentrated the residue was purified by hplc to afford 0.370 g (50%) of the desired product as its TFA salt. ¹H NMR (CD₃OD) δ 7.54 (m, 1H), 7.36 (m, 1H), 7.09 (m, 1H), 7.00 (m, 1H), 6.73 (m. 1H), 5.56 (m, 1H), 4.23 (m, 1H), 4.04 (s, 2H), 3.27-3.44 (m, 4H), 2.85 (m, 2H). Anal. Calcd. for C₂₂H₂₄Br₂N₆O₆: Mol. wt, 626.0124 Found: Mol. Wt, 627.0202 (M+H, HRMS).

### Example 60

### (R) 5-Chloro-3-iodo-2-hydroxy-β-[[2-[[[3-hydroxy-5-[(1,4,5,6-tetrahydro-5-hydroxy-pyrimidin-2-yl)amino]phenyl]carbonyl]amino]acetyl]amino]-benzenepropanoic acid, trifluroacetate salt

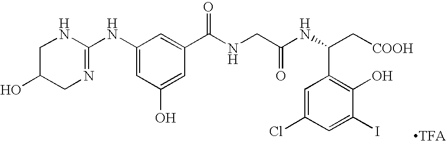 Step 1

### 2-O-(MEM)-3-iodo-5-chlorosalicylaldehyde

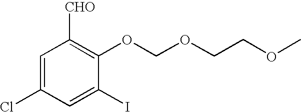

This compound was prepared as reported in U.S. Pat. No. 6,100,423. Potassium carbonate (81.4 g, 5894 mole) was added to a solution of 3-iodo-5-chlorosalicylaldehyde (166.6 g, 0.5894 mole) in DMF (400 mL) at 20° C. This resulted in yellow slurry and MEM-Cl (75.3 g, 0.589 mole) was added maintaining the reaction temperature. After 2 h, additional MEM-Cl (1.5 g) was added. After stirring for further 1 h, the reaction mixture was poured in to ice-water mixture and was stirred. The precipitate formed was filtered and was dried in vacuo to afford the desired protected aldehyde. Yield: 212.7 g (98%). ¹H NMR (CDCl₃) δ 10.19 (s, 1H), 7.96 (d, 1H, J=3.5 Hz), 7.75 (d, 1H, J=3.5 Hz), 5.21 (s, 2H), 3.87 and 3.51 (m, 4H), 3.33 (s, 3H). Anal. Calcd for C₁₁H₁₂ClIO₄: Mol. Wt, 387.9813 (M+NH₄). Found: Mol. Wt, 387.9800 (M+NH₄, HRMS).

Step 2

### 2-O-(MEM)-3-iodo-5-chlorosalicylaidehyde with (R)-phenyl glycinol

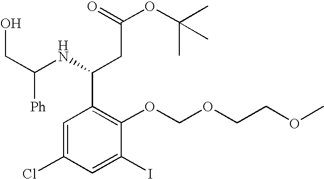

(R)-Phenyl glycinol (78.68 g, 0.574 mole) was added to a solution of 2-O-(MEM)-3-iodo-5-chlorosalicylaldehyde (212.7 g, 0.574 mole) in THF (1 L) at room temperature. An endothermic reaction resulted. After 1 h of stirring MgSO4 (100 g) was added and the stirring was continued for 2 h. The reaction mixture was filtered and the filtrate was concentrated and was dried in vacuo for 2 h. A 2-neck round bottom flask was charged with the Reformatsky reagent (420 g, 1.7 mole) and N-methylpyrrolidone (1.7 L) and was stirred at −10° C. A solution of the imine in N-methyl-pyrrolidone (100 mL) was slowly added maintaining the temperature at −10° C. The mixture was maintained at this temperature for 2 h and for 1 h at −5° C. After cooling the reaction mixture to −10° C., a solution of conc. HCl in saturated ammonium chloride (32 ml/400 mL). Ethyl ether (900 mL) was added and was stirred for 2 h at rt. The ether layer was separated, and the aqueous layer was further extracted with ether (800 mL). The combined ether layers was washed with saturated ammonium chloride (200 mL), water (200 mL), brine (200 mL), dried (MgSO4) and was concentrated to afford 332 g (95%) of an oil. ¹H NMR (CDCl₃) δ 7.60 (d, 1H, J=3.2 Hz) 7.19-7.29 (m, 6H), 5.15 (s, 2H), 4.68 (m, 1H), 3.99 (m, 2H), 3.93 (m, 1H), 3.62 (m, 4H), 3.42 (s, 3H), 2.48-2.72 (m, 2H), 1.48 (s, 9H). Anal. Calcd for C₂₅H₃₃ClINO₆: Mol. Wt, 605.1041. Found Mol. Wt, 606.1098 (M+H, HRMS).

Step 3

### Ethyl 3-amino-3-(R)-(5-chloro-2-hydroxy-3-iodophenyl)propionate p-toluene-sulfonic acid salt

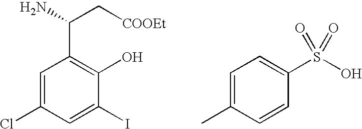

A solution of the crude ester (332.0 g) was dissolved in ethanol (3.5 L) and was cooled to 0° C. Lead tetra acetate (344.0 g, 0.776 mole) was added in one lot and the solution turned from orange to bright red orange before going back to orange. After 3 h, 15% solution of NaOH (800 mL) was added to the reaction mixture. Most of the ethanol was removed under reduced pressure. The residue was added 15% solution of NaOH (800 mL) and was extracted with ether (1600 mL). The ether layer was washed with water (500 mL), brine (500 mL), dried and was concentrated to afford orange oil. This was dissolved in ethanol (500 mL) and p-toluenesulfonic acid (192 g) was added and the solution was heated at reflux for 8 h and was concentrated under reduced pressure. The residue was diluted with THF (600 mL) and was heated at reflux and was cooled. The precipitate was filtered, washed with hexane/THF (300 mL, 1:1) and dried to afford 90.25 g the desired product as the p-toluenesulfonic acid salt. ¹H NMR (CD₃OD) δ 7.8 (d, 1H, J=3.2 Hz), 7.74 (d, 2H, J=10.7 Hz), 7.66 (d, 1H, J=3.2 Hz), 7.27 (d, 2H, J=10.7 Hz), 5.17 (m, 1H), 4.17 (m, 2H), 3.30 (m, 2H), 2.43 (s, 3H), 1.25 (t, 3H, J=9.4 Hz). Anal. Calcd for C₁₁H₁₃ClINO₃: Mol. Wt, 368.9629. Found Mol. Wt, 426.9908 (M+H, HRMS).

Step 4

### Ethyl 3-(N—BOC-gly)-amino-3-(R)-(5-chloro-2-hydroxy-3-iodophenyl)propionate

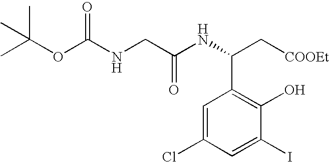

A mixture of BOC-gly-OSu (45.36 g, 166.6 mmol), ethyl 3-(R)-amino-3-(5-chloro-2-hydroxy-3-iodophenyl)propionate PTSA salt (90.25 g, 166.6 mmol) in DMF (500 mL) was added triethylamine (25 mL). The reaction mixture was stirred for 18 h at room temperature. DMF was removed in vacuo and the residue was partitioned between ethyl acetate (600 mL) and dil. Hydrochloric acid (100 mL). The organic layer was washed with sodium bicarbonate (200 mL), brine (200 mL), dried (MgSO4) and was concentrated to afford 85 g (97%) of the desired product as a solid. ¹H NMR (CDCl₃) δ 7.62 (d, 1H, J=3.1 Hz), 7.15 (d, 1H, J=3.1 Hz), 5.22 (m, 1H), 4.18 (m, 2H), 3.81 (m, 2H), 2.90 (m, 2H), 1.45 (s, 9H), 1.24 (t, 3H, J=7.5 Hz). Anal. Calcd for C₁₈H₂₄ClIN₂O₆: Mol. Wt, 526.0368. Found Mol. Wt, 527.0451 (M+H, HRMS).

Step 5

### Ethyl 3-(R)—(N-gly)-amino-3-(5-chloro-2-hydroxy-3-iodophenyl)propionate hydrochloride

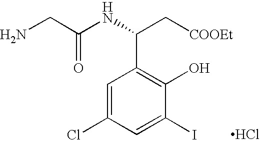

Ethanolic HCl (700 mL) was added to ethyl 3-(R)—(N—BOC-gly)-amino-3-(5-chloro-2-hydroxy-3-iodophenyl)propionate (84.5 g, 160.4 mmol) at 0° C. and was stirred at room temperature for 3 h. The reaction mixture was concentrated, and concentrated once more after addition of toluene (100 mL). The residue obtained was suspended in ether and was filtered and dried to afford 72.0 g (97%) of the desired product as a crystalline powder. ¹H NMR (CD₃OD) δ 7.67 (d, 1H, 3.5 Hz), 7.29 (d, 1H, J=3.2 Hz), 5.61 (m, 1H), 4.14 (q, 2H, J=9.7 Hz), 3.74 (s, 2H), 2.91 (m, 2H), 1.23 (t, 3H, J=9.7 Hz). Anal. Calcd for C₁₃H₁₆ClIN₂O₄: Mol. Wt, 425.9843. Found Mol. Wt, 426.9908 (M+H, HRMS).

Step 6

### R 5-Chloro-3-iodo-2-hydroxy-β-[[2-[[[3-hydroxy-5-[(1,4,5,6-tetrahydro-5-hydroxy-pyrimidin-2-yl)amino]phenyl]carbonyl]amino]-acetyl]amino]benzenepropanoic acid, trifluroacetate salt

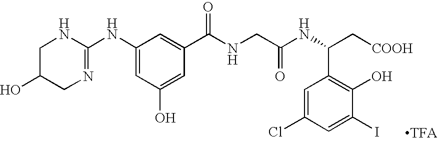

A solution of ³-N-(5-hydroxytetrahydropyrimidino)amino-5-hydroxybenzoic acid hydrochloride, prepared as in Example 1, (1.65 g, 5.94 mmol) in dimethylacetamide (25 mL) was heated until all the material had dissolved. This was then cooled to 0° C. and isobutylchloro-formate (1.20 mL) was added in one portion followed by N-methylmorpholine (1.0 mL). After 10 min, ethyl R-3-(N-gly)-amino-3-(3-iodo-5-chloro-2-hydroxyphenyl)-propionate hydrochloride (2.5 g, 5.40 mmol) was added in one portion followed by N-methylmorpholine (0.6 mL). The reaction mixture was stirred for 18 h at room temperature. The reaction mixture was concentrated and the residue was dissolved in ethanol/water (1:1, 20 mL) and was chromatographed (reverse phase, 95:5 water: acetonitrile over 60 min to 30:70 water: acetonitrile containing 0.1% TFA). The combined fractions were concentrated. The residue was dissolved in ethanol/water and was added sodium hydroxide until basic and was stirred for 2 h. The reaction mixture was concentrated and was purified as above by hplc to afford 0.84 g (19%) of the desired acid as the TFA salt. ¹H NMR (CD₃OD) δ 7.64 (d, 1H, J=3.2 Hz), 7.21 (m, 2H), 6.85 (t, 1H, J=2.9 Hz), 5.56 (m, 1H), 4.26 (m, 1H), 4.08 (s, 2H), 3.32-3.50 (m, 4H), 2.89 (m, 2H). Anal. Calcd for C₂₂H₂₃ClIN₅O₇: Mol. wt, 631.0331. Found: Mol. Wt, 632.0379 (M+H, HRMS).

### Example 61

### (R) 5-Chloro-3-iodo-2-hydroxy-β-[[2-[[[5-[(1,4,5,6-tetrahydro-5-hydroxy-pyrimidin-2-yl)amino]pyridyl]-3-carbonyl]amino]acetyl]amino]benzenepropanoic acid, trifluroacetate salt

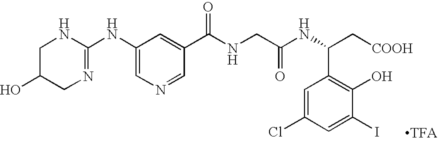

A solution of 5-N-(5-hydroxytetrahydropyrimidino)aminonicotinic acid hydro-chloride, prepared as in Example 2, (0.976 g, 3.37 mmol) in dimethylacetamide (10 mL) was heated until all the material had dissolved. This was then cooled to 0° C. and isobutylchloro-formate (0.48 mL) was added in one portion followed by N-methylmorpholine (0.41 mL). After 10 min, ethyl R-3-(N-gly)-amino-3-(3-iodo-5-chloro-2-hydroxy-phenyl)propionate hydrochloride, prepared as in Example 60, (1.56 g, 3.37 mmol) was added in one portion followed by N-methylmorpholine (0.41 mL). The reaction mixture was stirred for 18 h at room temperature. The reaction mixture was concentrated and the residue was dissolved in ethanol/water and was added sodium hydroxide until basic and was stirred for 2 h. The reaction mixture was concentrated and was purified as above by hplc to afford 0.5 g (20%) of the desired acid as the TFA salt. ¹H NMR (CD₃OD) δ 8.93 (s, 1H), 8.66 (d, 1H, J=3Hz), 8.19 (t, 1H, J=2.7 Hz), 7.64 (d, 1H, J=3.2 Hz), 7.31 (d, 1H, J=3.2 Hz), 5.57 (m, 1H), 4.29 (m, 1H), 4.14 (s, 2H), 3.32-3.53 (m, 4H), 2.90 (m, 2H). Anal. Calcd for C₂₁H₂₂ClIN₆O₆: Mol. wt, 616.0334. Found: Mol. Wt, 617.0401(M+H, HRMS).

### Example 62

### (R) 5-Chloro-3-iodo-2-hydroxy-β-[[2-[[[3-hydroxy-5-[(1,4,5,6-tetrahydro-pyrimidin-2-yl)amino]phenyl]carbonyl]amino]acetyl]amino]benzenepropanoic acid, trifluroacetate salt

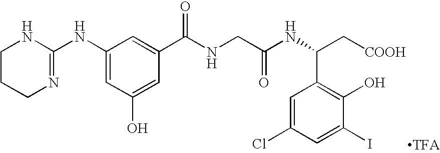

A solution of 3-N-(tetrahydropyrimidino)amino-5-hydroxybenzoic acid hydro-chloride, prepared as in Example 24, (1.61 g, 5.94 mmol) in dimethylacetamide (25 mL) was heated until all the material had dissolved. This was then cooled to 0° C. and isobutylchloro-formate (1.20 mL) was added in one portion followed by N-methylmorpholine (1.0 mL). After 10 min, ethyl R-3-(N-gly)-amino-3-(3-iodo-5-chloro-2-hydroxyphenyl)-propionate hydrochloride, prepared as in Example 60, (2.5 g, 5.40 mmol) was added in one portion followed by N-methylmorpholine (0.6 mL). The reaction mixture was stirred for 18 h at room temperature. The reaction mixture was concentrated and the residue was dissolved in ethanol/water and was added sodium hydroxide until basic and was stirred for 2 h. The reaction mixture was concentrated and was purified as above by hplc to afford 1.70 g (38%) of the desired acid as the TFA salt. ¹H NMR (CD₃OD) δ 7.64 (d, 1H, J=3.5 Hz), 7.30 (d, 1H, J=3.2 Hz), 7.17-7.22 (m, 2H), 6.83 (t, 1H, J=2.9 Hz), 5.56 (m, 1H), 4.08 (s, 2H), 3.32-3.43 (m, 4H), 2.91 (m, 2H), 2.02 (m, 2H). Anal. Calcd for C₂₂H₂₃ClIN₅O₆: Mol. wt, 615.0382. Found: Mol. Wt, 616.0444(M+H, HRMS).

### Example 63

### (R) 5-Chloro-3-iodo-2-hydroxy-β-[[2-[[[5-[3-amino-(5-hydroxy-1,4,5,6-tetrahydro-pyrimidin-2-yl)amino]phenyl]carbonyl]amino]acetyl]amino]benzenepropanoic acid, trifluroacetate salt

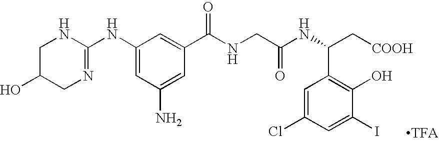

A solution of 5-N-trifluoroacetylamino-3-N-(5-hydroxytetrahydro-pyrimidino)-aminobenzoic acid hydrochloride, prepared as in Example 3, (1.40 g, 3.75 mmol) in dimethylacetamide (25 mL) was heated until all the material had dissolved. This was then cooled to 0° C. and isobutylchloroformate (0.54 mL) was added in one portion followed by N-methylmorpholine (0.45 mL). After 10 min, ethyl R-3-(N-gly)-amino-3-(3-iodo-5-chloro-2-hydroxyphenyl)-propionate hydrochloride, prepared as in Example 60, (1.74 g, 3.75 mmol) was added in one portion followed by N-methylmorpholine (0.45 mL). The reaction mixture was stirred for 18 h at room temperature. The reaction mixture was concentrated and the residue was dissolved in ethanol/water and was added sodium hydroxide until basic and was stirred for 2 h. The reaction mixture was concentrated and was purified as above by hplc to afford 0.82 g (29%) of the desired acid as the TFA salt. ¹H NMR (CD₃OD) δ 7.65 (m, 1H), 7.30 (m, 1H), 7.18 (m, 1H), 7.11 (m, 1H), 6.85 (m, 1H), 5.57 (m, 1H), 4.25 (m, 1H), 4.08 (s, 2H), 3.32-3.49 (m, 4H), 2.90 (m, 2H). Anal. Calcd for C₂₂H₂₄ClIN₆O₆: Mol. wt, 630.0491. Found: Mol. Wt, 631.0557 (M+H, HRMS).

### Example 64

### (R) 5-Chloro-3-iodo-2-hydroxy-β-[[2-[[[5-[(5-hydroxy-1,4,5,6-tetrahydro-pyrimidin-2-yl)amino]phenyl]carbonyl]amino]acetyl]amino]benzenepropanoic acid, trifluroacetate salt.

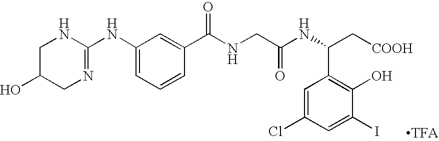

A solution of 3-N-(5-hydroxytetrahydropyrimidino)-aminobenzoic acid hydro-chloride, prepared as in Example 25, (1.61 g, 5.94 mmol) in dimethylacetamide (25 mL) was heated until all the material dissolved. This was then cooled to 0° C. and iso-butylchloroformate (1.20 mL) was added in one portion followed by N-methylmorpholine (1.0 mL). After 10 min, ethyl R-3-(N-gly)-amino-3-(3-iodo-5-chloro-2-hydroxyphenyl)-propionate hydrochloride, prepared as in Example 60, (2.5 g, 5.40 mmol) was added in one portion followed by N-methylmorpholine (0.6 mL). The reaction mixture was stirred for 18 h at room temperature. The reaction mixture was concentrated and the residue was dissolved in ethanol/water and was added sodium hydroxide until basic and was stirred for 2 h. The reaction mixture was concentrated and was purified as above by hplc to afford 1.60 g (36%) of the desired acid as the TFA salt. ¹H NMR (CD₃OD) δ 7.76-7.81 (m, 2H), 7.64 (d,1H, J=3.2 Hz), 7.57 (m, 1H), 7.46 (m, 1H), 7.30 (m,1H), 5.57 (m, 1H), 4.25 (m, 1H), 4.11 (s, 2H), 3.32-3.49 (m, 4H), 2.90 (m, 2H). Anal. Calcd for C₂₂H₂₃ClIN₅O₆: Mol. wt, 615.0382. Found: Mol. Wt, 616.0470(M+H, HRMS).

### Example 65

### (R) 5-Chloro-2-hydroxy-3-iodo-β-[[2-[[[5-[(1,4,5,6-tetrahydropyrimidin-2-yl)amino]-phenyl]carbonyl]amino]acetyl]amino]benzenepropanoic acid, trifluroacetate salt

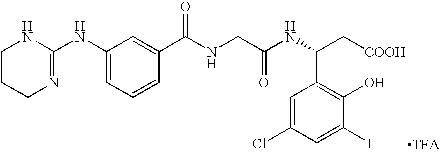

Trifluoroacetic acid (0.264 mL) was added to 3-N-(tetrahydropyrimidino)-aminobenzoic acid, prepared as in Example 30, (0.75 g, 3.43 mmol) in DMF (15 mL) and was stirred for 15 min. EDC (0.60 g, 3.43 mmol) followed by HOBt (0.463 g, 3.43 mmol) and the reaction mixture was stirred for 30 min. Ethyl R-3-(N-gly)-amino-3-(5-chloro-2-hydroxy-3-iodophenyl)propionate hydrochloride, prepared as in Example 60, (1.59 g, 3.43 mmol) followed by N-methylmorpholine (0.367 mL) was added to the reaction mixture and was stirred for 18 h. The reaction mixture was concentrated in vacuo and the residue was dissolved in ethanol/water and treated with lithium hydroxide until basic. After the hydrolysis has been complete, (18 h), the reaction mixture was concentrated at room temperature and the residue was purified by hplc to afford 0.97 g (40%) of the desired product as its TFA salt. ¹H NMR (CD₃OD) δ 7.87 (m, 1H), 7.81 (m, 1H), 7.71 (m, H), 7.63 (m, 1H), 7.49 (m, 1H), 7.37 (m, 1H), 5.64 (m, 1H), 4.18 (s, 2H), 3.47-3.50 (m, 4H), 2.98 (m, 2H) 2.10 (m, 2H). Anal. Calcd for C₂₂H₂₃BrClN₅O₆: Mol. wt, 599.0432. Found: Mol. Wt, 600.0477 (M+H, HRMS).

### Example 66

### (3R)-3-(3,5-dichloro-2-hydroxyphenyl)-3-{[N-({5-[(5-hydroxy-1,4,5,6-tetra-hydropyrimidin-2-yl)amino]6-oxo-1,6-dihydropyridin-3-yl}carbonyl)glycyl]-amino}propanoic acid

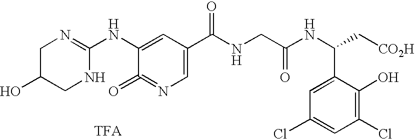 Step 1

### Ethyl (3R)-3-(3,5-dichloro-2-hydroxyphenyl)-3-{[N-({5-[(5-hydroxy-1,4,5,6-tetrahydropyrimidin-2-yl)amino]-6-oxo-1,6-dihydropyridin-3-yl}carbonyl)-glycyl]amino}propanoate

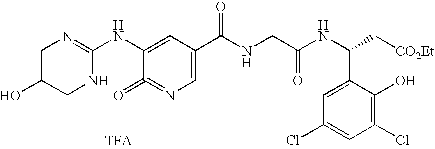

To a solution of 5-[(5-hydroxy-1,4,5,6-tetrahydropyrimidin-2-yl)amino]-6-oxo-1,6-dihydropyridine-3-carboxylic acid prepared as reported in WO 9952896, Example 33, (0.527 g ,1.3 mmol) in dimethyl-acetamide (DMA), asolution of CDMT (0.244 g , 1.4 mmol) in DMA (8 mL) was added and the mixture was stirred at 0° C. under argon atmosphere. Then added NMM (0.15 mL, 1.4 mmol) over 5 min and the mixture was stirred at 0° C. After 3 h, a solution of the amine prepared as in Example 3, (0.486g, 1.3 mmol), and NMM (0.15 mL) in DMA (10.0 mL) was added and the resulting mixture was stirred overnight at room temperature. The reaction was quenched with TFA (2 mL), and stirred for 1.5 h. After concentration in vacuo, the crude reaction mixture was purified by RP-HPLC using a gradient elution of 90:10 H₂O/TFA: CH₃CN at 254 nm. The title compound was isolated as a white solid (725 mg, 75%): ¹H NMR (DMSO d₆) δ 12.44 (1H, br d), 9.92 (1H, br s), 9.11 (1H, s), 8.65 (1H, t), 8.54 (1H, d), 8.09 (2H, br s), 8.00 (1H, br s), 8.80 (1H, d), 7.41 (1H, d), 7.26 (1H, d), 5.49 (1H, m), 4.05 (3H, m), 3.86 (2H, m), 3.34 (2H, br d), 3.15 (2H, dt), 2.71 (2H, m), 1.14 (3H, t); Anal. Calcd for C₂₃H₂₆N₆O₇Cl₂.1.5 TFA: C, 42.26; H, 3.87; N, 11.45. Found: C, 42.18; H, 3.74; N, 11.35; HRMS calcd for C₂₃H₂₆N₆O₇Cl₂ 569.1318. Found 569.1323.

Step 2

### (3R)-3-(3,5-dichloro-2-hydroxyphenyl)-3-{[N-({5-[(5-hydroxy-1,4,5,6-tetrahydropyrimidin in-2-yl)amino]-6-oxo-1,6-dihydropyridin-3-yl}carbonyl)-glycyl]amino}propanoic acid, trifluroacatate

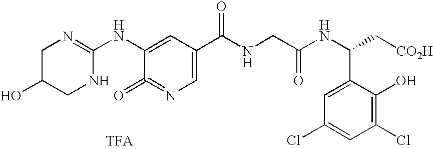

The ethyl ester obtained from Step 1 (0.725 g, 0.98 mmol) was dissolved in THF (5 mL), added a solution of 1 M NaOH (6.5 mL, 6.5 mmol), and stirred overnight at room temperature. The reaction mixture was then neutralized with 1 M HCl (6.5 mL), concentrated under reduced pressure, and the desired product was isolated by RP-HPLC using a gradient elution of 95:5 H₂O/TFA: CH₃CN at 254 nm. The acid was obtained as a white solid (589 mg, 79): ¹H NMR (DMSO d₆) δ 12.44 (1H, br d), 9.90 (1H, br s), 9.07 (1H, s), 8.64 (1H, t), 8.53 (1H, d), 8.06 (2H, br s), 8.00 (1H, m), 7.80 (1H, d), 7.41 (1H, d), 7.24 (1H, d), 5.44 (1H, m), 4.07 (2H, m), 3.88 (2H, m), 3.34 (2H, br d), 3.16 (2H, dt), 2.65 (2H, m); %): Anal calcd for C₂₁H₂₂N₆O₇Cl₂.1.9 TFA: C, 39.22; H, 3.16; N, 11.39. Found: C, 39.30; H, 3.18; N, 11.09; HRMS calcd for C₂₁H₂₂N₆O₇Cl₂541.1005. Found: 541.1000.

### Example 67

### (3R)-3-(3-Bromo-5-chloro-2-hydroxyphenyl)-3-({N-[3-hydroxy-5-(3,4,5,6-tetrahydro-2H-azepin-7-ylamino)benzoyl]glycyl}amino)propanoic acid trifluoroacetic acid salt

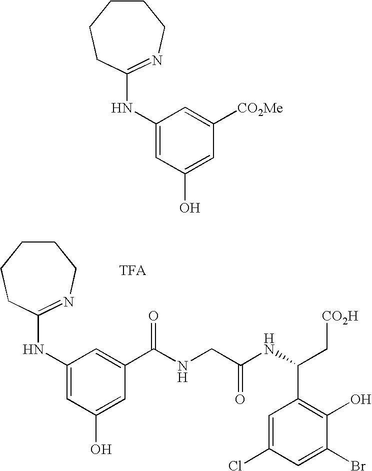 Step 1

### Methyl 3-hydroxy-5-(3,4,5,6-tetrahydro-2H-azepin-7-ylamino)benzoate

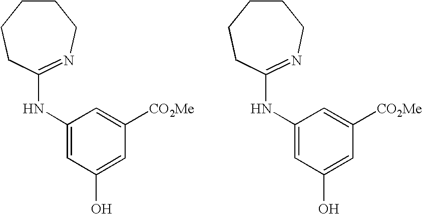

A mixture of methyl 3-hydroxy-5-aminobenzoate, prepared from the corresponding acid and ethanol and hydrochloric acid (2 g, 11.96 mmol) and 1-aza-2-methoxy-1-cycloheptene (2 g, 15.7 mmol) was heated neat in an oil bath at 140° C. for a period of 1 hour. The resulting solid mass was cooled to room temperature and triturated with ethyl acetate. The solid was filtered and dried and used in the next step without further purification. The yield was 2.7 g (86%) of the title compound.

| | | |
|---|---|---|
| ESI MS (MH+) for C₁₄H₁₈N₂O₃ | calculated | 263 |
| | found | 263 |

Step 2

### 3-Hydroxy-5-(3,4,5,6-tetrahydro-2H-azepin-7-ylamino)benzoic acid hydrochloride salt

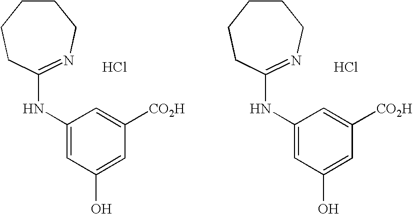

The product of STEP 1 (1 g, 3.8 mmol) was treated with 2N hydrochloric acid solution. The solution was heated to reflux for 5 hours. The solution was then cooled to room temperature during which time a precipitate formed. The solid was filtered and dried and used in the next step without further purification. The yield was 0.8 g (74%) of the title compound. ESI MS (free base MH+) for C₁₃H₁₆N₂O₃, Calculated: 263. Found: 263

Step 3

### Ethyl (3R)-3-(3-bromo-5-chloro-2-hydroxyphenyl)-3-({N-[3-hydroxy-5-(3,4,5,6-tetrahydro-2H-azepin-7-ylamino)benzoyl]glycyl}amino)propanoate trifluoroacetic acid salt

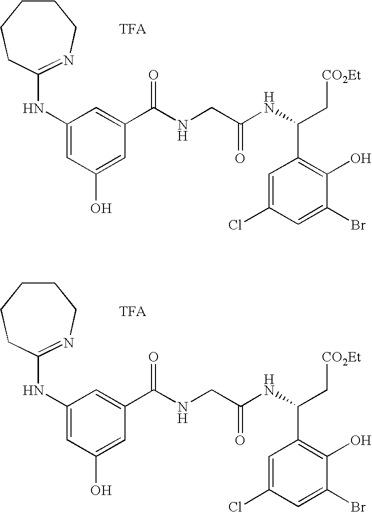

To a stirred and cooled (0° C.) solution of the product from Step 2 (0.15 g, 0.53 mmol) and N-methylmorpholine (0.058 mL, 0.53 mmol) in DMF (3 mL) was added isobutyl chloroformate (0.069 mL, 0.53 mmol). The mixture was stirred for 30 minutes. To this solution was added a solution of the product from Example 1 Step 3, ethyl (3R)-3-(3-bromo-5-chloro-2-hydroxyphenyl)-3-(glycylamino)-propanoate, hydrochloride (0.22 g, 0.53 mmol) and N-methylmorpholine (0.058 g, 0.53 mmol) in DMF (2 mL). The reaction mixture was then allowed to warm to room temperature and stirred 18 hours. The volatile components were removed at reduced pressure and the residue was chromatographed (reverse phase HPLC, gradient elution with water/acetonitrile/trifluoroacetic acid). This produced 130 mg (33.9%) of the title compound. ESI MS (free base MH+) for C₂₆H₃₀N₄O₆ BrCl, Calculated: 611. Found: 611.

Step 4

### (3R)-3-(3-Bromo-5-chloro-2-hydroxyphenyl)-3-({N-[3-hydroxy-5-(3,4,5,6-tetrahydro-2H-azepin-7-ylamino)benzoyl]glycyl}amino)propanoic acid trifluoroacetic acid salt

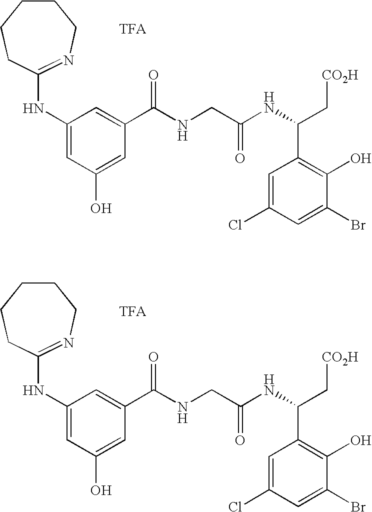

The product of Step 3 (0.125 g, 0.17 mmol) in THF (3 mL) was cooled (0° C.) and treated with 1N lithium hydroxide solution (0.6 mL, 0.6 mmol). The solution was warmed to room temperature and stirred 18 hours. The volatile components were removed at reduced pressure on a rotary evaporator. The crude product was chromatographed (reverse phase C18-HPLC, gradient elution with water/acetonitrile/trifluoroacetic acid). This produced 90 mg (76%) of the title compound. ESI MS (free base MH+) for C₂₄H₂₆N₄O₆ BrCl, Calculated: 583. Found: 583.

### Example 68

### (3R)-3-(3,5-Dichloro-2-hydroxyphenyl)-3-({N-[3-hydroxy-5-(3,4,5,6-tetrahydro-2H-azepin-7-ylamino)benzoyl]glycyl}amino)propanoic acid. trifluoroacetate

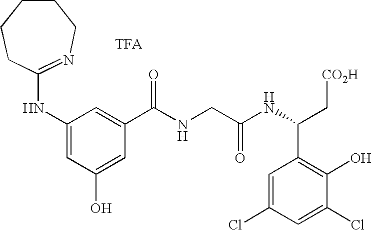 Step 1

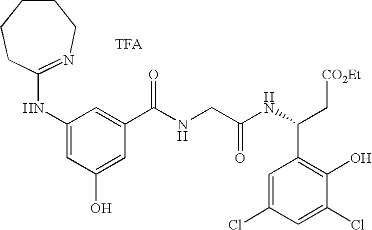

Using substantially the same procedures and materials of EXAMPLE 67, STEP 3 but substituting ethyl (3R)-3-(3,5-dichloro-2-hydroxyphenyl)-3-(glycylamino)-propanoate, hydrochloride for ethyl (3R)-3-(3-bromo-5-chloro-2-hydroxyphenyl)-3-(glycylamino)-propanoate, hydrochloride, results in formation of ethyl (3R)-3-(3,5-dichloro-2-hydroxyphenyl )-3-({N-[3-hydroxy-5-(3,4,5,6-tetrahydro-2H-azepin-7-ylamino)benzoyl]glycyl}amino)propanoate trifluoroacetate after isolation by C-18 reverse phase hplc.

Step 2

### (3R)-3-(3,5-Dichloro-2-hydroxyphenyl)-3-({N-[3-hydroxy-5-(3,4,5,6-tetrahydro-2H-azepin-7-ylamino)benzoyl]glycyl}amino)propanoic acid. trifluoroacetate

The product from STEP 1 is hydrolyzed according to the procedures of EXAMPLE 67, STEP 4 to afford the corresponding acid, (3R)-3-(3,5-dichloro-2-hydroxyphenyl)-3-({N-[3-hydroxy-5-(3,4,5,6-tetrahydro-2H-azepin-7-ylamino)benzoyl]glycyl}amino)propanoic acid trifluoroacetate, which may be isolated by rphplc.

### Example 69

### (3R)-3-(5-Bromo-3-chloro-2-hydroxyphenyl)-3-({N-[3-hydroxy-5-(3,4,5,6-tetrahydro-2H-azepin-7-ylamino)benzoyl]glycyl}amino) propanoic acid

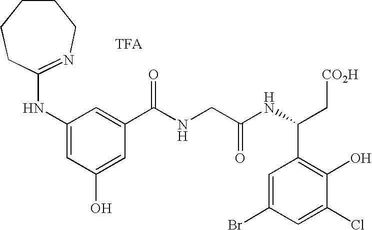 Step 1

### Ethyl (3R)-3-(5-bromo-3-chloro-2-hydroxyphenyl)-3-({N-[3-hydroxy-5-(3,4,5,6-tetrahydro-2H-azepin-7-ylamino)benzoyl]glycyl}amino)propanoate, trifluoroacetate

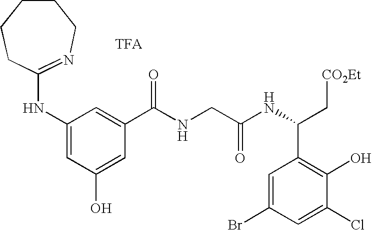

Using substantially the same procedures and materials of EXAMPLE 67, STEP 3 but substituting the product of EXAMPLE 18, STEP 5 for ethyl (3R)-3-(3-bromo-5-chloro-2-hydroxyphenyl)-3-(glycylamino)-propanoate, hydrochloride, results in formation of ethyl (3R)-3-(5-bromo-3-chloro-2-hydroxyphenyl)-3-({N-[3-hydroxy-5-(3,4,5,6-tetrahydro-2H-azepin-7-ylamino)benzoyl]glycyl}amino)propanoate, trifluoroacetate

Step 2

### (3R)-3-(5-Bromo-3-chloro-2-hydroxyphenyl)-3-({N-[3-hydroxy-5-(3,4,5,6-tetrahydro-2H-azepin-7-ylamino)benzoyl]glycyl}amino) propanoic acid

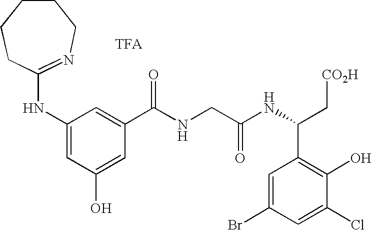

The product of STEP 1 is hydrolyzed according to the procedures of EXAMPLE 67, STEP 4 to afford the corresponding acid, (3R)-3-(5-bromo-3-chloro-2-hydroxyphenyl)-3-({N-[3-hydroxy-5-(3,4,5,6-tetrahydro-2H-azepin-7-ylamino)benzoyl]glycyl}amino)propanoic acid, which may be isolated by rphplc.

### Example 70

### (3R)-3-(5-Chloro-2-hydroxy-3-iodophenyl)-3-({N-[3-hydroxy-5-(3,4,5,6-tetrahydro-2H-azepin-7-ylamino)benzoyl]glycyl}amino)propanoic acid trifluoroacetate

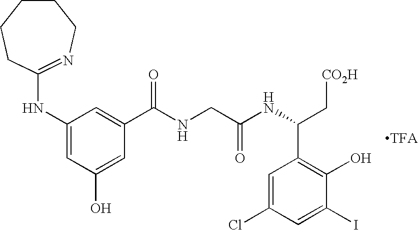 Step 1

### Ethyl (3R)-3-(5-chloro-2-hydroxy-3-iodophenyl)-3-({N-[3-hydroxy-5-(3,4,5,6-tetrahydro-2 H-azepin-7-ylamino)benzoyl]glycyl}amino)propanoate trifluoroacetate

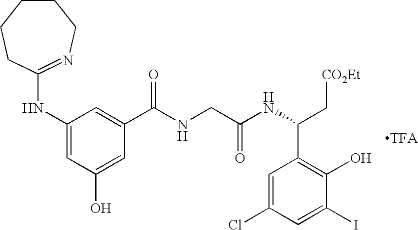

Using substantially the same procedures and materials of Example 67, Step 3 but substituting ethyl R-3-(N-gly)-amino-3-(5-chloro-2-hydroxy-3-iodophenyl)-propionate hydrochloride, prepared as in Example 60, Step 5 for ethyl (3R)-3-(3-bromo-5-chloro-2-hydroxyphenyl)-3-(glycylamino)-propanoate, hydrochloride, results in formation of ethyl (3R)-3-(5-chloro-2-hydroxy-3-iodophenyl)-3-({N-[3-hydroxy-5-(3,4,5,6-tetrahydro-2H-azepin-7-ylamino)benzoyl]glycyl}amino)-propanoate trifluoroacetate after isolation by C-18 reverse phase hplc.

Step 2

### (3R)-3-(5-Chloro-2-hydroxy-3-iodophenyl)-3-({N-[3-hydroxy-5-(3,4,5,6-tetrahydro-2H-azepin-7-ylamino)benzoyl]glycyl}amino)propanoic acid trifluoroacetate

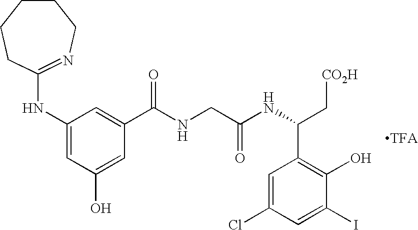

The product of STEP 1 is hydrolyzed according to the procedures of EXAMPLE 67, STEP 4 to afford the corresponding acid, (3R)-3-(5-chloro-2-hydroxy-3-iodophenyl)-3-({N-[3-hydroxy-5-(3,4,5,6-tetrahydro-2H-azepin-7-ylamino)benzoyl]glycyl}amino)propanoic acid , which may be isolated by rphplc.

### Example 71

### (3R)-3-(3,5-Dibromo-2-hydroxyphenyl)-3-({N-[3-hydroxy-5-(3,4,5,6-tetrahydro-2H-azepin-7-ylamino)benzoyl]glycyl}amino)propanoic acid

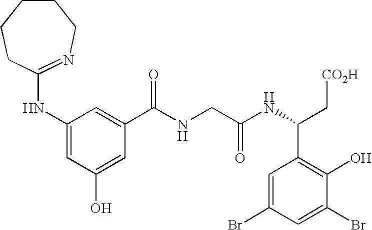 Step 1

### Ethyl (3R)-3-(3,5-dibromo-2-hydroxyphenyl)-3-({N-[3-hydroxy-5-(3,4,5,6-tetrahydro-2H-azepin-7-ylamino)benzoyl]glycyl}amino)propanoate trifluoroacetate

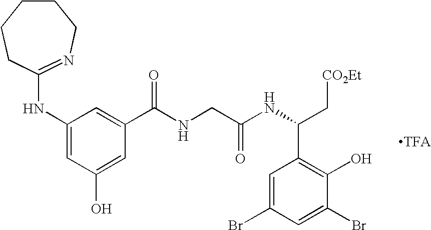

Using substantially the same procedures and materials of EXAMPLE 67, STEP 3 but substituting ethyl 3-R-(N-gly)-amino-3-(3,5-dibromo-2-hydroxyphenyl)-propionate hydrochloride, prepared as in Example 51, for ethyl (3R)-3-(3-bromo-5-chloro-2-hydroxyphenyl)-3-(glycylamino)-propanoate, hydrochloride, results in formation of ethyl (3R)-3-(3,5-dibromo-2-hydroxyphenyl)-3-({N-[3-hydroxy-5-(3,4,5,6-tetrahydro-2H-azepin-7-ylamino)benzoyl]glycyl}amino) propanoate trifluoroacetate after isolation by C-18 reverse phase hplc.

Step 2

### (3R)-3-(3,5-Dibromo-2-hydroxyphenyl)-3-({N-[3-hydroxy-5-(3,4,5,6-tetrahydro-2H-azepin-7-ylamino)benzoyl]glycyl}amino)propanoic acid

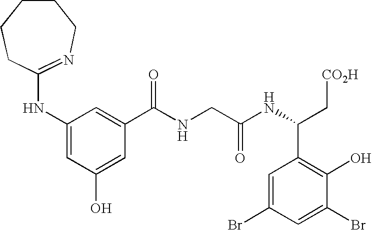

The product of Step 1 is hydrolyzed according to the procedures of Example 67, Step 4 to afford the corresponding acid, (3R)-3-(3,5-dibromo-2-hydroxyphenyl)-3-({N-[3-hydroxy-5-(3,4,5,6-tetrahydro-2H-azepin-7-ylamino)benzoyl]glycyl}-amino)propanoic acid , which may be isolated by rphplc.

The activity of the compounds of the present invention was tested in the following assays. In one embodiment, compounds of the present invention antagonize the αᵥβ₃ integrin with an IC₅₀ of 0.1nM to 100 μM in the 293-cell assay. In another embodiment, compounds of the present invention antagonize the αᵥβ₃ integrin with an IC₅₀ of 0.1 nM to 0.2 μM in the 293-cell assay. Similarly these compounds also antagonized the αᵥβ₅ integrin with an IC₅₀ of about 0.1 nM to about 100 μM in the cell adhesion assay, and in another embodiment, from 0.1 nM to 0.2 μM. In yet another embodiment, the compounds of the present invention also antagonized the IIb-IIIa integrin with an IC₅₀ of greater than about 0.1 μM to about 100 μM in the HT-29 cell-based adhesion assay.

In another embodiment, the compounds further have a selectivity ratio of αᵥβ₃ integrin antagonism over the IIb3a integrin antagonism of at least 10, and in another embodiment, of at least 100. In another embodiment, the compounds further have a selectivity ratio of αᵥβ₃ integrin antagonism over the αᵥβ₆ integrin antagonism of at least 10, and in another embodiment, of at least 100.

### Vitronectin Adhesion Assay

### Materials

Human vitronectin receptors αᵥβ₃ and αᵥβ₅ were purified from human placenta as previously described [Pytela et al., Methods in Enzymology, 144:475-489 (1987)]. Human vitronectin was purified from fresh frozen plasma as previously described [Yatohgo et al., Cell Structure and Function, 13:281-292 (1988)]. Biotinylated human vitronectin was prepared by coupling NHS-biotin from Pierce Chemical Company (Rockford, Ill.) to purified vitronectin as previously described [Charo et al., J. Biol. Chem., 266(3):1415-1421 (1991)]. Assay buffer, OPD substrate tablets, and RIA grade BSA were obtained from Sigma (St. Louis, Mo.). Anti-biotin antibody was obtained from Sigma (St. Luois, Mo.). Nalge Nunc-Immuno microtiter plates were obtained from Nalge Company (Rochester, N.Y.).

### Methods

### Solid Phase Receptor Assays

This assay was essentially the same as previously reported [Niiya et al., Blood, 70:475-483 (1987)]. The purified human vitronectin receptors αᵥβ₃ and αᵥβ₅ were diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptors were immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 μL was added to each well. After 45 minutes, the plates were washed and incubated with OPD/H₂O₂ substrate in 100 mM/L Citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCEL spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm [Rodbard et al., Int. Atomic Energy Agency, Vienna, pp 469 (1977)], plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. β-[[2-[[5-[(aminoiminomethyl)amino]-1-oxopentyl]amino]-1-oxoethyl]amino]-3-pyridinepropanoic acid [U.S. Pat. No. 5,602,155 Example 1] which is a potent αᵥβ₃ antagonist (IC₅₀ in the range 3-10 nM) was included on each plate as a positive control.

### Purified IIb/IIIa Receptor Assay

Materials

Human fibrinogen receptor (IIb/IIIa) was purified from outdated platelets. (Pytela, R., Pierschbacher, M. D., Argraves, S., Suzuki, S., and Rouslahti, E. “Arginine-Glycine-Aspartic acid adhesion receptors”, Methods in Enzymology 144(1987):475-489). Human vitronectin was purified from fresh frozen plasma as described in Yatohgo, T., Izumi, M., Kashiwagi, H., and Hayashi, M., “Novel purification of vitronectin from human plasma by heparin affinity chromatography,” Cell Structure and Function 13(1988):281-292. Biotinylated human vitronectin was prepared by coupling NHS-biotin from Pierce Chemical Company (Rockford, Ill.) to purified vitronectin as previously described. (Charo, I. F., Nannizzi, L., Phillips, D. R., Hsu, M. A., Scarborough, R. M., “Inhibition of fibrinogen binding to GP IIb/IIIa by a GP IIIa peptide”, J. Biol. Chem. 266(3)(1991): 1415-1421.) Assay buffer, OPD substrate tablets, and RIA grade BSA were obtained from Sigma (St. Louis, Mo.). Anti-biotin antibody was obtained from Sigma (St. Louis, Mo.). Nalge Nunc-Immuno microtiter plates were obtained from (Rochester, N.Y.). ADP reagent was obtained from Sigma (St. Louis, Mo.).

### Methods

### Solid Phase Receptor Assays

This assay is essentially the same reported in Niiya, K., Hodson, E., Bader, R., Byers-Ward, V. Koziol, J. A., Plow, E. F. and Ruggeri, Z. M., “Increased surface expression of the membrane glycoprotein IIb/IIIa complex induced by platelet activation: Relationships to the binding of fibrinogen and platelet aggregation”, Blood 70(1987):475-483. The purified human fibrinogen receptor (IIb/IIIa) was diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptor was immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 ,μL were added to each well. After 45 minutes, the plates were washed and incubated with ODD/H₂O₂ substrate in 100 mM/L citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCELJ spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm, [Robard et al., Int. Atomic Energy Agency, Vienna, pp 469 (1977)], plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. β-[[2-[[5-[(aminoimino-methyl)amino]-1-oxopentyl]amino]-1-oxoethyl]amino]-3-pyridinepropanoic acid, bistrifluoroacetate salt [U.S. Pat. No. 5,602,155 Example 1] which is a potent IIb/IIIa antagonist (IC₅₀ in the range 8-18 nM) was included on each plate as a positive control.

### Human Platelet Rich Plasma Assays

Healthy aspirin free donors were selected from a pool of volunteers. The harvesting of platelet rich plasma and subsequent ADP induced platelet aggregation assays were performed as described in Zucker, M. B., “Platelet Aggregation Measured by the Photometric Method”, Methods in Enzymology 169(1989):117-133. Standard venipuncture techniques using a butterfly allowed the withdrawal of 45 mL of whole blood into a 60 mL syringe containing 5 mL of 3.8% trisodium citrate. Following thorough mixing in the syringe, the anti-coagulated whole blood was transferred to a 50 mL conical polyethylene tube. The blood was centrifuged at room temperature for 12 minutes at 200×g to sediment non-platelet cells. Platelet rich plasma was removed to a polyethylene tube and stored at room temperature until used. Platelet poor plasma was obtained from a second centrifugation of the remaining blood at 2000×g for 15 minutes. Platelet counts are typically 300,000 to 500,000 per microliter. Platelet rich plasma (0.45 mL) was aliquoted into siliconized cuvettes and stirred (1100 rpm) at 37° C. for 1 minute prior to adding 50 uL of pre-diluted test compound. After 1 minute of mixing, aggregation was initiated by the addition of 50 uL of 200 uM ADP. Aggregation was recorded for 3 minutes in a Payton dual channel aggregometer (Payton Scientific, Buffalo, N.Y.). The percent inhibition of maximal response (saline control) for a series of test compound dilutions was used to determine a dose response curve. All compounds were tested in duplicate and the concentration of half-maximal inhibition (IC₅₀) was calculated graphically from the dose response curve for those compounds which exhibited 50% or greater inhibition at the highest concentration tested; otherwise, the IC₅₀ is reported as being greater than the highest concentration tested.

### Cell Assays for Potency and Selectivity

While the β₃ subunit of αᵥβ₃ only known to complex with αᵥ or α_{IIb}, the αᵥ subunit complexes with multiple β subunits. The three αᵥ integrins most homologous with αᵥβ₃ are αᵥβ₁, αᵥβ₅ and αᵥβ₆, with 43%, 56% and 47% amino acid identity in the β subunits, respectively. To evaluate the selectivity of compounds between the integrins αᵥβ₃ and αᵥβ₆, cell-based assays were established using the 293 human embryonic kidney cell line. 293 cells express αᵥβ₁, but little to no detectable αᵥβ₃ or αᵥβ₆. cDNAs for β₃ and β₆ were transfected separately into 293 cells to generate 293-β3 and 293-β6 cells, respectively. High surface expression of αᵥβ₃ and αᵥβ₆ was confirmed by flow cytometry. Conditions were established for each cell line in which cell adhesion to immobilized human vitronectin was mediated by the appropriate integrin, as determined by a panel of integrin-specific, neutralizing monoclonal antibodies. Briefly, cells were incubated with inhibitor in the presence of 200 uM Mn²⁺, allowed to adhere to immobilized vitronectin, washed, and adherent cells are detected endogenous alkaline phosphatase and para-nitrophenyl phosphate. An 8-point dose-response curve using either 10-fold or 3-fold dilutions of compound was evaluated by fitting a four-parameter logistic, nonlinear model (using SAS).

To evaluate compound potency for membrane-bound αᵥβ₆ an additional cell-based adhesion assay was established using the HT-29 human colon carcinoma cell line. High surface expression of αᵥβ₆ on HT-29 cells was confirmed by flow cytometry. Conditions were established in which cell adhesion to immobilized human latency associated peptide (LAP) was mediated by the αᵥβ₆, as determined by a panel of integrin-specific, neutralizing monoclonal antibodies. Briefly, cells were incubated with inhibitor in the presence of 200 uM Mn²⁺, allowed to adhere to immobilized LAP, washed, and adherent cells are detected by quantifying endogenous alkaline phosphatase using para-nitrophenyl phosphate. An 8-point dose-response curve using either 10-fold or 3-fold dilutions of compound was evaluated by fitting a four-parameter logistic, nonlinear model (using SAS). The compounds evaluated were relatively ineffective at inhibition of αᵥβ₆-mediated cell adhesion.

1. A compound having the structure of Formula I 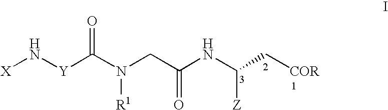
or a pharmaceutically acceptable salt or tautomer thereof;
wherein:
X has the structure of Formula Ia: 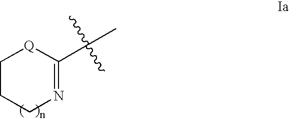
and wherein X is optionally substituted with one or more substituents independently selected from the group consisting of OH, alkyl, alkenyl, alkynyl, haloalkyl, alkylaryl, arylalkyl, alkoxy, dialkylamino, thioalkyl, cycloalkyl, CN, NO₂, and halogen;
n is a number from zero to two;
Y is a six-membered aryl or heterocycyl ring; wherein Y is optionally substituted with one or more moieties independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, methylenedioxy, ethylenedioxy, thioalkyl, alkylamino, arylamino, alkylsulfonamido, acyl, acylamino, alkylsulfone, sulfonamido, alkenyl, alkynyl, carboxamide, NHCOCF₃, and —(CH₂)ₘCOR²;
m is a number from zero to two;
R² is selected from the group consisting of hydroxy, alkoxy, and amino;
Z is an aryl or heterocyclyl ring having about five to about six members, or a bicyclic aryl ring having about nine to about twelve members, wherein Z optionally contains one to five heteroatoms independently selected from the group consisting of O, N and S; wherein Z is optionally substituted with one or more substituents selected from the group consisting of alkyl, haloalkyl, aryl, heterocycyl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, NHCOCF₃, and —(CH₂)ₚCOR⁷⁹; wherein the aryl and heterocycyl substituents are optionally substituted with one or more substituents selected from the group consisting of alkyl, haloalkyl, halogen, alkoxyalkyl, aminoalkyl, cycloalakyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)_{q}COR⁸⁰;
p is a number from zero to two;
R⁷⁹ is selected from the group consisting of hydroxy, alkoxy, and amino;
q is a number from zero to two;
R⁸⁰ is selected from the group consisting of hydroxy, alkoxy, and amino;
Q is selected from the group consisting of NH and CH₂;
R is selected from the group consisting of OH, alkoxy, and NHR³;
R³ is selected from the group consisting of H and alkyl group;
R¹ is selected from the group consisting of H, CN, NO₂, acyl, haloalkyl, alkenyl, alkynyl, and alkyl;
and carbon atom 3 of Formula I is in the (R) conformation.

2. A compound according to claim 1 wherein Z is a substituted phenyl ring.

3. A compound according to claim 1 wherein Y is a six-membered ring with zero to two nitrogen atoms substituted with a moiety selected from the group consisting of O, NH₂, NO₂, OH, and CH₃.

4. A compound according to claim 3 wherein Y is selected from the group consisting of phenyl and pyridine.

5. A compound according to claim 1 wherein n is 1-2.

6. A compound according to claim 5 wherein X contains 1-2 nitrogen atoms, and is substituted with a moiety selected from the group consisting of H, OH, alkyl, CN, NO₂, aminoalkyl, halogen, haloalkyl, and alkoxy.

7. A compound according to claim 6 wherein X is selected from the group consisting of azepine and diazepine.

8. A compound according to claim 1 wherein
X has the structure of Formula Ib: 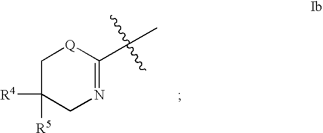
and
R⁴ and R⁵ are independently selected from the group consisting of H, OH, alkyl, CN, NO₂, aminoalkyl, halogen, haloalkyl, and alkoxy.

9. A compound according to claim 1 wherein
X has the Formula Ie: 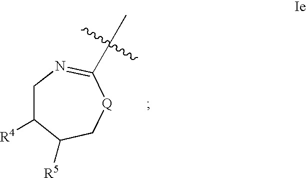
and
R⁴ and R⁵ are independently selected from the group consisting of H, OH, alkyl, CN, NO₂, aminoalkyl, halogen, haloalkyl, and alkoxy; and
Y and Z are each a 6-membered aryl ring.

10. A compound according to claim 8 wherein
Z has the formula: 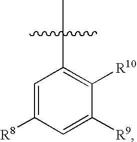
wherein R⁸ is H or OH, and R⁹, R¹⁰ are halogen.

11. A compound according to claim 10 wherein
Q is NH;
R⁴ is OH or CH₃;
R⁵ is H or methyl.
R⁸ is Cl or Br;
R⁹ is selected from the group consisting of I, Br, and Cl; and
R¹⁰ is OH.

12. A compound according to claim 10 wherein Q is CH₂
R⁴ is OH or CH₃;
R⁵ is H or methyl.
R⁸ is Cl or Br;
R⁹ is selected from the group consisting of I, Br, and Cl; and
R¹⁰ is OH.

13. A compound according to claim 1 having the structure of formula I 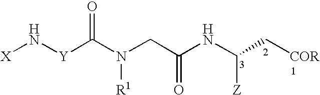
and pharmaceutically acceptable salts or tautomers thereof;
wherein
X has the structure of Formula Ib: 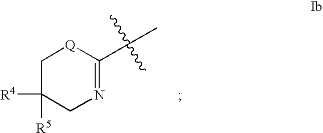
R⁴ and R⁵ are independently selected from the group consisting of H, OH, alkyl, CN, NO₂, aminoalkyl, halogen, haloalkyl, and alkoxy;
Y is a pyridine; optionally substituted with one or more substitutents independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, thioalkyl, alkylamino, arylamino, alkylsulfonamide, acyl, acylamino, alkylsulfone, sulfonamide, allyl, alkenyl, alkynyl, carboxamide, NHCOCF₃, and —(CH₂)ₘCOR²
m is zero to two; and
R² is selected from hydroxy, alkoxy, and amino.
R₅ is H or OH.
R and R¹ are independently CH₃ or H;
Q is NH or CH₂.

14. A compound according to claim 13 wherein
R⁸ is Cl;
R⁹ is I; and
R¹⁰ is OH.

15. A compound according to claim 1 having the structure of formula I 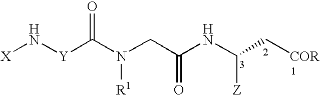
and pharmaceutically acceptable salts or tautomers thereof;
wherein
X has the structure of Formula Ib: 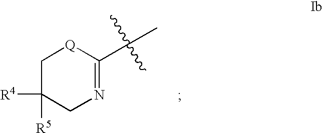
R₂ and R₃ are independently selected from the group consisting of H, OH, alkyl, CN, NO₂, aminoalkyl, halogen, haloalkyl, and alkoxy;
Y has the structure of Formula If: 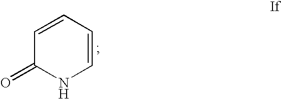
optionally substituted with one or more substitutents independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, thioalkyl, alkylamino, arylamino, alkylsulfonamide, acyl, acylamino, alkylsulfone, sulfonamide, allyl, alkenyl, alkynyl, carboxamide, NHCOCF₃, and —(CH₂)ₘCOR′;
m is zero to two;
R′ is selected from the group consisting of hydroxy, alkoxy, and amino.

16. A compound having the structure of Formula I 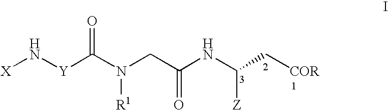
or a pharmaceutically acceptable salt or tautomer thereof;
wherein:
X is a pyrimidinyl or imidazolyl;
and wherein X is optionally substituted with one or more substituents independently selected from the group consisting of OH, alkyl, alkenyl, alkynyl, haloalkyl, alkylaryl, arylalkyl, alkoxy, dialkylamino, thioalkyl, cycloalkyl, CN, NO₂, and halogen;
n is a number from zero to two;
Y is a six-membered aryl or heterocycyl ring; wherein Y is optionally substituted with one or more moieties independently selected from the group consisting of OH, alkyl, alkoxy, NO₂, NH₂, CN, NHCOCF₃, COCF₃, haloalkyl, aryl, heterocycyl, halogen, alkoxyalkyl, aminoalkyl, hydroxyalkyl, methylenedioxy, ethylenedioxy, thioalkyl, alkylamino, arylamino, alkylsulfonamido, acyl, acylamino, alkylsulfone, sulfonamido, alkenyl, alkynyl, carboxamide, NHCOCF₃, and —(CH₂)ₘCOR²;
m is a number from zero to two;
R² is selected from the group consisting of hydroxy, alkoxy, and amino;
Z is an aryl ring having about five to about six members, or a bicyclic aryl ring having about nine to about twelve members, wherein Z optionally contains one to five heteroatoms independently selected from the group consisting of O, N and S; wherein Z is optionally substituted with one or more substituents selected from the group consisting of alkyl, haloalkyl, aryl, heterocycyl, arylalkyl, aryloxy, phenethyl, arylsulfone, halogen, alkoxyalkyl, aminoalkyl, cycloalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, NHCOCF₃, and —(CH₂)ₚCOR⁷⁹; wherein the aryl and heterocycyl substituents are optionally substituted with one or more substituents selected from the group consisting of alkyl, haloalkyl, halogen, alkoxyalkyl, aminoalkyl, cycloalakyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)_{q}COR⁸⁰;
p is a number from zero to two;
R⁷⁹ is selected from the group consisting of hydroxy, alkoxy, and amino;
q is a number from zero to two;
R⁸⁰ is selected from the group consisting of hydroxy, alkoxy, and amino;
Q is selected from the group consisting of NH and CH₂;
R is selected from the group consisting of OH, alkoxy, and NHR³;
R³ is selected from the group consisting of H and alkyl group;
R¹ is selected from the group consisting of H, CN, NO₂, acyl, haloalkyl, alkenyl, alkynyl, and alkyl;
and carbon atom 3 of Formula I is in the (R) conformation.

17. The compound according to claim 16 wherein the point of attachment of X is a carbon.

18. The compound according to claim 17 wherein the point of attachment is adjacent to one or both nitrogen atoms.

19. The compound according to claim 1 wherein the compound is selected from the group consisting of
(3R)-3-(3-bromo-5-chloro-2-hydroxyphenyl)-3-[(N-{3-hydroxy-5-[(5-hydroxy-1,4,5,6-tetrahydropyrimidinyl)amino]benzoyl}glycyl)amino]-propanoic acid;
(3R)-3-(3-bromo-5-chloro-2-hydroxyphenyl)-3-{[N-({5-[(5-hydroxy-1,4,5,6-tetrahydropyrimidin-2-yl)amino]pyridin-3-yl}carbonyl)glycyl]amino}-propanoic acid;
(3R)-3-[(N-{3-amino-5-[(5-hydroxy-1,4,5,6-tetrahydropyrimidin-2-yl)amino]-benzoyl}glycyl)amino]-3-(3,5-dichloro-2-hydroxyphenyl)propanoic acid;
(3R)-3-(3,5-dichloro-2-hydroxyphenyl)-3-[(N-{3-hydroxy-5-[(5-hydroxy-1,4,5,6-tetrahydropyrimidinyl)amino]benzoyl}glycyl)amino]propanoic acid;
(3R)-3-[(N-{3-(aminocarbonyl)-5-[(5-hydroxy-1,4,5,6-tetrahydropyrimidin-2-yl)amino]benzoyl}glycyl)amino]3-(3,5-dichloro-2-hydroxyphenyl)propanoic acid;
(3R)-3-(3-bromo-5-chloro-2-hydroxyphenyl)-3-[(N-{3-hydroxy-5-[(5-hydroxy-1,4,5,6-tetrahydropyrimidin-2-yl)amino]benzoyl}-N-methyl-glycyl)amino]propanoic acid;
(3R)-3-(3-bromo-5-chloro-2-hydroxyphenyl)-3-{[N-({5-[(5-hydroxy-1,4,5,6-tetrahydropyrimidin-2-yl)amino]pyridin-3-yl}carbonyl)-N-methylglycyl]-amino}propanoic acid;
(β¹R)-3-bromo-5-chloro-β-[[[[3-[(5-fluoro-1,4,5,6-tetrahydro-2-pyrimidinyl)-amino]-5-hydroxybenzoyl]amino]acetyl]amino]-2-hydroxybenzene-propanoic acid;
(β¹R)-3-bromo-5-chloro-β-[[[[3-[(5-fluoro-1,4,5,6-tetrahydro-2-pyrimidinyl)-amino]benzoyl]amino]acetyl]amino]-2-hydroxybenzenepropanoic acid;
(β¹R)-3,5-dichloro-β-[[[[3-[(5-fluoro-1,4,5,6-tetrahydro-2-pyrimidinyl)amino]-benzoyl]amino]acetyl]amino]-2-hydroxybenzenepropanoic acid;
(β¹R)-3-lodo-5-chloro-β-[[[[3-[(5-fluoro-1,4,5,6-tetrahydro-2-pyrimidinyl)-amino]-5-hydroxybenzoyl]amino]acetyl]amino]-2-hydroxybenzene-propanoic acid;
(β¹R)-3,5-dichloro-β-[[[[3-[(5-fluoro-1,4,5,6-tetrahydro-2-pyrimidinyl)amino]-5-hydroxybenzoyl]amino]acetyl]amino]-2-hydroxybenzenepropanoic acid;
(β¹R)-3-bromo-5-chloro-β-[[[[3-[(5,5-difluoro-1,4,5,6-tetrahydro-2-pyrimidinyl)amino]5-hydroxybenzoyl]amino]acetyl]amino]2-hydroxy-benzenepropanoic acid;
(β¹R)-3-bromo-5-chloro-β-[[[[3-[(5-fluoro-1,4,5,6-tetrahydro-2-pyrimidinyl)-amino]5-nitrobenzoyl]amino]acetyl]amino]2-hydroxybenzenepropanoic acid;
(β¹R)-β-[[[[3-amino-5-[(5-fluoro-1,4,5,6-tetrahydro-2-pyrimidinyl)amino]-benzoyl]amino]acetyl]amino]bromo-5-chloro-2-hydroxybenzenepropanoic acid;
(β¹R)-3-bromo-5-chloro-β-[[[[[5-[(5-fluoro-1,4,5,6-tetrahydro-2-pyrimidinyl)-amino]3-pyridinyl]carbonyl]amino]acetyl]amino]2-hydroxybenzene-propanoic acid;
(β¹R)-3-chloro-5-chloro-β-[[[[[5-[(5-fluoro-1,4,5,6-tetrahydro-2-pyrimidinyl)-amino]3-pyridinyl]carbonyl]amino]acetyl]amino]2-hydroxybenzene-propanoic acid;
(β¹R)-5-bromo-3-chloro-\tilde{β}-[[[[3-[(5-hydroxy-1,4,5,6-tetrahydro-2-pyrimidinyl)-amino]5-hydroxybenzoyl]amino]acetyl]amino]2-hydroxy-benzenepropanoic acid;
(β¹R)-5-bromo-3-chloro-β-[[[[[5-[(5-hydroxy -1,4,5,6-tetrahydro-2-pyrimidinyl)-amino]3-pyridinyl]carbonyl]amino]acetyl]amino]2-hydroxy-benzenepropanoic acid;
(β¹R)-3-bromo-5-chloro-β-[[[[[5-[(5,5-dimethyl-1,4,5,6-tetrahydro-2-pyrimidinyl)-amino]3-pyridinyl]carbonyl]amino]acetyl]amino]2-hydroxy-benzenepropanoic acid;
(R)-β-[[2-[[[3-hydroxy-5-[4,5-(dihydro-1H-imidazol-2 yl)amino]phenyl]-carbonyl]amino]acetyl]amino]3-bromo-5-chloro-2-hydroxybenzene-propanoic acid;
(β¹R)-3,5-dimethyl-β-[[[[3-[(5-fluoro-1,4,5,6-tetrahydro-2-pyrimidinyl)-amino]-5-nitrobenzoyl]amino]acetyl]amino]2-hydroxybenzenepropanoic acid;
(β¹R)-3,5-dimethyl-β-[[[[3-[(5-fluoro-1,4,5,6-tetrahydro-2-pyrimidinyl)-amino]5-aminobenzoyl]amino]acetyl]amino]-2-hydroxybenzenepropanoic acid;
(R)-3-Bromo-5-chloro-2-hydroxy-β-[[2-[[[3-hydroxy-5-[(1,4,5,6-tetrahydro-pyrimidin-2-yl)amino]phenyl]carbonyl]amino]acetyl]amino]benzene-propanoic acid;
(R)-5-Chloro-3-bromo-2-hydroxy-β-[[2-[[5[(1,4,5,6-tetrahydro-5-hydroxy-pyrimidin-2-yl)amino]phenyl]carbonyl]amino]acetyl]amino]benzene-propanoic acid;
(β¹R)-3-methyl-5-chloro-β-[[[[3-[(5-hydroxy-1,4,5,6-tetrahydro-2-pyrimidinyl)amino]-5-hydroxybenzoyl]amino]acetyl]amino]2-hydroxy-benzenepropanoic acid;
(β¹R)-3,5-dimethyl-β-[[[[3-[(5-hydroxy-1,4,5,6-tetrahydro-2-pyrimidinyl)-amino]-5-hydroxybenzoyl]amino]acetyl]amino]2-hydroxybenzene-propanoic acid;
(R)-β-[[2-[[[3-hydroxy-5-[4,5-(dihydro-1H-imidazol-2yl[)amino]phenyl]-carbonyl]amino]acetyl]amino]3,5-dichloro-2-hydroxybenzenepropanoic acid;
(R) 5-chloro-3-methyl-2-hydroxy-β-[[2-[[[3-hydroxy-5-[imidazolidine-2-amino]-phenyl]carbonyl]amino]acetyl]amino]benzenepropanoic acid;
(R) 3,5-Dichloro-2-hydroxy-β-[[2-[[5[(1,4,5,6-tetrahydro-5-hydroxy-pyrimidin-2-yl)amino]phenyl]carbonyl]amino]acetyl]amino]benzene-propanoic acid;
(R) 3-Bromo-5-chloro-2-hydroxy-β-[[2-[[[3-hydroxy-5-[(1,4,5,6-tetrahydro-pyrimidin-2-yl)amino]phenyl]carbonyl]amino]acetyl]amino]benzene-propanoic acid
(β¹R)-3,5-dibromo-β-[[[[3-[(5-fluoro-1,4,5,6-tetrahydro-2-pyrimidinyl)-amino]5-nitrobenzoyl]amino]acetyl]amino]benzenepropanoic acid;
(β¹R)-3,5-dimethyl-β-[[[[3-[(5-fluoro-1,4,5,6-tetrahydro-2-pyrimidinyl)-amino]5-nitrobenzoyl]amino]acetyl]amino]benzenepropanoic acid;
(β¹R)-3-bromo-5-chloro-β-[[[[3-[(5-fluoro-1,4,5,6-tetrahydro-2-pyrimidinyl)-amino]5-nitrobenzoyl]amino]acetyl]amino]benzenepropanoic acid;
(β¹R)-3,5-dichloro-β-[[[[3-[(5-fluoro-1,4,5,6-tetrahydro-2-pyrimidinyl)amino]-5-nitrobenzoyl]amino]acetyl]amino]benzenepropanoic acid;
(β¹R)-3,iodo-5-bromo-β-[[[[3-[(5-fluoro-1,4,5,6-tetrahydro-2-pyrimidinyl)-amino]-5-nitrobenzoyl]amino]acetyl]amino]benzenepropanoic acid;
(R) 3,5-Dichloro-2-hydroxy-β-[[2-[[[3-[(1,4,5,6-tetrahydro-5-hydroxy-pyrimidin-2-yl)amino]phenyl]carbonyl]amino]acetyl]amino]benzene-propanoic acid;
(R) 3,5-Dichloro-2-hydroxy-β-[[2-[[[3-hydroxy-5-[(1,4,5,6-tetrahydro-5-hydroxypyrimidin-2-yl)amino]phenyl]carbonyl]amino]acetyl]amino]-benzenepropanoic acid;
(R) 3-Bromo-5-chloro 2-hydroxy-β-[[2-[[[5-[(1,4,5,6-tetrahydropyrimidin-2-yl)-amino]phenyl]carbonyl]amino]acetyl]amino]benzenepropanoic acid;
(R) 3-Bromo-5-chloro-2-hydroxy-β-[[2-[[[5-(5-hydroxy-1,4,5,6-tetrahydro-pyrimidin-2-yl)amino]-3-nitrophenyl]carbonylamino]acetyl]amino]benzene-propanoic acid;
(R) 3-Bromo-5-chloro-2-hydroxy-β-[[2-[[[5-(5-hydroxy-1,4,5,6-tetrahydro-pyrimidin-2-yl)amino]-3-aminophenyl]carbonyl]amino]acetyl]amino]-benzenepropanoic acid;
(R) 5-Bromo-3-chloro 2-hydroxy-β-[[2-[[[5-[(5-hydroxy-1,4,5,6-tetrahydro-pyrimidin-2-yl)amino]phenyl]carbonyl]amino]acetyl]amino]benzene-propanoic acid;
(R) 5-Bromo-3-chloro 2-hydroxy-β-[[2-[[[5-[(1,4,5,6-tetrahydropyrimidin-2-yl)amino]-3-hydroxyphenyl]carbonyl]amino]acetyl]amino]benzene-propanoic acid;
(R) 5-Bromo-3-chloro 2-hydroxy-β-[[2-[[[5-[(1,4,5,6-tetrahydropyrimidin-2-yl)amino]phenyl]carbonyl]amino]acetyl]amino]benzenepropanoic acid;
(R) 5-Bromo-3-chloro 2-hydroxy-β-[[2-[[[5-[5-fluoro-(1,4,5,6-tetrahydro-pyrimidin-2-yl)amino]phenyl]carbonyl]amino]acetyl]amino]benzene-propanoic acid;
(R) 5-Bromo-3-chloro-2-hydroxy-β-[[2-[[[3-hydroxy-5-[5-fluoro-(1,4,5,6-tetrahydropyrimidin-2-yl)amino]phenyl]carbonyl]amino]acetyl]amino]-benzenepropanoic acid;
(R) 5-Bromo-3-chloro 2-hydroxy-β-[[2-[[[5-[5-hydroxy-(1,4,5,6-tetrahydro-pyrimidin-2-yl)amino]-4-methylphenyl]carbonyl]amino]acetyl]amino]-benzenepropanoic acid;
(R) 5-Bromo-3-chloro-2-hydroxy-β-[[2-[[[5-(5-hydroxy-1,4,5,6-tetrahydro-pyrimidin-2-yl)amino]-3-nitrophenyl]carbonylamino]acetyl]amino]-benzenepropanoic acid;
(R) 5-Bromo-3-chloro-2-hydroxy-β-[[2-[[[5-(5-hydroxy-1,4,5,6-tetrahydro-pyrimidin-2-yl)amino]3-aminophenyl]carbonyl]amino]acetyl]amino]-benzenepropanoic acid;
(R) 3,5-dibromo-2-hydroxy-β-[[2-[[[3-hydroxy-5-[5-hydroxy-(1,4,5,6-tetrahydro-pyrimidin-2-yl)amino]phenyl]carbonyl]amino]acetyl]amino]-benzenepropanoic acid;
(R) 3,5-Dibromo-2-hydroxy-β-[[2-[[[5-[5-hydroxy-(1,4,5,6-tetrahydro-pyrimidin-2-yl)amino]phenyl]carbonyl]amino]acetyl]amino]benzene-propanoic acid;
(R) 3,5-Dibromo-2-hydroxy-β-[[2-[[[5-(1,4,5,6-tetrahydropyrimidin-2-yl)-amino]-3-hydroxy]phenyl]carbonyl]amino]acetyl]amino]benzenepropanoic acid;
(R) 3,5-Dibromo-2-hydroxy-p-[[2-[[[3-(5-hydroxy-1,4,5,6-tetrahydro-pyrimidin-2-yl)amino]pyridyl]5-carbonyl]amino]acetyl]amino]benzene-propanoic acid
(R) 3,5-Dibromo-2-hydroxy-β-[[2-[[[5-(5-fluoro-1,4,5,6-tetrahydropyrimidin-2-yl)amino]-3-hydroxy]phenyl]carbonyl]amino]acetyl]amino]benzene-propanoic acid;
(R) 3,5-Dibromo-2-hydroxy-β-[[2-[[[5-(1,4,5,6-tetrahydropyrimidin-2-yl)-amino]phenyl]carbonyl]amino]acetyl]amino]benzenepropanoic acid;
(R) 3,5-Dibromo-2-hydroxy-β-[[2-[[[5-(5-hydroxy-1,4,5,6-tetrahydro-pyrimidin-2-yl)-amino]-4-methyl]phenyl]carbonylamino]acetyl]amino]-benzenepropanoic acid;
(R) 3,5-Dibromo-2-hydroxy-β-[[2-[[[5-(5-hydroxy-1,4,5,6-tetrahydro-pyrimidin-2-yl)amino]-3-nitro]phenyl]carbonyl]amino]acetyl]amino]-benzenepropanoic acid;
(R) 3,5-Dibromo-2-hydroxy-β-[[2-[[[5-(5-hydroxy-1,4,5,6-tetrahydro-pyrimidin-2-yl)amino]3-aminophenyl]carbonyl]amino]acetyl]amino]-benzenepropanoic acid;
(R) 5-Chloro-3-iodo-2-hydroxy-β-[[2-[[[3-hydroxy-5-[(1,4,5,6-tetrahydro-5-hydroxypyrimidin-2-yl)amino]phenyl]carbonyl]amino]acetyl]amino]-benzenepropanoic acid;
(R) 5-Chloro-3-iodo-2-hydroxy-β-[[2-[[[5-[(1,4,5,6-tetrahydro-5-hydroxy-pyrimidin-2-yl)amino]pyridyl]-3-carbonyl]amino]acetyl]amino]benzene-propanoic acid;
(R) 5-Chloro-3-iodo-2-hydroxy-β-[[2-[[[3-hydroxy-5-[(1,4,5,6-tetrahydro-pyrimidin-2-yl)amino]phenyl]carbonyl]amino]acetyl]amino]benzene-propanoic acid;
(R) 5-Chloro-3-iodo-2-hydroxy-β-[[2-[[[5-[3-amino-(5-hydroxy-1,4,5,6-tetrahydro-pyrimidin-2-yl)amino]phenyl]carbonyl]amino]acetyl]amino]-benzenepropanoic acid;
(R) 5-Chloro-3-iodo-2-hydroxy-β-[[2-[[[5-[(5-hydroxy-1,4,5,6-tetrahydro-pyrimidin-2-yl)amino]phenyl]carbonyl]amino]acetyl]amino]benzene-propanoic acid;
(R) 5-Chloro-2-hydroxy-3-iodo-β-[[2-[[[5-[(1,4,5,6-tetrahydropyrimidin-2-yl)amino]-phenyl]carbonyl]amino]acetyl]amino]benzenepropanoic acid;
(R) 5-Chloro-3-iodo-2-hydroxy-β-[[2-[[[3-hydroxy-5-[(1,4,5,6-tetrahydro-5-hydroxy-pyrimidin-2-yl)amino]phenyl]carbonyl]amino]acetyl]amino]-benzenepropanoic acid;
(3R)-3-(3,5-dichloro-2-hydroxyphenyl)-3-{[N-({5-[(5-hydroxy-1,4,5,6-tetra-hydropyrimidin-2-yl)amino]6-oxo-1,6-dihydropyridin-3-yl}carbonyl)glycyl]-amino}propanoic acid;
(3R)-3-(3-Bromo-5-chloro-2-hydroxyphenyl)-3-({N-[3-hydroxy-5-(3,4,5,6-tetrahydro-2H-azepin-7-ylamino)benzoyl]glycyl}amino)propanoic acid trifluoroacetic acid;
(3R)-3-(3,5-Dichloro-2-hydroxyphenyl)-3-({N-[3-hydroxy-5-(3,4,5,6-tetrahydro-2H-azepin-7-ylamino)benzoyl]glycyl}amino)propanoic acid;
(3R)-3-(5-Bromo-3-chloro-2-hydroxyphenyl)-3-({N-[3-hydroxy-5-(3,4,5,6-tetrahydro-2H-azepin-7-ylamino)benzoyl]glycyl}amino) propanoic acid;
(3R)-3-(5-Chloro-2-hydroxy-3-iodophenyl)-3-({N-[3-hydroxy-5-(3,4,5,6-tetrahydro-2H-azepin-7-ylamino)benzoyl]glycyl}amino)propanoic acid; and
(3R)-3-(3,5-Dibromo-2-hydroxyphenyl)-3-({N-[3-hydroxy-5-(3,4,5,6-tetrahydro-2H-azepin-7-ylamino)benzoyl]glycyl}amino)propanoic acid.

20. A compound having the structure of formula I 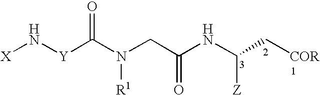
wherein
X is a 6-membered heterocyclic ring of the formula Ib: 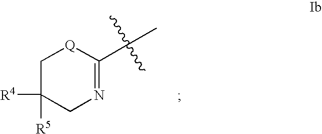
R⁴ and R⁵ are independently selected from the group consisting of H, OH, alkyl, CN, NO₂, aminoalkyl, halogen, haloalkyl, and alkoxy;
Y is a substituted phenyl ring of the formula Ic: 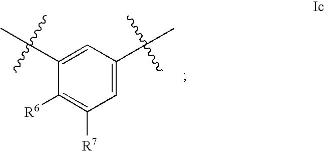
R⁶ and R⁷ are independently selected from the group consisting of OH, CH₃, NO₂, NH₂, COOH, CONH₂, COCF₃, and NHCOCF₃; or R⁶and R⁷ are linked together with a methylenedioxy and ethylenedioxy group to form a five- or six-membered ring, respectively;
Z is a substituted phenyl ring of the formula Id: 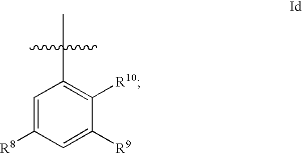
R⁸, R⁹ and R¹⁰ are independently selected from the group consisting of H, OH, methyl, or halogen;
Q is selected from the group consisting of NH and CH₂;
R is selected from the group consisting of OH, alkoxy, and NHR³;
R³ is selected from the group consisiting of H and alkyl group;
R¹ is selected from the group consisting of H and methyl
and carbon atom 3 of Formula I is in the (R) conformation.

21. A compound according to claim 20 wherein
R⁴ and R⁵ are independently selected from the group consisting of H, OH, F, and CH₃;
R⁶ and R⁷ linked together with a methylenedioxy group to form a five-membered ring; and
R⁶ and R⁷ linked together with a methylenedioxy group to form a five-membered ring.

22. A pharmaceutical composition comprising a compound of claims 1 or 19 and a pharmaceutically acceptable carrier.

23. A method for treating or preventing an αᵥβ₃—mediated condition in a mammal in need of such treatment or prevention comprising administering to the mammal a therapeutically effective amount of a compound of claim 1 or 19.

24. The method according to claim 23 wherein the αᵥβ₃—mediated condition treated or prevented is selected from the group consisting of tumor metastasis, tumor growth, solid tumor growth, angiogenesis, osteoporosis, humoral hypercalcemia of malignancy, smooth muscle cell migration, restenosis, atheroscelorosis, macular degeneration, retinopathy, and arthritis.

25. A compound of claims 1 or 19 that has a selectivity ratio of about 10 to about 1000 for the αᵥβ₃ and the αᵥβ₅ integrins, over the αᵥβ_{≢}integrin.

26. A method for the preparation of a compound having the structure of Formula A: 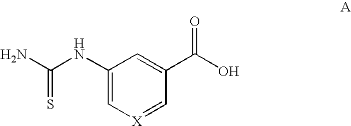
wherein × is CH, COH, or N;
wherein the method comprises contacting a compound having the structure of Formula B: 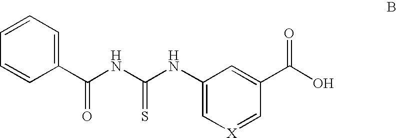
with a base alkoxide and an alcoholic solvent, thereby producing the compound of Formula A.

27. A method for the preparation of a compound having the structure of Formula A: 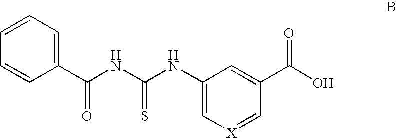
wherein × is CH, COH, or N;
wherein the method comprises contacting a compound having the structure of Formula C: 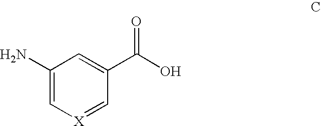
with benzoylisothiocyanate and a solvent, thereby producing the compound of Formula B.
